(19) United States
(12) Patent Application Publication
(10) Pub. No.: US 2005/0287647 A9
(43) Pub. Date: Dec. 29, 2005

(54) Plant artificial chromosomes, uses thereof and methods of preparing plant artificial chromosomes

(76) Inventors: Carl Perez, Richmond (CA); Steven Fabijanski, Ottawa (CA); Edward Perkins, Burnaby (CA)

Correspondence Address: HELLER EHRMAN LLP, 4350 LA JOLLA VILLAGE DRIVE, 7TH FLOOR, SAN DIEGO, CA, 92122-1246, US

(21) Appl. No.: 10/161,408

(22) Filed: May 30, 2002

Related U.S. Application Data
(60) Provisional application No. 60/294,687, filed on May 30, 2001.
(60) Provisional application No. 60/296,329, filed on Jun. 4, 2001.

(51) Int. Cl. C12P 19/34; A01H 1/00; C12N 15/82

(52) U.S. Cl. 435/91.1; 435/468; 800/288

(57) ABSTRACT

Methods for preparing cell lines that contain plant artificial chromosomes, methods for preparation of plant artificial chromosomes, methods for targeted insertion of heterologous DNA into plant artificial chromosomes, and methods for delivery of plant chromosomes to selected cells and tissues are provided. In particular, plant artificial chromosomes that are substantially composed of repeated nucleic acid units of varying amounts of heterochromatin and euchromatin are provided. Also provided are methods of using plant and animal artificial chromosomes in the production of valuable transgenic plants. Methods for identifying plant genes encoding particular traits using artificial chromosomes and for producing an acrocentric plant chromosome are also provided.

### RELATED APPLICATIONS

Benefit of priority under 35 U.S.C. §119(e) is claimed to U.S. Provisional Application No. 60/294,687, filed May 30, 2001, by CARL PEREZ AND STEVEN FABIJANSKI entitled PLANT ARTIFICIAL CHROMOSOMES, USES THEREOF AND METHODS FOR PREPARING PLANT ARTIFICIAL CHROMOSOMES and to U.S. Provisional Application No. 60/296,329, filed Jun. 4, 2001, by CARL PEREZ AND STEVEN FABIJANSKI entitled PLANT ARTIFICIAL CHROMOSOMES, USES THEREOF AND METHODS FOR PREPARING PLANT ARTIFICIAL CHROMOSOMES.

This application is related to U.S. Provisional Application No. 60/294,758, filed May 30, 2001, by EDWARD PERKINS et al. entitled CHROMOSOME-BASED PLATFORMS and to U.S. Provisional Application No. 60/366,891, filed Mar. 21, 2002, by by EDWARD PERKINS et al. entitled CHROMOSOME-BASED PLATFORMS. This application is also related to U.S. Provisional Application Attorney Docket No. 24601-420, filed May 30, 2002, by EDWARD PERKINS et al. entitled CHROMOSOME-BASED PLATFORMS and to PCT International Patent Application Attorney Docket No. 24601-420PC, filed May 30, 2002, by EDWARD PERKINS et al., entitled CHROMOSOME-BASED PLATFORMS. This application is related to U.S. application Ser. No. 08/695,191, filed Aug. 7, 1996 by GYULA HADLACZKY and ALADAR SZALAY, entitled ARTIFICIAL CHROMOSOMES, USES THEREOF AND METHODS FOR PREPARING ARTIFICIAL CHROMOSOMES, now U.S. Pat. No. 6,025,155. This application is also related to U.S. application Ser. No. 08/682,080, filed Jul. 15, 1996 by GYULA HADLACZKY and ALADAR SZALAY, entitled ARTIFICIAL CHROMOSOMES, USES THEREOF AND METHODS FOR PREPARING ARTIFICIAL CHROMOSOMES, now U.S. Pat. No. 6,077,697. This application is also related U.S. application Ser. No. 08/629,822, filed Apr. 10, 1996 by GYULA HADLACZKY and ALADAR SZALAY, entitled ARTIFICIAL CHROMOSOMES, USES THEREOF AND METHODS FOR PREPARING ARTIFICIAL CHROMOSOMES (now abandoned), and is also related to copending U.S. application Ser. No. 09/096,648, filed Jun. 12, 1998, by GYULA HADLACZKY and ALADAR SZALAY, entitled ARTIFICIAL CHROMOSOMES, USES THEREOF AND METHODS FOR PREPARING ARTIFICIAL CHROMOSOMES and to U.S. application Ser. No. 09/835,682, Apr. 10, 1997 by GYULA HADLACZKY and ALADAR SZALAY, entitled ARTIFICIAL CHROMOSOMES, USES THEREOF AND METHODS FOR PREPARING ARTIFICIAL CHROMOSOMES (now abandoned). This application is also related to copending U.S. application Ser. No. 09/724,726, filed Nov. 28, 2000, U.S. application Ser. No. 09/724,872, filed Nov. 28, 2000, U.S. application Ser. No. 09/724,693, filed Nov. 28, 2000, U.S. application Ser. No. 09/799,462, filed Mar. 5, 2001, U.S. application Ser. No. 09/836,911, filed Apr. 17, 2001, and U.S. application Ser. No. 10/125,767, filed Apr. 17, 2002, each of which is by GYULA HADLACZKY and ALADAR SZALAY, and is entitled ARTIFICIAL CHROMOSOMES, USES THEREOF AND METHODS FOR PREPARING ARTIFICIAL CHROMOSOMES. This application is also related to International PCT application No. WO 97/40183. The subject matter of each of these applications, provisional applications and international applications is incorporated by reference in its entirety.

### FIELD OF THE INVENTION

Artificial chromosomes and methods of producing artificial chromosomes, particularly for use in delivery of nucleic acids and expression thereof in plants are provided. Also provided are methods of use of artificial chromosomes in the delivery of nucleic acids to host cells, including plant cells, and the expression of the nucleic acids therein. The resulting plant cells, tissues, organs and whole plants containing the artificial chromosomes, plant cell-based methods for production of heterologous proteins and methods of producing transgenic organisms, particularly plants, using the artificial chromosomes are provided.

### BACKGROUND OF THE INVENTION

The stable transfer of nucleic acids into plant cells and the expression of the nucleic acids therein poses many challenges. Many efforts at the stable introduction of nucleic acids into plant cells have utilized Agrobacterium-mediated transformation. Agrobacterium is a free-living Gram-negative soil bacterium. Virulent strains of this bacterium are able to infect plant tissue and induce the production of a neoplastic growth commonly referred to as a crowngall. Virulent strains of Agrobacterium contain a large plasmid DNA known as a Ti-plasmid that contains genes required for DNA transfer (vir genes) and replication as well as a region of DNA that is transferred to plant cells called T-DNA. The T-DNA region is bordered by T-DNA border sequences that are crucial to the DNA transfer process. These T-DNA border sequences are recognized by the vir genes encoded on the Ti-plasmid and the vir genes are responsible for the DNA transfer process.

Most wild-type Agrobacterium have a relatively broad dicot plant host range and are capable of transferring T-DNA regions up to 25 kilobases of DNA (e.g., nopaline strains) or more (e.g., octopine strains). Accordingly, numerous methods of using Agrobacterium to transfer DNA into plant cells have been developed based on the engineering of the Ti-plasmid to no longer contain the genes responsible for altered morphology and replacing these genes with a recombinant gene encoding a trait of interest. There are two primary types of Agrobacterium-based plant transformation systems, binary [see, e.g., U.S. Pat. No. 4,940,838] and co-integrate [see, e.g., Fraley et al. (1985) Biotechnology 3:629-635] methods. The T-DNA border repeats are maintained in both systems and the natural DNA transfer process is used to transfer the portion of DNA located between the T-DNA borders into the plant cell.

Another plant cell transformation system, termed biolistics, involves the bombardment of plant cells with microscopic particles coated with DNA encoding a new trait. The particles are rapidly accelerated, typically by gas or electrical discharge, through the cell wall and membranes, whereby the DNA is released into the cell and is incorporated into the genome of the cell. This method is used for transformation of many crops, including corn, wheat, barley, rice, woody tree species and others.

A significant number of crop species of commercial interest have been transformed using either Agrobacterium-mediated or biolistic systems. However, these methods have many limitations that limit their utility. For example, there are limits to the size of the heterologous DNA that can be transferred using these methods; typically, only one to two genes may be transferred. Thus, although these methods may have utility in producing crop products modified to contain a single new trait, such as insect or herbicide tolerance, they may not be sufficient to transfer DNA that will provide for multiple traits, or very large DNA segments encoding a multiplicity of traits.

In addition, the genetically modified plant cells produced by these methods tend to contain the transferred DNA in euchromatic regions of the genomic DNA. Typically, a large number of independent transgenic insertion events must be screened before a suitable event (such as insertion of a gene into the host genomic DNA such that it provides a sufficient level of gene expression within temporal and spatial expectations and without evidence of gene rearrangement) is identified.

Another limitation of these methods is the effort required to utilize them in the genetic modification of many commercially important crops. For example, transformation efficiency can vary with the crop and can be low, notably in cereal crops such as corn and wheat. Often the inserted genes are rearranged and unstable over generations.

Furthermore Agrobacterium tumefaciens relies on host-parasite interaction in order to be successful. This has the effect that Agrobacterium has a preference for some dicots, while other dicots, monocots and conifers are resistant to transformation via Agrobacterium.

Self-replicating vectors have also been used in the transfer of nucleic acids into plant cells. Such episomal vectors contain DNA sequences that are required for DNA replication and sustainability of the vector in a living cell. In higher plants, very few episomal vectors have been developed. These episomal vectors have the drawback of having a very limited capacity for carrying genetic information and are unstable. One example of an episomal plant vector is the Cauliflower Mosaic Virus [Brisson et al. (1984) Nature 310:51].

Limitations of these gene delivery technologies necessitate the development of alternative vector systems suitable for transferring large (up to Mb size or larger) genes, gene complexes, and multiple genes together with regulatory elements for safe, controlled, and persistent expression of the desired genetic material in higher organisms, particularly plants, without rearrangement caused by insertion or mutagenesis. Therefore, it is an object herein to provide artificial chromosomes for the introduction of large nucleic acids into eukaryotic cells and methods using the artificial chromosomes, particularly for the introduction and expression of nucleic acids in plants.

### SUMMARY OF THE INVENTION

Provided herein are plant artificial chromosomes and methods for producing plant artificial chromosomes. The artificial chromosomes are fully functional stable chromosomes. Plant artificial chromosomes provided herein have a particular composition that makes them ideal vectors for stable, controlled, high-level expression of heterologous nucleic acids in plant cells. The artificial chromosomes are capable of independent, extra-genomic maintenance, replication and segregation within cells and can carry multiple, large heterologous genes.

Artificial plant chromosomes provided herein are non-natural chromosomes that exhibit an ordered segmentation that distinguishes them from naturally occurring chromosomes. The segmented appearance can be visualized using a variety of chromosome analysis techniques and correlates with the unique structure of these artificial chromosomes, which, in particular methods of producing these chromosomes, can arise through amplification of chromosomal segments (i.e., amplification-based artificial chromosomes). The artificial chromosomes, throughout the region or regions of segmentation, are predominantly made up of one or more nucleic acid units that is (are) repeated in the region (referred to as the repeat region) and that have a similar gross structure. Repeats of a nucleic acid unit tend to be of similar size and share some common nucleic acid sequences, for example, a replication site involved in amplification of chromosome segments and/or some heterologous nucleic acid. Although the size of a repeating nucleic acid unit can vary, typically they tend to be greater than about 100 kb, greater than about 500 kb, greater than about 1 Mb, greater than about 5 Mb or greater than about 10 Mb. Typically, repeats of a nucleic acid unit are substantially similar in nucleic acid composition and can be nearly identical. The common nucleic acid sequences can contain sequences that represent euchromatic and heterochromatic nucleic acid. The composition of the amplification-based artificial chromosomes can be such that substantially the entire chromosome exhibits a segmented appearance or such that only one or more portions that make-up less than the entire chromosome appear segmented.

The composition of the plant artificial chromosomes provided herein can vary. For example, in some of the artificial chromosomes provided herein, the repeat region or regions can be made up predominantly of heterochromatic DNA (i.e., the repeat region or regions contain more heterochromatic DNA than other types of DNA, e.g., euchromatic DNA). In other artificial chromosomes provided herein, the repeat region or regions can be made up predominantly of euchromatic DNA (i.e., the repeat region or regions contain more euchromatic DNA than other types of DNA, e.g., heterochromatic DNA) or can be made up of substantially equivalent amounts of heterochromatic and euchromatic DNA, e.g., about 40% to about 50% of one type of nucleic acid and about 50% to about 60% of the other type of nucleic acid. The repeat region or regions thus can be entirely heterochromatic (while still containing one or more heterologous genes), or can contain increasing amounts of euchromatic DNA, such that, for example, the region contains about 10%, 20%, 30%, 40%, 50%, 60%, 70%, 80%, 90% or greater than 90% euchromatic DNA. Common nucleic acid sequences within repeated nucleic acid units in a repeat region can contain DNA that represents euchromatic nucleic acid and DNA that represents heterochromatic nucleic acid. Because the entire artificial chromosome can be made up predominantly of a repeat region or regions (e.g., the composition of the chromosome is such that the repeat region or regions make up greater than about 50% or greater than about 60% of the chromosome), it is thus possible for the artificial chromosome to be made up predominantly of heterochromatin or euchromatin, or to be made up of substantially equivalent amounts of heterochromatin and euchromatin, e.g., about 40% to about 50% of one type of nucleic acid and about 50% to about 60% of the other type of nucleic acid. Plant artificial chromosomes provided herein can be isolated or contained within cells or vesicles.

Also provided herein are cells containing plant artificial chromosomes as described herein, including plant cells and animal cells. Included among the cells containing the plant artificial chromosomes are any cells that include one or more plant chromosomes. Included, for example, are plant cells, including plant protoplasts, in culture and within plant tissues, organs, seeds, pollen or whole plants. Plant cells containing the plant artificial chromosomes can be from any type of plant, including monocots and dicots. For example, the plant cells can be from Arabidopsis, Nicotiana, Solanum, Lycopersicon, Daucus, Hordeum, Zea mays, Brassica, Triticum, Helianthus, Oryza, Glycine (soybean), gossypium (cotton). Also contemplated are mammalian and other animal cells that contain plant ACs

Plant cells containing artificial chromosomes of any species are also provided herein. Thus, for example, such plant cells can contain an artificial chromosome containing an animal, e.g., mammalian, centromere or an insect or avian centromere. Included among the artificial chromosomes contained within plant cells as provided herein are predominantly heterochromatic [formerly referred to as satellite artificial chromosomes (SATACs); see, e.g., U.S. Pat. Nos. 6,077,697 and 6,025,155 and published International PCT application No. WO 97/40183], minichromosomes which contain a de novo centromere, artificial chromosomes containing one or more regions of repeating nucleic acid units wherein the repeat region(s) contain substantially equivalent amounts of euchromatic and heterochromatic nucleic acid and in vitro assembled artificial chromosomes, each from any species. An exemplary artificial chromosome is a mammalian satellite artificial chromosome containing a mouse centromere. Included among the plant cells containing artificial chromosomes of any species are plant cells, including plant protoplasts, in culture and within plant tissues, organs, seeds, pollen or whole plants. Plant cells containing the artificial chromosomes can be from any type of plant, including monocots and dicots. For example, the plant cells can be from Arabidopsis, Nicotiana, Solanum, Lycopersicon, Daucus, Hordeum, Zea mays, Brassica, Triticum, Helianthusand Oryza.

Further provided herein are methods of producing plant artificial chromosomes. One embodiment of these methods includes the steps of introducing nucleic acid into a cell containing plant chromosomes and selecting a cell containing an artificial chromosome that contains one or more repeat regions in which one or more nucleic acid units is (are) repeated. The repeats of a nucleic acid unit in a repeat region can contain common nucleic acid sequences and can be substantially identical. In some embodiments of this method, the repeat region(s) of the artificial chromosome contain substantially equivalent amounts of euchromatic and heterochromatic nucleic acid. The artificial chromosome can be predominantly made up of one or more repeat regions. In further embodiments of this method, the artificial chromosome is made up of substantially equivalent amounts of euchromatic and heterochromatic nucleic acid. In further embodiments of this method, the repeats of a nucleic acid unit have common nucleic acid sequences which contain sequences that represent euchromatic and heterochromatic nucleic acid.

Any cell containing plant chromosomes can be used in these embodiments of methods of producing plant artificial chromosomes described herein. For example, the cell can be any cell that contains chromosomes from Arabidopsis, tobacco, Solanum, Lycopersicon, Daucus, Hordeum, Zea mays, Brassica, Triticum, Oryza, Capsicum, lentil and/or Helianthus, including cells or protoplasts of Arabidopsis, tobacco and/or Helianthus.

The nucleic acid that is introduced into a cell containing plant chromosomes in methods of producing a plant artificial chromosome as provided herein can be any nucleic acid, including, but not limited to, satellite DNA, rDNA and lambda phage DNA. Satellite DNA and rDNA includes such DNA from plants, such as, for example, Arabidopsis, Nicotiana, Solanum, Lycopersicon, Daucus, Hordeum, Zea mays, Brassica, Triticum and Oryza, and from animals, such as mammals. The rDNA can contain sequences of an intergenic spacer region, such as can be obtained, for example, from DNA of Arabidopsis, Solanum, Lycopersicon, Hordeum, Zea, Oryza, rye, wheat, radish and mung bean. In some embodiments of the method, the nucleic acid contains a nucleic acid sequence that facilitates amplification of a region of a plant chromosome or targets it to an amplifiable region of a plant chromosome.

In further embodiments of methods of producing plant artificial chromosomes provided herein, the nucleic acid that is introduced into a cell containing one or more plant chromosomes includes nucleic acid that for identification of cells containing the nucleic acid. Such nucleic acids include nucleic acid encoding a fluorescent protein, such as a green, blue or red fluorescent protein, and nucleic acid encoding a selectable marker, such as, for example, proteins that confer resistance to phosphinothricin, ammonium glufosinate, glyphosate, kanamycin, hydromycin, dihydrofolate or sulfonylurea.

In embodiments of methods of producing plant artificial chromosomes in which nucleic acid is introduced into a cell containing one or more plant chromosomes, the cell can be cultured through two or more cell doublings, and typically from about 5 to about 60, or about 5 to about 55, or about 10 to about 55, or about 25 to about 55, or about 35 to about 55 cell doublings following introduction of nucleic acid into a cell. The step of selecting a cell containing a plant artificial chromosome can include sorting of cells into which nucleic acid was introduced. For example, cells can be sorted on the basis of the presence of a selectable marker, such as a reporter protein, or by growing (culturing) the cells under selective conditions. The selection step can include fluorescent in situ hybridization (FISH) analysis of cells into which nucleic acid is introduced.

Also provided are methods of producing a transgenic plant using artificial chromosomes that function in plants and transgenic plants containing artificial chromosomes. Artificial chromosomes used in the methods of producing transgenic plants can be of any species. For example, the artificial chromosomes can contain a centromere from species such as animals, e.g., mammals, birds, plants, or insects, that functions to segregate nucleic acids to daughter cells through cell division. In some embodiments of the methods for producing a transgenic plant, the artificial chromosomes contain repeat regions predominantly made up of repeats of one or more nucleic acid units. Repeats of a nucleic acid unit can share some common nucleic acid sequences, for example, a replication site involved in amplification of chromosome segments and/or some heterologous nucleic acid. Repeats of a nucleic acid unit can be substantially identical. Common nucleic acid sequences of repeats of a nucleic acid unit can contain sequences that represent euchromatic and heterochromatic nucleic acid.

Repeat regions of artificial chromosomes that can be used in the methods of producing a transgenic plant can be made up of substantially equivalent amounts of heterochromatic and euchromatic DNA or can be made up predominantly of heterochromatic DNA or can be made up predominantly of euchromatic DNA. The artificial chromosome can be made up predominantly of heterochromatic or euchromatic DNA or can be made up of substantially equivalent amounts of heterochromatin and euchromatin. Such artificial chromosomes that contain plant centromeres can contain a plant centromere from any species of plant, including monocots and dicots. For example, the centromere can be from Arabidopsis, tobacco, Helianthus, Solanum, Lycopersicon, Daucus, Hordeum, Zea, Brassica, Triticum, rye, wheat, radish, mung bean or Oryza. The artificial chromosomes can be made using methods described herein.

In a method of producing a transgenic plant provided herein, an artificial chromosome, such as those described above and elsewhere herein, is introduced into a plant cell. The artificial chromosome can contain heterologous nucleic acid encoding a gene product such as, for example, an enzyme, antisense RNA, tRNA, rDNA, a structural protein, a marker or reporter protein, a ligand, a receptor, a ribozyme, a therapeutic protein, a biopharmaceutical protein, a vaccine, a blood factor, an antigen, a hormone, a cytokine, a growth factor or an antibody. The product can be one that provides for resistance to diseases, insects, herbicides or stress in the plant. The product can be one that provides for an agronomically important trait in the plant and/or that alters the nutrient utilization and/or improves the nutrient quality of the plant. Heterologous nucleic acid of an artificial chromosome can be contained within a bacterial artificial chromosome (BAC) or a yeast artificial chromosome (YAC).

The plant cell into which such artificial chromosomes can be introduced in methods of producing a transgenic plant provided herein can be any species of plant cell, including, but not limited to, Arabidopsis, tobacco, Helianthus, Solanum, Lycopersicon, Daucus, Hordeum, Zea, Brassica, Triticum, rye, wheat, radish, mung bean, Capsicum, lentil and Oryza. Any cell that can develop into a plant can be used, including plant cells and protoplasts of plant embryos, calli, tissues, meristem, organs, seeds, seedlings, pollen, pollen tubes or whole plants.

Artificial chromosomes can be introduced into plant cells in the methods of producing a transgenic plant using any process for transfer of nucleic acids into plant cells, including, but not limited to chemical, physical and electrical processes and combinations thereof. For example, the artificial chromosomes can be transferred into plant cells via direct contact in the absence or presence of a fusogen, e.g., polyethylene glycol (PEG), calcium phosphate and/or lipid or they can be encapsulated in a lipid structure (e.g., a liposome) or contained within a protoplast or microcell which is then allowed to fuse (in the presence or absence of a fusogen such as PEG) with a plant cell for introduction of the artificial chromosome into the cell in a method of producing a transgenic plant. Artificial chromosomes can be transferred to plant cells that are subjected to electrical pulses (e.g., electroporation) and/or ultrasound (e.g., sonoporation) before, during and/or after exposure of the cells to the artificial chromosomes. Use of electrical pulses and/or ultrasound can be in combination with any other agents, e.g., PEG and/or lipids, used in transferring nucleic acids into plant cells. Artificial chromosomes can also be physically injected into plant cells through a micropipette or needle or introduced into plant cells through bombardment of the cells with microprojectiles coated with the chromosomes. To facilitate transfer of nucleic acids into plant cells, the recipient cells or tissue can be subjected to mechanical wounding.

Plant cells into which artificial chromosomes have been introduced for purposes of producing a transgenic plant are cultured under conditions that permit generation of a whole plant therefrom. The transformed cells can be analyzed prior to use in the generation of whole plants to determine suitability. For example, the cells can be analyzed for the presence of artificial chromosomes and/or regenerative capacity. Plant regeneration techniques, many of which are known to those of skill in the art, can be used to generate whole plants from, for example, cells, embryos and calli containing artificial chromosomes. For example, plants can be regenerated from cells containing artificial chromosomes by the planting of transformed roots, plantlets, seed, seedlings, and any structure capable of growing into a whole plant.

Further provided herein are methods for producing an acrocentric plant chromosome and methods for producing plant chromosomes containing adjacent regions of rDNA and heterochromatin, in particular, pericentric and/or satellite heterochromatin. Also provided herein are methods for generating acrocentric plant chromosomes containing adjacent regions of heterochromatin, such as pericentric heterochromatin and/or satellite DNA, and rDNA on the short arm of the chromosome.

One embodiment of these methods includes steps of introducing nucleic acid containing two site-specific recombination sites into a cell containing one or more plant chromosomes, recombining nucleic acids of the two site-specific recombination sites, and selecting a cell containing an acrocentric plant chromosome and/or a plant chromosome containing adjacent regions of rDNA and heterochromatin. The two site-specific recombination sites can be contained on separate nucleic acid fragments which are introduced into the cell simultaneously or sequentially.

Other embodiments of the methods of producing an acrocentric plant chromosome and/or a plant chromosome that contains adjacent regions of rDNA and heterochromatin include steps of introducing a first nucleic acid containing a site-specific recombination site into a first plant chromosome, introducing a second nucleic acid containing a site-specific recombination site into a second plant chromosome, recombining nucleic acids of the first and second chromosomes and selecting a plant chromosome that is acrocentric or that contains adjacent regions of rDNA and heterochromatin. For example, to produce an acrocentric plant chromosome, the first nucleic acid can be introduced into or adjacent to the pericentric heterochromatin of the first chromosome and/or the second nucleic acid can be introduced into the distal end of the arm of the second chromosome. To produce an acrocentric plant chromosome containing adjacent regions of rDNA and heterochromatin, for example, the first nucleic acid can be introduced into or adjacent the pericentric heterochromatin on the short arm of an acrocentric plant chromosome and the second nucleic acid can be introduced into or adjacent to rDNA. To produce a plant chromosome containing adjacent regions of rDNA and heterochromatin, for example, the first nucleic acid can be introduced into or adjacent to heterochromatin, such as pericentric heterochromatin or satellite DNA, and the second nucleic acid can be introduced into or adjacent to rDNA. When the chromosomes are located within a cell, the method can include selecting a cell containing a plant chromosome that is acrocentric and/or that contains adjacent regions of rDNA and heterochromatin.

Another embodiment of the methods of producing an acrocentric plant chromosome includes steps of introducing a first nucleic acid containing a site-specific recombination site into the pericentric heterochromatin of a plant chromosome, introducing a second nucleic acid containing a site-specific recombination site into the distal end of the chromosome in which the first and second recombination sites are located on the same arm of the chromosome, recombining nucleic acids of the first and second recombination sites in the chromosome and selecting a plant chromosome that is acrocentric.

Another method of producing an acrocentric plant chromosome or a plant chromosome containing adjacent regions of rDNA and heterochromatin includes steps of introducing nucleic acid containing a recombination site adjacent to or sufficiently near nucleic acid encoding a selectable marker into a first plant cell for recombination and introduction of the marker into the chromosome, generating a first transgenic plant from the first plant cell, introducing nucleic acid containing a promoter functional in a plant cell and a recombination site in operative linkage into a second plant cell, generating a second transgenic plant from the second plant cell, crossing the first and second plants, obtaining plants resistant to an agent that selects for cells containing the nucleic acid encoding the selectable marker, and selecting a resistant plant that contains cells containing an acrocentric plant chromosome or a plant chromosome containing adjacent regions of rDNA and heterochromatin. Methods of this embodiment can optionally include steps of selecting first and second transgenic plants such that one of the plants contains a chromosome containing a recombination site in a region within or adjacent to the pericentric heterochromatin and the other plant contains a chromosome containing a recombination site located within or adjacent to rDNA of the chromosome. These methods can further include the steps of selecting first and second transgenic plants where one of the plants contains a chromosome containing a recombination site located on a short arm of the chromosome in a region adjacent to the pericentric heterochromatin; and the other plant contains a chromosome containing a recombination site located in rDNA of the chromosome. In one embodiment, the recombination sites on the two chromosomes are in the same orientation.

In methods of producing an acrocentric plant chromosome, one or both of these recombination sites is located on a short arm of the chromosome. For example, one of the one of the plants contains a chromosome containing a recombination site in region within or adjacent to the pericentric heterochromatin located on the short arm of the chromosome. The selecting steps can further include selecting first and second transgenic plants such that the recombination sites on the two chromosomes are in the same orientation.

In any of these methods of producing an acrocentric plant chromosome or a plant chromosome containing adjacent regions of rDNA and heterochromatin (in particular, pericentric heterochromatin and/or satellite DNA), recombination between the first and second site-specific recombination sites can be provided for in a number of ways. For example, a recombinase activity can be introduced into a cell containing one or more chromosomes containing the sites which catalyzes the recombination reaction. The recombinase activity can be encoded by nucleic acid that is introduced into the cell simultaneously with nucleic acid containing a site-specific recombination site or that is introduced into the cell at a different time. Recombinase activity occurs within the cell upon expression of the nucleic acid encoding a recombinase activity, which can be operatively linked to a promoter functional in the cell. The recombinase activity can be constitutively expressed or can be induced, for example, by linking the nucleic acid encoding the recombinase to an inducible promoter. It is also possible that a cell into which nucleic acid containing site-specific recombination sites is introduced contains a recombinase enzyme which can be constitutively or inducibly expressed. Alternatively, a transgenic plant can be generated from cells containing the recombination sites and crossed with a transgenic plant containing nucleic acid encoding a recombinase.

Any site-specific recombinase system known to those of skill in the art is contemplated for use herein. It is contemplated that one or a plurality of sites that direct the recombination by the recombinase are introduced into the ACes (or other ACs) and then heterologous genes linked to the cognate site are introduced into an ACes to produce platform ACes. The resulting ACes are introduced into cells with nucleic acid encoding the cognate recombinase, typically on a vector, and nucleic acid encoding heterologous nucleic acid of interest linked to the appropriate recombination site for insertion into the ACes chromosome. The recombinase encoding nucleic acid may be introduced into the AC, includes ACes, or on the same or a difference vector from the heterologous nucleic acid.

For the methods herein any recombinase enzyme that catalyzes site-specific recombination can be used to facilitate recombination between the first and second site-specific recombination sites. A variety of recombinases and attachment/recombination sites therefor are available and/or known to those of skill in the art. These include, but not limited to: the Cre/lox recombination system using CRE recombinase from the Escherichia coli phage P1 , the FLP/FRT system of yeast using the FLP recombinase from the 2μ episome of Saccharomyces cerevisiae, the resolvases, including Gin recombinase of phage Mu, Cin, Hin, αδ Tn3; the Pin recombinase of E. coli, the R/RS system of the pSR1 plasmid of Zygosaccharomyces rouxii site specific recombinases from Kluyveromyces drosophilarium and Kluyveromyces waltii and other systems are Also contempalted is the E. coli phage lambda integrase system, the phage lambda integrase and the cognate att sites (see, also copending application U.S. application Serial No. (attorney docket No. 24601-420, filed on the same day herewith)).

In any of these methods of producing acrocentric plant chromosomes, nucleic acid containing a site-specific recombination site can also contain nucleic acid encoding a selectable marker. The nucleic acids used in the methods can be designed such that expression of the selectable marker occurs only upon the desired recombination event.

Acrocentric plant chromosomes produced by the methods provided herein can be of any composition. For example, the DNA of the short arm of the acrocentric chromosome can contain less than 5% or less than 1% euchromatic DNA or can contain no euchromatic DNA. Acrocentric plant artificial chromosomes in which the short arm of the acrocentric chromosome does not contain euchromatic DNA are provided.

In another embodiment, a method of producing a plant artificial chromosome, that includes the steps of introducing nucleic acid into a plant cell acrocentric chromosome in which the short arm does not contain euchromatic DNA; culturing the cell through at least one cell division; and selecting a cell containing an artificial chromosome, such as one that is predominantly heterochromatic, is provided. The acrocentric chromosome is produced by the method of any the methods described herein or other suitable methods.

In another embodiment, a method for producing an artificial chromosome, that includes the steps of introducing nucleic acid into a plant cell; and selecting a plant cell that includes an artificial chromosome that contains one or more repeat regions is provided. In this AC, one or more nucleic acid units is (are) repeated in a repeat region; repeats of a nucleic acid unit have common nucleic acid sequences; and the common sequences of nucleotides include sequences that represent euchromatic and heterochromatic nucleic acid. The nucleic acid can include plant rDNA from a dicot plant species or plant rDNA from a monocot plant species. The intergenic spacer region can be from DNA from a Nicotiana plant or other suitable source of such DNA. The rDNA can be plant rDNA, and the plant can be a dicot or a monocot.

Also provided are isolated plant artificial chromosomes that contain one or more repeat regions. In these ACs one or more nucleic acid units is (are) repeated in a repeat region; repeats of a nucleic acid unit have common nucleic acid sequences; and the common sequences of nucleotides include sequences that represent euchromatic and heterochromatic nucleic acid. The artificial chromosome can be produced by a method that includes the steps of: introducing nucleic acid into a plant cell; and selecting a plant cell containing an artificial chromosome that contains one or more repeat regions. The repeats of a nucleic acid unit have common nucleic acid sequences; and the common nucleic acid sequences contain sequences that represent euchromatic and heterochromatic nucleic acid.

In another embodiment, another method for producing an acrocentric plant chromosome is provided. The method includes the steps of: introducing nucleic acid containing two site-specific recombination sites into a cell containing one or more plant chromosomes; introducing into the cell a recombinase activity that catalyzes recombination between the two recombination sites to produce a plant acrocentric chromosome. In the embodiment, the two site-specific recombination sites can be on separate nucleic acid fragments, which optionally can be introduced into the cell simultaneously or sequentially. The resulting artificial chromosome can be one that is predominantly heterochromatic.

In another embodiment, a method of producing a plant artificial chromosome is provided. The method includes the steps of: introducing nucleic acid into a plant chromosome, such as but not limited to, an acrocentric chromosome, in a cell that contains adjacent regions of rDNA and heterochromatic DNA; culturing the cell through at least one cell division; and selecting a cell containing an artificial chromosome. The resulting artificial chromosome can be predominantly heterochromatic. The acrocentric chromosome can be one where the short arm of the chromosome contains adjacent regions of rDNA and heterochromatic DNA, such as, but not limited to, pericentric heterochromatin.

Also provided are a variety of vectors. Among these are vectors containing nucleic acid encoding a selectable marker that is not operably associated with any promoter, wherein the selectable marker permits growth of animal cells in the presence of an agent normally toxic to the animal cells; and wherein the agent is not toxic to plant cells; a recognition site for recombination; and a sequence of nucleotides that facilitates amplification of a region of a plant chromosome or targets the vector to an amplifiable region of a plant chromosome. Exemplary of such vectors is pAgIIa and pAgIIb.

Another vector provided herein contains nucleic acid encoding a selectable marker that is not operably associated with any promoter, wherein the selectable marker permits growth of animal cells in the presence of an agent normally toxic to the animal cells; and wherein the agent is not toxic to plant cells; a recognition site for recombination; and nucleic acid encoding a protein operably linked to a plant promoter. Exemplary of these vectors is pAg1 and pAg2.

Another vector that is provided contains: nucleic acid encoding a selectable marker that is not operably associated with any promoter, where the selectable marker permits growth of plant cells in the presence of an agent normally toxic to the plant cells but not toxic to animal cells; a recognition site for recombination; and nucleic acid encoding a protein operably linked to a plant promoter.

Another vector is a plant transformation vector that contains nucleic acid encoding a recognition site for recombination; a sequence of nucleotides that facilitates or causes amplification of a region of a plant chromosome; one or more selectable markers that are expressed in plant cells to permit the selection of cells containing the vector, and Agrobacterium nucleic acid. The vector is for Agrobacterium-mediated transformation of plants.

Another vector that is provided contains a recognition site for recombination; and a sequence of nucleotides that facilitates amplification of a region of a plant chromosome or targets the vector to an amplifiable region of a plant chromosome, wherein the plant is selected from the group consisting of Arabidopsis, Nicotiana, Solanum, Lycopersicon, Daucus, Hordeum, Zea mays, Brassica, Triticum, Helianthus, soybean, cotton and Oryza.

In these vectors, the amplifiable region can contain heterochromatic nucleic acid; the amplifiable region can contain rDNA. Exemplary sequences of nucleotides that facilitates amplification of a region of a plant chromosome or targets the vector to an amplifiable region of a plant chromosome are any that contain a sufficient portion of an intergenic spacer region of rDNA to facilitate amplification or effect the targeting. Such sufficient portion can be at least 14, 20, 30, 50, 100, 150, 300, 500, 1 kB, 2 kB, 3 kB, 5 kB, 10 kB or more contiguous nucleotides from an intergenic spacer region and/or other rDNA region. An exemplary selectable marker encodes a product confers resistance to zeomycin. The protein in the vectors include a protein that is a selectable marker that permits growth of plant cells in the presence of an agent normally toxic to the plant cells, such as, for example, resistance to hygromycin or to phosphothricin. Other such protein markers include, but are not limited to, fluorescent proteins, such as, for example, green, blue and red fluorescent proteins. An exemplary recognition site contains an att site. Exemplary promoters for inclusion in the vectors, include, but are not limited to, nopaline synthase (NOS) or CaMV35S.

Cell, containing any of the vectors or mixtures thereof are provided. The cells include any cells that have at least one plant chromosome, such as a plant cell. The cells can be protoplasts.

Methods using these vectors are provided. The methods includes a step of introducing one of the vectors into a cell, such as a cell that contains at least one plant chromosome. Such vector is for example, a vector that contains nucleic acid encoding a selectable marker that is not operably associated with any promoter, where the selectable marker permits growth of animal cells in the presence of an agent normally toxic to the animal cells but is not toxic to plant cells; a recognition site for recombination; and nucleic acid encoding a protein operably linked to a plant promoter. In this method, the cell contains an animal, such as a mammal, platform ACes that contains a recognition site, such as, for example, an att site, that recombines with the recognition site in the vector in the presences of the recombinase therefor, thereby incorporating the selectable marker that is not operably associated with any promoter and the nucleic acid encoding a protein operably linked to a plant promoter into the platform ACes to produce a resulting platform ACes. The platform ACes can contain a promoter that, upon recombination, is operably linked to the selectable marker that in the vector is not operably associated with a promoter. The method can further include transferring the resulting platform ACes into a plant cell to produce a plant cell that contains the platform Aces. The method optionally further includes culturing the plant cell that contains the platform Aces under conditions whereby the protein encoded by the nucleic acid that is operably linked to a plant promoter is expressed.

The resulting platform ACes optionally is isolated prior to transfer. The Aces can be introduced into a plant cell by any suitable method, such as one selected from among protoplast transfection, lipid-mediated delivery, liposomes, electroporation, sonoporation, microinjection, particle bombardment, silicon carbide whisker-mediated transformation, polyethylene glycol (PEG)-mediated DNA uptake, lipofection and lipid-mediated carrier systems. The resulting platform ACes can be transferred by fusion of the cells, which, for example, are plant protoplasts. In another embodiment, the cell can be an animal cell, such as a mammalian, including human, cell.

In another, method a vector is introduced into plant cells. Such vector, for example, can be a vector that includes nucleic acid encoding a selectable marker that is not operably associated with any promoter, where the selectable marker permits growth of animal cells in the presence of an agent normally toxic to the animal cells but is not toxic to plant cells; a recognition site for recombination; and a sequence of nucleotides that facilitates amplification of a region of a plant chromosome or targets the vector to an amplifiable region of a plant chromosome. The plant cells are cultured and a plant cell(s) containing an artificial chromosome that contains one or more repeat regions is selected. In this method, a sufficient portion of the vector can integrates into a chromosome in the plant cell to result in amplification of chromosomal DNA. The resulting selected artificial chromosome can be on in which one or more nucleic acid units is (are) repeated in a repeat region; repeats of a nucleic acid unit have common nucleic acid sequences; and the repeat region(s) contain substantially equivalent amounts of euchromatic and heterochromatic nucleic acid. The resulting artificial chromosome produced in the method optionally can be isolated.

Anther method is also provided. This method includes the steps of introducing a vector into a cell, and culturing the resulting cell under conditions, whereby the protein encoded by nucleic acid operably linked to an animal promoter is expressed. In the method the vector can contains: nucleic acid encoding a selectable marker that is not operably associated with any promoter, where the selectable marker permits growth of animal cells in the presence of an agent normally toxic to the animal cells but is not toxic to plant cells; a recognition site for recombination; and nucleic acid encoding a protein operably linked to an animal promoter. The cell can contain a platform plant artificial chromosome (PAC) that contains a recombination site and an animal promoter that upon recombination is operably linked to the selectable marker that in the vector is not operably associated with a promoter. Introduction can be effected under conditions whereby the vector recombines with the PAC to produce a plant platform PAC that contains the selectable marker operably linked to the promoter. In this method, the artificial chromosome can be an ACes. In addition, the plant platform PAC can be an ACes.

The vectors, such as those that contain nucleic acid encoding a selectable marker that is not operably associated with any promoter, where the selectable marker permits growth of animal cells in the presence of an agent normally toxic to the animal cells but is not toxic to plant cells; a recognition site for recombination; and a sequence of nucleotides that facilitates amplification of a region of a plant chromosome or targets the vector to an amplifiable region of a plant chromosome, and the plant transformation vectors that contain nucleic acid for Agrobacterium-mediated transformation of plants, can be used to produce artificial chromosomes. In one exemplary method, such vector is introduced into a cell containing one or more plant chromosomes; and a cell containing an artificial chromosome that contains one or more repeat regions is selected. The artificial chromosome contains one or more nucleic acid units that is (are) repeated in a repeat region; the repeats of a nucleic acid unit have common nucleic acid sequences; and the common nucleic acid sequences contain sequences that represent euchromatic and heterochromatic nucleic acid. In another method, a cell containing an artificial chromosome that contains one or more repeat regions is selected. The artificial chromosome contains one or more nucleic units that is (are) repeated in a repeat region; repeats of a nucleic acid unit have common nucleic acid sequences; and the repeat region(s) contain substantially equivalent amounts of euchromatic and heterochromatic nucleic acid.

### DESCRIPTION OF THE DRAWINGS

FIG. 1 provides a map of plasmid pAg1.

FIG. 2 provides a schematic representation of the construction of plasmid pAg1.

FIG. 3 provides a map of plasmid pAg2.

FIG. 4 provides a schematic representation of the construction of plasmid pAg2.

FIG. 5 provides a schematic representation of the construction of plasmids pAgIIa and pAgIIb.

FIG. 6A-6B provide restriction maps of the DNA inserted into pAg1 to form plasmids pAgIIa and pAgIIb.

FIG. 7 provides a map of plasmid pSV40193attPsensePUR.

FIG. 8 depicts a method for formation of a chromosome platform with multiple recombination integration sites, such as attP sites.

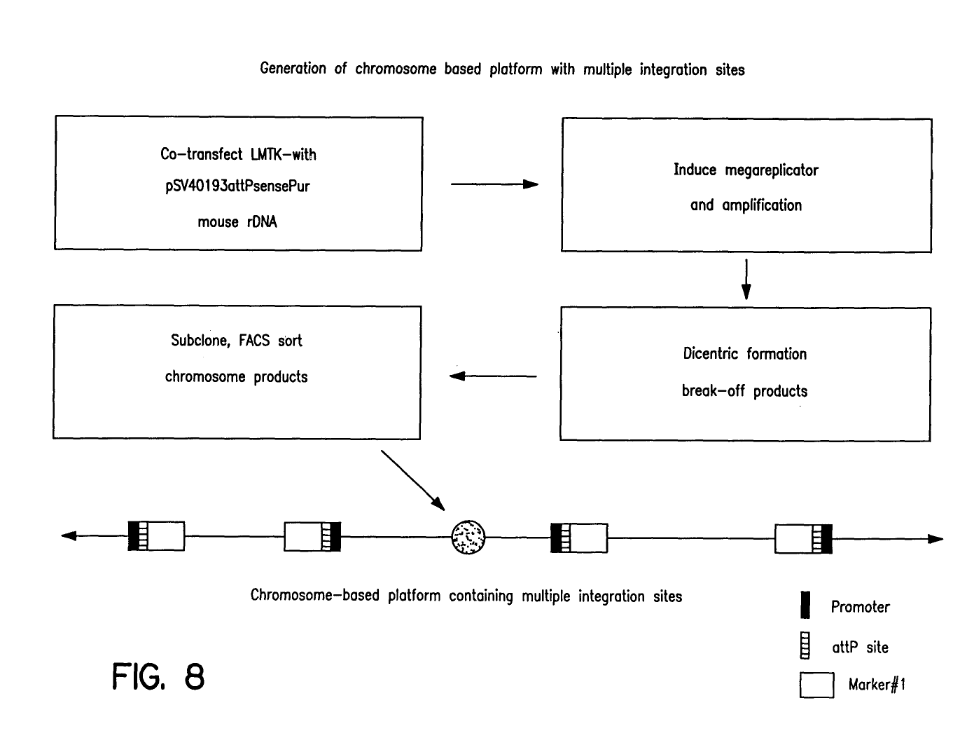

FIG. 9 diagrammatically summarizes the platform technology; marker 1 permits selection of the artificial chromosomes containing the integration site; marker 2, which is promoterless in the donor vector permits selection of recombinants. Upon recombination with the platform marker 2 is expressed under the control of a promoter resident on the platform.

### DETAILED DESCRIPTION OF PREFERRED EMBODIMENTS

Definitions

Unless defined otherwise, all technical and scientific terms used herein have the same meaning as is commonly understood by one of skill in the art to which this invention belongs. All patents, patent applications, published applications and other publications and published nucleotide and amino acid sequences (e.g., sequences available in GenBank or other databases) referred to herein are incorporated by reference in their entirety. Where reference is made to a URL or other such identifier or address, it is understood that such identifiers can change and particular information on the internet can come and go, but equivalent information can be found by searching the internet. Reference thereto evidences the availability and public dissemination of such information.

As used herein, a chromosome is a defined composition of nucleic acid that is capable of replication and segregation within a cell upon cell division. Typically, a chromosome may contain a centromeric region, telomeric regions and a region of nucleic acid between the centromeric and telomeric regions.

As used herein, a centromere is a molecular composition that includes a nucleic acid sequence that confers an ability to segregate to daughter cells through cell division. A centromere may confer stable segregation of a nucleic acid sequence, including an artificial chromosome containing the centromere, through mitotic and/or meiotic divisions. A plant centromere is not necessarily derived from plants, but has the ability to promote DNA segregation in plant cells.

As used herein, euchromatin and heterochromatin have their recognized meanings. Euchromatin refers to chromatin that stains diffusely and that typically contains genes, and heterochromatin refers to chromatin that remains unusually condensed and that has been thought to be transcriptionally inactive or has low transcriptional activity relative to euchromatin. Highly repetitive DNA sequences (satellite DNA) are usually located in regions of the heterochromatin surrounding the centromere (pericentric or pericentromeric heterochromatin). Constitutive heterochromatin refers to heterochromatin that contains the highly repetitive DNA which is constitutively condensed and genetically inactive.

As used herein, an acrocentric chromosome refers to a chromosome with arms of unequal length.

As used herein, endogenous chromosomes refer to genomic chromosomes as found in the cell prior to generation or introduction of an artificial chromosome.

As used herein, artificial chromosomes are nucleic acid molecules, typically DNA, that stably replicate and segregate alongside endogenous chromosomes in cells and have the capacity to accommodate and express heterologous genes contained therein. A mammalian artificial chromosome (MAC) refers to a chromosome that has an active mammalian centromere(s). Plant artificial chromosomes (PAC), insect artificial chromosomes and avian artificial chromosomes refer to chromosomes that include centromeres that function in plant, insect and avian cells, respectively. Human artificial chromosomes (HAC) refers to chromosomes that include centromeres that function in human cells. For exemplary artificial chromosomes, see, e.g., U.S. Pat. Nos. 6,025,155; 6,077,697; 5,288,625; 5,712,134; 5,695,967; 5,869,294; 5,891,691 and 5,721,118 and published International PCT application Nos, WO 97/40183 and WO 98/08964.

As used herein, amplification, with reference to DNA, is a process in which segments of DNA are duplicated to yield two or multiple copies of substantially similar or identical or nearly identical DNA segments that are typically joined as substantially tandem or successive repeats or inverted repeats.

As used herein, amplification-based artificial chromosomes are artificial chromosomes derived from natural or endogenous chromosomes by virtue of an amplification event, such as one that may be initiated by introduction of heterologous nucleic acid into heterochromatin, for example, pericentric heterochromatin, in a chromosome. As a result of such an event, chromosomes and/or fragments thereof exhibiting segmented or repeating patterns arise. Artificial chromosomes can be formed from these chromosomes and fragments. Hence, amplification-based artificial chromosomes refer to non-natural or isolated chromosomes that exhibit an ordered segmentation that is not typically observed in naturally occurring chromosomes and that can be a basis for distinguishing them from naturally occurring chromosomes. Amplification-based artificial chromosomes can also be distinguished from naturally occurring chromosomes by virtue of their typically smaller size and often segmented appearance when visualized. The segmented appearance, which can be visualized using a variety of chromosome analysis techniques as described herein and known to those of skill in the art, correlates with the unique structure of these artificial chromosomes. In addition to containing one or more centromeres, the amplification-based artificial chromosomes, throughout the region or regions of segmentation, are predominantly made up of one or more nucleic acid units, also referred to as “amplicons”, that is (are) repeated in the region and that have a similar gross structure. Thus, a region of segmentation may be referred to as a repeat region. Repeats of an amplicon tend to be of similar size and share some common nucleic acid sequences. For example, each repeat of an amplicon may contain a replication site involved in amplification of chromosome segments and/or some heterologous nucleic acid that was utilized in the initial production of the artificial chromosome. Typically, the repeating units are substantially similar in nucleic acid composition and may be nearly identical. The common nucleic acid sequences may contain sequences that represent euchromatic and heterochromatic nucleic acid. Amplicon sizes vary but typically tend to be greater than about 100 kb, greater than about 500 kb, greater than about 1 Mb, greater than about 5 Mb or greater than about 10 Mb. The composition of the amplification-based artificial chromosomes may be such that substantially the entire chromosome exhibits a segmented appearance or such that only one or more portions that make-up less than the entire chromosome appear segmented. The amplification-based artificial chromosomes can also differ depending on the chromosomal region that has undergone amplification in the process of artificial chromosome formation. The structures of the resulting chromosomes can vary depending upon the initiating event and/or the conditions under which the heterologous nucleic acid is introduced, including modification to the endogenous chromosomes. For example, in some of the artificial chromosomes provided herein, the region or regions of segmentation may be made up predominantly of heterochromatic DNA. In other artificial chromosomes provided herein, the region or regions of segmentation may be made up predominantly of euchromatic DNA or may be made up of similar amounts of heterochromatic and euchromatic DNA. The region or regions of segmentation thus may be entirely heterochromatic (while still containing one or more heterologous nucleic acid sequences), or may contain increasing amounts of euchromatic DNA, such that, for example, the region contains about 10%, 20%, 30%, 40%, 50%, 60%, 70%, 80%, 90% or greater than 90% euchromatic DNA. Because the entire artificial chromosome can be made up predominantly of a region or regions of segmentation, it is thus possible for the artificial chromosome to be made up predominantly of heterochromatin or euchromatin, or to be made up of substantially equivalent amounts of heterochromatin and euchromatin, e.g., about 40% to about 50% of one type of nucleic acid and about 50% to about 60% of the other type of nucleic acid.

As used herein the term “predominantly” with respect to a composition generally refers to a state of the composition in which it can be characterized as being or having more of the predominant feature than other features which are not predominant. The predominant feature may represent more than about 50%, more than about 60%, more than about 70%, more than about 80%, more than about 90%, more than about 95% or essentially 100% of the composition. Thus, for example, a repeat region that is predominantly made up of heterochromatic DNA contains more heterochromatic DNA than other types, e.g., euchromatic, of DNA. The repeat region may be more than about 50%, more than about 60%, more than about 70%, more than about 80%, more than about 90% or more than about 95% heterochromatic DNA or may be essentially 100% heterochromatic DNA. An artificial chromosome predominantly made up of heterochromatin contains more heterochromatic DNA than other types, e.g., euchromatic, of DNA and may be more than about 50%, more than about 60%, more than about 70%, more than about 80%, more than about 90% or more than about 95% heterochromatic DNA or may be essentially 100% heterochromatic DNA.

As used herein an amplicon is a repeated nucleic acid unit. In some of the artificial chromosomes described herein, an amplicon may contain a set of inverted repeats of a megareplicon. A megareplicon represents a higher order replication unit. For example, with reference to some of the predominantly heterochromatic artificial chromosomes, particularly eukaryotic chromosomes, described herein, the megareplicon may contain a set of tandem DNA blocks (e.g., ˜7.5 Mb DNA blocks) each containing satellite DNA flanked by non-satellite DNA or may substantially be made up of rDNA. Contained within the megareplicon is a primary replication site, referred to as the megareplicator, which may be involved in organizing and facilitating replication of segments of chromosomes, including, for example, heterochromatin, pericentric heterochromatin, rDNA and/or possibly the centromeres. Within the megareplicon there may be smaller (e.g., 50-300 kb) secondary replicons.

As used herein, amplifiable, when used in reference to a chromosome, particularly the method of generating artificial chromosomes provided herein, refers to a region of a chromosome that is prone to amplification. Amplification typically occurs during replication and other cellular events involving recombination (e.g., DNA repair). Included among such regions are regions of the chromosome that contain tandem repeats, such as satellite DNA, rDNA, and other such sequences.

Among the artificial chromosome systems provided herein are those that are predominantly heterochromatic [formerly referred to as satellite artificial chromosomes (SATACs); see, e.g., U.S. Pat. Nos. 6,077,697 and 6,025,155 and published International PCT application No. WO 97/40183], minichromosomes which contain a de novo centromere, artificial chromosomes containing one or more regions of repeating nucleic acid units wherein the repeat region(s) contain substantially equivalent amounts of euchromatic and heterochromatic nucleic acid and in vitro assembled artificial chromosomes. Of particular interest herein are artificial chromosomes that introduce and express heterologous nucleic acids in plants. These include artificial chromosomes that have a centromere derived from a plant, and, also, artificial chromosomes that have centromeres that may be derived from other organisms but that function in plants. Methods for the construction, isolation, and delivery to target cells of each type of artificial chromosome are provided herein.

As used herein, to target nucleic acid to a locus on a chromosome means that the nucleic acid integrates at or near the targeted locus. Any method or means for effecting such integration, including, but not limited to, homologous recombination, is contemplated.

As used herein, a dicentric chromosome is a chromosome that contains two centromeres. A multicentric chromosome contains more than two centromeres.

As used herein, a formerly dicentric chromosome is a chromosome that is produced when a dicentric chromosome fragments and acquires new telomeres so that two chromosomes, each having one of the centromeres, are produced. Each of the fragments are replicable chromosomes. If one of the chromosomes undergoes amplification of primarily euchromatic DNA to produce a fully functional chromosome that is predominantly (more than about 50%, more than about 70% or more than about 90% euchromatin) euchromatin, it is a minichromosome. The remaining chromosome is a formerly dicentric chromosome. If one of the chromosomes undergoes amplification, whereby heterochromatin (such as, for example, satellite DNA) is amplified and a euchromatic portion (such as, for example, an arm) remains, it is referred to as a sausage chromosome. A chromosome that is substantially all heterochromatin, except for portions of heterologous DNA, is called a predominantly heterochromatic artificial chromosome. Predominantly heterochromatic artificial chromosomes can be produced from other partially heterochromatic artificial chromosomes by culturing the cell containing such chromosomes under conditions that destabilize the chromosome and/or under selective conditions so that a predominantly heterochromatic artificial chromosome is produced. For purposes herein, it is understood that the artificial chromosomes may not necessarily be produced in multiple steps, but may appear after the initial introduction of the heterologous DNA. Typically, artificial chromosomes appear after about 5 to about 60, or about 5 to about 55, or about 10 to about 55 or about 25 to about 55 or about 35 to about 55 cell divisions following introduction of nucleic acid into a cell. Artificial chromosomes may, however, appear after only about 5 to about 15 or about 10 to about 15 cell divisions.

As used herein, the term “satellite DNA-based artificial chromosome (SATAC)” is interchangable with the term “artificial chromosome expression system (ACes)”. These artificial chromosomes (ACes) include those that are substantially all neutral non-coding sequences (heterochromatin) except for foreign heterologous, typically gene or protein-encoding, nucleic acid, that may be interspersed within the heterochromatin for the expression therein (see U.S. Pat. Nos. 6,025,155 and 6,077,697 and International PCT application No. WO 97/40183), or that is in a single locus as provided herein. The delineating structural feature is the presence of repeating units, which are generally predominantly heterochromatin. The precise structure of the ACes will depend upon the structure of the chromosome in which the initial amplification event occurs; all share the common feature of including a defined pattern of repeating units. Generally ACes have more heterochromatin than euchromatin. Foreign nucleic acid molecules (heterologous genes) contained in these artificial chromosome expression systems can include any nucleic acid whose expression is of interest in a particular host cell.

As used herein, an artificial chromosome that is predominantly heterochromatic (i.e., containing more heterochromatin than euchromatin, typically more than about 50%, more than about 60%, more than about 70%, more than about 80% or more than about 90% heterochromatin) may be produced by introducing nucleic acid molecules into cells, particularly plant cells, and selecting cells that contain a predominantly heterochromatic artificial chromosome. Any nucleic acid may be introduced into cells in the methods of producing the artificial chromosomes. For example, the nucleic acid may contain a selectable marker and/or a sequence that targets nucleic acid to a heterochromatic region of a chromosome, particularly a plant chromosome, such as in the pericentric heterochromatin, in the short arm of acrocentric chromosomes, rDNA or nucleolar organizing regions. Targeting sequences include, but are not limited to, lambda phage DNA and rDNA (e.g., a sequence of an intergenic spacer of rDNA), particularly plant rDNA, for production of predominantly heterochromatic artificial chromosomes in plant cells.

After introducing the nucleic acid into cells, a cell containing a predominantly heterochromatic artificial chromosome is selected. Such cells may be identified using a variety of procedures. For example, repeating units of heterochromatic DNA of these chromosomes may be discerned by G- and/or C-banding and/or fluorescence in situ hybridization (FISH) techniques. Prior to such analyses, the cells to be analyzed may be enriched with artificial chromosome-containing cells by sorting the cells on the basis of the presence of a selectable marker, such as a reporter protein, or by growing (culturing) the cells under selective conditions. Selection of cells containing amplified nucleic acids may also be facilitated by use of techniques such as PCR and Southern blotting to identify cell lines with amplified regions. It is also possible, after introduction of nucleic acids into cells, to select cells that have a multicentric, typically dicentric, chromosome, a formerly multicentric (typically dicentric) chromosome and/or various heterochromatic structures and to treat them such that desired artificial chromosomes are produced. Conditions for generation of a desired structure include, but are not limited to, further growth under selective conditions, introduction of additional nucleic acid molecules and/or growth under selective conditions and treatment with destabilizing agents, and other such methods (see International PCT application No. WO 97/40183 and U.S. Pat. Nos. 6,025,155 and 6,077,697).

As used herein, heterologous and foreign are used interchangeably with respect to nucleic acid and refer to any nucleic acid, including DNA and RNA, that does not occur naturally as part of the genome in which it is present or which is found in a location or locations in the genome that differ from that in which it occurs in nature. Thus, heterologous or foreign nucleic acid that is not normally found in the host genome in an identical context. It is nucleic acid that is not endogenous to the cell and has been exogenously introduced into the cell. Examples of heterologous DNA include, but are not limited to, DNA that encodes a gene product or gene product(s) of interest, introduced for purposes of modification of the endogenous genes or for production of an encoded protein. For example, a heterologous or foreign gene may be isolated from a different species than that of the host genome, or alternatively, may be isolated from the host genome but operably linked to one or more regulatory regions which differ from those found in the unaltered, native gene. Other examples of heterologous DNA include, but are not limited to, DNA that encodes traceable marker proteins, and DNA that encodes a protein that confers an input trait including, but not limited to, herbicide, insect, or disease resistance or an output trait, including, but not limited to, oil quality or carbohydrate composition. Antibodies that are encoded by heterologous DNA may be secreted, sequestered, stored in an organ or tissue, accumulate in the cytoplasm or cellular organelles or expressed on the surface of the cell in which the heterologous DNA has been introduced.

As used herein, a “selectable marker” is a composition that can be used to distinguish one cell from another cell. For example, a selectable marker may be a nucleic acid encoding a readily detected protein that has been introduced into some cells but not others. Detection of the expressed protein in cells facilitates identification of cells containing the marker nucleic acid by distinguishing them from cells that do not contain the nucleic acid. Thus, for example, a selectable marker may be a fluorescent protein, such as green fluorescent protein (GFP), or β-galactosidase (or a nucleic acid encoding either of these proteins). Selectable markers such as these, which are not required for cell survival and/or proliferation in the presence of a selection agent, may also be referred to as reporter molecules. Other selectable markers, e.g., the neomycin phosphotransferase gene, provide for isolation and identification of cells containing them by conferring properties on the cells that make them resistant to an agent, e.g., a drug such as an antibiotic, that inhibits proliferation of cells that do not contain the marker.

As used herein, growth under selective conditions means growth of a cell under conditions that require expression of a selectable marker for survival.

As used herein, an agent that destabilizes a chromosome is any agent known by those of skill in the art to enhance amplification events, and/or mutations. Such agents, which include BrdU, are well known to those of skill in the art.

In order to generate an artificial chromosome containing a particular heterologous nucleic acid of interest, it is possible to include the nucleic acid of interest in the nucleic acid that is being introduced into cells to initiate production of the artificial chromosome. Thus, for example, a nucleic acid of interest could be introduced into a cell along with nucleic acid encoding a selectable marker and/or a nucleic acid that targets to a heterochromatic region of a chromosome. For example, the nucleic acid of interest can be linked to targeting nucleic acid(s). Alternatively, heterologous nucleic acid of interest can be introduced into an artificial chromosome at a later time after the initial generation of the artificial chromosome.

As used herein, the minichromosome refers to a chromosome derived from a multicentric, typically dicentric, chromosome that contains more euchromatic than heterochromatic DNA. For purposes herein, the minichromosome contains a de novo centromere, preferably a centromere that replicates in plants, more preferably a plant centromere.

As used herein, de novo with reference to a centromere, refers to generation of an excess centromere in a chromosome as a result of incorporation of a heterologous nucleic acid fragment using the methods herein.

As used herein, in vitro assembled artificial chromosomes or synthetic chromosomes are artificial chromosomes produced by joining essential components of a chromosome in vitro. These components include at least a centromere, a telomere and an origin of replication. An in vitro assembled artificial chromosome may include one or more megareplicators. In particular embodiments, the megareplicator contains sequences of rDNA, particularly plant rDNA.

As used herein, in vitro assembled plant artificial chromosomes are produced by joining components (e.g., the centromere, telomere(s) megareplicator and an origin of replication) that function in plants, and preferably, one or more of which is derived from a plant. In vitro assembled artificial chromosomes may contain any amount of heterochromatic and/or euchromatic nucleic acid. For example, an in vitro assembled artificial chromosome may be substantially all heterochromatin, or may contain increasing amounts of euchromatic DNA, such that, for example, it contains about 10%, 20%, 30%, 40%, 50%, 60%, 70%, 80%, 90% or greater than about 90% euchromatic DNA. In vitro assembled artificial chromosomes may contain one or more regions of segmentation as described with reference to amplification-based artificial chromosomes.

As used herein, an artificial chromosome platform refers to an artificial chromosome that has been engineered to include one or more sites for site specific recombination-directed integration. Included within the artificial chromosome platforms are ACes, particularly plant ACes, that are so-engineered. Any sites, including but not limited to any described herein, that are suitable for such integration are contemplated. Among the ACes contemplated herein are those that are predominantly heterochromatic (formerly referred to as satellite artificial chromosomes (SATACs); see, e.g., U.S. Pat. Nos. 6,077,697 and 6,025,155 and published International PCT application No. WO 97/40183), artificial chromosomes predominantly made up of repeating nucleic acid units and that contain substantially equivalent amounts of euchromatic and heterochromatic DNA or wherein the repeat regions of the chromosomes contain substantially equivalent amounts of euchromatic and heterochromatic nucleic acid. Included among the ACes for use in generating platforms are artificial chromosomes that introduce and express heterologous nucleic acids in plants as described herein. These include artificial chromosomes that have a centromere derived from a plant, and, also, artificial chromosomes that have centromeres that may be derived from other organisms but that function in plants.

As used herein, recognition sequences are particular sequences of nucleotides that a protein, DNA, or RNA molecule, or combinations thereof, (such as, but not limited to, a restriction endonuclease, a modification methylase and a recombinase) recognizes and binds. For example, a recognition sequence for Cre recombinase (see, e.g., SEQ ID No. 30) is a 34 base pair sequence containing two 13 base pair inverted repeats (serving as the recombinase binding sites) flanking an 8 base pair core and designated loxP (see, e.g., Sauer (1994) Current Opinion in Biotechnology 5:521-527). Other examples of recognition sequences, include, but are not limited to, attB and attP, attR and attL and others (see, e.g., SEQ ID Nos. 32-48), that are recognized by the recombinase enzyme Integrase (see, SEQ ID Nos. 49 and 50) for the nucleotide and encoded amino acid sequences of an exemplary lambda phage integrase).

The recombination site designated attB is an approximately 33 base pair sequence containing two 9 base pair core-type Int binding sites and a 7 base pair overlap region; attP (SEQ ID No. 48) is an approximately 240 base pair sequence containing core-type Int binding sites and arm-type Int binding sites as well as sites for auxiliary proteins IHF, FIS, and Xis (see, e.g., Landy (1993) Current Opinion in Biotechnology 3:699-707| see, e.g., SEQ ID Nos. 32 and 48).

As used herein, a recombinase is an enzyme that catalyzes the exchange of DNA segments at specific recombination sites. An integrase herein refers to a recombinase that is a member of the lambda (λ) integrase family.

As used herein, recombination proteins include excisive proteins, integrative proteins, enzymes, co-factors and associated proteins that are involved in recombination reactions using one or more recombination sites (see, Landy (1993) Current Opinion in Biotechnology 3:699-707).

As used herein the expression “lox site” means a sequence of nucleotides at which the gene product of the cre gene, referred to herein as Cre, can catalyze a site-specific recombination event. A LoxP site is a 34 base pair nucleotide sequence from bacteriophage P1 (see, e.g., Hoess et al. (1982) Proc. Natl. Acad. Sci. U.S.A. 79:3398-3402). The LoxP site contains two 1 3 base pair inverted repeats separated by an 8 base pair spacer region as follows: (SEQ ID NO. 51):

ATAACTTCGTATA ATGTATGC TATACGAAGTTAT

E. coliDH5Δ lac and yeast strain BSY23 transformed with plasmid pBS44 carrying two loxP sites connected with a LEU2 gene are available from the American Type Culture Collection (ATCC) under accession numbers ATCC 53254 and ATCC 20773, respectively. The lox sites can be isolated from plasmid pBS44 with restriction enzymes EcoRI and Sa/l, or XhoI and BamHI. In addition, a preselected DNA segment can be inserted into pBS44 at either the SaA or BamHI restriction enzyme sites. Other lox sites include, but are not limited to, LoxB, LoxL, LoxC2 and LoxR sites, which are nucleotide sequences isolated from E. coli (see, e.g., Hoess et al. (1982) Proc. Natl. Acad. Sci. U.S.A. 79:3398). Lox sites can also be produced by a variety of synthetic techniques (see, e.g., Ito et al. (1982) Nuc. Acid Res. 10:1755 and Ogilvie et al. (1981) Science 270:270).

As used herein, the expression “cre gene” means a sequence of nucleotides that encodes a gene product that effects site-specific recombination of DNA in eukaryotic cells at lox sites. One cre gene can be isolated from bacteriophage P1 (see, e.g., Abremski et al. (1983) Cell 32:1301-1311). E. coil DH1 and yeast strain BSY90 transformed with plasmid pBS39 carrying a cre gene isolated from bacteriophage P1 and a GAL1 regulatory nucleotide sequence are available from the American Type Culture Collection (ATCC) under accession numbers ATCC 53255 and ATCC 20772, respectively. The cre gene can be isolated from plasmid pBS39 with restriction enzymes XhoI and SalI.

As used herein, site-specific recombination refers to site-specific recombination that is effected between two specific sites on a single nucleic acid molecule or between two different molecules that requires the presence of an exogenous protein, such as an integrase or recombinase.

For example, Cre-lox site-specific recombination can include the following three events:
a. deletion of a pre-selected DNA segment flanked by lox sites;
b. inversion of the nucleotide sequence of a pre-selected DNA segment flanked by lox sites; and
c. reciprocal exchange of DNA segments proximate to lox sites located on different DNA molecules.

This reciprocal exchange of DNA segments can result in an integration event if one or both of the DNA molecules are circular. DNA segment refers to a linear fragment of single- or double-stranded deoxyribonucleic acid (DNA), which can be derived from any source. Since the lox site is an asymmetrical nucleotide sequence, two lox sites on the same DNA molecule can have the same or opposite orientations with respect to each other. Recombination between lox sites in the same orientation results in a deletion of the DNA segment located between the two lox sites and a connection between the resulting ends of the original DNA molecule. The deleted DNA segment forms a circular molecule of DNA. The original DNA molecule and the resulting circular molecule each contain a single lox site. Recombination between lox sites in opposite orientations on the same DNA molecule result in an inversion of the nucleotide sequence of the DNA segment located between the two lox sites. In addition, reciprocal exchange of DNA segments proximate to lox sites located on two different DNA molecules can occur. All of these recombination events are catalyzed by the gene product of the cre gene. Thus, the Cre-lox system can be used to specifically delete, invert, or insert DNA. The precise event is controlled by the orientation of lox DNA sequences, in cis the lox sequences direct the Cre recombinase to either delete (lox sequences in direct orientation) or invert (lox sequences in inverted orientation) DNA flanked by the sequences, while in trans the lox sequences can direct a homologous recombination event resulting in the insertion of a recombinant DNA.

As used herein, a plant refers to an organism that is taxonomically classifed as being in the kingdom Plantae. Such organisms include eukaryotic organisms that contain chloroplasts capable of carrying out photosynthesis. A plant can be unicellular or multicellular and can contain multiple tissues and/or organs. Plants can reproduce sexually and/or asexually and include species that are perennial or annual in growth habit. A plants can be found to exist in a variety of habitats, including terrestrial and aquatic environments. The term “plant” includes a whole plant, plant cell, plant protoplast, plant calli, plant seed, plant organ, plant tissue, and other parts of a whole plant.

As used herein, reproductive mode with reference to a plant refers to any and all methods by which a plant produces progeny. Reproductive modes include, but are not limited to, sexual and asexual reproduction. Plants may produce progeny by one or multiple reproductive modes. Sexual reproduction can include union of cells derived from haploid gametophytes (e.g., eggs produced from ovules and sperm produced from pollen in seed plants) to form diploid zygotes. Zygotes may be formed from gametophytes from different plants or from gametophytes of the same plant (e.g., through self-fertilization). Asexual reproduction can occur when offspring are produced through modifications of the sexual life cycle that do not include meiosis and syngamy. For example, when vascular plants reproduce asexually, they may do so by vegetative reproduction, such as budding, branching, and tillering, or by producing spores or seed genetically identical to the sporophytes that produced them.

As used herein, stable maintenance of chromosomes occurs when at least about 85%, preferably 90%, more preferably 95%, of the cells retain the chromosome. Stability is measured in the presence of a selective agent. Preferably these chromosomes are also maintained in the absence of a selective agent. Stable chromosomes also retain their structure during cell culturing, suffering no unintended intrachromosomal nor interchromosomal rearrangements.

As used herein, BrdU refers to 5-bromodeoxyuridine, which during replication is inserted in place of thymidine. BrdU is used as a mutagen; it also inhibits condensation of metaphase chromosomes during cell division.

As used herein, ribosomal RNA (rRNA) is the specialized RNA that forms part of the structure of a ribosome and participates in the synthesis of proteins. Ribosomal RNA is produced by transcription of genes which, in eukaryotic cells, are present in multiple copies. In human cells, the approximately 250 copies of rRNA genes (i.e., genes which encode rRNA) per haploid genome are spread out in clusters on at least five different chromosomes (chromosomes 13, 14, 15, 21 and 22). In mouse cells, the presence of ribosomal DNA (rDNA, which is DNA containing sequences that encode rRNA) has been verified on at least 11 pairs out of 20 mouse chromosomes (chromosomes 5, 6, 7, 9, 11, 12, 15, 16, 17, 18, and 19) [see e.g., Rowe et al. (1996) Mamm. Genome 7:886-889 and Johnson et al. (1993) Mamm. Genome 4:49-52]. In Arabidopsis thaliana the presence of rDNA has been verified on chromosomes 2 and 4 (18S, 5.8S, and 25S rDNA) and on chromosomes 3,4, and 5 (5S rDNA)[see The Arabidopsis Genome Initiative (2000) Nature 408:796-815]. In eukaryotic cells, the multiple copies of the highly conserved rRNA genes are located in a tandemly arranged series of rDNA units, which are generally about 40-45 kb in length and contain a transcribed region and a nontranscribed region known as spacer (i.e., intergenic spacer) DNA which can vary in length and sequence. In the human and mouse, these tandem arrays of rDNA units are located adjacent to the pericentric satellite DNA sequences (heterochromatin). The regions of these chromosomes in which the rDNA is located are referred to as nucleolar organizing regions (NOR) which loop into the nucleolus, the site of ribosome production within the cell nucleus. In higher plants, the rDNA is arragened in long tandem repeating units, similar to those of other higher eukaroytes. The 18S, 5.8S and 25S rRNA genes are clustered and are transcribed as one unit, while the 5S genes are located elsewhere in the genome. Between the 3′ end of the 25S gene and the 5′ end of the 18S gene is located a DNA spacer that ranges from 1 kb to greater than 12 kb in length for different species. Therefore, the rDNA repeat ranges from about 4 kb to about 15 kb for different plant species [see, e.g., Rogers and Bendich (1987) Plant Mol. Biol. 9:509-520].

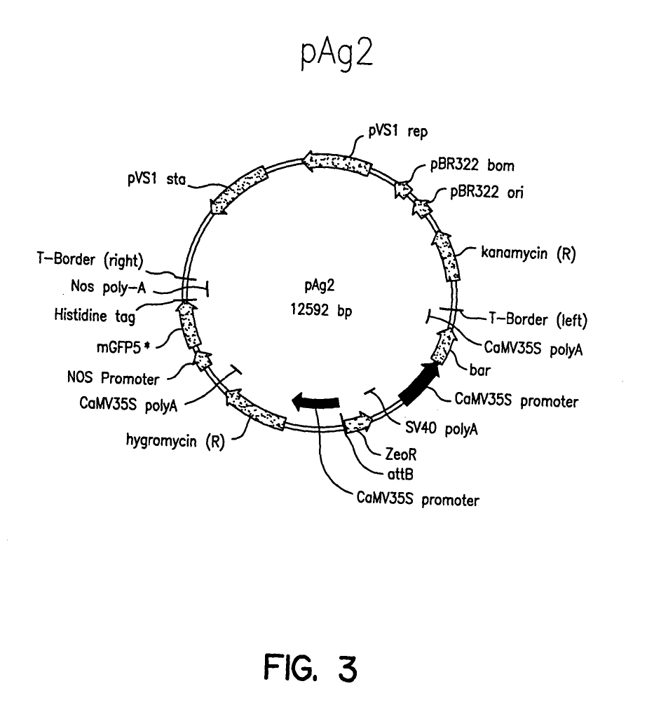

As used herein, a megachromosome refers to a chromosome that, except for introduced heterologous DNA, is substantially composed of heterochromatin. Megachromosomes are made up of an array of repeated amplicons that contain two inverted megareplicons bordered by introduced heterologous DNA [see, e.g., FIG. 3 of U.S. Pat. No. 6,077,697 for a schematic drawing of a megachromosome]. For purposes herein, a megachromosome is about 50 to 400 Mb, generally about 250-400 Mb. Shorter variants are also referred to as truncated megachromosomes [about 90 to 120 or 150 Mb], dwarf megachromosomes [˜150-200 Mb] and cell lines, and a micro-megachromosome [˜50-90 Mb, typically 50-60 Mb]. For purposes herein, the term megachromosome refers to the overall repeated structure based on an array of repeated chromosomal segments (amplicons) that contain two inverted megareplicons bordered by any inserted heterologous DNA.

As used herein, transformation and transfection are used interchangeably to refer to the process of introducing nucleic acid introduced into cells. The terms transfection and transformation refer to the taking up of exogenous nucleic acid, e.g., an expression vector, by a host cell whether or not any coding sequences are in fact expressed. Numerous methods of introducing nucleic acids into cells are known to the ordinarily skilled artisan, for example, by Agrobacterium-mediated transformation, protoplast transfection (including polyethylene glycol (PEG)-mediated transfection, electroporation, protoplast fusion, and microcell fusion), lipid-mediated delivery, liposomes, electroporation, microinjection, particle bombardment and silicon carbide whisker-mediated transformation (see, e.g., Paszkowski et al. (1984) EMBO J. 3:2717-2722; Potrykus et al. (1985) Mol. Gen. Genet. 199:169-177; Reich et al. (1986) Biotechnology 4:1001-1004; Klein et al. (1987) Nature 327:70-73; U.S. Pat. No. 6,143,949; Paszkowski et al. (1989) in Cell Culture and Somatic Cell Genetics of Plants, Vol. 6, Molecular Biology of Plant Nuclear Genes, eds. Schell, J and Vasil, L. K. Academic Publishers, San Diego, Calif., p. 52-68; and Frame et al. (1994) Plant J. 6:941-948), direct uptake using calcium phosphate [CaPO4; see,e.g., Wigler et al. (1979) Proc. Natl. Acad. Sci. U.S.A. 76:1373-1376], polyethylene glycol [PEG]-mediated DNA uptake, lipofection [see, e.g., Strauss (1996) Meth. Mol. Biol. 54:307-327], microcell fusion [see Lambert (1991) Proc. Natl. Acad. Sci. U.S.A. 88:5907-5911; U.S. Pat. No. 5,396,767, Sawford et al. (1987) Somatic Cell Mol. Genet. 13:279-284; Dhar et al. (1984) Somatic Cell Mol. Genet. 10:547-559; and McNeill-Killary et al. (1995) Meth. Enzymol. 254:133-152], lipid-mediated carrier systems [see, e.g., Teifel et al. (1995) Biotechniques 19:79-80; Albrecht et al. (1996) Ann. Hematol. 72:73-79; Holmen et al. (1995) In Vitro Cell Dev. Biol. Anim. 31:347-351; Remy et al. (1994) Bioconjug. Chem. 5:647-654; Le Bolch et al. (1995) Tetrahedron Lett. 36:6681-6684; Loeffler et al. (1993) Meth. Enzymol. 217:599-618] or other suitable method. Successful transfection is generally recognized by detection of the presence of the heterologous nucleic acid within the transfected cell, such as, for example, any visualization of the heterologous nucleic acid or any indication of the operation of a vector within the host cell.

As used herein, injected refers to the microinjection (use of a small syringe, needle, or pipette) of nucleic acid into a cell.

As used herein, gene therapy involves the transfer or insertion of nucleic acid molecules into certain cells, which are also referred to as target cells, to produce products that are involved in preventing, curing, correcting, controlling or modulating diseases, disorders and/or deleterious conditions. The nucleic acid is introduced into the selected target cells in a manner such that the nucleic acid is expressed and a product encoded thereby is produced. Alternatively, the nucleic acid may in some manner mediate expression of DNA that encodes a therapeutic product. This product may be a therapeutic compound, which is produced in therapeutically effective amounts or at a therapeutically useful time. It may also encode a product, such as a peptide or RNA, that in some manner mediates, directly or indirectly, expression of a therapeutic product. Expression of the nucleic acid by the target cells within an organism afflicted with a disease or disorder thereby enables modulation of the disease or disorder. The nucleic acid encoding the therapeutic product may be modified prior to introduction into the cells of the afflicted host in order to enhance or otherwise alter the product or expression thereof.

For use in gene therapy, cells can be transfected in vitro, followed by introduction of the transfected cells into an organism. This is often referred to as ex vivo gene therapy. Alternatively, the cells can be transfected directly in vivo within an organism.

As used herein, a therapeutically effective product is a product that effectively ameliorates or eliminates the symptoms or manifestations of an inherited or acquired disease or disorder or that cures said disease or disorder in an organism. For example, therapeutically effective products include a product that is encoded by heterologous DNA expressed in a diseased organism and a product produced from heterologous DNA in a host cell and to which a diseased organism is exposed.

As used herein, a transgenic plant refers to a plant (e.g., a plant cell, tissue, organ or whole plant) containing heterologous or foreign nucleic acid or in which the expression of a gene naturally present in the plant has been altered. Heterologous nucleic acid within a transgenic plant may be transiently or stably maintained within the plant. Stable maintenance of heterologous nucleic acid may be maintenance of the nucleic acid through one or more, or two or more, or five or more, or ten or more, or 25 or more, or 50 or more or 60 or more cell divisions. A transgenic plant may contain heterologous nucleic acid in one cell, multiple cells or all cells. A transgenic plant may produce progeny that contain or do not contain the heterologous nucleic acid.

As used herein, a promoter, with respect to a region of DNA, refers to a sequence of DNA that contains a sequence of bases that signals RNA polymerase to associate with the DNA and initiate transcription of messenger RNA (mRNA) from a template strand of the DNA. A promoter thus generally regulates transcription of DNA into mRNA.

As used herein, operative linkage of heterologous DNA to regulatory and effector sequences of nucleotides, such as promoters, enhancers, transcriptional and translational stop sites, and other signal sequences refers to the relationship between such DNA and such sequences of nucleotides. For example, operative linkage of heterologous DNA to a promoter refers to the physical relationship between the DNA and the promoter such that the transcription of such DNA is initiated from the promoter by an RNA polymerase that specifically recognizes, binds to and transcribes the DNA in reading frame.

As used herein, isolated, substantially pure nucleic acid, such as, for example, DNA, refers to nucleic acid fragments purified according to standard techniques employed by those skilled in the art, such as that found in Maniatis et al. [(1982) Molecular Cloning: A Laboratory Manual, Cold Spring Harbor Laboratory Press, Cold Spring Harbor, N.Y.].

As used herein, expression refers to the transcription and/or translation of nucleic acid. For example, expression can be the transcription of a gene into an RNA molecule, such as a messenger RNA (mRNA) molecule. Expression may further include translation of an RNA molecule into peptides, polypeptides, or proteins. If the nucleic acid is derived from genomic DNA, expression may, if an appropriate eukaryotic host cell or organism is selected, include splicing of the mRNA. With respect to an antisense construct, expression may refer to the transcription of the antisense DNA.

As used herein, vector or plasmid refers to discrete elements that are used to introduce heterologous nucleic acids into cells for either expression of the heterologous nucleic acid or for replication of the heterologous nucleic acid. Selection and use of such vectors and plasmids are well within the level of skill of the art.

As used herein, substantially homologous DNA refers to DNA that includes a sequence of nucleotides that is sufficiently similar to another such sequence to form stable hybrids under specified conditions.

It is well known to those of skill in this art that nucleic acid fragments with different sequences may, under the same conditions, hybridize detectably to the same “target” nucleic acid. Two nucleic acid fragments hybridize detectably, under stringent conditions over a sufficiently long hybridization period, because one fragment contains a segment of at least about 14 nucleotides in a sequence which is complementary (or nearly complementary) to the sequence of at least one segment in the other nucleic acid fragment. If the time during which hybridization is allowed to occur is held constant, at a value during which, under preselected stringency conditions, two nucleic acid fragments with exactly complementary base-pairing segments hybridize detectably to each other, departures from exact complementarity can be introduced into the base-pairing segments, and base-pairing will nonetheless occur to an extent sufficient to make hybridization detectable. As the departure from complementarity between the base-pairing segments of two nucleic acids becomes larger, and as conditions of the hybridization become more stringent, the probability decreases that the two segments will hybridize detectably to each other.

Two single-stranded nucleic acid segments have “substantially the same sequence,” within the meaning of the present specification, if (a) both form a base-paired duplex with the same segment, and (b) the melting temperatures of said two duplexes in a solution of 0.5×SSPE differ by less than 10° C. If the segments being compared have the same number of bases, then to have “substantially the same sequence”, they will typically differ in their sequences at fewer than 1 base in 10. Methods for determining melting temperatures of nucleic acid duplexes are well known [see, eg., Meinkoth and Wahl (1984) Anal. Biochem. 138:267-284 and references cited therein].

As used herein, a nucleic acid probe is a DNA or RNA fragment that includes a sufficient number of nucleotides to specifically hybridize to DNA or RNA that includes identical or closely related sequences of nucleotides. A probe may contain any number of nucleotides, from as few as about 10 and as many as hundreds of thousands of nucleotides. The conditions and protocols for such hybridization reactions are well known to those of skill in the art as are the effects of probe size, temperature, degree of mismatch, salt concentration and other parameters on the hybridization reaction. For example, the lower the temperature and higher the salt concentration at which the hybridization reaction is carried out, the greater the degree of mismatch that may be present in the hybrid molecules.

To be used as a hybridization probe, the nucleic acid is generally rendered detectable by labelling it with a detectable moiety or label, such as ³²P, ³H and ¹⁴C, or by other means, including chemical labelling, such as by nick-translation in the presence of deoxyuridylate biotinylated at the 5′-position of the uracil moiety. The resulting probe includes the biotinylated uridylate in place of thymidylate residues and can be detected (via the biotin moieties) by any of a number of commercially available detection systems based on binding of streptavidin to the biotin. Such commercially available detection systems can be obtained, for example, from Enzo Biochemicals, Inc. (New York, N.Y.). Any other label known to those of skill in the art, including non-radioactive labels, may be used as long as it renders the probes sufficiently detectable, which is a function of the sensitivity of the assay, the time available (for culturing cells, extracting DNA, and hybridization assays), the quantity of DNA or RNA available as a source of the probe, the particular label and the means used to detect the label.

Once sequences with a sufficiently high degree of homology to the probe are identified, they can readily be isolated by standard techniques, which are described, for example, by Maniatis et al. [(1982) Molecular Cloning: A Laboratory Manual, Cold Spring Harbor Laboratory Press, Cold Spring Harbor, N.Y.].

As used herein, conditions under which DNA molecules form stable hybrids and are considered substantially homologous are such that DNA molecules with at least about 60% complementarity form stable hybrids. Such DNA fragments are herein considered to be “substantially homologous”. For example, DNA that encodes a particular protein is substantially homologous to another DNA fragment if the DNA forms stable hybrids such that the sequences of the fragments are at least about 60% complementary and if a protein encoded by the DNA retains its activity.

For purposes herein, the following stringency conditions are defined:
1) high stringency: 0.1×SSPE, 0.1% SDS, 65° C.
2) medium stringency: 0.2×SSPE, 0.1% SDS, 500° C.
3) low stringency: 1.0×SSPE, 0.1% SDS, 50° C.
   or any combination of salt and temperature and other reagents that result in selection of the same degree of mismatch or matching.

As used herein, all assays and procedures, such as hybridization reactions and antibody-antigen reactions, unless otherwise specified, are conducted under conditions recognized by those of skill in the art as standard conditions.

A. Amplification of Chromosomal Segments and Use Thereof in the Generation of Artificial Chromosomes

The methods, cells and artificial chromosomes provided herein are produced by virtue of the discovery of the existence of a higher-order replication unit (megareplicon) of the centromeric region, including the pericentric DNA, of a chromosome. This megareplicon is delimited by a primary replication initiation site (megareplicator), and appears to facilitate replication of the centromeric heterochromatin, and, most likely, centromeres. Integration of heterologous nucleic acid into the megareplicator region, or in close proximity thereto, initiates a large-scale amplification of megabase-size chromosomal segments. Products of such amplification may be used as artificial chromosomes or in the generation of artificial chromosomes as described herein.

Included among the DNA sequences that may provide a megareplicator are the rDNA units that give rise to ribosomal RNA (rRNA). In plants and animals, particularly mammals such as mice and humans, these rDNA units can contain specialized elements, such as the origin of replication (or origin of bidirectional replication, i.e., OBR, in mouse) and amplification promoting sequences (APS) and amplification control elements (ACE) [see, e.g., with respect to plant rDNA, U.S. Pat. No. 6,096,546 (to Raskin) and U.S. Pat. No. 6,100,092 (to Borysyuk et al.); PCT International Application Publication No. WO99/66058; Genbank Accession no. YO8422 (containing the central AT-rich region of a tobacco rDNA intergenic spacer); Borysyuk et al. (1997) Plant Mol. Biol. 35:655-660); Borysyuk et al. (2000) Nature Biotechnology 18:1303-1306; Hernandez et al. (1993) EMBO J. 12:1475-1485; Van't Hof and Lamm (1992) Plant Mol. Biol. 20:377-382; Hernandez et al. (1988) Plant Mol. Biol. 10:413-322; and with respect to mammalian rDNA, Gogel et al. (1996) Chromosoma 104:511-518; Coffman et al. (1993) Exp. Cell. Res. 209:123-132; Little et al. (1993) Mol. Cell. Biol. 13:6600-6613; Yoon et al. (1995) Mol. Cell. Biol. 15:2482-2489; Gonzalez and Sylvester (1995) Genomics 27:320-328; Miesfeld and Arnheim (1982) Nuc. Acids Res. 10:3933-3949; Maden et al. (1987) Biochem. J. 246:519-527].

As described herein, without being bound by any theory, specialized elements such as these may facilitate replication and/or amplification of megabase-size chromosomal segments in the de novo formation of chromosomes, such as the artificial chromosomes described herein, in cells. These specialized elements are typically located in the nontranscribed intergenic spacer region upstream of the transcribed region of rDNA. The intergenic spacer region may itself contain internally repeated sequences which can be classified as tandemly repeated blocks and nontandem blocks (see e.g., Gonzalez and Sylvester (1995) Genomics 27:320-328). In mouse rDNA, an origin of bidirectional replication may be found within a 3-kb initiation zone centered approximately 1.6 kb upstream of the transcription start site (see, e.g., Gogel et al. (1996) Chromosoma 104:511-518). The sequences of these specialized elements tend to have an altered chromatin structure, which may be detected, for example, by nuclease hypersensitivity or the presence of AT-rich regions that can give rise to bent DNA structures.

Sequences of intergenic spacer regions of plant rDNA include, but are not limited to, sequences contained in GenBank Accession numbers S70723 (from the 5S rDNA of barley (Hordeum vulgare)), AF013103 and X03989 (from maize (Zea mays)), X65489 (from potato (Solanum tuberosum)), X52265 (from tomato (Lycopersicon esculentum)), AF177418 (from Arabidopsis neglecta), AF177421 and AF17422 (from Arabidopsis halleri), A71562, X15550, and X52631 (from Arabidopsis thaliana; see Gruendler et al. (1991) J. Mol. Biol. 221:1209-1222 and Gruendler et al. (1989) Nucleic Acids Res. 17:6395-6396), X54194 (from rice (Oryza sativa)) and Y08422 and D76443 (from tobacco (Nicotiana tabacum). Sequences of intergenic spacer regions of plant rDNA further include sequences from rye (see Appels et al. (1986) Can. J. Genet. Cytol. 28:673-685), wheat (see Barker et al. (1988) J. Mol. Biol. 201:1-17 and Sardana and Flavell (1996) Genome 39:288-292), radish (see Delcasso-Tremousaygue et al. (1988) Eur. J. Biochem. 172:767-776), Vicia faba and Pisum sativum (see Kato et al. (1990) Plant Mol. Biol. 14:983-993), mung bean (see Gerstner et al. (1988) Genome 30:723-733; and Schiebel et al. (1989) Mol. Gen. Genet. 218:302-307), tomato (see Schmidt-Puchta et al. (1989) Plant Mol. Biol. 13:251-253), Hordeum bulbosum (see Procunier et al. (1990) Plant Mol. Biol. 15:661-663) and Lens culinaris Medik, and other legume species (see Fernandez et al. (2000) Genome 43:597-603). Nucleic acids containing intergenic spacer sequences from plants can be obtained by nucleic acid amplification of DNA from plant cells using oligonucleotide primers corresponding to the 3′ end of the conserved 25S mature rRNA encoding region and the 5′ end of the conserved 18S mature rRNA encoding region (see e.g., PCT Application Publication No. WO98/13505).

An exemplary sequence encompassing a mammalian origin of replication is provided in GENBANK accession no. X82564 at about positions 2430-5435. Exemplary sequences encompassing mammalian amplification-promoting sequences include nucleotides 690-1060 and 1105-1530 of GENBANK accession no. X82564 and are also provided in PCT Application Publication No. WO 97/40183. Exemplary sequences encompassing plant amplification-promoting sequences (APS) include those provided in U.S. Pat. No. 6,100,092.

In human rDNA, a primary replication initiation site may be found a few kilobase pairs upstream of the transcribed region and secondary initiation sites may be found throughout the nontranscribed intergenic spacer region (see, e.g., Yoon et al. (1995) Mol. Cell. Biol. 15:2482-2489). A complete human rDNA repeat unit is presented in GENBANK as accession no. U13369. Another exemplary sequence encompassing a replication initiation site may be found within the sequence of nucleotides 35355-42486 in GENBANK accession no. U13369 particularly within the sequence of nucleotides 37912-42486 and more particularly within the sequence of nucleotides 37912-39288 of GENBANK accession no. U 13369 (see Coffman et al. (1993) Exp. Cell. Res. 209:1 23-132).

B. Preparation of Plant Artificial Chromosomes

Cell lines containing artificial chromosomes can be prepared by transforming cells, preferably a stable cell line, with heterologous nucleic acid and identifying cells that contain an artificial chromosome as described herein. The artificial chromosome is a chromosomal structure that is distinct from any chromosome that existed in the cell prior to introduction of the heterologous nucleic acid. A cell containing an artificial chromosome may be identified using a variety of procedures, alone or in combination, as described in detail herein. In particular embodiments of the methods described herein, the heterologous nucleic acid contains a sequence that targets the nucleic acid to an amplifiable region of a chromosome in the cell, such as, for example, the pericentric heterochromatin and/or rDNA. A variety of targeting sequences are provided herein.

Prior to analyzing transformed cells for the presence of an artificial chromosome, the cells to be analyzed may be enriched with artificial chromosome-containing cells using a variety of techniques depending on the heterologous nucleic acid that was introduced into the host cell to initiate generation of the artificial chromosomes. For example, if nucleic acid encoding a selectable marker was included in the heterologous nucleic acid, cells containing the marker may be selected for analysis. If the selectable marker is one that confers resistance to a cytotoxic agent, e.g., bialaphos, hygromycin or kanamycin, the transformed cells may be cultured under selective conditions which include the agent. Cells surviving growth under selective conditions are then analyzed for the presence of artificial chromosomes. If the selectable marker is a readily detectable reporter molecule, such as, for example, a fluorescent protein, the transformed cells may be selected on the basis of fluorescent properties. For example, cells containing the fluorescent protein may be isolated from nontransformed cells using a fluorescence-activated cell sorter (FACS).

In analyzing transformed cells for the presence of artificial chromosomes, it is also possible to identify cells that have a multicentric, typically dicentric, chromosome, formerly multicentric (typically dicentric) chromosome, minichromosome and/or heterochromatic structures, such as a megachromosome and a sausage chromosome. If cells containing multicentric chromosomes or formerly mulitcentric (typically formerly dicentric) chromosomes are initially selected, these cells can then be manipulated, if need be, as described herein to produce the minichromosomes and other artificial chromosomes, particularly the heterochromatic artificial chromosomes and other segmented, repeat region-containing artificial chromosomes, as described herein.

1. Cells Used in the Generation of Plant Artificial Chromosomes

Any cells harboring plant centromere-containing chromosomes may be used in the generation of plant artificial chromosomes (PACs). Such cells include, but are not limited to, plant cells, protoplasts, and cells that are hybrid cells of one or more plant species. Preferred cells are those that harbor plant centromere-containing chromosomes and are readily susceptible to the introduction of heterologous nucleic acids therein.

Cells for use in the generation of plant artificial chromosomes include cells that harbor acrocentric plant chromosomes. Examples of acrocentric plant chromosomes include chromosomes 2 and 4 of the plant Arabidopsis thaliana (see, e.g., Mayer et al. (1999) Nature 402:769-777; Murata et al. (1997) The Plant Journal 12:31-37; The Arabidopsis Genome Initiative (2000) Nature 408:796-815), four acrocentric chromosome pairs in Helianthus annuus (sunflower; see Schrader et al. (1997) Chromosome Res. 5:451-456), two pairs of acrocentric chromosomes in domesticated pepper plant (Capsicum annuum) and a nearly acrocentric chromosome in lentil plant. In particular embodiments of the methods described herein, cells harboring acrocentric plant chromosomes containing rDNA are used in generating plant artificial chromosomes.

Plant species from which cells may be obtained include, but are not limited to, vegetable crops, fruit and vine crops, field plants, bedding plants, trees, shrubs, and other nursery stock. Examples of vegetable crops include artichokes, kohlrabi, arugula, leeks, asparagus, lettuce, bok choy, malanga, broccoli, melons (e.g., muskmelon, watermelon, crenshaw, honeydew, cantaloupe), brussel sprouts, cabbage, cardoni, carots, napa, cauliflower, okra, onions, celery, parsley, chick peas, parsnips, chicory, chinese cabbage, peppers, collards, potatoes, cucumber plants, pumpkins, cucurbits, radishes, dry bulb onions, rutabaga, eggplant, salsify, escarole, shallots, endive, garlic, spinach, green onions, squash, greens, beet, sweet potatoes, swiss chard, horseradish, tomatoes, kale, turnips and spices. Fruit and vine crops include apples, apricots, cherries, nectarines, peaches, pears, plums, prunes, quince, almonds, chestnuts, filberts, pecans, pistachios, walnuts, citrus, blueberries, boysenberries, cranberries, currants, loganberries, raspberries, strawberries, blackberries, grapes, avocados, bananas, kiwi, persimmons, pomegrante, pineapple, tropical fruits, pomes, melon, mango, papaya and lychee.

Field crop plants include evening primrose, meadow foam, corn, maize, hops, jojoba, peanuts, rice, safflower, small grains (barley, oats, rye, wheat, and others) sorghum, tobacco, kapok, leguminous plants (beans, lentils, peas, soybeans), oil plants (canola, rape, mustard, poppy, olives, sunflowers, coconut, castor oil plants, cocoa beans, groundnuts), fibre plants (cotton, flax, hemp, jute), lauraceae (cinnamon, camphor) and plants such as coffee, sugarcane, tea and natural rubber plants. Other examples of plants include bedding plants such as flowers, cactus, succulents and ornamental plants, as well as trees such as forest (broad-leaved trees and evergreens, such as conifers), fruit, ornamental and nut-bearing trees, shrubs, algae, moss, and duckweed.

2. Heterologous Nucleic Acids for Use in Generating Plant Artificial Chromosomes

a. Selectable Markers

The heterologous nucleic acid that is introduced into a cell in the generation of artificial chromosomes as described herein may include nucleic acid encoding a selectable marker. Any nucleic acid that includes a selectable marker sequence may be introduced into cells harboring plant centromere-containing chromosomes for the generation of plant artificial chromosomes. Examples of selectable markers include, but are not limited to, DNA encoding a product that confers resistance to a cytotoxic or cytostatic agent and DNA encoding a readily detectable product, such as a reporter protein.

### (1) Nucleic Acids Encoding Products that Confer Resistance to a Selection Agent

Examples of selectable markers include the dihydrylfolate reductase (dhfr) gene, hygromycin phosphotransferase genes, the phosphinothricin acetyl transferase gene (bar gene) and neomycin phosphotransferase genes. Selectable markers that can be used in animal, e.g., mammalian cells include, but are not limited to the thymidine kinase gene and the cellular adenine-phosphribosyltransferase gene.

Of particular interest for purposes herein are nucleic acid selectable markers that, upon expression in the host cell, confer antibiotic or herbicide resistance to the cell, sufficient to provide for the maintenance of heterologous nucleic acids in the cell, and which facilitate the transfer of artificial chromosomes containing the marker DNA into new host cells. Examples of such markers include DNA encoding products that confer cellular resistance to hygromycin, kanamycin, G418, bialaphos, Basta, methotrexate, glyphosate, and puromycin. For example, neo (or nptII) provides kanamycin resistance and can be selected for using kanamycin, G418, paromomycin and other agents [see, e.g., Messing and Vierra (1982) Gene 19:259-268; and Bevan et al. (1983) Nature 304:184-187]; bar from Steptomyces hygroscopicus, which encodes the enzyme phosphinothricin acetyl transferase (PAT) confers bialaphos, glufosinate, Basta or phosphinothricin resistance [see e.g., White et al. (1990) Nuc. Acids Res. 18:1062; Spencer et al. (1990) Theor. Appl. Genet. 79:625-631; Vickers et al. (1996) Plant Mol. Biol. Reporter 14:363-368; and Thompson et al. (1987) EMBO J. 6:2519-25231; the hph gene which confers resistance to the antibiotic hygromycin (see, e.g., Blochinger and Diggelmann, Mol. Cell. Biol. 4:2929-2931); a mutant EPSP synthase protein [see Hinchee et al. (1988) Bio/technol 6:915-922] confers glyphosate resistance (see also U.S. Pat. Nos. 4,940,935 and 5,188,642); and a nitrilase such as bxn from Klebsiella ozaenae confers resistance to bromoxynil [see Stalker et al. (1988) Science 242:419-42]. DNA encoding cystathionine gamma-synthase (CGS) can be used as a marker that confers resistance to ethionine (see PCT Application Publication No. WO 00/55303). Examples of markers that can be used in animal, e.g., mammalian cells, include but are not limited to DNA encoding products that confer cellular resistance to streptomycin, zeocin, chloramphenicol and tetracycline.

### (2) Reporter Molecules

Nucleic acids encoding reporter molecules may also be included in the nucleic acid that is introduced into a recipient cell in the generation of artificial chromosomes. Reporter genes provide a means for identifying cells and chromosomes into which heterologous nucleic acids have been transferred and further provide a means for assessing whether or not, and to what extent, transferred DNA is expressed.

Nucleic acids encoding reporter molecules that may be used in monitoring transfer and expression of heterologous nucleic acids into cells, particularly plant cells include, but are not limited to, nucleic acid encoding β-glucuronidase (GUS) or the uidA gene product, which is an enzyme for which various chromogenic substrates are known [see Novel and Novel (1973) Mol. Gen. Genet. 120:319-335; Jefferson et al. (1986) Proc. Natl. Acad. Sci. USA 83:8447-8451; U.S. Pat. No. 5,268,463; commercially available from Clontech Laboratories, Palo Alto, Calif.], DNA from an R-locus gene, which encodes a product that regulates the production of anthocyanin pigments (red color) in plant tissues [see, e.g., Dellaporta et al. (1988) In “Chromosome Structure and Function: Impact of New Concepts, 18th Stadler Genetics Sympsium” 11:263-282], nucleic acid encoding β-lactamase [Sutcliffe (1978) Proc. Natl. Acad. Sci. U.S.A. 75:3737-3741] which is an enzyme for which various chromogenic substrates are known (e.g., PADAC, a chromogenic cephalosporin), DNA from a xy/E gene [see, e.g., Zukowsky et al. (1983) Proc. Natl. Acad. Sci. U.S.A. 80:1101-1105], which encodes a catechol dioxygenase that can convert chromogenic catechols; nucleic acid encoding α-amylase [see, e.g., Ikuta et al. (1990) Bio/technol. 8:241-242], nucleic acid encoding tyrosinase [see, e.g., Katz et al. (1983) J. Gen. Microbiol. 129:2703-2714], an enzyme capable of oxidizing tyrosine to DOPA and dopaquinone which in turn condenses to form the readily detectable compound melanin, nucleic acid encoding β-galactosidase, an enzyme for which there are chromogenic substrates, nucleic acid encoding luciferase (lux) gene [see, e.g., Ow et al. (1986) Science 234:856-859] which allows for bioluminesence detection, nucleic acid encoding aequorin [see, e.g., Prasher et al. (1985) Biochem. Biophy. Res. Commun. 126:1259-1268] which may be employed in calcium-sensitive bioluminescence detection, nucleic acid encoding a green fluorescent protein (GFP) [see, e.g., Sheen et al. (1995) Plant J. 8:777-784; Haselhoff et al. (1997) Proc. Natl. Acad. Sci. U.S.A. 94:2122-2127; Hasseloff and Amos (1995) Trends Genet 11:328-329; Reichel et al. (1996) Proc. Natl. Acad. Sci. U.S.A. 93:5888-5893; Tian et al. (1997) Plant Cell Rep. 16:267-271; Prasher et al. (1992) Gene 111:229-233; Chalfie et al. (1994) Science 263:802; PCT Application Publication Nos. WO97/41228 and WO 95/07463; and commercially available from Clontech Laboratoreis, Palo Alto, Calif.), nucleic acid encoding a red or blue fluorescent protein (RFP or BFP, respectively), or nucleic acid encoding chloramphenicol acetyltransferase (CAT).

Enhanced GFP (EGFP) is a mutant of GFP with a 35-fold increase in fluorescence. This variant has mutations of Ser to Thr at amino acid 65 and Phe to Leu at position 64 and is encoded by a gene with optimized human codons (see, e.g., U.S. Pat. No. 6,054,312). EGFP is a red-shifted variant of wild-type GFP (Yang et al. (1996) Nucl. Acids Res. 24:4592-4593; Haas et al. (1996) Curr. Biol. 6:315-324; Jackson et al. (1990) Trends Biochem. 15:477-483) that has been optimized for brighter fluorescence and higher expression in mammalian cells (excitation maximum=488 nm; emission maximum=507 nm). EGFP encodes the GFPmut1 variant (Jackson (1990) Trends Biochem. 15:477-483) which contains the double-amino-acid substitution of Phe-64 to Leu and Ser-65 to Thr. Sequences flanking EGFP have been converted to a Kozak consensus translation initiation site (Huang et al. (1990) Nucleic Acids Res. 18: 937-947) to further increase the translation efficiency in eukaryotic cells.

Nucleic acid from the maize R gene complex can also be used as nucleic acid encoding a reporter molecule. The R gene complex in maize encodes a protein that acts to regulate the production of anthocyanin pigments in most seed and plant tissue. Maize strains can have one, or as many as four, R alleles which combine to regulate pigmentation in a developmental and tissue-specific manner. Thus, an R gene introduced into such cells will cause the expression of a red pigment and, if stably incorporated, can be visually scored as a red sector. If a maize line carries dominant alleles for genes encoding for the enzymatic intermediates in the anthocyanin biosynthetic pathway (C2, A1, A2, Bz1 and Bz2), but carries a recessive allele at the R locus, the transformation of any cell from that line with R will result in red pigment formation. Exemplary lines include Wisconsin 22 which contains the rg-Stadler allele and TR112, a K55 derivative which is r-g, b, Pl. Alternatively, any genotype of maize can be utilized if the C1 and R alleles are introduced together.

b. Promoters and Other Sequences that Influence Gene Expression

Expression of nucleic acid encoding a selectable marker (or any heterologous nucleic acid) in a recipient cell can be regulated by a variety of promoters. Promoters for use in regulating transcription of DNA in cells, particularly plant cells, include, but are not limited to, the nopaline synthase (NOS) and octopine synthase (OCS) promoters, cauliflower mosaic virus (CaMV) 19S and 35S promoters, the light-inducible promoter from the small subunit of ribulose bis-phosphate carboxylase (ssRUBISCO, an abundant plant polypeptide), the mannopine synthase (MAS) promoter [see, e.g., Velten et al. (1984) EMBO J. 3:2723-2730; and Velten and Schell (1985) Nuc. Acids Res. 13:6981-6998], the rice actin promoter, the ubiquitin promoter, for example, from Z. mays (see e.g., PCT Application Publication No. WO00/60061), Arabidopsis thaliana UBI 3 promoter [see e.g., Norris et al. (1993) Plant Mol. Biol. 22:895-906] and the chemically inducible PR-1 promoter from tobacco or Arabidopsis (see e.g., U.S. Pat. No. 5,689,044).

Selection of a suitable promoter may include several considerations, for example, recipient cell type (such as, for example, leaf epidermal cells, mesophyll cells, root cortex cells), tissue- or organ-specific (e.g., roots, leaves or flowers) expression of genes linked to the promoter, and timing and level of expression (as may be influenced by constitutive vs. regulatable promoters and promoter strength).

Additional sequences that may also be included in the nucleic acid containing a selectable marker include, but are not restricted to, transcription terminators and extraneous sequences to enhance expression such as introns. A variety of transcription terminators may be used which are responsible for termination of transcription beyond a coding region and correct polyadenylation. Appropriate transcription terminators include those that are known to function in plants such as, for example, the CaMV 35S terminator, the tml terminator, the nopaline synthase terminator and the pea rbcS E9 terminator, all of which may be used in both monocotyledonous and dicotyledonous plants.

Numerous sequences have been found to enhance gene expression from within the transcriptional unit and these sequences can be used in conjunction with selectable marker and other genes to increase expression of the genes in plant cells. For example, various intron sequences such as introns of the maize AdhI gene have been shown to enhance expression, particularly in monocotyledonous cells. In addition, a number of non-translated leader sequences derived from viruses are also known to enhance exprssion, and these are particularly effective in dicotyledonous cells.

C. Nucleic Acids Containing Targeting Sequences

Development of a multicentric, particularly dicentric, chromosome typically is effected through integration of heterologous nucleic acid into heterochromatin, such as the pericentric heterochromatin, near or within the centromeric regions of chromosomes and/or into rDNA sequences. Thus, the development of artificial chromosomes may be facilitated by targeting the heterologous nucleic acid for integration into these regions, such as by introducing DNA, including, but not limited to, rDNA (e.g., rDNA intergenic spacer sequence), satellite DNA, pericentric DNA and lambda phage DNA, into the recipient host cell. The targeting sequence may be introduced alone or with other nucleic acids, including but not limited to selectable markers. For example, a targeting sequence can be linked to a selectable marker.

Examples of plant pericentric DNA and satellite DNA include, but are not limited to, pericentromeric sequences on tomato chromosome 6 [see, e.g., Weide et al. (1998) Mol. Gen. Genet. 259:190-197], satellite DNA of soybean [see, e.g., Morgante et al. (1997) Chromosome Res. 5:363-373; and Vahedian et al. (1995) Plant Mol. Biol. 29:857-862], pericentromeric DNA of Arabidopsis thaliana [see, e.g., Tutois et al. (1999) Chromosome Res. 7:143-156], satellite DNA of arabidopsis thaliana (GenBank accession nos. AB033593 and X58104), pericentric DNA of the chickpea [Cicer arietinum L.; see e.g., Staginnus et al. (1999) Plant Mol. Biol. 39:1037-1050], satellite DNA on the rye B chromosome [see, e.g., Langdon et al. (2000) Genetics 154:869-884], subtelomeric satellite DNA from Silene latifolia [see, e.g., Garrido-Ramos et al. (1999) Genome 42:442-446] and satellite DNA in the Saccharum complex [see, e.g., Alix et al. (1998) Genome 41:854-864].

Examples of rDNA targeting sequences include nucleic acids from plant and animal rDNA. Plant rDNA sequences include, but are not limited to, sequences contained in GENBANK Accession numbers D16103 [from rDNA of carrot (Daucus carota)], M23642 and M11585 [from rDNA encoding 24S rRNA of rice (Oryza sativa)], M26461 [from from rDNA encoding 18S rRNA of rice (Oryza sativa)], M16845 [from rDNA encoding 17S, 5.8S and 25S rRNA of rice (Oryza sativa)], X82780 and X82781 [from rDNA encoding 5S rRNA of potato (Solanum tuberosum)], AJ131161, AJ131162, AJ131163, AJ131164, AJ131165, AJ131166 and AJ 131167 [from rDNA encoding 5S rRNA of tobacco (Nicotiana tabacum], L36494 and U31016 through U31030 [from rDNA encoding 5S rRNA of barley (Hordeum spontaneum)], U31004 through U31015 and U31031 [from rDNA encoding 5S rRNA of barley (Hordeum bulbosum)], Z 11759 [from rDNA encoding 5.8S rRNA of barley (Hordeum vulgare)], X16077 (from rDNA encoding 18S rRNA of Arabidopsis thaliana), M65137 (rDNA encoding 5S rRNA of Arabidopsis thaliana), AJ232900 (from rDNA encoding 5.8S rRNA of Arabidopsis thaliana) and X52320 (from Arabidopsis thaliana genes for 5.8S and 25S rRNA with an 18S rRNA fragment).

Intergenic spacer regions of plant rDNA include, but are not limited to sequences contained in GENBANK Accession numbers S70723 (from the 5S rDNA of barley (Hordeum vulgare)), AF013103 and X03989 (from maize (Zea mays)), X65489 (from potato (Solanum tuberosum)), X52265 (from tomato (Lycopersicon esculentum)), AF177418 (from Arabidopsis neglecta), AF177421 and AF17422 (from Arabidopsis halleri), A71562, X15550, X52631, U43224, X52320, X52636 and X52637 (from Arabidopsis thaliana; see Gruendler et al. (1991) J. Mol. Biol. 221:1209-1222 and Gruendler et al. (1989) Nucleic Acids Res. 17:6395-6396), X54194 [from rice (Oryza sativa)] Y08422 and D76443 [from tobacco (Nicotiana tabacum)], AJ243073 [from wheat (Triticum boeoticum)] and X07841 [from wheat (Triticum aestivum)]. Sequences of intergenic spacer regions of plant rDNA further include sequences from rye [see Appels et al. (1986) Can. J. Genet. Cytol. 28:673-685], wheat [see Barker et al. (1988) J. Mol. Biol. 201:1-17 and Sardana and Flavell (1996) Genome 39:288-292], radish [see Delcasso-Tremousaygue et al. (1988) Eur. J. Biochem. 172:767-776], Vicia faba and Pisum sativum [see Kato et al. (1990) Plant Mol. Biol. 14:983-993], mung bean [see Gerstner et al. (1988) Genome 30:723-733; and Schiebel et al. (1989) Mol. Gen. Genet. 218:302-307], tomato [see Schmidt-Puchta et al. (1989) Plant Mol. Biol. 13:251-253], Hordeum bulbosum [see Procunier et al. (1990) Plant Mol. Biol. 15:661-663], Lens culinaris Medik., and other legume species [see Fernandez et al. (2000) Genome 43:597-603] and tobacco [see U.S. Pat. Nos. 6,100,092 and 6,096,546 and PCT Application Publication No. WO99/66058; Borysyuk et al. (1997) Plant Mol. Biol. 35:655-660); Borysyuk et al. (2000) Nature Biotechnology 18:1303-1306].

Mammalian rDNA sequences include, but are not limited to, DNA of GENBANK accession no. X82564 and portions thereof, the DNA of GENBANK accession no. U13369 and portions thereof and DNA sequences provided in PCT Application Publication No. WO97/40183 (particularly SEQ. ID. NOS. 18-24 of WO97/40183). A particular vector for use in directing integration of heterologous nucleic acid into chromosomal rDNA is pTERPUD (see PCT Application Publication No. WO97/40183). Satellite DNA sequences can also be used to direct the heterologous DNA to integrate into the pericentric heterochromatin. For example, vectors pTEMPUD and pHASPUD, which contain mouse and human satellite DNA, respectively (see PCT Application Publication No. WO97/40183), are examples of vectors that may be used for introduction of heterologous nucleic acid into cells for de novo chromosome formation leading to artificial chromosomes. 3. Methods for Introduction of Heterologous Nucleic Acids into Host Cells

Any methods known in the art for introducing heterologous nucleic acids into host cells may be used in the methods of preparing artificial chromosomes. The particular method used may depend on the type of cell into which the heterologous nucleic acid is being transferred. For example, methods for the physical introduction of nucleic acids into plant cells, for example, protoplasts and plant cells in culture, include, but are not limited to polyethylene glycol (PEG)-mediated DNA uptake, electroporation, lipid-mediated delivery, including liposomes, calcium phosphate-mediated DNA uptake, microinjection, particle bombardment, silicon carbide whisker-mediated transformation and combinations of these methods, for example methods utilizing combinations of calcium phosphate and PEG for DNA uptake or methods utilizing a combination of electroporation, PEG and heat shock (see, e.g., U.S. Pat. Nos. 5,231,019 and 5,453,367). Physical methods such as these are known in the art and are effective in introducing DNA into a variety of dicotyledonous and monocotyledonous plants [see, e.g., Paszkowski et al. (1984) EMBO J. 3:2717-2722; Potrykus et al. (1985) Mol. Gen. Genet. 199:169-177; Reich et al. (1986) Biotechnology 4:1001-1004; Klein et al. (1987) Nature 327:70-73; U.S. Pat. No. 6,143,949; Paszkowski et al. (1989) in Cell Culture and Somatic Cell Genetics of Plants, Vol. 6, Molecular Biology of Plant Nuclear Genes, eds. Schell, J and Vasil, L. K. Academic Publishers, San Diego, Calif., p. 52-68; and Frame et at. (1994) Plant J. 6:941-948].

In addition to these methods for the introduction of nucleic acids into plant cells based on physically, mechanically or chemically meidated processes, it is possible to introduce nucleic acids into plant cells by biological methods, such as those utilizing Agrobacterium. In this method, nucleic acid sequences located adjacent to T-DNA border repeats can be inserted into the genome of a plant cell, typically dicotyledonous plant cells, by utilizing the encoded function for DNA transfer found in the genus Agrobacterium. This method has also been shown to work for some monocotyledonous plant cells, such as rice cells.

Any method for introducing nucleic acids into plant cells can be used in the generation of artificial chromosomes, provided the method is capable of introducing the nucleic acid into an amplifiable region of a chromosome, for example, heterochromatin, and particularly in close proximity to a megareplicator region of a plant chromosome.

a. Agrobacterium-Mediated Introduction of Nucleic Acids into Plant Cells

Agrobacterium-mediated transformation is particularly well-suited for transformation of dicotyledons because of its high efficiency of transformation and its broad utility with many different species, including tobacco, tomato (see, e.g., European Patent Application no. 0 249 432), sunflower, cotton (see, e.g., European Patent Application no. 0 317 511), oilseed rape, potato, soybean, alfalfa and poplar (see, e.g., U.S. Pat. No. 4,795,855) (see also PCT Application Publication no. WO87/07299 with respect to transformation of Brassica). Agrobacterium-mediated transformation has also been used to transfer nucleic acids into monocotyledonous plants. Agrobacterium-mediated transformation of Chlorophytum capense and Narcissus cv “Paperwhite” [see, e.g., Hooykaas-Van Slogteren et al. (1984) Nature 311:763-764], corn and wheat [see, e.g., U.S. Pat. Nos. 5,164,310, 5,187,073 and 5,177,010 and Mooney et al. (1991) Plant Cell, Tissue, Organ Culture 25:209-218], rice [see, e.g., Raineri et al. (1990) Bio/Technology 8:33-38 and Chan et al. (1993) Plant Mol. Biol. 22:491-506] and barley [see, e.g., Tingay et al. (1997) The Plant J. 11:1369-1376 and Qureshi et al. (1998) Proc. 42nd Conference of Australian Society for Biochemistry and Molecular Biology, Sep. 28-Oct. 1, 1998, Adelaide Australia] has been reported.

Agrobacterium-mediated delivery of nucleic acids is based on the capacity of certain Agrobacterium strains to introduce a part of their Ti (tumor-inducing) plasmid, i.e., the transforming DNA or T-DNA, into plant cells and to integrate this T-DNA into the genome of the cells. The part of the Ti plasmid that is transferred and integrated is delineated by specific DNA sequences, the left and right T-DNA border sequences. The natural T-DNA sequences between these border sequences can be replaced by foreign DNA [see, e.g., European Patent Publication 116 718 and Deblaere et al. (1987) Meth. Enzymol. 153:277-293].

When Agrobacterium is used for transformation, the heterologous nucleic acid being transferred typically is cloned into a plasmid that contains T-DNA border regions and is replicated independently of the Ti plasmid (referred to as the binary vector system) or the heterologous nucleic acid is inserted between the T-DNA borders of the Ti plasmid (referred to as the co-integrate method). In co-integrate methods, these vectors are be integrated into the Ti or Ri plasmid by homologous recombination owing to sequences that are homologus to sequences within the T-DNA region of the Ti or Ri plasmid. The Ti or Ri plasmid also contains the vir region necessary for transfer of the T-DNA.

Intermediate vectors cannot replicate in Agrobacteria. The intermediate vector can be transferred into Agrobacterium by means of a helper plasmid (conjugation, see Fraley et al. (1983) Proc. Natl. Acad. Sci. USA 80:4803). This method, typically referred to as triparental mating, introduces the heterologous nucleic acid sequence into the bacterium and allows for selection of a homologous recombination event that produces the desired Agrobacterium genotype. The triparental mating procedure typically employs Escherichia coli carrying the recombinant intermediate vector and a helper E. coli strain which carries a plasmid that is able to mobilize the recombinant intermediate vector to the target Agrobacterium strain. A modified Ti or Ri plasmid is obtained from the transfer and selection process, which contains a heterologous nucleic acid sequence located within the T-DNA region. The resultant Agrobacterium strain is capable of transferring the heterologous nucleic acid to plant cells.

Binary vectors can replicate both in E. coli and Agrobacterium. They typically contain a selection marker gene and a linker or polylinker which are flanked by the right and left T-DNA border regions and can be transformed directly into Agrobacterium [see, e.g., Hofgen and Wilmitzer (1988) Nuc. Acids. Res. 16:9877 and Holsters et al. (1978) Mol. Gen. Genet. 163:181-187] or introduced through triparental mating. The Agrobacterium host cell contains a plasmid carrying a vir region needed for transfer of the T-DNA into a plant cell [see, e.g., White in Plant Biotechnology, eds. Kung, S. and Arntzen, C. J., Butterworth Publishers, Boston, Mass., (1989) p. 3-34 and Fraley in Plant Biotechnology, eds. Kung, S. and Arntzen, C. J., Butterworth Publishers, Boston, Mass., (1989) p. 395-407].

Agrobacterium-mediated transformation typically involves the transfer of a binary vector carrying the heterologous nucleic acid of interest to an appropriate Agrobacterium strain, which may depend on the complement of vir genes carried by the host Agrobacterium strain either on a co-resident Ti plasmid or chromosomally (see, e.g., Uknes et al. (1993) Plant Cell 5:159-169). The transfer of a recombinant binary vector to Agrobacterium is acomplished by a triparental mating procedure using Eschreichia coli carrying the recombinant binary vector, a helper E. coli strain which carries a plasmid which is able to mobilize the recombinant binary vector to the target Agrobacterium strain. Alternatively, the recombinant binary vector can be transferred to Agrobacterium by DNA transformation (see, e.g., Hofgen & Willmitzer (1988) Nuc. Acids. Res. 16:9877).

Many vectors are available for transfer of nucleic acids into Agrobacterium tumefaciens [see, e.g., Rogers et al. (1987) Methods in Enzymol. 153:253-277]. These typically carry at least one T-DNA border sequence and include vectors such as pBIN19 [see, e.g., Bevan (1984) Nuc. Acids. Res. 12:8711-8721]. Typical vectors suitable for Agrobacterium transformation include the binary vectors pCIB200 and pCIB2001, as well as the binary vector pCIB10 and hygromycin selection derivatives thereof (see, e.g., U.S. Pat. No. 5,639,949). Other vectors that can be employed are the pCambia vectors (see www.cambia.org), including, for example, pCambia 3300 and pCambia 1302 (GenBank Accession No. AF234298).

A particularly useful Ti plasmid cassette vector for the transformation of dicotyledonous plants contains the enhanced CaMV35S promoter (EN35S) and the 3′ end, including polyadenylation signals, of a soybean gene encoding the a subunit of β-conglycinin. Between these two elements is a multilinker containing multiple restriction sites for the insertion of genes of interest (see, e.g., U.S. Pat. No. 6,023,013). The vector can contain a segment of pBR322 which provides an origin of replication in E. coi and a region for homologous recombination with the disarmed T-DNA in Agrobacterium strain ACO; the oriV region from the broad host range plasmid RK1; the streptomycin/spectinomycin resistance gene from Tn7; and a chimeric NPTII gene, containing the CaMV35S promoter and the nopaline synthase (NOS) 3′ end, which provides kanamycin resistance in transformed plant cells. Optionally, the enhanced CaMV35S promoter may be replaced with the 1.5 kb mannopine synthase (MAS) promoter (see, e.g., Velton et al. (1984) EMBO J. 3:2723-2730). After incorporation of a DNA construct into the vector, it is introduced into A. tumefaciens strain ACO which contains a disarmed Ti plasmid. Cointegrate Ti plasmid vectors are selected and subsequentally may be used to transform a dicotyledenous plant.

Transformation of the target plant species by recombinant Agrobacterium usually involves co-cultivation of the Agrobacterium with explants from the plant and follows published protocols. Methods of inoculation of the plant tissue vary depending upon the plant species and the Agrobacterium delivery system. The plant tissue can be either protoplast, callus or organ tissue, depending on the plant species. A widely used approach is the leaf disc procedure which can be performed with any tissue explant that provides a good source for initiation of whole plant differentiation (see, e.g., Horsch et al. in Plant Molecular Biology Manual A5, Kluwer Academic Publishers, Dordrecht (1988) p. 1-9 and U.S. Pat. No. 6,136,320). The addition of nurse tissue may be desirable under certain conditions. There are multiple choices of Agrobacterium strains (including, but not limited to, A. tumefaciens and A. rhizogenes) and plasmid construction strategies that can be used to optimize genetic transformation of plants. Transformed tissue carrying an antibiotic or herbicide resistance marker present between the binary plasmid and T-DNA borders can be regenerated on selectable medium.

A. tumefaciens ACO is a disarmed strain similar to pTiB6SE (see Fraley et al. (1985) Bio/Technology 3:629-635). For construction of ACO, the starting Agrobacterium strain was A208 which contains a nopaline-type Ti plasmid. The Ti plasmid was disarmed in a manner similar to that described by Fraley et al. (1985) Bio/Technology 3:629-635) so that essentially all of the native T-DNA was removed except for the left border and a few hundred base pairs of T-DNA inside the left border. The remainder of the T-DNA extending to a point just beyond the right border was replaced with a piece of DNA including (from left to right) a segment of pBR322, the oriV region from plasmid RK2, and the kanamycin resistance gene from Tn601. The pBR322 and oriV segments are similar to these segments and provide a region of homology for cointegrate formation (see U.S. Pat. No. 6,023,013). Another useful strain of Agrobacterium is A. tumefaciens strain GV3101/pMP90 [see, e.g., Koncz and Schell (1986) Mol. Gen. Genet. 204:383-396].

Advances in Agrobacterium-mediated transfer allow introduction of larger segments of nucleic acids [see, e.g., Hamilton (1997) Gene 4:200(1-2):107-116; Hamilton et al. (1996) Proc. Natl. Acad. Sci. U.S.A. 93:9975-9979; Liu et al. (1999) Proc. Natl. Acad. Sci. U.S.A. 96:6535-6540]. The vectors used in these methods are designed to have the characteristics of both bacterial artificial chromosomes (BACs) and binary vectors for Agrobacterium-mediated transformation. Therefore, somewhat larger DNA fragments cloned in the T-DNA region can be transferred into a plant genome by Agrobacterium. Binary bacterial artificial chromosome (BIBAC) vector BIBAC2 (see U.S. Pat. No. 5,733,744; available from the Plant Science Center, Cornell University) and the transformation-competent bacterial artificial chromosome (TAC) vector pYLTAC7 (available from the Plant Cell Bank of the RIKEN Gene Bank, Tsukuba, Japan) are examples of the types of vectors that may be used in transferring larger segments of nucleic acids, particularly heterologous nucleic acids containing targeting and/or selectable marker sequences as described herein, into plants via Agrobacterium-mediated DNA transfer processes.

Introduction of heterologous nucleic acids into plant cells without the use of Agrobacterium circumvents the requirements for T-DNA sequences in the transformation vector and consequently vectors lacking these sequences can be utilized in addition to vectors containing T-DNA sequences. Techniques for nucleic acid transfer that do not rely on Agrobacterium include transformation via particle bombardment, direct DNA uptake (e.g., PEG, lipids, electroporation) and mechanical methods such as microinjection or silicon “whiskers”. The choice of vector that may be used in introduction of heterologous nucleic acids into plant cells can involve largely on the preferred selection for the species being transformed. Typical vectors suitable for transformation without Agrobacterium include pCIB3064, pSOG19 and pSOG35 (see, e.g., U.S. Pat. No. 5,639,949), or common plasmid, phage or cosmid vectors.

b. Direct DNA Uptake

Introduction of heterologous nucleic acids into plant cells may be achieved using a variety of methods that facilitate direct DNA uptake, including calcium phosphate precipitation, polyethylene glycol (PEG) treatment, electroporation, and combinations thereof [see, e.g., Potrykus et al. (1985) Mol. Gen. Genet. 199:183; Lorz et al. (1985) Mol. Gen. Genet. 199:178; Fromm et al. (1985) Proc. Natl. Acad. Sci. U.S.A. 82:5824-5828; Uchimiya et al. (1986) Mol. Gen. Genet. 204:204; Callis et al. (1987) Genes Dev. 1:1183-2000; Callis et al. (1987) Nuc. Acids Res. 15:5823-5831; Marcotte et al. (1988) Nature 355:454, Toriyama et al. (1988) Bio/Technology 6:1072-1074; Haim et al. (1985) Mol. Gen. Genet, 199:161-168; Deshayes et al. (1985) EMBO J. 4:2731-2737; Krens et al. (1982) Nature 296:72-74; Crossway et al. (1986) Mol. Gen. Genet. 20:179].

Typically, plant protoplasts are used for direct DNA uptake, or in some instances plant tissue that has been treated to remove a portion or the majority of the cell wall (see, e.g., PCT Publication No. WO93/21335 and U.S. Pat. No. 5,472,869). Removal of the cell wall is believed to facilitate entry of DNA into plant cells, although in some instances electroporation may be used to introduce DNA into specialized plant cells, e.g., electroporation of pollen, without first removing the cell wall.

Techniques for the preparation of callus and protoplasts from maize, transformation of protoplasts using PEG or electroporation, and the regeneration of maize plants from transformed protoplasts are found, for example, in European Patent Application nos. 0 292 435 and 0 392 225 and PCT Application Publication no. WO93/07278. Transformation of rice can also be undertaken by direct gene transfer techniques utilizing protoplasts [see, e.g., Zhang et al. (1988) Plant Cell Rep. 7:379-384; Shimamoto et al. (1989) Nature 338:274-277; Datta et al. (1990) Biotechnology 8:736-740]. The regeneration of fertile transgenic barley by direct DNA transfer to protoplasts is described, for example, by Funatsuki et al. [(1995) Theor. Appl. Genet. 91:707-712]. Other plant species, including tobacco and Arabidopsis, may also serve as sources of protoplasts for use in introduction of heterologous nucleic acids into plant cells.

C. Particle Bombardment-m Diated Introduction of Nucleic Acids into Plant Cells

Microprojectile bombardment of plant cells can be an effective method for the introduction of nucleic acids into plant cells. In these methods, nucleic acids are carried through the cell wall and into the cytoplasm on the surface of small, typically metal, particles [see, e.g., Klein et al. (1987) Nature 327:70; Klein et al. (1988) Proc. Natl. Acad. Sci. U.S.A. 85:8502-8505, Klein et al. in Progress in Plant Cellular and Molecular Biology, eds. Nijkamp, H. J. J., Van der Plas, J. H. W., and Van Aartrijk, J., Kluwer Academic Publishers, Dordrecht, (1988), p. 56-66; Seki et al. (1999) Mol. Biotechnol. 11:251-255; and McCabe et al. (1988) Bio/Technology 6:923-926]. Particles may be coated with nucleic acids and delivered into cells by a propelling force. Exemplary particles include those containing tungsten, gold or plantinum, as well as magnesium sulfate crystals. The metal particles can penetrate through several layers of cells and thus allow the transformation of cells within tissue explants.

In an illustrative embodiment [see, e.g., U.S. Pat. No. 6,023,013] of a method for delivering nucleic acids into plant cells, e.g., maize cells, by acceleration, a Biolistics Particle Delivery System may be used to propel particles coated with DNA or cells through a screen, such as a stainless steel or Nytex screen, onto a filter surface covered with plant (e.g., corn) cells cultured in suspension. The screen disperses the particles so that they are not delivered to the recipient cells in large aggregates. The intervening screen between the projectile apparatus and the cells to be bombarded may reduce the size of projectile aggregates and may contribute to a higher frequency of transformation by reducing damage inflicted on the recipient cells by projectiles that are too large.

For the bombardment, cells in suspension may be concentrated on filters or solid culture medium. Alternatively, immature embryos or other target cells may be arranged on solid culture medium. The cells to be bombarded are typically positioned at an appropriate distance below the macroprojectile stopping plate. If desired, one or more screens may also be positioned between the acceleration device and the cells to be bombarded.

The prebombardment culturing conditions and bombardment parameters may be optimized to yield the maximum numbers of stable transformants. Both the physical and biological parameters for bombardment can be important in this technology. Physical factors include those that involve manipulating the DNA/microprojectile precipitate or those that affect the flight and velocity of either the macro-or microprojectiles. Biological factors include all steps involved in manipulation of cells before and immediately after bombardment, the osmotic adjustment of target cells to help alleviate the trauma associated with bombardment, and also the nature of the transforming nucleic acid, such as linearized DNA or intact supercoiled plasmids.

Physical parameters that may be adjusted include gap distance, flight distance, tissue distance and helium pressure. In addition, transformation may be optimized by adjusting the osmotic state, tissue hydration and subculture stage or cell cycle of the recipient cells.

Techniques for transformation of A188-derived maize line using particle bombardment are desribed in Gordon-Kamm et al. [(1990) Plant Cell 2:603-618] and Fromm et al. [(1990) Biotechnology 8:833-839]. Transformation of rice may also be accomplished via particle bombardment [see, e.g., Christou et al. (1991) Biotechnology 9:957-962]. Particle bombardment may also be used to transform wheat [see, e.g., Vasil et al. (1992) Biotechnology 10:667-674 for transformation of cells of type C long-term regenerable callus; and Weeks et al. (1993) Plant Physiol. 102:1077-1084 for transformation of wheat using particle bombardment of immature embryos and immature embryo-derived callus]. The production of transgenic barley using bombardment methods is described, for example, by Koprek et al. [(1996) Plant Sci. 119:79-91].

d. Electroporation-Mediated Introduction of Nucleic Acids into Plant Cells

The application of brief, high-voltage electric pulses to a variety of animal and plant cells leads to the formation of nanometer-sized pores in the plasma membrane. Nucleic acids are taken directly into the cell cytoplasm either through these pores or as a consequence of the redistribution of membrane components that accompanies closure of the pores. Electroporation can be extremely efficient and can be used both for transient expression of cloned genes and for the establishment of cell lines that carry integrated copies of the gene of interest.

Certain cell wall-degrading enzymes, such as pectin-degrading enzymes, may be employed to render the target recipient cells more susceptible to transformation by electroporation than untreated cells. Alternatively, recipient cells may be more susceptible to transformation by mechanical wounding. To effect transformation by electroporation, friable tissues such as a suspension culture of cells or embryonic callus may be used or immature embryos or other organized tissues may be directly transformed [see, e.g., Fromm et al. (1986) Nature 319:791-793; and Neuman et al. (1982) EMBO J. 1:841-845].

e. Microinjection-Mediated Introduction of Nucleic Acids into Plant Cells

In microinjection techniques, nucleic acids are mechanically injected directly into cells using very small micropipettes. For example, microinjection of protoplast cells with foreign DNA for transformation of plant cells has been reported for barley and tobacco [see, e.g., Holm et al. (2000) Transgenic Res. 9:21-32 and Schnorf et al. Transgenic Res. 1:23-30].

f. Lipid-Mediated Introduction of Nucleic Acids into Plant Cells

In lipid-mediated transfer, nucleic acids are contacted with lipids and/or encapsulated in lipid-containing structures, including but not limited to liposomes, and the liposome-containing nucleic acids are fused with plant protoplasts. The fusion can occur in the presence or absence of a fusogen, such as PEG. Lipid-mediated transformation of plant protoplasts has been reported [see e.g., Fraley and Papahadjopoulos (1982) Curr. Top. Microbiol. Immunol. 96:171-191; Deshayes et al. (1985) EMBO J. 4:2731-2737 and Spoerlein and Koop (1991) Theor. Appl. Genetics 83:1-5].

g. Other Methods of Introduction of Nucleic Acids into Plant Cells

Other methods to physically introduce nucleic acid into plant cells may be used, including silicon carbide fibers (“whiskers”) that are used to pierce plant cell walls thereby facilitating nucleic acid uptake, the use of sound waves to introduce holes in plant cell membranes to facilitate nucleic acid uptake (e.g., sonoporation) and the use of laser beams to open holes in cell membranes facilitating the entry of nucleic acids (e.g., laser poration).

Nucleic acids may also be imbibed by hydrating plant tissue, providing another method for nucleic acid uptake into plant cells [see, e.g., Simon (1974) New Phytologist 37:377-420]. For example, nucleic acids may be taken into cereal and legume seed embryos by inhibition [see, e.g., Toepfer et al. (1989) The Plant Cell 1:133-139].

4. Treatment of Cells into which Heterologous Nucleic Acids have been Introduced

Cells into which heterologous nucleic acids have been introduced may be analyzed for de novo formation of artificial chromosomes described herein such as may result from amplification of chromosomal segments occurring in connection with integration of heterologous nucleic acids into chromosomes. Typically, amplification occurs over multiple generations of cell division leading to the formation of detectable changes in chromosome structure. Therefore, transfected cells are typically cultured through multiple cell divisions, from about 5 to about 60, or about 5 to about 55, or about 10 to about 55, or about 25 to about 55, or about 35 to about 55 cell divisions following introduction of nucleic acid into a cell. Artificial chromosomes may, however, appear after only about 5 to about 15 or about 10 to about 15 cell divisions. Cells into which heterologous nucleic have been introduced may be treated in a variety of ways prior to or during analysis thereof for the presence of artificial chromosomes.

For example, cells into which nucleic acid encoding a selectable marker required for growth in the presence of a selection agent has been transferred can be treated as the exemplified cells herein to facilitate generation of multicentric chromosomes, and fragmentation thereof, and/or the generation of artificial chromosomes. The cells may be grown in the presence of an appropriate concentration of selection agent, which may be determined empirically by growing untransfected cells in varying concentrations of the agent and identifying concentrations sufficient to prevent cell growth and/or facilitate amplification of chromosomal segments. Transfected cells may be grown in selective media for numerous generations and cell lines can be established that contain the introduced nucleic acid. The concentration of selection agent may also be increased over several generations to promote amplification of a region of a chromosome into which heterologous nucleic acid integrated. Transfected cells may also be treated to destabilize the chromosomes to facilitate generation and fragmentation of a multicentric, typically dicentric, chromosome.

Additional heterologous nucleic acid, e.g., nucleic acid encoding a selectable marker, may also be introduced into the transfected cells to facilitate amplification of chromosomal segments, such as the pericentric heterochromatin, contained in, for example, a fragment released from a multicentric chromosome (e.g., a formerly dicentric chromosome), and generation of a heterochromatic artificial chromosome. The resulting transformed cells can then be grown in the presence of a selection agent, which may be a second agent (if the heterologous nucleic acid introduced into the transfected cells encodes a selectable marker different from any selectable marker encoded by heterologous nucleic acid initially transferred into the original host cells), with or without the first selection agent.

Cells into which nucleic acids have been introduced may also be subjected to cell sorting. For example, protoplasts may be prepared from transfected plant cells or calli and subjected to sorting. If the sorting is conducted prior to chromosomal analysis of the cells for the presence of artificial chromosomes, it provides a population of transfected cells that may be enriched for artificial chromosomes and thus facilitates the subsequent chromosomal analysis of the cells.

The sorting is based on the presence of a detectable marker in the cells, as provided for by the introduced nucleic acid, which can provide the basis for isolating such cells from cells that do not contain the heterologous nucleic acid. For example, the nucleic acid introduced into the plant cells may contain nucleic acid encoding a fluorescent protein, such as a green, red or blue fluorescent protein, which may be used for selection, by flow cytometry and other methods, of recipient cells that have taken up and express the nucleic acid at readily detected levels.

In an exemplary protocol, GFP fluorescence of transfected cell cultures may be monitored visually during culture using an inverted microscope equipped with epifluorescence illumination (Axiovert 25; Zeiss, (North York ON) and #41017 Endow GFP filter set (Chroma Technologies, Brattleboro, Vt.). Enrichment of GFP expressing populations can be carried out as follows. Cell sorting may be carried out, for example, using a FACS Vantage flow cytometer (Becton Dickinson Immunocytometry Systems, San Jose, Calif.) equipped with turbo-sort option and 2 Innova 306 lasers (Coherent, Palo Alto Calif.). For cell sorting a 70 μm nozzle can be used. The buffer can be changed to PBS (maintained at 20 p.s.i.). GFP may be excited with a 488 nm laser beam and excitation detected in FL1 using a 500 EFLP filter. Forward and side scattering can be adjusted to select for viable cells. Gating parameters may be adjusted using untransfected cells as negative control and GFP CHO cells as positive control.

For the first round of sorting, transfected cells may be harvested post-transfection (e.g., about 7-14 days post-transfection), converted to protoplasts, resuspended in about 10 ml of growth medium and sorted for GFP-expressing populations using parameters described above. GFP-positive cells may be dispensed into a volume of about 5-10 ml of protoplast medium while non-expressing cells are directed to waste. The expressing cells may be cultured. Plant cells or calli can then be analyzed, for fluorescence in-situ hybridization screening.

5. Analysis of Transformed Cells and Identification and Manipulation of Artificial Chromosomes

Cells into which nucleic acids have been introduced, and which may or may not have been further treated as described herein, may be analyzed for indications of amplification of chromosomal segments, the presence of structures that may arise in connection with amplification and de novo artificial chromosome formation and/or the presence of desired artificial chromosomes as described herein. Analysis of the cells typically involves methods of visualizing chromosome structure, including, but not limited to, G- and C-banding, PCR, Southern blotting and FISH analyses, using techniques described herein and/or known to those of skill in the art. Such analyses can employ specific labelling of particular nucleic acids, such as satellite DNA sequences, heterochromatin, rDNA sequences and heterologous nucleic acid sequences, that may be subject to amplification. During analysis of transfected cells, a change in chromosome number and/or the appearance of distinctive, for example, by increased segmentation arising from amplification of repeat units, chromosomal structures will also assist in identification of cells containing artificial chromosomes. The following description of events and structures that may be observed in analyzing cells for evidence of chromosomal amplification and/or the presence of artificial chromosomes is intended to be illustrative of the observations and considerations that may occur in the analysis of cells of any type, including mammalian and plant cells. It should be recognized that numerous types of structures may be formed during amplification of chromosomal segments and treatment of the cells. Additional, yet related, structures and variations of these structures are contemplated herein and are recognizable based on the descriptions and teachings of the generation and identification of artificial chromosomes presented herein. Each structure can be further manipulated, for example using procedures described herein, to derive additional chromosomal structures and compositions.

Typically, de novo centromere formation occurs in cells upon integration of heterologous nucleic acids into the cell chromosomes and amplification of chromosomal and heterologous nucleic acids. The integration and amplification that gives rise to de novo centromere formation typically occurs at the centromeric region of the short arm of a chromosome, typically an acrocentric chromosome. By employing methods such as chromosome-staining methods, including FISH and G- and C-banding, it may be possible to identify a chromosome at which the process occurs.

The amplification can lead to the formation of multicentric, typically dicentric, chromosomes. Because of the presence of two or more functionally active centromeres on the same chromosome, regular breakages occur between the centromeres. Such specific chromosome breakages can give rise to the appearance of a chromosome fragment carrying a neo-centromere. The neo-centromere may be found on a minichromosome (neo-minichromosome), while a formerly dicentric chromosome may carry traces of the heterologous nucleic acid.

a. The Neo-Minichromosome

Breakage of a dicentric chromosome between the two functional centromeres can form at least two chromosomes, for example, a so-called minichromosome, and a formerly dicentric chromosome. Treatment of cells containing a dicentric chromosome, such as, for example, recloning, treatment with agents that destabilize the chromosomes, e.g., BrdU, and/or culturing under selective conditions, may facilitate breakage of the dicentric chromosome. Selection of transformed cells can yield cell lines containing a stable neo-minichromosome. The breakage of a multicentric, typically dicentric, chromosome in transformed cells, which separates the neo-centromere from the remainder of the endogenous chromosome, may occur, for example, in the G-band positive heterologous nucleic acid region as is suggested if traces of the heterologous nucleic acid sequences at the broken end of the formerly dicentric chromosome are observed.

Multiple E-type amplification (amplification of euchromatin) may form a neo-chromosome, which separates from the remainder of the dicentric chromosome through a specific breakage between the centromeres of the dicentric chromosome. Inverted duplication of the fragment bearing the neo-centromere can result in the formation of a stable neo-minichromosome. The minichromosome is generally about at least 20-30 Mb in size.

The presence of inverted chromosome segments can be associated with the chromosomes formed de novo at the centromeric region of a chromosome. During the formation of the neo-minichromosome, the event leading to the stabilization of the distal segment of the chromosome that bears the duplicated neo-centromere may be the formation of its inverted duplicate.

Although the neo-minichromosome typically carries only one functional centromere, both ends of the minichromosome can be heterochromatic, carrying, for example, satellite DNA sequences as discernable by in situ hybridization. Comparison of the G-band pattern of a chromosome fragment carrying the neo-centromere with that of a stable neo-minichromosome, can indicate that the neo-minichromosome is an inverted duplicate of the chromosome fragment that bears the neo-centromere.

Cells containing a de novo-formed minichromosome, which contains multiple repeats of the heterologous nucleic acids, can be used as recipient cells in cell transfection. Donor nucleic acids, such as heterologous nucleic acids containing DNA encoding a desired protein and DNA encoding a second selectable marker, can be introduced into the cells and integrated into the de novo-formed minichromosomes. To facilitate integration into the de novo-formed minichromosomes, the heterologous DNA may also contain sequences that are homologous to nucleic acids already present in the minichromosomes, which can, through homologous recombination, provide targeted integration into the minichromosome. Nucleic acids can also be integrated into the minichromosome through the use of site-specific recombinases by producing minichromosomes containing site-specific recombination sites as described herein. Integration can be verified by in situ hybridization and Southern blot analyses. Transcription and translation of heterologous DNA can be confirmed by primer extension, immunoblot analyses and reporter gene assays, if a reporter gene has been included in the heterologous DNA, using, for example, appropriate nucleic acid probes and/or product-specific antibodies.

The resulting engineered minichromosome that contains the heterologous DNA can also be transferred, for example by cell fusion, into a recipient cell line to further verify correct expression of the heterologous DNA. Following production of the cells, metaphase chromosomes can be obtained, such as by addition of colchicine, and the minichromosomes purified using methods as described herein. The resulting minichromosomes can be used for delivery to specific cells of interest using any known method or methods for transferring heterologous nucleic acids into cells, particularly plant cells, and/or methods described herein.

Thus, the neo-minichromosome is stably maintained in cells, replicates autonomously, and permits the persistent, long-term expression of genes under non-selective culture conditions, and in a whole, intact, regenerated plant. It also can contain megabases of heterologous known DNA that can serve as target sites for homologous recombination and integration of DNA of interest. The neo-minichromosome is, thus, a vector for the delivery and expression of nucleic acids to cells.

Cell lines that contain artificial chromosomes, such as the minichromosome, the neo-chromosome, and the heterochromatic artificial chromosomes, are a convenient source of these chromosomes and can be manipulated, such as by cell fusion or production of microcells for fusion with selected cell lines, to deliver the chromosome of interest into a multiplicity of cell lines, including cells from a variety of different plant species.

b. Heterochromatin-Containing and Predominantly Heterochromatic Artificial Chromosomes

Manipulation of cells containing a fragment released upon breakage of the dicentric chromosome (e.g., a formerly dicentric chromosome), for example, by introducing additional heterologous nucleic acids, including, for example, DNA encoding a second selectable marker and growth under selective conditions, can yield heterochromatic structures. Included among such structures are compositions referred to as sausage chromosomes and megachromosomes. For example, a formerly dicentric chromosome may translocate to the end of another chromosome, such as an acrocentric chromosome. Additional heterologous nucleic acids added to cells containing a formerly dicentric chromosome can integrate into the pericentric heterochromatin of the formerly dicentric chromosome and be amplified several times with megabases of pericentric heterochromatic satellite DNA sequences forming a “sausage” chromosome carrying a newly formed heterochromatic chromosome arm. The size of this heterochromatic arm can vary, for example, between ˜150 and ˜800 Mb in individual metaphases. The chromosome arm can contain four to five satellite segments rich in satellite DNA, and evenly spaced integrated heterologous “foreign” DNA sequences. At the end of the compact heterochromatic arm of the sausage chromosome, a less condensed euchromatic terminal segment may be observed. By capturing a euchromatic terminal segment, this new chromosome arm is stabilized in the form of the “sausage” chromosome. In subclones of sausage chromosome-containing cell lines, the heterochromatic arm of the sausage chromosome may become unstable and show continuous intrachromosomal growth, particularly after treatment with BrdU and/or drug selection to induce further H-type amplification. In extreme cases, the amplified chromosome arm can exceed 500 Mb or even 1000 Mb in size (gigachromosome). Thus, the gigachromsome is a structure in which a heterochromatic arm has amplified but not broken off from a euchromatic arm.

In situ hybridization with, for example, biotin-labeled subfragments of the added heterologous nucleic acids may show a hybridization signal only in the heterochromatic arm of the sausage chromosome, indicating that the heterologous nucleic acid sequences are localized in the pericentric heterochromatin.

Gene expression, however, may be possible in the heterochromatic environment of a sausage chromosome. The level of heterologous gene expression may be determined by Northern hybridization with a subfragment of the selectable marker gene. Reporter genes included in heterologous nucleic acids also provide a readily detectable product for use in evaluating gene expression in a sausage or other heterochromatic or predominantly heterochromomatic chromosome. Southern hybridization of DNA isolated from subclones of sausage chromosome-containing cells with subfragments of reporter (and selectable marker) genes can show a close correlation between the intensity of hybridization and the length of the sausage chromosome.

Cell lines containing sausage chromosomes can be manipulated to yield additional heterochromatic structures and artificial chromosomes, including, for example, an artificial chromosome referred to as a megachromosome. Such manipulation includes fusion of the cell line with other cells and growth in the presence of one or more selection agents and/or BrdU.

Cells with a structure, such as the sausage chromosome, can be selected and fused with a second cell line, including other plant and non-plant species [see, e.g., Dudits et al. (1976) Heriditas 82:121-123 for the fusion of human cells with carrot protoplasts and Wiegand et al. (1987) J. Cell. Sci. (Pt. 2):145-149 for laser-induced fusion of plant protoplasts with mammalian cells] to eliminate other chromosomes that are not of interest. Structures such as sausage chromosomes formed during this process may be further manipulated, for example, by treating the cells with agents that destabilize chromosomes, e.g., BrdU, so that the heterochromatic arm forms a chromosome that is substantially heterochromatic (e.g., a megachromosome). Structures such as the gigachromosome in which the heterochromatic arm has amplified but not broken off from the euchromatic arm, may also be observed. Further manipulation, such as fusions and growth in selective conditions and/or BrdU treatment or other such treatment, can lead to fragmentation of the megachromosome to form smaller chromosomes that have the amplicon as the basic repeating unit.

If a cell with a sausage chromosome is selected, it can be treated with an agent, such as BrdU, that destabilizes the chromosome so that the heterochromatic arm forms a chromosome that is substantially heterochromatic (e.g., a megachromosome). Prior to treating the cell with BrdU, it can be fused with another cell line carrying chromosomes of another species, in order to eliminate chromosomes of the original host cell and obtain a cell in which the only chromosome from the host cell is the sausage chromosome. The resulting hybrid cells can be grown in the presence of multiple selection agents to select for those that carry the sausage chromosome. In situ hybridization with chromosome painting probes that detect chromosomes of both the host cell species and the species of cell to which the host cell was fused can provide an indication of the chromosomal make up of the hybrid cells.

Cell lines containing a sausage chromosome can be treated with a destabilizing agent, such as BrdU, followed by growth in selective medium and retreatment with BrdU. The BrdU treatments appear to destabilize the genome, resulting in a change in the sausage chromosome as well. A cell population in which a further amplification has occurred will arise. In addition to the heterochromatic arm (which may, for example, be ˜100-150 Mb) of the sausage chromosome, an extra centromere and another (for example, ˜150-250 Mb) heterochromatic chromosome arm may be formed. By the acquisition of another euchromatic terminal segment, a new submetacentric chromosome (e.g., megachromosome) can form.

Megachromosomes may also be produced through regrowth and establishment of sausage chromosome-containing cells in selective medium. Repeated BrdU treatment can produce cell lines that have a dwarf megachromosome (for example, about 150-200 Mb), a truncated megachromosome (for example, about 90-120 Mb), or a micro-megachromosome (for example, about 50-90 Mb). Cell lines containing smaller truncated megachromosomes can be used to generate even smaller megachromosomes, e.g., ˜10-30 Mb in size. This may be accomplished, for example, by breakage and fragmentation of a micro-megachromosome through exposing the cells to X-ray irradiation, BrdU or telomere-directed in vivo chromosome fragmentation.

Apart from the euchromatic terminal segments and the integrated foreign nucleic acid, the whole megachromosome, as well as other related types of predominantly heterochromatic artificial chromosomes, is constitutive heterochromatin. This can be demonstrated by C-banding of the megachromosome, which results in positive staining characteristic of constitutive heterochromatin. It can contain tandem arrays of satellite DNA. In a particular example, satellite DNA blocks are organized into a giant palindrome (amplicon) carrying integrated exogenous nucleic acid sequences at each end. It is of course understood that the specific organization and size of each component can vary among species, and also the chromosome in which the amplification event initiates.

In general, a clear segmentation may be observed in one or more arms of an amplification-based chromosome. For example, a megachromosome may contain building units that are amplicons of, for example, ˜30 Mb containing satellite DNA with the integrated “foreign” DNA sequences at both ends. The ˜30 Mb amplicons may be composed of two ˜15 Mb inverted doublets of ˜7.5 Mb satellite DNA blocks, which are separated from each other by a narrow band of non-satellite sequences. The wider non-satellite regions at the amplicon borders may contain integrated, exogenous (heterologous) nucleic acid, while any narrow bands of non-satellite DNA sequences within the amplicons may be integral parts of the pericentric heterochromatin of the host chromosomes. The sizes of the building units of a megachromosome or other amplification-based chromosome may vary depending on the species of the host chromosome from which the artificial chromosome was generated.

Further BrdU treatment can produce cell and/or calli that include cells with a truncated megachromosome. The megachromosome can be further fragmented in vivo using a chromosome fragmentation vector to ultimately produce a chromosome that comprises a smaller stable replicable unit, for example, about 15 Mb-60 Mb, containing one to four megareplicons.

Apart from the euchromatic terminal segments, the whole megachromosome is heterochromatic, and has structural homogeneity. Therefore, artificial chromosomes such as the megachromosome offer a unique possibility for obtaining information about the amplification process, and for analyzing some basic characteristics of the pericentric constitutive heterochromatin, as a vector for heterologous DNA, and as a target for further fragmentation.

C. Isolation of Artificial Chromosomes

The artificial chomosomes provided herein can be isolated by any suitable method known to those of skill in the art. Also, methods are provided herein for effecting substantial purification, particularly of the artificial chromosomes.

Artificial chromosomes, may be sorted from endogenous chromosomes using any suitable procedures, and typically involve isolating metaphase chromosomes, distinguishing the artificial chromosomes from the endogenous chromosomes, and separating the artificial chromosomes from endogenous chromosomes. Such procedures will generally include the following basic steps for animal cells and protoplasts: (1) culture of a sufficient number of cells (typically about 2×10⁷ mitotic cells) to yield, preferably on the order of 1×10⁶ artificial chromosomes, (2) arrest of the cell cycle of the cells in a stage of mitosis, preferrably metaphase, using a mitotic arrest agent such as colchicine, (3) treatment of the cells, particularly by cell wall dissolution for plant cells and/or swelling of the cells in hypotonic buffer, to increase susceptibility of the cells to disruption, (4) by application of physical force to disrupt the cells in the presence of isolation buffers for stabilization of the released chromosomes, (5) dispersal of chromosomes in the presence of isolation buffers for stabilization of free chromosomes, (6) separation of artificial chromosomes from endogenous chromosomes and (7) storage (and shipping if desired) of the isolated artificial chromosomes in appropriate buffers. Modifications and variations of the general procedure for isolation of artificial chromosomes, for example to accommodate different cell types with differing growth characteristics and requirements and to optimize the duration of mitotic block with arresting agents to obtain the desired balance of chromosome yield and level of debris, may be empirically determined (see Examples).

Steps 1-5 relate to isolation of metaphase chromosomes. The separation of artificial from endogenous chromosomes (step 6) may be accomplished in a variety of ways. For example, the chromosomes may be stained with DNA-specific dyes such as Hoeschst 33258 and chromomycin A₃ and sorted into artificial chromosomes and endogenous chromosomes on the basis of dye content by employing fluorescence-activated cell sorting (FACS).

Artificial chromosomes have been isolated by fluorescence-activated cell sorting (FACS). This method takes advantage of the nucleotide base content of the artificial chromosomes. In the case of predominantly heterochromatic artificial chromosomes, by virtue of their high heterochromatic DNA content, they will differ from any other chromosomes in a cell. In a particular embodiment, metaphase chromosomes are isolated and stained with base-specific dyes, such as Hoechst 33258 and chromomycin A3. Fluorescence-activated cell sorting will separate artificial chromosomes from the endogenous chromosomes. A dual-laser cell sorter (such as, for example, a FACS Vantage Becton Dickinson Immunocytometry Systems) in which two lasers were set to excite the dyes separately, allowed a bivariate analysis of the chromosomes by base-pair composition and size. Cells containing such artificial chromosomes can be similarly sorted.

Preparative amounts of artificial chromosomes (for example, 5×10⁴-5×10⁷ chromosomes/ml) at a purity of 95% or higher can be obtained. The resulting artificial chromosomes are used for delivery to cells by methods such as, for example, microinjection, liposome-mediated transfer, and electroporation.

Additional methods provided herein for isolation of artificial chromosomes from endogenous chromosomes include procedures that are particularly well suited for large-scale isolation of artificial chromosomes. In these methods, the size and density differences between artificial chromosomes and endogenous chromosomes are exploited to effect separation of these two types of chromosomes. To facilitate larger scale isolation of the artificial chromosomes, different separation techiniques may be employed such as swinging bucket centrifugation (to effect separation based on chromosome size and density) [see, e.g., Mendelsohn et al. (1968) J. Mol. Biol. 32:101-108], zonal rotor centrifugation (to effect separation on the basis of chromosome size and density) [see, e.g., Burki et al. (1973) Prep. Biochem. 3:157-182; Stubblefield et al. (1978) Biochem. Biophys. Res. Commun. 83:1404-1414, velocity sedimentation (to effect separation on the basis of chromosome size and shape) [see e.g., Collard et al. (1984) Cytometry 5:9-19].

Affinity-, particularly immunoaffinity-, based methods for separation of ACs from endogenous chromosomes are also provided herein. For example, artificial chromosomes which are predominantly heterochromatin may be separated from endogenous chromosomes through immunoaffinity procedures involving antibodies that specifically recognize heterochromatin, and/or the proteins associated therewith, when the endogenous chromosomes contain relatively little heterochromatin.

Immuno-affinity purification may also be employed in larger scale artificial chromosomes isolation procedures. In this process, large populations of artificial chromosome-containing cells (asynchronous or mitotically enriched) are harvested en masse and the mitotic chromosomes (which can be released from the cells using standard procedures such as by incubation of the cells, such as freshly isolated protoplasts, in hypotonic buffer and/or detergent treatment of the cells in conjunction with physical disruption of the treated cells) are enriched by binding to antibodies that are bound to solid state matrices (e.g. column resins or magnetic beads). Antibodies suitable for use in this procedure bind to condensed centromeric proteins or condensed and DNA-bound histone proteins. For example, autoantibody LU851 (see Hadlaczky et al. (1989) Chromosoma 97:282-288), which recognizes mammalian centromeres, may be used for large-scale isolation of chromosomes prior to subsequent separation of artificial chromosomes from endogenous chromosomes using methods such as FACS. The bound chromosomes would be washed and eventually eluted for sorting.

Immunoaffinity purification may also be used directly to separate artificial chromosomes from endogenous chromosomes. For example, in the case of artificial chromosomes that are predominantly heterochromatic, the artificial chromsomes may be generated in or transferred to (e.g., by microinjection or microcell fusion as described herein) a cell line that has chromosomes that contain relatively small amounts of heterochromatin, such as hamster cells (e.g., V79 cells or CHO-K1 cells). The predominantly heterochromatic artificial chromosomes are then separated from the endogenous chromosomes by utilizing anti-heterochromatin binding protein (Drosophila HP-1) antibody conjugated to a solid matrix. Such matrix preferentially binds artificial chromosomes relative to hamster chromosomes. Unbound hamster chromosomes are washed away from the matrix and the artificial chromosomes are eluted by standard techniques. Similarly, artificial chromosomes of one species, e.g., a plant-derived artificial chromosome, may be separated from a background of endogenous chromosomes of another species, e.g., animal, such as mammalian, chromosomes, based on immunological differences of the two species, provided that antibodies that specifically recognize one species and not the other are available or can be generated.

D. Generation of Artificial Chromosomes Through Assembly of Component Elements

Artificial chromosomes can be constructed in vitro by assembling the structural and functional elements that contribute to a complete chromosome capable of stable replication and segregation alongside endogenous chromosomes in cells. The identification of the discrete elements that in combination yield a functional chromosome has made possible the in vitro assembly of artificial chromosomes. The process of in vitro assembly of artificial chromosomes, which can be rigidly controlled, provides advantages that may be desired in the generation of chromosomes that, for example, are required in large amounts or that are intended for specific use in transgenic organism systems.

For example, in vitro assembly may be advantageous when efficiency of time and scale are important considerations in the preparation of artificial chromosomes. Because in vitro assembly methods do not involve extensive cell culture procedures, they may be utilized when the time and labor required to transform, feed, cultivate, and harvest cells used in de novo cell-based production systems is unavailable.

Provided herein are in vitro assembly methods that include the joining of essential components, such as a centromere, telomere and an origin of replication, to yield an artificial chromosome, in particular, an artificial chromosome that functions in plants and that may contain components derived from plant chromosomes. Also provided are artificial chromosomes produced by the methods. Particular embodiments of the methods and chromosomes include a megreplicator. The megareplicator may contain rDNA, for example, mammalian or plant rDNA. In vitro assembled artificial chromosomes may contain any amount of heterochromatic and/or euchromatic nucleic acid. For example, an in vitro assembled artificial chromosome may be substantially all heterochromatin, while still containing protein-encoding DNA, or may contain increasing amounts of euchromatic DNA, such that, for example, it contains about 10%, 20%, 30%, 40%, 50%, 60%, 70%, 80%, 90% or greater than about 90% euchromatic DNA.

In vitro assembly may also be rigorously controlled with respect to the exact manner in which the several elements of the desired artificial chromosome are combined and in what sequence and proportions they are assembled to yield a chromosome of precise specifications. This feature is of particular significance in the generation of plant artificial chromosomes containing one or more regions of segmentation as described herein with reference to amplification-based artificial chromosomes. For example, certain plant chromosome structures (such as acrocentric chromosomes and/or chromosomes containing adjacent regions of heterochromatin and rDNA) that may be desirable for use in the generation of particular types of plant artificial chromosomes via amplification-based methods as described herein may be limited in number or may not exist. These particular types of plant artificial chromosomes, e.g., certain predominantly heterochromatic plant artificial chromosomes, may also be generated via in vitro assembly of artificial chromosomes as described herein.

For example, plant artificial chromosomes containing regions of repeated nucleic acid units that are predominantly heterochromatic may be assembled by joining essential chromosomal components and repeat regions, or may be generated from an in vitro assembled artificial chromosome via amplification of heterochromatic DNA contained within an in vitro assembled artificial chromosome. For generation of such chromosomes via amplification of heterochromatic DNA contained within an in vitro assembled artificial chromosome, nucleic acids are introduced into a cell containing an in vitro assembled artificial chromosome and a resulting cell is selected that contains an artificial chromosome containing one or more regions of repeated nucleic acid units that are predominantly heterochromatic. The in vitro assembled artificial chromosome either contains a megareplicator to faciliate amplification of chromosomal DNA in connection with integration of nucleic acid into the chromosome or megareplicator-containing DNA is included in the nucleic acid that is integrated into thee in vitro assembled artificial chromosome.

The following describes the processes involved in the assembly of artificial chromosomes in vitro, utilizing a megachromosome as exemplary starting material.

1. Identification and Isolation of the Components of the Artificial Chromosome

The chromosomes provided herein are elegantly simple chromosomes for use in the identification and isolation of components to be used in the in vitro assembly of expression systems or artificial chromosomes. The ability to purify artificial chromosomes to a very high level of purity, as described herein, facilitates their use for these purposes. For example, the megachromosome, particularly truncated forms thereof, serve as starting materials. With respect to the construction of an artificial chromosome containing at least some mammalian cell derived components, possible starting materials can be obtained from, for example, cell lines such as 1B3 and mM2C1, which are derived from H1 D3 (deposited at the European Collection of Animal Cell Culture (ECACC) under Accession No. 96040929). With respect to the construction of an artificial chromosome containing at least some plant cell derived components, possible starting materials include cells containing PACs, e.g., megachromosomes, generated as described herein.

For example, the mM2C1 cell line contains a micro-megachromosome (˜50-60 kB), which advantageously contains only one centromere, two regions of integrated heterologous DNA with adjacent rDNA sequences, with the remainder of the chromosomal DNA being mouse major satellite DNA. Other truncated megachromosomes can serve as a source of telomeres, or telomeres can be provided. The centromere of the mM2C1 cell line contains mouse minor satellite DNA, which provides a useful tag for isolation of the centromeric DNA.

Additional features of particular ACs provided herein, such as the micro-megachromosome of the mM2C1 cell line, that make them uniquely suited to serve as starting materials in the isolation and identification of chromosomal components include the fact that the centromeres of each megachromosome within a single specific cell line are identical. The ability to begin with a homogeneous centromere source (as opposed to a mixture of different chromosomes having differing centromeric sequences) greatly facilitates the cloning of the centromere DNA. By digesting purified megachromosomes, particularly truncated megachromosomes, such as the micro-megachromosome, with appropriate restriction endonucleases and cloning the fragments into commercially available and well known YAC vectors (see, e.g, Burke et al. (1987) Science 236:806-812), BAC vectors (see, e.g., Shizuya et al. (1992) Proc. Natl. Acad. Sci. U.S.A. 89: 8794-8797 bacterial artificial chromosomes which have a capacity of incorporating 0.9-1 Mb of DNA) or PAC vectors (the P1 artificial chromosome vector which is a P1 plasmid derivative that has a capacity of incorporating 300 kb of DNA and that is delivered to E. coli host cells by electroporation rather than by bacteriophage packaging; see, e.g., loannou et al. (1994) Nature Genetics 6:84-89; Pierce et al. (1992) Meth. Enzymol. 216:549-574; Pierce et al. (1992) Proc. Natl. Acad. Sci. U.S.A. 89:2056-2060; U.S. Pat. No. 5,300,431 and International PCT application No. WO 92/14819) vectors, it is possible for as few as 50 clones to represent the entire micro-megachromosome.

a. Centromeres

An exemplary centromere for use in the construction of an artificial chromosome is that contained within a megachromosome, such as those described herein. One example of a particular megachromosome-containing cell line provided is, for example, H1 D3 and derivatives thereof, such as mM2C1 cells. Megachromosomes are isolated from such cell lines utilizing, for example, the procedures described herein, and the centromeric sequence is extracted from the isolated megachromosomes. For example, the megachromosomes may be separated into fragments utilizing selected restriction endonucleases that recognize and cut at sites that, for instance, are primarily located in the replication and/or heterologous DNA integration sites and/or in the satellite DNA. Based on the sizes of the resulting fragments, certain undesired elements may be separated from the centromere-containing sequences. The centromere-containing DNA could be as large as 1 Mb.

Probes that specifically recognize centromeric sequences, such as mouse minor satellite DNA-based probes [see, e.g., Wong et al. (1988) Nucl. Acids Res. 16:11645-11661], pCT4.2 probe, a 3.5 kb fragment of Arabidopsis 5S rDNA (Campbell et al. (1992) Gene 112:225-228), Arabidopsis cosmids E4.11 (30 kb) adn E4.6 (33 kb, Bent et al. (1994) Science 265:1856-1860; and 180 bp pAL1 repeat sequence (Maluszynska et al. (1991) Plant J. 1:159-166; and Martinez-Zapater et al. (1986) Mol. Gen. Genet. 204:417-423) may be used to isolate a centromere-containing YAC, BAC or PAC clone derived from the megachromosome. Alternatively, or in conjunction with the direct identification of centromere-containing megachromosomal DNA, probes that specifically recognize the non-centromeric elements, such as probes specific for mouse major satellite DNA, plant satellite DNA, the heterologous DNA and/or rDNA, may be used to identify and eliminate the non-centromeric DNA-containing clones.

Additionally, centromere cloning methods described herein may be utilized to isolate the centromere-containing sequence of the megachromosome.

Once the centromere fragment has been isolated, it may be sequenced and the sequence information may in turn be used in PCR amplification of centromere sequences from megachromosomes or other sources of centromeres. Isolated centromeres may also be tested for function in vivo by transferring the DNA into a host cell. Functional analysis may include, for example, examining the ability of the centromere sequence to bind centromere-binding proteins. The cloned centromere will be transferred to cells with a selectable marker gene and the binding of a centromere-specific protein, such as anti-centromere antibodies (e.g., LU851, see, Hadlaczky et al. (1986) Exp. Cell Res. 167:1-15) can be used to assess function of the centromeres.

b. Telomeres

Telomeres that may be used in assembly of an artificial chromosome include a 1 kB synthetic telomere (see, e.g., PCT Application Publication No. WO 97/40183). A double synthetic telomere construct, which contains a 1 kB synthetic telomere linked to a dominant selectable marker gene that continues in an inverted orientation may be used for ease of manipulation. Such a double construct contains a series of TTAGGG repeats 3′ of the marker gene and a series of repeats of the inverted sequence, i.e., GGGATT, 5′ of the marker gene as follows: (GGGATTT)ₙ—dominant marker gene—(TTAGGG)ₙ. Using an inverted marker provides an easy means for insertion, such as by blunt end ligation, since only properly oriented fragments will be selected.

Telomere sequences also include sequences described in plants, for example, an Arabidopsis sequence containing head-to-tail arrays of the monomer repeat CCCTAAA totaling a few, for example 3-4, kb in length. Telomere sequences vary in length and do not appear to have a strict length requirement. An example of a cloned telomere is found in GenBank accession no. M20158 (Richards and Ausubel (1988) Cell 53:127-136) and in U.S. Pat. No. 5,270,201. Yeast telomere sequences include those provided in GenBank accession no. S70807 (Louis et al. (1994) Yeast 10:271-274). Additionally, a method for isolating a higher eukaryotic telomere from A. thaliana has been reported (Richards and Ausubel (1988) Cell 53:127-136; and U.S. Pat. No. 5,270,201).

C. Megareplicator

The megareplicator sequences, such as those containing rDNA, provided herein are preferred for use in artificial chromosomes generated by assembly of component elements in vitro. The rDNA provides an origin of replication and also provides sequences that facilitate amplification of the artificial chromosome in vivo to increase the size of the chromosome to, for example, accommodate increasing copies of a heterologous gene of interest as well as continuous high levels of expression of the heterologous genes.

d. Filler Heterochromatin

Filler heterochromatin, particularly satellite DNA, is included to maintain structural integrity and stability of the artificial chromosome and provide a structural base for carrying genes within the chromosome. The satellite DNA is typically A/T-rich DNA sequence, such as mouse major satellite DNA, or G/C-rich DNA sequence, such as hamster natural satellite DNA. Sources of such DNA include any eukaryotic organisms that carry non-coding satellite DNA with sufficient A/T or G/C composition to promote ready separation by sequence, such as by FACS, or by density gradients. Examples of plant satellite DNA include, but are not limited to, satellite DNA of soybean (see, e.g., Morgante et al. (1997) Chromosome Res. 5:363-373; and Vahedian et al. (1995) Plant Mol. Biol. 29:857-862), satellite DNA on the rye B chromosome (see, e.g., Langdon et al. (2000) Genetics 154:869-884) and satellite DNA in the Saccharum complex (see, e.g., Alix et al. (1 998) Genome 41:854-864). The satellite DNA may also be synthesized by generating sequence containing monotone, tandem repeats of highly A/T- or G/C-rich DNA units.

The most suitable amount of filler heterochromatin for use in construction of the artificial chromosome may be empirically determined by, for example, including segments of various lengths, increasing in size, in the construction process. Fragments that are too small to be suitable for use will not provide for a functional chromosome, which may be evaluated in cell-based expression studies, or will result in a chromosome of limited functional lifetime or mitotic and structural stability.

e. Selectable Marker

Any convenient selectable marker, including specific examples described herein, may be used and at any convenient locus in the expression system.

2. Combination of the Isolated Chromosomal Elements

Once the isolated elements are obtained, they may be combined to generate the complete, functional artificial chromosome expression system. This assembly can be accomplished for example, by in vitro ligation either in solution, LMP agarose or on microbeads. The ligation is conducted so that one end of the centromere is directly joined to a telomere. The other end of the centromere, which serves as the gene-carrying chromosome arm, is built up from a combination of satellite DNA and megareplicator sequences, e.g., rDNA sequence, and may also contain a selectable marker gene. Another telomere is joined to the end of the gene-carrying chromosome arm. The gene-carrying arm is the site at which any heterologous genes of interest, for example, in expression of desired proteins encoded thereby, are incorporated either during in vitro assembly of the chromosome or sometime thereafter.

3. Analysis and Testing of the Artificial Chromosome Expression Systems

Artificial chromosomes assembled in vitro may be tested for functionality in cell systems, such as plant and animal cells, using any of the methods described herein for the artificial chromosomes, minichromosomes, or known to those of skill in the art.

4. Introduction of Desired Heterologous DNA into the In Vitro Assembled Chromosome

Heterologous DNA may be introduced into the in vitro synthesized chromosome using routine methods of molecular biology, may be introduced using the methods described herein for the artificial chromosomes, or may be incorporated into the in vitro assembled chromosome as part of one of the synthetic elements, such as the heterochromatin. The heterologous DNA may be linked to a selected repeated fragment, and then the resulting construct may be amplified in vitro using the methods for such in vitro amplification provided herein.

In a particular embodiment of these in vitro assembly methods, a site-specific recombination site is included in the assembly DNA or is added into the assembled chromosome, such as a plant in vitro assemble artificial chromosome, after initial assembly. The presence of a recombination site in the in vitro assembled artificial chromosome facilitates recombinase-catalyzed introduction of heterologous nucleic acid into the chromosome if the heterologous nucleic acid also contains a complementary recombination site. Such recombination systems include, but are not limited to, Cre/lox [see, e.g., Dale and Ow (1995) Gene 91:79-85], FLP/FRT [see, e.g., Nigel et al. (1995) The Plant Journal 8:637-652], R/RS [see, e.g., Onouchi et al. (1991) Nuc. Acids Res. 19:6373-6378], Gin/gix [see, e.g., Maeser and Kahman (1991) Mol. Gen. Genet. 230:170-1 76] and int/att. The introduction of att recombination sites into a chromosome and the use of lambda phage integrase recombinase in conjunction therewith to permit engineering of natural and artificial chromosomes is desribed in copending U.S. provisional application Serial No. 60/294,758, by Perkins et al. entitled “CHROMOSOME-BASED PLATFORMS” filed on May 30, 2001, U.S. provisional application Serial No. 60/366,891, by Perkins et al. entitled “CHROMOSOME-BASED PLATFORMS” filed on Mar. 21, 2002, U.S. patent application Ser. No. ______ , by Perkins et al. entitled “CHROMOSOME-BASED PLATFORMS” filed on May 30, 2002, under attorney docket no. 24601-420, and PCT International Application No. ______ , by Perkins et al. entitled “CHROMOSOME-BASED PLATFORMS” filed on May 30, 2002, under attorney docket no. 24601-420PC, each of which is incorporated herein in its entirety by reference thereto. Thus, also contemplated herein are in vitro assembled artificial chromosomes, in particular such chromosomes containing plant chromosome-derived components, that contain one or more recombination sites, such as an att site.

E. Methods for the Production of Plant Acrocentric Chromosomes and Plant Chromosomes Containing Adjacent Regions of rDNA and Heterochromatin

Acrocentric human and mouse chromosomes in which the short arm contains only pericentric heterochromatin, an rDNA array, and telomeres can be used in the de novo formation of a satellite DNA based artificial chromosome (SATAC, also referred to as ACes). In some embodiments of the methods of producing a plant artificial chromosome provided herein, it may be desirable to introduce heterologous nucleic acids into a plant chromosome with arms of unequal length (e.g., into the short arm of an acrocentric chromosome) and/or containing adjacent regions of rDNA and heterochromatin, such as pericentric heterochromatin or satellite DNA. Of particular interest in such methods are plant acrocentric chromosomes that contain rDNA located adjacent to the pericentric heterochromatin or satellite DNA, and, in particular, on the short arm of the chromosome with little to no euchromatic DNA between the rDNA and the pericentric heterochromatin. Utilizing such structures as the initial composition in the generation of plant artificial chromosomes may facilitate generation of plant artificial chromosomes that are predominantly heterochromatic. For example, introduction of heterologous nucleic acid into a cell containing such an acrocentric plant chromosome such that the nucleic acid integrates into the pericentric heterochromatin and/or rDNA of the short arm of the chromosome may be associated with amplification (possibly through “megareplicator” DNA sequences such as may reside in plant rDNA arrays, also known as the nucleolar organizing regions (NOR)) of heterochromatin that leads to the formation of a predominantly heterochromatic plant artificial chromosome.

Naturally occurring acrocentric plant chromosomes are limited in number, and plant chromosomes with a structure that includes adjacent regions of heterochromatin and rDNA may not exist or may not exist for a variety of plant species. Provided herein are methods for generating acrocentric plant chromosomes and plant chromosomes containing adjacent regions of rDNA and heterochromatin, in particular, pericentric and/or satellite heterochromatin. Further provided herein are methods for generating acrocentric plant chromosomes containing adjacent regions of heterochromatin, such as pericentric heterochromatin and/or satellite DNA, and rDNA on the short arm of the chromosome.

Also provided herein are plant acrocentric chromosomes in which the nucleic acid of one or both arms of the chromosome contains less than about 50%, or less than about 40%, or less than about 30%, or less than about 20%, or less than about 10%, or less than about 5%, or less than about 2%, or less than about 1%, or less than about 0.5% or less than about 0.1% euchromatin. In some embodiments of these chromosomes, the nucleic acid of only one arm, either the short arm or the long arm, contains less than these specified amounts of euchromatin. In a particular embodiment of these chromosomes, the nucleic acid of the short arm contains less these specified amounts of euchromatin.

Further provided herein are plant chromosomes containing adjacent regions of heterochromatin, in particular pericentric heterochromatin or satellite DNA, and rDNA with little to no euchromatin between the two regions. With reference to such plant chromosomes, “litte to no” means that the amount of euchromatic DNA, if any, located between the rDNA and heterochromatin (such as pericentric heterochromatin and/or satellite DNA), generally does not stain diffusely and recognizably as euchromatin and/or does not contain protein-encoding genes. Thus, in these chromosomes, between the heterochromatin (such as pericentric heterochromatin and/or satellite DNA) and the rDNA, there is substantially no chromatin that is less condensed than the heterochromatin (e.g., pericentric heterochromatin). The plant chromosomes containing adjacent regions of rDNA and heterochromatin (such as pericentric heterochromatin) provided herein may be acrocentric chromosomes. In a particular embodiment of these plant chromosomes, the adjacent regions of rDNA and heterochromatin, in particular pericentric heterochromatin, are contained on the short arm of the chromosome.

Further provided are methods of utilizing such plant chromosomes in the generation of plant artificial chromosomes, and, in particular, predominantly heterochromatic plant artificial chromosomes, such as ACes (also referred to as SATACs). In particular methods of producing plant artificial chromosomes provided herein, nucleic acids are introduced into a cell containing a plant chromosome that is acrocentric and/or contains adjacent regions of rDNA and heterochromatin, such as pericentric heterochromatin, the cells are cultured through at least one cell division and a cell comprising an artificial chromosome, such as a predominantly heterochromatic artificial chromosome, is selected. In these methods, the plant chromosome into which nucleic acid is introduced may be an acrocentric chromosome containing adjacent regions of rDNA and heterochromatin on the short or long arm, and, in particular, on the short arm.

The plant chromosomes provided herein can be generated using site-specific recombination between plant chromosome regions. The regions may be on the same chromosome or separate chromosomes. Through site-specific recombination, sections of plant chromosomes may be altered to remove, invert and/or insert sequences such that a desired plant chromosome results. The resulting plant chromosome is acrocentric and/or contains adjacent regions of heterochromatic DNA and rDNA, which may or may not be on the short arm of an acrocentric chromosome. Thus, the starting chromosome in these methods may be a plant chromosome or may be a plant acrocentric chromosome that does not contain adjacent regions of rDNA and heterochromatin, such as pericentric heterochromatin or satellite DNA. If the starting chromosome is acrocentric, then it may be used in the generation of a plant acrocentric chromosome that contains adjacent regions of heterochromatic DNA (e.g., pericentric heterochromatin and/or satellite DNA) and rDNA, particularly on the short arm of the chromosome, or to generate a plant acrocentric chromosome in which the nucleic acid of one or both arms contains less than about 50%, or less than about 40%, or less than about 30%, or less than about 20%, or less than about 10%, or less than about 5%, or less than about 2%, or less than about 1%, or less than about 0.5% or less than about 0.1% euchromatin.

In one of the methods provided herein for producing a plant chromosome that is acrocentric and/or contains adjacent regions of rDNA and heterochromatin, nucleic acid containing a site-specific recombination site and nucleic acid containing a complementary site-specific recombination site are introduced into a cell containing one or more plant chromosomes. The nucleic acids may be introduced into the cell sequentially or simultaneously. The nucleic acids may also be targeted to particular chromosomes and/or particular sequences of a chromosome. Such targeting may be accomplished by including in the nucleic acids sequences homologous to particular sequences in the chromosome(s).

The cell is then exposed to a recombinase activity. The recombinase activity can be provided by introduction of nucleic acid encoding the activity into the cell for expression of the activity therein, or may be added to the cell from an exogenous source. The recombinase activity is one that catalyzes recombination between sequences at the two recombination sites. An appropriate recombination event produces a plant chromosome that is acrocentric and/or contains adjacent regions of rDNA and heterochromatin (such as pericentric heterochromatin and/or satellite DNA) which may be readily identified therein based on its particular structure (e.g., arms of unequal length if the chromosome is acrocentric) and/or other features, e.g., the presence of particular added sequences, such as recombination sites and DNA encoding a selectable marker, the absence of particular sequences, such as excised euchromatic DNA, and the arrangement of sequences, such as the placement of rDNA segments adjacent to pericentric heterochromatin and/or satellite DNA. Such attributes may be detected using techniques known in the art for the analysis of nucleic acids and chromosomes, such as, for example, in situ hybridization.

A number of site-specific recombination systems may be used in the production of plant chromosomes that are acrocentric and/or contain rDNA adjacent to heterochromatin, such as pericentric heterochromatin, as described herein. Such systems include, but are not limited to, Cre/lox [see, e.g., Dale and Ow (1995) Gene 91:79-85], FLP/FRT [see, e.g., Nigel et al. (1995) The Plant Journal 8:637-652], R/RS [see, e.g., Onouchi et al. (1991) Nuc. Acids Res. 19:6373-6378], Gin/gix [see, e.g., Maeser and Kahman (1991) Mol. Gen. Genet. 230:170-176] and int/att. The introduction of att recombination sites into a chromosome and the use of lambda phage integrase recombinase in conjunction therewith to permit engineering of natural chromosomes is desribed in copending U.S. provisional application Serial No. 60/294,758 by Perkins et al. entitled “CHROMOSOME-BASED PLATFORMS” filed on May 30, 2001, U.S. provisional application Serial No. 60/366,891, by Perkins et al. entitled “CHROMOSOME-BASED PLATFORMS” filed on Mar. 21, 2002, U.S. patent application Ser. No. ______ , by Perkins et al. entitled “CHROMOSOME-BASED PLATFORMS” filed on May 30, 2002, under attorney docket no. 24601-420, and PCT International Application No. ______ , by Perkins et a;. entitled “CHROMOSOME-BASED PLATFORMS” filed on May 30, 2002, under attorney docket no. 24601-420PC, each of which is incorporated herein in its entirety by reference thereto. These systems, as well as others known in the art, can be used to specifically excise or invert DNA (for example, in an intrachromosomal recombination), exchange regions of DNA (for example, in an inter-chromosomal recombination) or insert DNA (for example, through recombination between homologous sequences at a recombination site and the DNA to be inserted). The precise event is controlled by the orientation of the recombination site DNA sequences.

In particular embodiments of the methods for producing an acrocentric plant chromosome provided herein, nucleic acid containing complementary recombinase recognition sites for site-specific recombination is introduced into a cell containing one or more plant chromosomes wherein one of the sites integrates into, or close to, the pericentric heterochromatin and/or satellite DNA (in particular, proximal satellite DNA) of one plant chromosome in the cell. In a further embodiment, nucleic acid containing complementary recombinase recognition sites for site-specific recombination is introduced into a cell containing one or more plant chromosomes wherein one of the sites integrates into the distal end of an arm of a plant chromosome in the cell. In these embodiments, recombination between the sites in the presence of a recombinase that recognizes the sites can result in deletion of a portion of an arm of a chromosome, reciprocal translocation between a distal portion of a chromosome arm and a more proximal portion of another chromosome arm or reciprocal translocation between pericentric heterochromatin and/or satellite DNA of one chromosomal arm and a more distal portion of another chromosome arm. Each of these recombination events can serve to reduce the length of a chromosome arm and give rise to an acrocentric chromosome.

In another embodiment, a nucleic acid containing a site-specific recombination site is introduced into a cell containing plant chromosomes wherein it integrates into the pericentric heterochromatin and/or satellite DNA of one plant chromosome in the cell and nucleic acid containing a complementary site-specific recombination site is introduced into the cell wherein it integrates into the distal end of an arm of another plant chromosome in the cell. In this embodiment, recombination between the sites in the presence of a recombinase that recognizes the sites can result in reciprocal translocation between the pericentric heterochromatin and/or satellite DNA of one chromosome and the distal portion of another chromosome arm thereby bringing these two regions into close proximity on one chromosomal arm and reducing the amount of DNA between the pericentric region of the arm and the end of the arm to generate an acrocentric plant chromosome.

These methods for producing an acrocentric plant chromosome may also be conducted such that nucleic acid containing a site-specific recombination site is introduced into a cell containing a plant chromosome wherein it integrates into, or close to, the pericentric heterochromatin and/or satellite DNA of a plant chromosome in the cell and nucleic acid containing a complementary site-specific recombination site is introduced into the cell wherein it integrates into the distal end of the same arm of the same chromosome. In this embodiment, recombination between the sites in direct (i.e., the same, or head-to-tail) orientation in the presence of a recombinase that recognizes the sites can result in intrachromosomal recombination between the pericentric heterochromatin (and/or satellite DNA) and the distal portion of the chromosomal arm thereby excising DNA between these two regions and reducing the amount of DNA between them to generate an acrocentric plant chromosome.

In particular embodiments of the methods provided herein for producing a plant chromosome containing adjacent regions of rDNA and heterochromatin, such as pericentric heterochromatin and/or satellite DNA, nucleic acid containing complementary recombinase recognition sites for site-specific recombination is introduced into a cell containing one or more plant chromosomes wherein one of the sites integrates into heterochromatin of one plant chromosome in the cell. In a further embodiment, nucleic acid containing complementary recombinase recognitions sites for site-specific recombination is introduced into a cell containing one or more plant chromosomes wherein one of the sites integrates into rDNA or a nucleolar organizing region (NOR) of a plant chromosome in the cell. In these embodiments, recombination between the sites in the presence of a recombinase that recognizes the sites can result in deletion of DNA between a heterochromatic region, such as the pericentric heterochromatin (and/or satellite DNA), and rDNA, inversion of DNA that includes heterochromatin or rDNA of a plant chromosome or reciprocal translocation between heterochromatin of one chromosomal arm and rDNA of another chromosomal arm. Each of these recombination events can serve to arrange chromosomal DNA such that a region of heterochromatic DNA, such as pericentric heterochromatin and/or satellite DNA, is adjacent to a region of rDNA on a plant chromosome.

In another embodiment, nucleic acid containing a site-specific recombination site is introduced into a cell containing plant chromosomes wherein it integrates into heterochromatin, such as, for example, pericentric heterochromatin and/or satellite DNA, of one plant chromosome in the cell and nucleic acid containing containing a complementary site-specific recombination site is introduced into the cell wherein it integrates into rDNA of another plant chromosome in the cell. In this embodiment, recombination between the sites can result in reciprocal translocation between the heterochromatin of one chromosome and the rDNA of another chromosome thereby bringing these two regions into close proximity on one plant chromosome with little to no euchromatin between them.

These methods for producing a plant chromosome containing adjacent regions of heterochromatic DNA and rDNA may also be conducted such that nucleic acid containing site-specific recombination sites is introduced into a cell containing a plant chromosome wherein it integrates into heterochromatin, for example, pericentric heterochromatin and/or satellite DNA, of a plant chromosome and nucleic acid containing a complementary site-specific recombination site is introduced into the cell wherein it integrates into rDNA of the same chromosome. In this embodiment, recombination between the sites in direct orientation in the presence of a recombinase that recognizes the sites can result in intrachromosomal recombination between heterochromatin, such as pericentric heterochromatin (and/or satellite DNA), and rDNA thereby excising DNA, including euchromatic DNA, between these two regions. Recombination of the sites in indirect (i.e., head-to-head) orientation in the presence of a recombinase can result in inversion of DNA between the sites thereby replacing DNA, such as euchromatin, located between pericentric heterochromatin (and/or satellite DNA) and rDNA on the chromosome with rDNA. Thus, in the resulting plant chromosome, rDNA is located adjacent to pericentric heterochromatin (and/or satellite DNA), and DNA that was present between the pericentric heterochromatin (and/or satellite DNA) and the rDNA is located distal to the rDNA in a position previously occupied by the rDNA.

In particular embodiments for producing an acrocentric plant chromosome containing adjacent regions of heterochromatin, such as pericentric heterochromatin (and/or satellite DNA), and rDNA, the short arm of the acrocentric chromosome may be generated in the same recombination event that places the heterochromatin and rDNA regions adjacent to each other or in a separate recombination event. For example, nucleic acid containing a site-specific recombination site may be introduced into a cell containing one or more plant chromosomes wherein it integrates into the pericentric heterochromatin of one plant chromosome and nucleic acid containing a complementary site-specific recombination site may be introduced into the cell wherein it integrates into rDNA that is located at a distal portion of another plant chromosome or the same arm of the same of the same chromosome. Recombination of the sites in the presence of a recombinase can result in intra- or inter-chromosomal recombination that not only brings the pericentric heterchromatin (and/or satellite DNA) and rDNA into close proximity on one chromosomal arm, but also sufficiently reduces the length of that arm such that the resulting chromosome is acrocentric.

If a single recombination event such as this does not generate an acrocentric plant chromosome, multiple recombination events may be used to produce an acrocentric plant chromosome containing adjacent regions of heterochromatic DNA and rDNA. For example, nucleic acid containing a site-specific recombination site may be introduced into a cell containing one or more plant chromosomes wherein it integrates into the pericentric heterochromatin (and/or satellite DNA) of one plant chromosome and nucleic acid containing a complementary site-specific recombination site may be introduced into the cell wherein it integrates into rDNA of the same or a different plant chromosome. As described abouve, recombination between the sites in the presence of a recombinase can result in deletion, inversion or reciprocal translocation of DNA to arrange chromosomal DNA such that pericentric heterochromatin (and/or satellite DNA) is adjacent to a region of rDNA on a plant chromosome. In order to reduce the length of the arm of the chromosome on which the adjacent regions of heterochromatin and rDNA are located, an additional recombination event can be induced by introducing nucleic acid containing a site-specific recombination site into a cell containing this plant chromosome wherein it integrates into a region of the chromosome distal to the rDNA and nucleic acid containing a complementary site-specific recombination site into the cell wherein it integrates into the distal end of the same chromosome arm or of another plant chromosome arm. Recombination between the recognition sites can result in deletion or reciprocal translocation of DNA to reduce the length of the chromosome arm distal to the rDNA and give rise to an acrocentric plant chromosome containing adjacent regions of heterochromatin and rDNA on the short arm of the chromosome.

In each of the aforementioned methods for producing a plant chromosome that is acrocentric and/or contains adjacent regions of heterochromatin and rDNA, the nucleic acid containing the two or more recombination sites may be introduced simultaneously or sequentially into a cell or cells using nucleic acid transfer methods described herein or known in the art. The nucleic acids may randomly integrate into plant chromosomes or may be targeted for integration into a particular region or site on a plant chromosome through homologous recombination between sequences in the nucleic acid and sequences within the chromosome. The recombinase activity may be provided by introduction of nucleic acid encoding an appropriate recombinase into the cell for expression therein. The recombinase-encoding nucleic acid may be introduced into the cell prior to, during or after introduction of nucleic acids encoding recombination sites.

To facilitate identification of cells containing the transferred nucleic acids and/or in which a recombination event has occurred, nucleic acid encoding a selectable marker may be introduced into the cell. For example, one or both of the nucleic acids containing a recombination site may also contain DNA encoding a selectable marker (e.g., a resistance-encoding marker or a reporter molecule) operatively linked to a promoter which is oriented such that integration of the nucleic acid into a chromosome places the marker DNA between two directly oriented recombination sites on an arm of a chromosome. A cell containing the nucleic acid will thus be resistant to a selection agent or will detectably express a reporter molecule. Exposure of the cell to the appropriate recombinase can result in a recombination event that excises the DNA between the two recombination sites, which includes DNA encoding the selectable marker. Thus, recombination could be detected as loss of reporter molecule expression or decreased resistance to a selection agent.

After exposure to a recombinase, the cells into which nucleic acids containing recombination sites have been transferred may be analyzed for the presence of acrocentric plant chromosomes using, for example, FISH analysis and other chromosome visualization techniques.

In another method provided herein for producing a plant chromosome that is acrocentric and/or contains adjacent regions of heterchromatin and rDNA, the recombination event or events that lead to formation of the chromosome occur through crossing of transgenic plants that contain chromosomes which contain complementary site-specific recombination sites. Thus, in one embodiment of these methods, nucleic acid containing a recombination site adjacent to nucleic acid encoding a selectable marker is introduced into a first plant cell and a first transgenic plant is generated from the first plant cell. Nucleic acid containing a promoter functional in a plant cell, a recombination site and a recombinase coding region in operative linkage is introduced into a second plant cell from which a second transgenic plant is generated. The first and second transgenic plants are crossed to obtain one or more plants resistant to an agent that selects for cells containing the nucleic acid encoding the selectable marker, and a resistant plant that contains cells comprising a plant chromosome that is acrocentric and/or contains adjacent regions of heterochromatin and rDNA is selected.

In an example of this method, nucleic acids containing site-specific recombination sites are introduced into cells of Nicotiana tabacum. The nucleic acids are introduced separately by infecting leaf explants with Agrobacterium tumefaciens which carries the kanamycin-resistance gene (Kan^{R}). Kanamycin-resistant transgenic plants are generated from the infected leaf explants. One transgenic plant contains nucleic acid encoding a promoterless hygromycin-resistance gene preceded by a lox-site specific recombination sequence (lox-hpt), the other plant contains a cauliflower mosaic virus 35S promoter linked to a lox sequence and the cre DNA recombinase coding region (35S-lox-cre). The resultant Kan^{R} transgenic plants are crossed (see, e.g., protocols of Qin et al. (1994) Proc. Natl. Acad. Sci. U.S.A. 91:1706-1710, 1994). Plants in which the appropriate DNA recombination event has occurred are identified by hygromycin-resistance.

The Kan^{R} cultivars initially may be screened, such as by FISH, to identify two sets of candidate transgenic plants. One set has one construct integrated in regions adjacent to the pericentric heterochromatin (and/or satellite DNA) on the short arm of any chromosome. The second set of candidate plants has the other construct integrated in rDNA, such as the NOR region, of appropriate chromosomes. To obtain reciprocal translocation both sites must be in the same orientation. Therefore a series of crosses may be required, marker-resistant plants generated, and FISH analyses performed to identify an “acrocentric” plant chromosome or chromosomes that contain adjacent regions of heterochromatin. As described above, such an acrocentric chromosome may be used for de novo plant artificial chromosome formation, particularly predominantly heterochromatic plant artificial chromosomes. The selection of appropriate plant lines can be done, for example, using marker-assisted selection.

F. Incorporation of Heterologous Nucleic Acids into Artificial Chromosomes

Heterologous nucleic acids can be introduced into artificial chromosomes during or after formation. Incorporation of particular desired nucleic acids into an artificial chromosome during generation thereof may be accomplished by including the desired nucleic acids along with the nucleic acid encoding a selectable marker and any other nucleic acids used in artificial chromosome generation (e.g., targeting sequences that direct the heterologous nucleic acid to the pericentric region of a chromosome) in the transformation of a cell to initiate amplification and formation of a artificial chromosomes.

Alternatively, heterologous nucleic acids may be incorporated into an artificial chromosome following formation thereof through transfection of a cell containing the artificial chromosome with the heterologous nucleic acids. In general, incorporation of such nucleic acids into the artificial chromosome is assured through site-directed integration, such as may be accomplished by including nucleic acids homologous or identical to DNA contained within the artificial chromosome in with the heterologous nucleic acid when transferring it to the artificial chromosome. An additional selective marker gene may also be included.

Additionally, introduction of nucleic acids, particularly DNA molecules to an artificial chromosome can be accomplished by the use of site-specific recombinases as described herein (see, also, copending U.S. provisional application Serial No. 60/294,758 by Perkins et al. entitled “CHROMOSOME-BASED PLATFORMS” filed on May 30, 2001, U.S. provisional application Serial No. 60/366,891, by Perkins et al. entitled “CHROMOSOME-BASED PLATFORMS” filed on Mar. 21, 2002, U.S. patent application Ser. No. ______, by Perkins et al. entitled “CHROMOSOME-BASED PLATFORMS” filed on May 30, 2002, under attorney docket no. 24601-420, and PCT International Application No. ______, by Perkins et al. entitled “CHROMOSOME-BASED PLATFORMS” filed on May 30, 2002, under attorney docket no. 24601-420PC; each of which is incorporated in its entirety by reference thereto). Artificial chromosomes can be produced containing recombinase recognition sequences, to allow the site-specific introduction of DNA molecules into the same. Another use for an introduced recombinase site is to provide a region for site-specific integration of a new trait by the use of recombinase mediated gene insertion.

G. Introduction of Artificial Chromosomes into Plant Cells and Recovery of Plants Containing Artificial Chromosomes

Artificial chromosomes can be introduced into plant cells by a variety of methods familiar to those skilled in the art. These methods include chemical and physical methods for introduction of foreign DNA, as well as cell culture methods to transfer chromosomes from one cell to another cell.

Any type of artificial chromosome can be used. Plant artificial chromosomes (PACs) can be prepared by the in vivo and in vitro methods described herein. PACs can be prepared inside plant protoplasts and then transferred to other plant species and tissues, in particular to other plant protoplasts, via fusion in the presence or absence of PEG as described herein (Draper et al. (1982) Plant Cell Physiol. 23:451-458; Krens et al. (1982) Nature 72-74). PACs can be isolated from the protoplasts in which they were prepared, encapsulated into liposomes, and delivered to other plant protoplasts (Deshayes et al. (1985) EMBO J. 4:2731-2737). Alternatively, the PACs can be isolated and delivered directly to plant protoplasts, plant cells, or other plant targets via a PEG-mediated process, calcium phosphate-mediated process, electroporation, microinjection, (particle bombardment), lipid-mediated method with or without sonoporation, sonoporation alone, or any method known in the art as described herein (Haim et al. (1985) Mol. Gen. Genet. 199:161-168; Fromm et al. (1986) Nature 319:791-793; Fromm et al. (1985) Proc. Nat. Acad. Sci. U.S. Pat. No.A 82:5824-5828; Klein et al. (1987) Nature 327:70; Klein et al. (1988) Proc. Nat. Acad. Sci. U.S. Pat. No.A 85:8502-8505; and International PCT application publication no. WO 91/00358). Plant artificial chromosomes can also be transferred to other plant species by preparation of protoplast-derived plant microcells, and fusion of the microcells containing the plant artificial chromosome with plant cells of other plant species.

Mammalian artificial chromosomes (MACs) can be transferred to plant cells. Mammalian artificial chromosomes are prepared by the in vivo and in vitro methods described in U.S. Pat. Nos. 6,025,155 and 6,077,697, and International PCT application No. WO 97/40183. MACs can be prepared as microcells, and the microcells can be fused with plant protoplasts in the presence or absence of PEG (Dudits et al. (1976) Hereditas 82:121-123; Wiegland et al. (1987) J. Cell. Sci. Pt. 2 145-149). Alternatively, the MACs can be isolated and delivered directly to plant cells, protoplasts, and other plant targets using a PEG-mediated process, calcium phosphate-mediated process, electroporation, microinjection, lipid-mediated method with or without sonoporation, sonoporation alone, or any method known in the art as described herein and in U.S. Pat. Nos. 6,025,155 and 6,077,697, and International PCT application publication No. WO 97/40183.

After PACs or MACs are introduced into plant targets and the plant targets are grown and analyzed for transfection, the plant transformed plant targets can be developed using standard conditions into roots, shoots, plantlets, or any structure capable of growing into a plant.

Accordingly, methods for the introduction of artificial chromosomes represent the first step in the production of plant cells and whole plants containing artificial chromosomes from a variety of sources.

The ability to introduce genes into plants, such that they are stably expressed and transmissible from generation to generation, has revolutionized plant biology and opens up new possibilities for using plants as green factories for the production of commercially useful products as well as for other applications described herein. There are several approaches to the generation of stably transformed plants, and the adopted approach varies according to the aims of the project. For introduction of artificial chromosomes into plants, a variety of methods may be employed. transgenic plants, the transformation process involves the methods of foreign DNA delivery to plant host cells, the growth and analysis of transformed plant host cells, and the generation and regeneration of transgenic plants from transformed plant host cells.

1. Introduction of Artificial Chromosomes into Plant Host Cells

Numerous methods for producing or developing transgenic plants are available to those of skill in the art. The method used is primarily a function of the species of plant. Artificial chromosomes containing heterologous DNA, such as artificial chromosomes prepared by the methods described herein, can be introduced into plant host cells, including, but not limited to, plant cells and protoplasts, by, for example, non-vector mediated DNA transfer processes (see, also copending U.S. application Ser. No. 09/815,979, which describes methods for delivery that can be adapted for use with plant cells and used with plant protoplasts).

Non-vector mediated, or direct, gene transfer systems involve the introduction of heterologous DNA, in particular artificial chromosomes, into host cells, including but not limited to plant cells and protoplasts, without the use of a biological vector. The artificial chromosome that is introduced into these plant host cells can lead to the development of transformed, regenerable transgenic plants. The direct gene transfer systems for transgenic plants are designed to overcome the barrier to DNA uptake caused by the cell wall and the plasma membrane of plant cells. The approaches for direct gene transfer include, but are not limited to, chemical, electrical, and physical methods, which can also be adapted to optimize transfer of artificial chromosomes (see, e.g., Uchimiya et al. (1989) J. of Biotech. 12: 1-20 for a review of such procedures, see also, e.g., U.S. Pat. Nos. 5,436,392; 5,489,520; Potrykus et al. (1985) Mol. Gen. Genet. 199:183; Lorz et al. (1985) Mol. Gen. Genet. 199:178; Fromm et al. (1985) Proc. Natl. Acad. Sci. U.S.A. 82:5824-5828; Uchimiya et al. (1986) Mol. Gen. Genet. 204:204; Callis et al. (1987) Genes Dev. 1:1183-2000; Callis et al. (1987) Nuc. Acids Res. 15:5823-5831; Marcotte et al. (1988) Nature 355:454 and Toriyama et al. (1988) Bio/Technology 6:1072-1074).

a. Chemical Methods

Uptake of artificial chromosomes into plant cells, such as protoplasts, can be accomplished in the absence or presence of polyethylene glycol (PEG), which is a fusogen, or by any variations of such methods known to those of skill in the art [see, e.g., U.S. Pat. No. 4,684,611 to Schilperoot et al.; Paskowski et al. (1984) EMBO J. 3:2717-2722; U.S. Pat. Nos. 5,231,019 and 5,453,367]. In one approach, plant protoplasts are incubated with a solution of foreign DNA, in particular artificial chromosomes, and PEG at a concentration that allows for high cell survival and high efficiency chromosome uptake. The protoplasts are then washed and cultured [Datta and Datta (1999) Meth. in Molecular Biol. 111:335-348]. In an alternative approach, plant protoplasts are incubated with artificial chromosomes in the presence of calcium phosphate for direct artificial chromosome uptake (Haim et al. (1985) Mol. Gen. Genet. 199:161-168). Alternatively, the artificial chromosome, in particular plant artificial chromosome (PAC), is formed in a plant protoplast which is, in turn, fused with another plant protoplast in the presence or absence of PEG to transfer the PAC to the plant host protoplast. Such methods for treating protoplasts with PEG and foreign DNA are well known in the art (Draper et al. (1982) Plant Cell Physiol. 23:451-458; Krens et al. (1982) Nature 72-74).

Another chemical direct gene transfer method involves lipid-mediated delivery of artificial chromosomes to plant protoplasts. In this process, liposomes with encapsulated artificial chromosomes are allowed to fuse with protoplasts alone or in the presence of PEG as the fusogen to transfer the foreign DNA, in particular artificial chromosome, to the plant host protoplast (Deshayes et al. (1985) EMBO J. 4:2731-2737; Fraley and Paphadjopoulos (1982) Curr Top Microbiol Immunol 96:171-191).

Another direct gene transfer method involves the use of microcells. The chromosomes can be transferred by preparing microcells containing artificial chromosomes and then fusing the microcells with plant protoplasts. Methods for the preparation and fusion of microcells with other cells are well known in the art (see Example No. 4 and see also, e.g., U.S. Pat. Nos. 5,240,840; 4,806,476; 5,298,429; 5,396,767; Fournier (1981) Proc. Natl. Acad. Sci. U.S.A. 78:6349-6353; and Lambert et al. (1991) Proc. Natl. Acad. Sci. U.S.A. 88:5907-59; Dudits et al. (1976) Hereditas 82:121-123; Wiegland et al. (1987) J. Cell. Sci. Pt. 2 145-149).

b. Electrical Methods

Electroporation, which involves high-voltage electrical pulses to a solution containing a mixture of protoplasts or plant cells and foreign DNA, in particular artificial chromosomes, to create nanometer-sized, reversible pores, is a common method to introduce DNA into plant cells or protoplasts. The exogenous DNA may be added to the protoplasts in any form such as, for example, naked linear, circular or supercoiled DNA, artificial chromosomes encapsulated in liposomes, DNA in spheroplasts, artificial chromosomes in other plant protoplasts, artificial chromosomes complexed with salts, and other methods. The foreign DNA, in particular artificial chromosome, can also include a phenotypic marker to identify plant cells that are successfully transformed.

When plant cells or protoplasts are subjected to short electrical DC (direct current) pulses, they may experience an increase in the permeability of the plasma membrane and/or cell wall to hydrophilic molecules such as nucleic acids, which are normally unable to enter the plant cell directly. Nucleic acids are taken directly into the cell cytoplasm either through these pores or as a consequence of the redistribution of membrane components that accompanies closure of the pores. Certain cell wall-degrading enzymes, such as pectin-degrading enzymes, may be employed to render the plant target recipient cells more susceptible to DNA or artificial chromosome uptake by electroporation than untreated cells. Plant recipient cells may also be susceptible to transformation by mechanical wounding. To effect transformation by electroporation, friable tissues such as a suspension culture of cells or embryonic callus may be used or immature embryos or other organized tissues may be directly transformed (see, e.g., Fromm et al. (1 986) Nature 319:791-793). Methods for effecting electroporation are well known in the art (see, e.g., U.S. Pat. Nos. 4,784,737; 4,970,154; 5,304,486; 5,501,967; 5,501,662; 5,019,034; 5,503,999; see, also Fromm et al. (1985) Proc. Natl. Acad. Sci. U.S.A. 82:5824-5828; Zimmerman et al. (1981) Biophys Biochem Acta 641:160-165; Neuman et al. (1982) EMBO J. 1:841-845; Riggs et al. (1986) Proc. Nat. Acad. Sci. U.S. Pat. No.A 83:5602-5606; Lurquin (1997) Mol. Biotechnol. 7:5-35; Bates (1999) Methods in Molecular Biology 111:359-366). Electroporation can be used to introduce nucleic acids into tobacco mesophyll cells (Morikawa et al. (1986) Gene 41:121-124; leaf bases of rice (Dekeyser et al. (1990) Plant Cell 2:591-602; immature maize embryos (Songstad et al. (1993) Plant Cell Tiss. Orgn. Cult. 40:1-15; macerated immature maize embryos (D'Halluin et al. (1992) Plant Cell 4:1495-1505; suspension cultured maize cells (Laursen et al. (1994) Plant Mol. Biol. 24: 51-61; and sugar cane (Arencibia et al. (1995) Plant Cell Rep. 14:305-309).

Artificial chromosomes may be delivered to plant cells, in particular plant seeds, by the use of electroporation and pollen to derive pollen comprising an artificial chromosome. Methods that may be used for delivery of artificial chromosomes into pollen include, for example, techniques described in U.S. Pat. No. 5,049,500 and by Negrutiu et al. [in Biotechnology and Ecology of Pollen, Mulcahy et al. eds., (1986) Springer Verlag, N.Y., pp. 65-69] and Fromm et al. [(1986) Nature 319:791; including methods for introducing DNA into mature pollen using various procedures such as heat shock, PEG and electroporation]. The pollen is capable of germinating and fertilizing an egg cell, leading to the formation of a plant seed comprising an artificial chromosome.

c. Physical Methods

The physical methods approach for introducing foreign DNA, in particular artificial chromosomes, into plant cells overcomes the cell wall barrier to DNA movement. Physical, or mechanical means, are used to introduce transgenes directly into protoplasts or plant cells and include, but are not limited to, microinjection, particle bombardment, and sonoporation.

### (1) Microinjection

Microinjection involves the mechanical injection of heterologous DNA, in particular artificial chromosomes, into plant cells, including cultured cells and cells in intact plant organs and embryoids in tissue culture via very small micropipettes, needles, or syringes (Neuhaus et al. (1987) Theor. Appl Genet. 75:30-36; Reich et al. (1986) Can. J. Bot. 64:1255-1258; Crossway et al. (1986) BioTechniques 4:320-334; Crossway et al. (1986) Mol. Gen. Genet. 20:179; U.S. Pat. No. 4,743,548; silicon carbide whiskers (Kaeppler et al. (1990) Plant Cell Rep. 9:415-418; Frame et al. (1994). For example, microinjection of protoplast cells with foreign DNA for transformation of plant cells has been reported for barley and tobacco (see, e.g., Holm et al. (2000) Transgenic Res. 9:21-32 and Schnorf et al. Transgenic Res. 1:23-30). Single artificial chromosomes may be front-loaded into microinjection needles and then injected into cells (“pick-and-inject”) following procedures as described by Co et al. [(2000) Chromosome Res. 8:183-191].

### (2) Particle Bombardment

Microprojectile bombardment (acceleration of small high density particles, which contain the DNA, to high velocity with a particle gun apparatus, which forces the particles to penetrate plant cell walls and membranes)have also been used to introduce heterologous DNA into plant cells. Microprojectile bombardment techniques for the introduction of nucleic acids into plant cells, in addition to being an effective means of reproducibly stably transforming plant cells, particularly monocots, do not require isolation of protoplasts or susceptibility of the host cell to Agrobacterium infection. In these methods, nucleic acids are carried through the cell wall and into the cytoplasm on the surface of small, typically metal, particles (see, e.g., Klein et al. (1987) Nature 327:70; Klein et al. (1988) Proc. Natl. Acad. Sci. U.S.A. 85:8502-8505, Klein et al. in Progress in Plant Cellular and Molecular Biology, eds. Nijkamp, H. J. J., Van der Plas, J. H. W., and Van Aartrijk, J., Kluwer Academic Publishers, Dordrecht, (1988), p. 56-66 and McCabe et al. (1988) Bio/Technology 6:923-926; Sautter et al. (1991) Biol. Technol. 9:1080-1085; Gordon-Kamm et al. (1990) Plant Cell 2:603-618; Finer et al. (1999) Curr. Top. Microbiol. Immunol. 240:59-80; Vasil and Vasil (1999) Methods in Molecular Biology 111:349-358; Seki et al. (1 999) Mo. Biotechnol. 11:251-255). Particles may be coated with nucleic acids and delivered into cells by a propelling force. Exemplary particles include those containing tungsten, gold or platinum, as well as magnesium sulfate crystals. The metal particles can penetrate through several layers of cells and thus allow the transformation of cells within tissue explants.

In an illustrative embodiment (see, e.g., U.S. Pat. No. 6,023,013) of a method for delivering foreign nucleic acids into plant cells, e.g., maize cells, by acceleration, a Biolistics Particle Delivery System may be used to propel particles coated with DNA or cells through a screen, such as a stainless steel or Nytex screen, onto a filter surface covered with plant (e.g., corn) cells cultured in suspension. The screen disperses the particles so that they are not delivered to the recipient cells in large aggregates. The intervening screen between the projectile apparatus and the cells to be bombarded may reduce the size of projectile aggregates and may contribute to a higher frequency of transformation by reducing damage inflicted on the recipient cells by projectiles that are too large.

For the bombardment, cells in suspension may be concentrated on filters or solid culture medium. Alternatively, immature embryos or other plant target cells may be arranged on solid culture medium. The cells to be bombarded are typically positioned at an appropriate distance below the microprojectile stopping plate. If desired, one or more screens may also be positioned between the acceleration device and the cells to be bombarded.

The prebombardment culturing conditions and bombardment parameters may be optimized to yield the maximum numbers of stable transformants. Both the physical and biological parameters for bombardment are important in this technology. Physical factors include those that involve manipulating the DNA/microprojectile precipitate or those that affect the flight and velocity of either the macro- or microprojectiles. Biological factors include all steps involved in manipulation of cells before and immediately after bombardment, the osmotic adjustment of target cells to help alleviate the trauma associated with bombardment, and also the nature of the transforming nucleic acid, such as linearized DNA, intact supercoiled plasmids, or artificial chromosomes.

Physical parameters that may be adjusted include gap distance, flight distance, tissue distance and helium pressure. In addition, transformation may be optimized by adjusting the osmotic state, tissue hydration and subculture stage or cell cycle of the recipient cells. Ballistic particle acceleration devices are available from Agracetus, Inc. (Madison, Wis.) and BioRad (Hercules, Calif.).

Techniques for transformation of A188-derived maize line using particle bombardment are described in Gordon-Kamm et al. (1990) Plant Cell 2:603-618 and Fromm et al. (1990) Biotechnology 8:833-839. Transformation of rice may also be accomplished via particle bombardment (see, e.g., Christou et al. (1991) Biotechnology 9:957-962). Particle bombardment may also be used to transform wheat (see, e.g., Vasil et al. (1992) Biotechnology 10:667-674 for transformation of cells of type C long-term regenerable callus; and Weeks et al. (1993) Plant Physiol. 102:1077-1084 for transformation of wheat using particle bombardment of immature embryos and immature embryo-derived callus). The production of transgenic barley using bombardment methods is described, for example, by Koprek et al. (1996) Plant Sci. 119:79-91.

### (3) Sonoporation

Foreign DNA, in paticular artificial chromosomes, may be introduced into plant protoplasts using ultrasound treatment, in particular mild ultrasound treatment (10-100 kHz), to create pores for DNA uptake (see e.g. International PCT application publication no. WO 91/00358) or may be introduced into plant protoplasts via a sonoporation machine (ImaRx Pharmaceutical Corp., Tucson, Ariz.).

Alternatively, the delivery of artificial chromosomes into plant host cells is performed by any method described herein or well known in the art. For example, needle-like whiskers (U.S. Pat. No. 5,302,523, 1994, U. S. Pat. No. 5,464,765) have been used to delivery foreign DNA.

Suitable plant targets into which foreign DNA, in particular artificial chromosomes, is transferred include, but are not limited to, protoplasts, cell culture cells, cells in plant tissue, meristem cells, microspores, callus, pollen, pollen tubes, microspores, egg-cells, embryo-sacs, zygotes or embryos in different stages of development, seeds, seedlings, roots, stems, leaves, whole plants, algae, or any plant part capable of proliferation and regeneration of plants. (see, e.g., U.S. Pat. Nos. 5,990,390; 6,037,526 and 5,990,390). The growth of the transformed plant targets described herein can done with tissue-culture or non-tissue culture methods, with the preferred methods being tissue culture methods.

All plant cells into which foreign DNA, in particular artificial chromosomes, are introduced and that is regenerated from the transformed cells are used directly for expressed purposes (e.g. herbicide resistance, insect/pest resistance, disease resistance, environmental/stress resistance, nutrient utilization, male sterility, improved nutritional content, production of chemicals or biologicals, non-protein expressing sequences, and preparation and screening of libraries) as described herein or are used to produce transformed whole plants for the applications and uses described herein. The particular protocol and means for the introduction of the artificial chromosome into the plant host is adapted or refined to suit the particular plant species or cultivar.

Chromosomes may be transferred to cells by microcell mediated chromosome transfer (MMCT) (Telenius et al., Chromosome Research 7:3-7, 1999; Ramulu et al., Methods in Molecular Biology 111: 227-242, 1999). In general, donor plant cultures or donor mammalian cell cultures are incubated in media supplemented with reagents that inhibit DNA synthesis (e.g., hydroxy urea, aphidicolin) and/or reagents that inhibit attachment of chromosomes to the mitotic spindle (e.g., colcemid, colchicines, amiprophos-methyl, cremart). The cell walls of plant cells are digested with enzymes (e.g., cellulase, maceroenzyme) producing protoplasts. Donor plant protoplasts or donor mammalian cells are loaded on a Percoll gradient in the presence of cytochalasin-B (which causes the cell cytoskeleton to depolymerize into monomer protein subunits) and centrifuged at 10⁵×g. During centrifugation the metaphase chromosomes are extruded through the plasma membrane forming plant ‘microprotoplasts’ or mammalian ‘microcells.’ The microprotoplasts/microcells are filtered through nylon sieves of decreasing pore size (8-3 μm) to isolate smaller ones that contain predominately 1 metaphase chromosome. The microprotoplasts/microcells are fused to recipient plant protoplasts or mammalian cells by polyethelene glycol (peg) treatment. The fusion mixture is cultured in appropriate media. If the chromosome of interest is expressing a selection marker gene the fusion mixtures may be cultured in appropriate media supplemented with the appropriate selection drug (e.g. hygromycin, kanamycin).

2. The growth of Transformed Plant Host Cells

In tissue culture methods, plant cells or protoplasts transformed by the chemical, physical, electrical methods described herein are grown, or cultured, under selective conditions. The selective markers are integrated into the heterologous DNA, in particular artificial chromosome, before its introduction to plant hosts or are integrated into the plant host after transfection. An additional marker can be used for double selection. Generally, the plant cells or protoplasts are grown for numerous generations, after which the transformed cells are identified.

The transformed cells are subjected to conditions known in the art for callus initiation. Tissue that develops during the initiation period is placed in a regeneration or selection medium where shoot and root development occur. The plantlets are analyzed for the determination of transformation (International PCT application publication no. WO 00/60061). In the case of maize, embryonic callus cultures are initiated from immature maize embryos, bombarded with genes, and transformed into plantlets by the methods described in International PCT application publication no. WO 00/60061. In tissue culture methods, Rice calli are transformed with DNA encoding insecticidal proteins CryIA(b) and CryIA(c) for insect resistance. Common tissue culture methods can also be used to transform tobacco and tomato (see, e.g., U.S. Pat. No. Pat. No. 6,136,320), embryogenic maize calli (U.S. Pat. Nos. 5,508,468; 5,538,877; 5,538,880; 5,780,708; 6,013,863; 5,554,798; 5,990,390; and 5,484,956;) and other crop species, e.g., potato and tobacco (Sijmons et al. (1990) Bio/Technol 8:217-221; tobacco (Vanderkerckhove et al. (1989) Bio/Technol 7:929-932 and Owen and Pen eds. Transgenic Plants: A Production System for Industrial and Pharmaceutical Proteins, John Wiley & Sons, Chichester, 1996) and rice (Zhu et al. (1994) Plant Cell Tiss Org Cult 36:197-204).

3. Analysis of Transformed Plant Host Cells

Once foreign DNA, in particular artificial chromosomes, is introduced into plant hosts and the cells or protoplasts are grown and developed under the conditions described herein, the plant cells or protoplasts which were transformed with artificial chromosomes are identified. The plant cell, protoplast, callus, leaf disc, or other plant target are screened for the presence of artificial chromosomes by various methods well known in the art including, but not limited to, assays for the expression of reporter genes, PCR of the isolated plant chromosomes or DNA, electron microscopy, visualization methods, and in situ hybridization of chromosome painting probe as described herein. Moreover, cells treated with artificial chromosomes are isolated during metaphase using a mitotic arrest agent, such as colchicine, and the artificial chromosome are distinguished from endogenous chromosomes by fluorescence-activated cell sorting, size and density differences, or by any method well known in the art. Alternatively, when a selectable marker gene is transmitted with or as part of the artificial chromosome, selective agents are used to detect the expression of the selectable marker (International PCT application publication no. WO 00/60061; U.S. Pat. No. 6,136,320; Owen and Pen Eds. Transgenic Plants: A Production System for Industrial and Pharmaceutical Proteins). Enzymatic assays, immunological assays, bioassays, germination assays, or chemical assays are used to assess the phenotypic effects of artificial chromosomes such as insect or fungal resistance or any other expression of genes in artificial chromosomes (Cheng et al. (1998) 95:2767-2772; U.S. Pat. No. 6,126,320; International PCT application publication no. WO 00/60061; Owen and Pen eds. Transgenic Plants: A Production System for Industrial and Pharmaceutical Proteins, John Wiley & Sons, Chichester, 1996). The plant cells, protoplasts, or other plant hosts that are successfully transformed with artificial chromosomes are used directly to express the gene of interest or are used to generate transgenic plants.

Fluorescent in situ hybridization (FISH) may be used to screen for the transfer of artificial chromosomes into plant cells. Using DNA probes specfic for the artificial chromosome (e.g., mouse major satellite DNA probe for murine satellite DNA based artificial chromosomes; or a kanamycin, hygromycin or GUS gene DNA probe for a plant artificial chromosome carrying such a gene) standard FISH techniques for plant cells have been described (de Jong et al., Trends in Plant Science 4: 258-263, 1999).

IdU labeling can be used to determine the optimum conditions for chromosome transfer (microcells) or isolated artificial chromosomes. The incorporated IdU increases the fragility of the chromosome and will increase the probability of cellular mutation. Hence, the cells are fixed within 48-hours after transfection/fusion and analyzed for chromosome uptake using various procedures. Once the optimum transfer conditions have been determined, long-term expression experiments are performed with unlabeled artificial chromosomes or microcells.

H. Re-Gen Ration of Transgenic Plants

Plants containing artificial chromosomes are generated from plant cells, protoplasts, calli, or other plant tissue targets into which foreign DNA, in particular artificial chromosomes, have been introduced. Regeneration techniques for many commercially important plant species are well-known in the art. The artificial chromosome that is inserted into plant hosts to produce transgenic plants are PACs or MACs.

Plants are re-generated by the planting of transformed roots, plantlets, seeds, seedlings and structures capable of growing into a whole plant capable of reproduction (see, e.g., U.S. Pat. Nos. 6,136,320 and International PCT application No. WO 00/60061). The re-generation of maize plants from transformed protoplasts is found, for example, in European Patent Application nos. 0 292 435 and 0 392 225 and International PCT Application Publication no. WO 93/07278; the regeneration of rice following gene transfer is found in Zhang et al. (1988) Plant Cell Rep. 7:379-384; Shimamoto et al. (1989) Nature 338:274-277; Datta et al. (1990) Biotechnology 8:736-740; and the re-generation of fertile transgenic barley by direct DNA transfer to protoplasts is described by Funatsuki et al. (1995) Theor. Appl. Genet. 91:707-712. Alternatively, plants containing artificial chromosomes are obtained by crossing a plant containing an artificial chromosome with another plant to produce plants having an artificial chromosome in their genomes (see e.g. U.S. Pat. No. 6,150,585).

Plants containing an artificial chromosome are propagated through seed, cuttings, or vegetatively. The seed from plants containing an artificial chromosome are grown in the field, in pots, indoors, outdoors, in greenhouses, on glass, or in or on any suitable medium, and the resulting sexually mature transgenic plants are self-pollinated to generate true breeding plants. The progeny from these transgenic plants become true breeding lines (International PCT application publication Nos. WO 00/60061 and EP 1017268; U.S. Pat. Nos. 5,631,152; 5,955,362; 6,015,940; 6,013,523; 6,096,546; 6,037,527; 6,153,812; Weissbach and Weissbach (1988) Methods for Plant Molecular Biology, Academic Press, Inc.; Fromm et al. (1990) Bio/Technology 8:833-839; Gordon-Kamm et al. (1990) Plant Cell 2:603-608; Koziel et al. (1993) Bio/Technology 11:194-200; and Golovkin et al. (1993) Plant Sci. 90:41-52).

1. PACs

Plant artificial chromosomes (PACs) are prepared by the in vivo and in vitro methods described herein. PACs may be prepared inside plant protoplasts and then transferred to plant targets, in particular to other plant protoplasts, via fusion in the presence or absence of PEG as described herein (Draper et al. (1982) Plant Cell Physiol. 23:451-458; Krens et al. (1982) Nature 72-74). PACs are isolated from the protoplasts in which they were prepared, encapsulated into liposomes, and delivered to other plant protoplasts (Deshayes et al. (1985) EMBO J. 4:2731-2737). Alternatively, the PACs are isolated and delivered directly to plant protoplasts, plant cells, or other plant targets via a PEG-mediated process, calcium phosphate-mediated process, electroporation, microinjection, sonoporation, or any method known in the art as described herein (Haim et al. (1985) Mol. Gen. Genet. 199:161-168; Fromm et al. (1986) Nature 319:791-793; Fromm et al. (1985) Proc. Nat. Acad. Sci. USA 82:5824-5828; Klein et al. (1987) Nature 327:70; Klein et al. (1988) Proc. Nat. Acad. Sci. USA 85:8502-8505; and International PCT application publication no. WO 91/00358).

2. MACs

Mammalian artificial chromosomes (MACs) are prepared by the in vivo and in vitro methods described in U.S. Pat. Nos. 6,025,155 and 6,077,697, and International PCT application No. WO 97/40183. MACs are prepared as microcells, and the microcells are fused with plant protoplasts in the presence or absence of PEG (Dudits et al. (1976) Hereditas 82:121-123; Wiegland et al. (1987) J. Cell. Sci. Pt. 2 145-149). Alternatively, the MACs are isolated and delivered directly to plant cells, protoplasts, and other plant targets a PEG-mediated process, calcium phosphate-mediated process, electroporation, microinjection, sonoporation, or any method known in the art as described herein and in U.S. Pat. Nos. 6,025,155 and 6,077,697, and International PCT application publication No. WO 97/40183.

After PACs or MACs are introduced into plant targets and the plant targets are grown and analyzed for transfection, the transformed plant targets are developed using standard conditions into roots, shoots, plantlets, or any structure capable of growing into a plant. Transgenic plants can, in turn, be generated by the planting of transformed roots, plantlets, seeds, seedlings and structures capable of growing into a plant.

Transgenic plants can be propagated, for example, through seed, cuttings, or vegetative propagation.

I. Applications and Uses of Artificial Chromosomes

Artificial chromosomes provide convenient and useful vectors, and in some instances (e.g., in the case of very large heterologous genes) the only vectors, for introduction of heterologous genes into hosts. Virtually any gene of interest is amenable to introduction into a host via artificial chromosomes.

As described herein, there are numerous methods for using artificial chromosomes to introduce coding sequences into plant cells. These include methods for using artificial chromosomes to express genes encoding commerically valuable enzymes and therapeutic compounds in plant cells, introduction of agronomically important traits or applications related to the manipulation of large regions of DNA.

The artificial chromosomes provided herein may be used in methods of protein and gene product production, particularly using plant cells as host cells for production of such products, and in cellular production systems in which the artificial chromosomes provide a reliable, stable and efficient means for optimizing the biomanufacturing of important compounds for medicine and industry. They are also intended for use in methods of gene therapy and for production of transgenic organisms, particularly plants (discussed above, below and in the EXAMPLES).

1. Production of Products in Plants

Methods for expression of heterologous proteins in plant cells (“molecular farming”) are provided. At present, many foreign proteins have been expressed in whole plants or selected plant organs. Plants can offer a highly effective and economical means to produce recombinant proteins as they can be grown on a large scale at modest cost. The production of heterologous proteins in plants has included genes that are fused to strong constitutive plant promoters (e.g., 35S from cauliflower mosaic virus (Sijmons et al., 1990, Bio/Technology, 8:217-221, Benfey and Chua, U.S. Pat. No. 5,110,732, Fraley et al., U.S. Pat. No. 5,858,742, McPherson and Kay, U.S. Pat. No. 5,359,142); seed specific promoters (Hall et al., U.S. Pat. No. 5,504,200, Knauf et al., U.S. Pat. No. 5,530,194, Thomas et al., U.S. Pat. No. 5,905,186, Moloney, U.S. Pat. No. 5,792,922, U.S. Pat. No. 5,948,682) or promoters active in other plant organs such as fruit (Radke et al., 1988, Theoret. Appl. Genet., 75:685-694, Bestwick et al., U.S. Pat. No. 5,783,394, Houck and Pear, U.S. Pat. No. 4,943,674) or storage organs such as tubers (Rocha-Sosa et al., U.S. Pat. No. 5,436,393, U.S. Pat. No. 5,723,757). The genes under the control of these promoters can be any protein and include, for example, genes that encode receptors, cytokines, enzymes, proteases, hormones, growth factors, antibodies, tumor suppressor genes, vaccines, therapeutic products and multigene pathways.

For example, industrial enzymes that can be produced include, for example, α-amylase, glucanase, phytase and xylanase (see, Goddijn and Pen (1995) Trends Biotechnol. 13:379-387; Pen et al. (1992) Bio/Technology 10:292-296; Horvath et al. (2000) Proc. Natl. Acad. Sci. U.S.A. 97:1914-1919; and e.g., Herbers and Sonnewald (1996) in Transgenic Plants: A Production System for Industrial and Pharmaceutical Proteins” Owen and Pen Eds., John Wiley & Sons, West Sussex, England), proteases such as subtilisin and other industrially important enzymes. Additional proteins that can be produced in crops by molecular farming include other industrial enzymes, for example, proteases, carbohydrate modifying enzymes such as glucose oxidase, cellulases, hemicellulases, xylanases, mannanases or pectinases, (e.g. Baszczynski et al., U.S. Pat. No. 5,824,870, U.S. Pat. No. 5,767,379, Bruce et al., U.S. Pat. No. 5,804,694). Additionally, the production of enzymes particularly valuable in the pulp and paper industry such as ligninases or xylanases also can be expressed, (Austin-Philips et al., U.S. Pat. No. 5,981,835). Other examples of enzymes include phosphatases, oxidoreductases and phytases, (van Ooijen et al., U.S. Pat. No. 5,714,474).

Additionally, expression and delivery of vaccines in plants has been proposed(Arntzen and Lam, U.S. Pat. No. 6,136,320, U.S. Pat. No., 5,914,123, Curtiss and Cardineau, U.S. Pat. No. 5,679,880, U.S. Pat. No. 5,679,880, U.S. Pat. No. 5,654,184, Lam and Arntzen, U.S. Pat. No. 5,612,487, U.S. Pat. No. 6,034,298, Rymerson et al., WO9937784A1, as well as antibodies (Conrad et al., WO 972900A1, Hein et al., U.S. Pat. No. 5,959,177, Hiatt and Hein, U.S. Pat. No. 5,202,422, U.S. Pat. No. 5,639,947, Hiatt et al., U.S. Pat. No. 6,046,037), peptide hormones (Vandekerckhove, J. S., U.S. Pat. No. 5,487,991, Brandle et al., WO9967401A2), blood factors and similar therapeutic molecules. Expression of vaccines in edible plants can provide a means for drug delivery which is cost effective and particularly suited for the administration of therapeutic agents in rural or under developed countries. The plant material containing the therapeutic agents could be cultivated and incorporated into the diet (Lam, D. M., and Arntzen, C. J., U.S. Pat. No. 5,484,719). Similarly, plants used for animal feed can be engineered to express veterinary biologics that can provide protection against animal disease, (Rymerson et al., WO9937784A1). Antibodies also can be produced in plants, including, for example, a gene fusion encoding an antigen-binding single chain Fv protein (scFv) that recognizes the hapten oxazolone (Fiedler and Conrad (1995) Bio/Technology 13:1090-1093) and IgG (Ma et al. (1995) Science 268:716-719). Monoclonal antibodies for therapeutic and diagnostic applications are of particular interest.

Examples of human biopharmaceuticals that can be expressed in plants include, but are not limited to, albumin (Sijmons et al. (1990)), enkephalins (Vandekerckhove et al. (1989)), interferon-á (Zhu et al. (1994) and GM-CSF (Ganz et al. (1996) in Transgenic Plants: A Production System for Industrial and Pharmaceutical Proteins, Owen and Pen Eds., John Wiley & Sons, West Sussex, England, pp. 281-297; and Sardana et al. (1998) in Methods in Biotechnology, Vol. 3: Recombinant Proteins from Plants: Production and Isolation of Clinically Useful Compounds, Cunningham and Porter, Eds., Humana Press, New Jersey; pp. 77-87).

Cells containing the artificial chromosomes provided herein can advantageously be used in in vitro plant cell-based systems for production of proteins, particularly several proteins from one cell line, such as multiple proteins involved in a biochemical pathway or multivalent vaccines. The genes encoding the proteins are introduced into the artificial chromosomes which are then introduced into plant cells. Plant cells useful for this purpose are those that grow well in culture, or most preferably, plant cells capable of being regenerated to whole plants. Plants can then be cultivated by common methods to produce plant material comprising said heterologous proteins. The heterologous proteins can be subject to purification or the plant tissue or extracts thereof can be used directly for vaccination, amelioration of disease, or processing of material, such as bleaching during pulp and paper processing or enzymatic conversion of industrial materials or feedstocks. Alternatively, the heterologous gene(s) of interest are transferred into a production cell line or plant line that already contains artificial chromosomes in a manner that targets the gene(s) to the artificial chromosomes. The cells or plants are grown under conditions whereby the heterologous proteins are expressed. Because the proteins are expressed at high levels in a stable permanent extra-genomic chromosomal system, selective conditions are not required.

Selection of host lines for use in artificial chromosome-based protein production systems is within the skill of the art, but often will depend on a variety of factors, including the properties of the heterologous protein to be produced, potential toxicity of the protein in the host cell, any requirements for post-translational modification (e,g., glycosylation, amination, phosphorylation) of the protein, transcription factors available in the cells, the type of promoter element(s) being used to drive expression of the heterologous gene, whether production is completely intracellular or the heterologous protein will preferably be secreted from the cell, or be sequestered or localized, and the types of processing enzymes in the cell.

Artificial chromosomes can be engineered as platforms for the production of specific molecules in plant cells. For example, production of complex mammalian molecules, such as multichain antibodies, requires a number of protein activities not normally found in plant species. It is possible to produce an artificial chromosome that comprises all of the mamalian activities needed to produce human antibodies, correctly modified and processed, by introducing into an artificial chromosome the genes needed to carry out these activities. Said genes would be modified, for example, by placing each gene under the control of a plant promoter, or by placing the master control gene, i.e., a gene that controls expression of the various genes, under the control of a plant promoter. Alternatively, mammalian transcriptional control factors could be introduced, under the control of plant active promoters, to be expressed in a plant cell and cause the expression of said target proteins, for example multichain antibodies.

In this fashion, plant artificial chromosomes are developed, each capable of supporting the efficient production of a specific class of valuable products, for example, antibodies, blood clotting factors, etc. Thus, production of products within a class, for example, human antibodies would simply involve the introduction of a specific antibody coding sequence, without modification into the artificial chromosome engineered specifically for the production of human antibodies. The artificial chromosome would comprise all of the required genetic activities for the proper expression, translation and post-translational modification of human antibodies. Such artificial chromosomes can be used in a variety of applications, such as, but are not limited to, large scale production of numerous specific human antibodies.

Advantages of plant cells as host cell lines in the production of recombinant proteins include, but are not limited to, the following: (1) proteins are post-translationally modified similar to mammalian systems, (2) plants can be directed to secrete proteins into stable, dry, intracellular compartments of seeds called endosperm protein bodies, which can easily be collected, (3) the amount of recombinant product that can be produced approaches industrial scale levels and (4) health risks due to contamination with potential pathogens/toxins are minimized.

The artificial chromosome-based system for heterologous protein production has many advantageous features. For example, as described above, because the heterologous DNA is located in an independent, extra-genomic artificial chromosome (as opposed to randomly inserted in an unknown area of the host cell genome or located as extrachromosomal element(s) providing only transient expression), it is stably maintained in an active transcription unit and is not subject to ejection via recombination or elimination during cell division. Accordingly, it is unnecessary to include a selection gene in the host cells and thus growth under selective conditions is also unnecessary. Furthermore, because the artificial chromosomes are capable of incorporating large segments of DNA, multiple copies of the heterologous gene and linked promoter element(s) can be retained in these chromosomes, thereby providing for high-level expression of the foreign protein(s). Alternatively, multiple copies of the gene can be linked to a single promoter element and several different genes can be linked in a fused polygene complex to a single promoter for expression of, for example, all the key proteins constituting a complete metabolic pathway (see, e., Beck von Bodman et al. (1995) Biotechnology 13:587-591). Alternatively, multiple copies of a single gene can be operatively linked to a single promoter, or each or one or several copies can be linked to different promoters or multiple copies of the same promoter. Additionally, because artificial chromosomes have an almost unlimited capacity for integration and expression of foreign genes, they can be used not only for the expression of genes encoding end-products of interest, but also for the expression of genes associated with optimal maintenance and metabolic management of the host cell, e.g., genes encoding growth factors, as well as genes that facilitate rapid synthesis of correct form of the desired heterologous protein product, e.g., genes encoding processing enzymes and transcription factors as described above.

The artificial chromosomes are suitable for expression of any proteins or peptides, including proteins and peptides that require in vivo posttranslational modification for their biological activity. Such proteins include, but are not limited to antibody fragments, full-length antibodies, and multimeric antibodies, tumor suppressor proteins, naturally occurring or artificial antibodies and enzymes, heat shock proteins, and others.

Thus, such cell-based “protein factories” employing artificial chromosomes can be generated using artificial chromosomes constructed with multiple copies (theoretically an unlimited number or at least up to a number such that the resulting artificial chromosome is about up to the size of a genomic chromosome (i.e., endogenous)) of protein-encoding genes with appropriate promoters, or multiple genes driven by a single promoter, i.e., a fused gene complex (such as a complete metabolic pathway in plant expression system; see, e.g., Beck von Bodman (1995) Biotechnology 13:587-591). Once such an artificial chromosome is constructed, it can be transferred to a suitable plant species capable of being propagated under field conditions, or under conditions that permit the recovery of the intended product. Plant cell cultures such as algae can be used in a system analogous to mammalian cell culture systems. The advantage of plant based systems such as this include low input costs for growth, rapid growth rates and ability to produce a large biomass economically.

The ability of artificial chromosomes to provide for high-level expression of heterologous proteins in host cells is demonstrated, for example, by analysis of mammalian cells containing a mammalian artificial chromosome, H1D3 and G3D5 cell lines described herein. Northern blot analysis of mRNA obtained from these cells reveals that expression of the hygromycin-resistance and β-galactosidase genes in the cells correlates with the amplicon number of the megachromosome(s) contained therein.

Transgenic plants producing these compounds are made by the introduction and expression of one or potentially many genes using the artificial chromosomes provided herein. The vast array of possibilities include, but are not limited to, any biological compound which is presently produced by any organism such as proteins, nucleic acids, primary and intermediary metabolites, carbohydrate polymers, enzymes for uses in bioremediation, enzymes for modifying pathways that produce secondary plant metabolites such as flavonoids or vitamins, enzymes that could produce pharmaceuticals and for introducing enzymes that could produce compounds of interest to the manufacturing industry such as specialty chemicals and plastics. The compounds are roduced by the plant, extracted upon harvest and/or processing, and used for any presently recognized useful purpose such as pharmaceuticals, fragrances, and industrial enzymes. Alternatively, plants produced in accordance with the methods and compositions provided herein can be made to metabolize certain compounds, such as hazardous wastes, thereby allowing bioremediation of these compounds.

The artificial chromosomes provided herein can be used in methods of protein and gene product production, particularly using plant cells as host cells for production of such products, and in cellular production systems in which the artificial chromosomes provide a reliable, stable and efficient means for optimizing the biomanufacturing of important compounds for medicine and industry.

2. Genetic Alteration of Organisms to Possess Desired Traits

Artificial chromosomes are ideally suited for preparing organisms, such as plants, that possess certain desired traits, such as, for example, disease resistance, resistance to harsh environmental conditions, altered growth patterns and enhanced physical characteristics. With respect to plants, the choice of the particular nucleic acid that will be delivered to recipient cells via artificial chromosomes often will depend on the purpose of the transformation. One of the major purposes of transformation of crop and tree species is to add some commercially desirable, agronomically important traits to the plant. Such traits include, but are not limited to, input and output traits such as herbicide resistance or tolerance, insect resistance or tolerance, disease resistance or tolerance (viral, bacterial, fungal or nematode), stress tolerance and/or resistance, as exemplified by resistance or tolerance to drought, heat, chilling, freezing, excessive moisture, salt stress and oxidative stress, increased yields, food content and makeup, physical appearance, male sterility, drydown, standability, prolificacy, starch quantity and quality, oil quantity and quality, protein quantity and quality and amino acid composition. It may be desirable to incorporate one or more genes conferring such desirable traits into host plants.

a. Herbicid Resistance

The genes encoding phosphinothricin acetyltransferase (bar and pat), glyphosate tolerant EPSP synthase genes, the glyphosate degradative enzyme gene gox encoding glyphosate oxidoreductase, deh (encoding a dehalogenase enzyme that inactivates dalapon), herbicide resistant (e.g., sulfonylurea and imidazolinone) acetolactate synthase, and bxn genes (encoding a nitrilase enzyme that degrades bromoxynil) are all examples of herbicide resistant genes for use in plant transformation. The bar and pat genes code for an enzyme, phosphinothricin acetyltransferase (PAT), which inactivates the herbicide phosphinothricin and prevents this compound from inhibiting gluatamine synthetase enzymes. The enzyme 5-enolpyruvylshikimate 3-phosphate synthase (EPSP synthase) is normally inhibited by the herbicide N-(phosphonomethyl)glycine (glyphosate). However, genes are known that encode glyphosate-resistant EPSP synthase enzymes. The deh gene encodes the enzyme dalapon dehalogenase and confers resistance to the herbicide dalapon. The bxn gene codes for a specific nitrilase enzyme that converts bromoxynil to a non-herbicidal degradation product.

b. Insect and Other Pest Resistance

Insect-resistant organisms may be prepared in which resistance or decreased susceptibility to insect-induced disease is conferred by introduction into the host organism or embryo of artificial chromosomes containing DNA encoding gene products (e.g., ribozymes and proteins that are toxic to certain pathogens) that destroy or attenuate pathogens or limit access of pathogens to the host. Potential insect resistance genes that can be introduced into plants via artificial chromosomes include Bacillus thuringiensis crystal toxin genes or Bt genes (see, e.g., Watrud et al. (1985) in Engineered Organisms and the Environment). Bt genes may provide resistance to lepidopteran or coleopteran pests such as the European Corn Borer (ECB). Such Bt toxin genes include the CrylA(b) and CrylA(c) genes. Endotoxin genes from other species of B. thuringiensis which affect insect growth or development also may be employed in this regard. Bt gene sequences can be modified to effect increased expression in plants, and particularly monocot plants. Means for preparing synthetic genes are well known in the art and are disclosed in, for example, U.S. Pat. Nos. 5,500,365 and 5,689,052. Examples of such modified Bt toxin genes include a synthetic Bt CrylA(b) gene (see, e.g., Perlak et al. (1991) Proc. Natl. Acad. Sci. U.S.A. 88:3324-3328) and the synthetic CrylA(c) gene termed 1800b (see PCT Application publication no. WO95/06128).

Examples of the types of genes that may be transferred into plants via artificial chromosomes to generate disease- and/or insect-resistant transgenic plants include, but are not limited to, the crylA(b) and crylA(c) genes which yield products that are highly toxic to two major rice insect pests (the striped stem borer and the yellow stem borer) (see, e.g., Cheng et al. (1998) Proc. Natl. Acad. Sci. U.S.A. 95:2767-2772), cry3 genes which encode products that are toxic to Coleopteran insects that attack a variety of plants, including grains and legumes (see, e.g., U.S. Pat. No. 6,023,013), genes (e.g., DNA encoding tricothecene 3-O-acetyltransferase) that confer resistance to tricothecenes such as those produced by plant fungi (e.g., Fusarium) in plants particularly susceptible to fungi (e.g., wheat, rye, barley, oats, and maize) (see, e.g., PCT Application publication no. WO 00/60061), and genes involved in multi-gene biosynthetic pathways that yield antipathogenic substances that have a deleterious effect on the growth of plant pathogens (see, e.g., U.S. Pat. No. 5,639,949).

Protease inhibitors may also provide insect resistance (see, e.g., Johnson et al. (1989) and will thus have utility in plant transformation. The use of a protease inhibitor II gene, pinII, from tomato or potato may be particularly useful. The combined effect of the use of a pinII gene with a Bt toxin gene can produce synergistic insecticidal activity. Other genes that encode inhibitors of the insect's digestive system, or those that encode enzymes or co-factors that facilitate the production of inhibitors, also may be useful. This group may be exemplified by oryzacystatin and amylase inhibitors such as those from wheat and barley.

Genes encoding lectins may confer additional or alternative insecticide properties. Lectins (originally termed phytohemagglutinins) are multivalent carbohydrate-binding proteins which have the ability to agglutinate red blood cells from a range of species. Lectins have been identified as insecticidal agents with activity against weevils, ECB and rootworm (see, e.g., Murdock et al. (1990) Phytochemistry 29:85-89; Czapla & Lang (1990) J. Econ. Entomol. 83:2480-2485). Lectin genes that may be useful include, for example, barley and wheat germ agglutinin (WGA) and rice lectins (Gatehouse et al. (1984) J. Sci. Food. Agric. 35:373-380).

Genes controlling the production of large and small polypeptides active against insects when introduced into the insect pests, such as, for example, lytic peptides, peptide hormones and toxins and venoms, may also be useful in generating pest-resistant plants. For example, expression of juvenile hormone esterase, directed toward specific insect pests, also may result in insecticidal activity, or cause cessation of metamorphosis (see, e.g., Hammock et al. (1990) Nature 344:458-461).

Transgenic plants expressing genes which encode enzymes that affect the integrity of the insect cuticle are additional examples of genes that may be transferred to plants via artificial chromosomes to confer resistance to insects. Such genes include those encoding, for example, chitinase, proteases, lipases and also genes for the production of nikkomycin, a compound that inhibits chitin synthesis, the introduction of any of which may be used to produce insect-resistant plants. Genes that affect insect molting, such as those affecting the production of ecdysteroid UDP-glucosyl transferase, also can be useful transgenes.

Genes that code for enzymes that facilitate the production of compounds that reduce the nutritional quality of the host plant to insect pests may also be used to confer insect resistance on plants. It may be possible, for instance, to confer insecticidal activity on a plant by altering its sterol composition. Sterols are obtained by insects from their diet and are used for hormone synthesis and membrane stability. Therefore, alterations in plant sterol composition by expression of genes that directly promote the production of undesirable sterols or those that convert desirable sterols into undesirable forms, could have a negative effect on insect growth and/or development and hence endow the plant with insecticidal activity. Lipoxygenases are naturally occurring plant enzymes that have been shown to exhibit anti-nutritional effects on insects and to reduce the nutritional quality of their diet. Therefore, transgenic plants with enhanced lipoxygenase activity may be resistant to insect feeding.

Tripsacum dactyloides is a species of grass that is resistant to certain insects, including corn root worm. Tripsacum may thus include genes encoding proteins that are toxic to insects or are involved in the biosynthesis of compounds toxic to insects. Such genes may be useful in conferring resistance to insects. It is known that the basis of insect resistance in Tripsacum is genetic, because said resistance has been transferred to Zea mays via sexual crosses (Branson and Guss, 1972). It is further anticipated that other cereal, monocot or dicot plant species may have genes encoding proteins that are toxic to insects which would be useful for producing insect resistant plants.

Further genes encoding proteins characterized as having potential insecticidal activity also may be used as transgenes in accordance herewith. Such genes include, for example, the cowpea trypsin inhibitor (CpT1: Hilder et al., 1987) which may be used as a rootworm deterrent, genes encoding avermectin (Avermectin and Abamectin., Campbell, W. C., Ed., 1989: Ikeda et al., 1987) which may prove particularly useful as a corn rootworm deterent, ribosome inactivating protein genes and even genes that regulate plant structures. Transgenic plants including anti-insect antibody genes and genes that code for enzymes that can convert a non-toxic insecticide (pro-insecticide) applied to the outside of the plant into an insecticide inside the plant also are contemplated.

C. Disease Resistance

Transgenic organisms, such as plants, that express genes that confer resistance or reduce susceptibility to disease are of particular interest. For example, the transgene may encode a protein that is toxic to a pathogen, such as a virus, fungus, mycotoxin-producing organism, nematode or bacterium, but that is not toxic to the transgenic host.

Because multiple genes can be introduced on an artificial chromosome, a series of genes encoding a genetic pathway involved in disease resistance or tolerance can be introduced into crop plants. For example, it is known that often numerous genes are expressed upon pathogen invasion, typically one or more “PR”, or pathogen related, proteins are expressed in response to invasion of a plant bacterial or fungal pathogen. One or more of the proteins involved in conferring resistance to pathogens can be contained within an artificial chromosome and therefore be expressed in a plant cell, in particular a whole transgenic plant as described herein. In addition, production of single-chain Fv recombinant antibodies in plants may extend the range of possibilities for the introduction of pathogen protection in crop plants (see, e.g., Tavladoraki et al. (1993) Nature 366:469-472).

It has been demonstrated that expression of a viral coat protein in a transgenic plant can impart resistance to infection of the plant by that virus and perhaps other closely related viruses (Cuozzo et al., 1988. Hemenway et al., 1988, Abel et al., 1986). Expression of antisense genes targeted at essential viral functions may also impart resistance to viruses. For example, an antisense gene targeted at the gene responsible for replication of viral nucleic acid may inhibit replication and lead to resistance to the virus. Interference with other viral functions through the use of antisense genes also may increase resistance to viruses. Further, it may be possible to achieve resistance to viruses through other approaches, including, but not limited to the use of satellite viruses. Artificial chromosomes are ideally suited for carrying a multiplicity of these genes and DNA sequences which are useful for conferring a broad range of resistance to many pathogens.

Genes encoding so-called “peptide antibiotics,” pathogenesis related (PR) proteins, toxin resistance, and proteins affecting host-pathogen interactions such as morphological may also be useful, particularly in conferring increased resistance to diseases caused by bacteria and fungi. Peptide antibiotics are polypeptide sequences which are inhibitory to growth of bacteria and other microorganisms. For example, the classes of peptides referred to as cepropins and magainins inhibit growth of may species of bacteria and fungi. Expression of PR proteins in monocotyledonous plants such as maize may be useful in conferring resistance to bacterial disease. These genes are induced following pathogen attack on a host plant and have been divided into at lease five classes of proteins (Bio. Linthorst, and Cornelissen, 1990). Included among the PR proteins are β-1,3-glucanases, chitinases, and osmotin and other proteins that are believed to function in plant resistance to disease organisms. Other genes have been identified that have antifungal properties, e.g., UDA (stinging nettle lectin) and hevein (Broakaert et al, 1989; Barkai-Golan et al, 1978). It is known that certain plant diseases are caused by the production of phytotoxins. Resistance to these diseases may be achieved through expression of a gene that encodes an enzyme capable of degrading or otherwise inactivating the phytotoxin. It also is contemplated that expression of genes that alter the interactions between the host plant and pathogen may be useful in reducing the ability of the disease organism to invade the tissues of the host plant, e.g., an increase in the waxiness of the leaf cuticle or other morphological characteristics.

d. Environment or Stress Resistance

Improvement of a plant's ability to tolerate various environmental stresses such as, but not limited to, drought, excess moisture, chilling, freezing, high temperature, salt, and oxidative stress, also can be effected through expression of genes therein. It is proposed that benefits may be realized in terms of increased resistance to freezing temperatures through the introduction of an “antifreeze” protein such as that of the Winter Flounder (Cutler et al., 1989) or synthetic gene derivatives thereof. Improved chilling tolerance also may be conferred through increased expression of glycerol-3-phosphate acetyltransferase in chloroplasts (Wolter et al., 1992). Resistance to oxidative stress in some crop species (often exacerbated by conditions such as chilling temperatures in combination with high light intensities) can be conferred by expression of superoxide dismutase (Gupta et al., 1993), and may be improved by glutathione reductase (Bowler et al., 1992). Such strategies may allow for tolerance to freezing in newly emerged fields as well as extending later maturity higher yielding varieties to earlier relative maturity zones.

It is contemplated that the expression of genes that favorably effect plant water content, total water potential, osmotic potential, and turgor will enhance the ability of the plant to tolerate drought. As used herein, the terms “drought resistance” and drought tolerance” are used to refer to a plant's increased resistance or tolerance to stress induced by a reduction in water availability, as compared to normal circumstances, and the ability of the plant to function and survive in lower-water environments. The expression of genes encoding for the biosynthesis of osmotically-active solutes, such as polyol compounds, may impart protection against drought. Within this class are genes encoding for mannitol-L-phosphate dehydrogenase (Lee and Saier, 1982) and trehalose-6-phosphate synthase (Kaasen et al., 1992). Through the subsequent action of native phosphatases in the cell or by the introduction and coexpression of a specific phosphatase, these introduced genes will result in the accumulation of either mannitol or trehalose, respectively, both of which have been well documented as protective compounds able to mitigate the effects of stress. Mannitol accumulation in transgenic tobacco has been verified and preliminary results indicate that plants expressing high levels of this metabolite are able to tolerate an applied osmotic stress (Tarczynski et al., 1992, 1993).

Similarly, the efficacy of other metabolites in protecting either enzyme function (e.g., alanopine or propionic acid) or membrane integrity (e.g., alanopine) has been documented (Loomis et al., 1989), and therefore expression of genes encoding for the biosynthesis of these compounds might confer drought resistance in a manner similar to or complimentary to mannitol. Other examples of naturally occurring matabolites that are osmotically active and/or provide some direct protective effect during drought and/or desiccation include fructose, erythritol (Coxson et al., 1992), sorbitol, dulcitol (Karsten et al., 1992), glucosylglycerol (Reed et al., 1984; ErdMann et al., 1992), sucrose, stachyose (Koster and Leopold, 1988: Blackman et al., 1992), raffinose (Bernal-Lugo and Leopold, 1992), proline (Rensburg et al., 1993), glycine betaine, ononitol and pinitol (Vernon and Bohnert, 1992). Continued canopy growth and increased reproductive fitness during times of stress will be augmented by introduction and expression of genes such as those controlling the osmotically active compounds discussed above and other such compounds. Genes which promote the synthesis of an osmotically active polyol compound include genes which encode the enzymes mannitol-1-phosphate dehydrogenase, trehalose-6-phosphate synthase and myoinositol O-methyltransferase. Artificial chromosomes can carry a multiplicity of genes to provide durable stress tolerance, for example, concominant expression of proline and ketane and/or poly-ols.

It is contemplated that the expression of specific proteins also may increase drought tolerance under certain conditions or in certain crop species. These may include proteins such as Late Embryogenic Proteins (see Dure et al., 1989). All three classes of LEAs have been demonstrated in maturing (i.e. desiccating) seeds. Within LEA proteins, the Type-II (dehydrin-type) have generally been implicated in drought and/or desiccation tolerance in vegetative plant parts (i.e. Mundy and Chua, 1988: Piatkowski et al., 1990: Yamaguchi-Shinozaki et al., 1992). Recently, expression of a Type-III LEA (HVA-1) in tobacco was found to influence plant height, maturity and drought tolerance (Fitzpatrick, 1993). In rice, expression of the HVA-1 gene influenced tolerance to water deficit and salinity (Xu et al., 1996). Expression of structural genes from all three LEA groups may therefore confer drought tolerance. Other types of proteins induced during water stress include thiol proteases, aldolases and transmembrane transporters (Guerrero et al., 1999), which may confer various protective and/or repair-type functions during drought stress. It is also is contemplated that genes that effect lipid biosynthesis and hence membrane composition might also be useful in conferring drought resistance on the plant.

Many of these genes for improving drought resistance have complementary modes of action. Thus, combinations of these genes might have additive and/or synergistic effects in improving drought resistance in plants. Many of these genes also improve freezing tolerance (or resistance): the physical stresses incurred during freezing and drought are similar in nature and may be mitigated in similar fashion. Benefit may be conferred via constitutive expression of these genes, but the preferred means of expressing these genes may be through the use of a turgor-induced promoter (such as the promoters for the turgor-induced genes described in Guerrero et al., 1990 and Shagan et al., 1993 which are incorporated herein by reference). Spatial and temporal expression patterns of these genes may enable plants to better withstand stress.

It is proposed that expression of genes that are involved with specific morphological traits that allow for increased water extractions from drying soil would be of benefit. For example, introduction and expression of genes that alter root characteristics may enhance water uptake. It also is contemplated that expression of genes that enhance reproductive fitness during times of stress would be of significant value. For example, expression of genes that improve the synchrony of pollen shed and receptiveness of the female flower parts, i.e., silks, would be of benefit. In addition it is proposed that expression of genes that minimize kernel abortion during times of stress would increase the amount of grain to be harvested and hence be of value.

Given the overall role of water in determining yield, it is contemplated that enabling plants to utilize water more efficiently, through the introduction and expression of genes, will improve overall performance even when soil water availability is not limiting. By introducing genes that improve the ability of plants to maximize water usage across a full range of stresses relating to water availability, yield stability or consistency of yield performance may be realized.

e. Plant Agronomic Characteristics

Plants possessing desired traits that might, for example, enhance utility, processibility and commercial value of the organisms in areas such as the agricultural and ornamental plant industries may also be generated using artificial chromosomes in the same manner as described above for production of disease-resistant organisms. In such instances, the artificial chromosomes that are introduced into the organism or embryo contain DNA encoding gene products that serve to confer the desired trait in the organism.

For example, transgenic plants having improved flavor properties, stability and/or quality are of commercial interest. One possible method for generating such plants may include the expression of transgenes, e.g., genes encoding cystathionine gamma synthase (CGS), that result in increased free methionine levels (see, e.g., PCT Application publication no. WO 00/55303).

Two of the factors determining where crop plants can be grown are the average daily temperature during the growing season and the length of time between frosts. Within the areas where it is possible to grow a particular crop, there are varying limitations on the maximal time it is allowed to grow to maturity and be harvested. For example, a variety to be grown in a particular area is selected for its ability to mature and dry down to harvestable moisture content within the required period of time with maximum possible yield. Therefore, crops of varying maturities are developed for different growing locations. Apart from the need to dry down sufficiently to permit harvest, it is desirable to have maximal drying take place in the field to minimize the amount of energy required for additional drying post-harvest. Also, the more readily a product such as grain can dry down, the more time there is available for growth and kernel fill. Genes that influence maturity and/or dry down can be identified and introduced into plant lines using transformation techniques to create new varieties adapted to different growing locations or the same growing location, but having improved yield to moisture ratio at harvest. Expression of genes that are involved in regulation of plant development may be especially useful.

Genes that would improve standability and other plant growth characteristics may also be introduced into plants. Expression of new genes in plants which confer stronger stalks, improved root systems, or prevent or reduce ear droppage would be of great value to the farmer. Introduction and expression of genes that increase the total amount of photoassimilate available by, for example, increasing light distribution and/or interception would be advantageous. In addition, the expression of genes that increase the efficiency of photosynthesis and/or the leaf canopy would further increase gains in productivity. Expression of a phytochrome gene in crop plants may be advantageous. Expression of such a gene may be reduce apical dominance, confer semidwarfism on a plant, and increase shade tolerance (U.S. Pat. No. 5,268,526). Such approaches would allow for increased plant populations in the field.

f. Nutrient Utilization

The ability to utilize available nutrients may be a limiting factor in growth of crop plants. It may be possible to alter nutrient uptake, tolerate pH extremes, mobilization through the plant, storage pools, and availability for metabolic activities by the introduction of new agents. These modifications would allow a plant such as maize to more efficiently utilize available nutrients. An increase in the activity of, for example, an enzyme that is normally present in the plant and involved in nutrient utilization may increase the availability of a nutrient. An example of such an enzyme would be phytase. It is further contemplated that enhanced nitrogen utilization by a plant is desirable. Expression of a glutamate dehydrogenase gene in plants, e.g., E. coli gdhA genes, may lead to enhanced resistance to the herbicide glufosinate by incorporation of excess ammonia into glutamate, thereby detoxifying the ammonia. Gene expression may make a nutrient source available that was previously not accessible, e.g., an enzyme that releases a component of nutrient value from a more complex molecule, perhaps a macromolecule. Alternatively, artificial chromosomes can carry the multiplicity of genes governing nodulation and nitrogen fixation in legumes. The artificial chromosomes could be used to promote nodulation in non-legume species.

g. Male Sterility

Male sterility is useful in the production of hybrid seed. Male sterility may be produced through gene expression. For example, it has been shown that expression of genes that encode proteins that interfere with development of the male inflorescence and/or gametophyte result in male sterility. Chimeric ribonuclease genes that express in the anthers of transgenic tobacco and oilseed rape have been demonstrated to lead to male sterility (Mariani et al., 1990). Other methods of conferring male sterility have been described, including gene encoding antisense RNA capable of causing male sterility (U.S. Pat. Nos. 6,184,439, 6,191,343 and 5,728,926) and methods utilizing two genes to confer sterility, see, e.g., U.S. Pat. No. 5,426,041.

A number of mutations were discovered in maize that confer cytoplasmic male sterility. One mutation in particular, referred to as T cytoplasm, also correlates with sensitivity to Southern corn leaf blight. A DNA sequence, designated TURF-13 (Levings, 1990), was identified that correlates with T cytoplasm. It is proposed that it would be possible through the introduction of TURF-13 via transformation, to separate male sterility from disease sensitivity. As it is necessary to be able to restore male fertility for breeding purposes and for grain production, it is proposed that genes encoding restoration of male fertility also may be introduced.

h. Improved Nutritional Content

Genes may be introduced into plants to improve the nutrient quality or content of a particular crop. Introduction of genes that alter the nutrient composition of a crop may greatly enhance the feed or food value. For example, the protein of many grains is suboptimal for feed and food purposes especially when fed to pigs, poultry, and humans. The protein is deficient in several amino acids that are essential in the diet of these species, requiring the addition of supplements to the grain. Limiting essential amino acids may include lysine, methionine, tryptophan, threonine, valine, arginine, and histidine. Some amino acids become limiting only after corn is supplemented with other inputs for feed formulations. The levels of these essential amino acids in seeds and grain may be elevated by mechanisms which include, but are not limited to, the introduction of genes to increase the biosynthesis of the amino acids, increase the storage of the amino acids in proteins, or increase transport of the amino acids to the seeds or grain.

The protein composition of a crop may be altered to improve the balance of amino acids in a variety of ways including elevating expression of native proteins, decreasing expression of those with poor composition changing the composition of native proteins, or introducing genes encoding entirely new proteins possessing superior composition.

The introduction of genes that alter the oil content of a crop plant may also be of value. Increases in oil content may result in increases in metabolizable-energy-content and density of seeds for use in feed and food. The introduced genes may encode enzymes that remove or reduce rate-limitations or regulated steps in fatty acid or lipid biosynthesis. Such genes may include, but are not limited to, those that encode acetyl-CoA carboxylase, ACP-acyltransferase, β-ketoacyl-ACP synthase, plus other well known fatty acid biosynthetic activities. Other possibilities are genes that encode proteins that do not possess enzymatic activity such as acyl-carrier proteins. Genes may be introduced that alter the balance of fatty acids present in the oil providing a more healthful or nutritive feedstuff. The introduced DNA also may encode sequences that block expression of enzymes involved in fatty acid biosynthesis, altering the proportions of fatty acids present in crops.

Genes may be introduced that enhance the nutritive value of the starch component of crops, for example by increasing, or in some cases decreasing, the degree of branching, resulting in improved utilization of the starch in livestock by delaying its metabolism. Additionally, other major constituents of a crop may be altered, including genes that affect a variety of other nutritive, processing, or other quality aspects. For example, pigmentation may be increased or decreased.

Feed or food crops may also possesses insufficient quantities of vitamins, requiring supplementation to provide adequate nutritive value. Introduction of genes that enhance vitamins biosynthesis may be envisioned including, for example, vitamins A (e.g. rice with Vitamin A or golden rice), E, B12 choline, and the like. Mineral content may also be sub-optimal. Thus genes that affect the accumulation or availability of compounds containing phosphorus, sulfur, calcium, manganese, zinc, and iron among others would be valuable.

Numerous other examples of improvements of crops may be effected using the artificial chromosomes, with appropriate heterologous genes contained therein, in accordance with the methods and compositions provided herein. The improvements may not necessarily involve grain, but may, for example, improve the value of a crop for silage. Introduction of DNA to accomplish this might include sequences that alter lignin production such as those that result in the “brown midrib” phenotype associated with superior feed value for cattle.

In addition to direct improvements in feed or food value, genes also may be introduced which improve the processing of crops and improve the value of the products resulting from the processing. One use of crops is via wetmilling. Thus, genes that increase the efficiency and reduce the cost of such processing, for example, by decreasing steeping time may also find use. Improving the value of wetmilling products may include altering the quantity or quality of starch, oil, corn gluten meal, or the components of gluten feed. Elevation of starch may be achieved through the identification and elimination of rate limiting steps in starch biosynthesis or by decreasing levels of the other components of crops resulting in proportional increases in starch.

Oil is another product of wetmilling, the value of which may be improved by introduction and expression of genes. Oil properties maybe be altered to improve its performance in the production and use of cooking oil, shortenings, lubricants or other oil-derived products or improvements of its health attributes when used in the food-related applications. Fatty acids also may be synthesized which upon extraction can serve as starting materials for chemical syntheses. The changes in oil properties may be achieved by altering the type, level, or lipid arrangement of the fatty acids present in the oil. This in turn may be accomplished by the addition of genes that encode enzymes that catalyze the synthesis of new fatty acids and the lipids possessing them or by increasing levels of native fatty acids while possibly reducing levels of precursors. Alternatively, DNA sequences may be introduced which slow or block steps in fatty acid biosynthesis resulting in the increase in precursor fatty acid intermediates. Genes that might be added include desaturases, epoxidases, hydratases, dehydratases and other enzymes that catalyze reactions involving fatty acid intermediates. Representative examples of catalytic steps that might be blocked include the desaturations from stearic to oleic acid and oleic to linolenic acid resulting in the respective accumulations of stearic and oleic acids. Another example is the blockage of elongation steps resulting in the accumulation of C8 to C12 saturated fatty acids.

i. Production of Chemicals or Biologicals

Transgenic plants can be used as protein production systems to generate recombinant products ranging from industrial enzymes, viral antigens, vaccines, antibodies, human blood proteins, cytokines, growth factors, enkephalins, serum albumin and other proteins of clinical relevance and pharmaceuticals. For example, enzymes including α-amylase, glucanase, phytase and xylanase (see, Goddijn and Pen (1995) Trends Biotechnol. 13:379-387; Pen et al. (1992) Bio/Technology 10:292-296; Horvath et al. (2000) Proc. Natl. Acad. Sci. U.S.A. 97:1914-1919; and e.g., Herbers and Sonnewald (1996) in Transgenic Plants: A Production System for Industrial and Pharmaceutical Proteins” Owen and Pen Eds., John Wiley & Sons, West Sussex, England).

Examples of medically relevant proteins that may be produced in plants include surface antigens of viral pathogens, such as hepatitis B virus and transmissible gastroenteritis virus spike protein, for use in vaccines. The proteins thus produced may be isolated and administered through standard vaccine introduction methods or through the consumption of the edible transgenic plant as food which can be taken orally (see, e.g., U.S. Pat. No. 6,136,320 and Mason et al. (1992) Proc. Natl. Acad. Sci. U.S.A. 89:11745-11749). HIV, rhinovirus, malarial and rabies virus antigens are additional examples of that may be expressed in plants as candidate vaccines (see, e.g., Porta et al. (1994) Virol 202:949-955; Turpen et al. (1995) Bio/Technology 13:53-57; and McGarvey et al. (1995) Bio/Technology 13:1484-1487). Antibodies may also be produced in plants, including, for example, a gene fusion encoding an antigen-binding single chain Fv protein (scFv) that recognizes the hapten oxazolone (Fiedler and Conrad (1995) Bio/Technology 13:1090-1093) and IgG (Ma et al. (1995) Science 268:716-719).

Examples of human biopharmaceuticals that may be expressed in plants include, but are not limited to, albumin (Sijmons et al. (1990)), enkephalins (Vandekerckhove et al. (1989) ), interferon-α (Zhu et al. (1994) and GM-CSF (Ganz et al. (1996) in Transgenic Plants: A Production System for Industrial and Pharmaceutical Proteins, Owen and Pen Eds., John Wiley & Sons, West Sussex, England, pp. 281-297; and Sardana et al. (1998) in Methods in Biotechnology, Vol. 3: Recombinant Proteins from Plants: Production and Isolation of Clinically Useful Compounds, Cunningham and Porter, Eds., Humana Press, N.J.; pp. 77-87).

Transgenic plants producing these compounds are made possible by the introduction and expression of one or potentially many genes using the artificial chromosomes provided herein. The vast array of possibilities include, but are not limited to, any biological compound which is presently produced by any organism such as proteins, nucleic acids, primary and intermediary metabolites, carbohydrate polymers, enzymes for uses in bioremediation, enzymes for modifying pathways that produce secondary plant metabolites such as flavonoids or vitamins, enzymes that could produce pharmaceuticals and for introducing enzymes that could produce compounds of interest to the manufacturing industry such as specialty chemicals and plastics. The compounds may be produced by the plant, extracted upon harvest and/or processing, and used for any presently recognized useful purpose such as pharmaceuticals, fragrances, and industrial enzymes to name a few. Alternatively, plants produced in accordance with the methods and compositions provided herein may be made to metabolize certain compounds, such as hazardous wastes, thereby allowing bioremediation of these compounds.

j. Non-Protein-Expressing Sequences

Nucleic acids may be introduced into plants that are designed to down-regulate or supress a plant-encoded gene. A number of different means to achieve down regulation have been demonstrated in the art, including antisense RNA, ribozymes and co-suppression. The use of antisense RNA to suppress plant genes is described, for example, in U.S. Pat. Nos. 4,801,540, 5,107,065 and 5,453,566. In such methods, an “antisense” gene is constructed that encodes an RNA that is complementary to the mRNA of a resident plant gene, such that expression of the antisense gene inhibits the translation of the mRNA of the resident plant gene. Thus, the activity of the resident gene is down-regulated.

An additional method of down regulating gene activities involves ribozymes, or catalytic hammerhead hairpin RNA structures. The use of ribozymes is described, for example, in U.S. Pat. Nos. 4,987,071, 5,037,746, 5,116,742 and 5,354,855. These methods rely on the expression of small catalytic “hammerhead” RNA molecules that are capable of binding to and cleaving specific RNA sequences. Ribozymes designed to specifically recognize a resident plant mRNA can be used to cleave the mRNA and prevent its proper expression.

Essentially a more or less equivalent down-regulation control of gene activities by ribozymes and antisense can be achieved by adding additional copies of the gene to be regulated. The process is referred to as co-suppression and is described in, for example, U.S. Pat. Nos. 5,034,323, 5,283,184 and 5,231,020.

Numerous plant genes may be targeted for down regulation. For example, a gene may be down-regulated that encodes an enzyme that catalyzes a reaction in a plant. Reduction of the enzyme activity may reduce or eliminate products of the reaction which include any enzymatically synthesized compound in the plnat such as fatty acids, amino acids, carbohydrates, nucleic acids and the like. Alternatively, the protein may be a storage protein, such as zein, or a structural protein, the decreased expression of which may lead to changes in seed amino acid composition or plant morphological changes, respectively. The possibilities cited above are provided only by way of example and do not represent the full range of applications.

### (1). Antisense RNA

Genes may be constructed, which when transcribed, produce antisense RNA that is complementary to all or part(s) of a targeted messenger RNA(s). The antisense RNA reduces production of the polypeptide product of the messenger RNA. The polypeptide product may be any protein encoded by the plant genome. The aforementioned genes will be referred to as antisense genes. An antisense gene may thus be introduced into a plant by transformation methods to produce a transgenic plant with reduced expression of a selected protein of interest. For example, the protein may be an enzyme that catalyzes a reaction in the plant. Reduction of the enzyme activity may reduce or eliminate products of the reaction which include any enzymatically synthesized compound in the plant such as fatty acids, amino acids, carbohydrates, nucleic acids and the like. Alternatively, the protein may be a storage protein, such as a zein, or a structural protein, the decreased expression of which may lead to changes in seed amino acid composition or plant morphological changes respectively. The possibilities cited above are provided only by way of example and do not represent the full range of applications.

### (2.) Ribozymes

Genes also may be constructed or isolated, which when transcribed, produce RNA enzymes (ribozymes) which can act as endoribonucleases and catalyze the cleavage of RNA molecules with selected sequences. The cleavage of selected messenger RNAs can result in the reduced production of their encoded polypeptide products. These genes may be used to prepare transgenic plants which possess them. The transgenic plants may possess reduced levels of polypeptides including, but not limited to, the polypeptides cited above.

Ribozymes are RNA-protein complexes that cleave nucleic acids in a site-specific fashion. Ribozymes have specific catalytic domains that possess endonuclease activity (Kim and Cech, 1987; Gerlach et al., 1987; Forster and Symons, 1987). For example, a large number of ribozymes accelerate phosphoester transfer reactions with a high degree of specificity, often cleaving only one of several phophoesters in an oligonucleotide substrate (Cech et al., 1981; Michel and Westhof, 1990); Reinhold-Hurek and Shub, 1992). This specificity has been attributed to the requirement that the substrate bind via specific base-pairing interactions to the internal guide sequence (“IGS”) of the ribozyme prior to chemical reaction.

Ribozyme catalysis has primarily been observed as part of sequence-specific cleavage/ligation reactions involving nucleic acids (Joyce, 1989; Cech et al., 1981). For example, U.S. Pat. No. 5,354,855 reports that certain ribozymes can act as endonucleases with a sequence specificity greater than that of known ribonucleases and approaching that of the DNA restriction enzymes.

Several different ribozyme motifs have been described with RNA cleavage activity (Symons, 1992). Examples include sequences from the Group I self splicing introns including Tobacco Ringspot Virus (Prody et al., 1986), Avacado Sunblotch Viroid (Palukaitis et al., 1979; Symons, 1981) and Lucerne Transient Streak Virus (Forster and Symons, 1987). Sequences from these and related viruses are referred to as hammerhead ribozyme based on a predicted folded secondary structure.

Other suitable ribozymes include sequences from RNase P with RNA cleavage activity (Yuan et al., 1992; Yuan and Altman, 1994; U.S. Pat. No. 5,168,053 and 5,624,824), hairpin ribozyme structures (Berzal-Herranz et al., 1992; Chowrira et al., 1993) and Hepatitis Delta virus based ribozymes (U.S. Pat. No. 5,625,047). The general design and optimization of ribozyme directed RNA cleavage activity has been discussed in detail (Haselhoff and Gerlach, 1988; Symons, 1 992; Chowrira et al., 1994; Thompson et al., 1995).

The other variable on ribozyme design is the selection of a cleavage site on a given target RNA. Ribozymes are targeted to a given sequence by virtue of annealing to a site by complementary base pair interactions. Two stretches of homology are required for this targeting. These stretches of homologous sequences flank the catalytic ribozyme structure defined above. Each stretch of homologous sequence can vary in length from 7 to 15 nucleotides. The only requirement for defining the homologous sequences is that, on the target RNA, they are separated by a specific sequence which is the cleavage site. For hammerhead ribozyme, the cleavage site is a dinucleotide sequence on the target RNA is a uracil (U) followed by either an adenine, cytosine or uracil (A, C or U) (Perriman et al., 1992; Thompson et al., 1995). The frequency of this dinucleotide occurring in any given RNA is statistically 3 out of 16. Therefore, for a given target messenger RNA of 1,000 bases, 187 dinucleotide cleavage sites are statistically possible.

Designing and testing ribozymes for efficient cleavage of a target RNA is a process well known to those skilled in the art. Examples of scientific methods for designing and testing ribozymes are described by Chowrira et al. (1994) and Lieber and Strauss (1995), each incorporated by reference. The identification of operative and preferred sequences for use in down regulating a given gene is simply a matter of preparing and testing a given sequence, and is a routinely practiced “screening” method known to those of skill in the art.

### (3.) Induction of Gene Silencing

It also is possible that genes may be introduced to produce transgenic plants which have reduced expression of a native gene product by the mechanism of co-suppression. It has been demonstrated in tobacco, tomato, and petunia (Goring et al., 1991; Smith et al., 1990; Napoli et al., 1990; van der Krol et al., 1990) that expression of the sense transcript of a native gene will reduce or eliminate expression of the native gene in a manner similar to that observed for antisense genes. The introduced gene may encode all or part of the targeting native protein but its translation may not be required for reduction of levels of that native protein.

### (4.) Non-RNA-Expressing Sequences

DNA elements including those of transposable elements such as Ds, Ac, or MU, may be inserted into a gene to cause mutations. These DNA elements may be inserted in order to inactivate (or activate) a gene and thereby “tag” a particular trait. In this instance the transposable element does not cause instability of the tagged mutation, because the utility of the element does not depend on its ability to move in the genome. Once a desired trait is tagged, the introduced DNA sequence may be used to clone the corresponding gene, e.g., using the introduced DNA sequence as a PCR primer together with PCR gene cloning techniques (Shapiro, 1983; Dellaporta et al., 1988). Once identified, the entire gene(s) for the particular trait, including control or regulatory regions where desired, may be isolated, cloned and manipulated as desired. The utility of DNA elements introduced into an organism for purposes of gene tagging is independent of the DNA sequence and does not depend on any biological activity of the DNA sequence, i.e., transcription into RNA or translation into protein. The sole function of the DNA element is to disrupt the DNA sequence of a gene.

It is contemplated that unexpressed DNA sequences, including synthetic sequences, could be introduced into cells as proprietary “labels” of those cells and plants and seeds thereof. It would not be necessary for a label DNA element to disrupt the function of a gene endogenous to the host organism, as the sole function of this DNA would be to identify the origin of the organism. For example, one could introduce a unique DNA sequence into a plant and this DNA element would identify all cells, plants, and progeny of these cells as having arisen from that labeled source. It is proposed that inclusion of label DNAs would enable one to distinguish proprietary germplasm or germplasm derived from such, from unlabelled germplasm.

Another possible element which may be introduced is a matrix attachment region element (MAR), such as the chicken lysozyme A element (Stief, 1989), which can be positioned around an expressible gene of interest to effect an increase in overall expression of the gene and diminish position dependent effects upon incorporation into the plant genome (Stief et al., 1989; Phi-Van et al., 1990). Sequences such as MARs can be included on the artificial chromosome to enhance gene expression.

3. Transgenic Models for Evaluation of Genes and Discovery of New Traits

Of significant interest is the use of plants and plant cells containing artificial chromosomes for the evaluation of new genetic combinations and discovery of new traits. Artificial chromosomes, by virtue of the fact that they can contain significant amounts of DNA can also therefore encode numerous genes and accordingly a multiplicity of traits. It is contemplated here that artificial chromosomes, when formed from one plant species, can be evaluated in a second plant species. The resultant phenotypic changes observed, for example, can indicate the nature of the genes contained within the DNA containing the artificial chromosome, and hence permit the identification of new genetic activities. Artificial chromsomes containing euchromatic DNA or partially containing euchromatic DNA can serve as a valuable source of new traits when transferred to an alien plant cell environment. For example, it is contemplated that artificial chromosomes derived from dicot plant species can be introduced into monocot plant species by transfering a dicot artificial chromosome. The dicot artificial chromosome containing a region of euchromatic DNA containing expressed genes.

The artificial chromosomes can be generated or manipulated in such a fashion that a large region of naturally occurring plant DNA becomes incorporated into the artificial chromosome. This allows the artificial chromosome to contain new genetic activities and hence carry new traits. For example, an artificial chromosome can be introduced into a wild relative of a crop plant under conditions whereby a portion of the DNA present in the chromosomes of the wild relative is transferred to the artificial chromosome. After isolation of the artificial chromosome, this naturally occurring region of DNA from the wild relative, now located on the artificial chromosome can be introduced into the domesticated crop species and the genes encoded within the transferred DNA expressed and evaluated for utility. New traits and gene systems can be discovered in this fashion.

Artificial chromosomes modified to recombine with plant DNA offer many advantages for the discovery and evaluation of traits in different plant species. When the artificial chromosome containing DNA from one plant species is introduced into a new plant species, new traits and genes can be introduced. This use of an artificial chromosome allows for the ability to overcome the sexual barrier that prevents transfer of genes from one plant species to another species. Using artificial chromosomes in this fashion allows for many potentially valuable traits to be identified including traits that are typically found in wild species. Other valuable applications for artificial chromosomes include the ability to transfer large regions of DNA from one plant species to another, DNA encoding potentially valuable traits such as altered oil, carbohydrate or protein composition, multiple genes encoding enzymes capable of producing valuable plant secondary metabolites, genetic systems encoding valuable agronomic traits such as disease and insect resistance, genes encoding functions that allow association with soil bacterium such as growth promoting bacteria or nitrogen fixing bacteria, or genes encoding traits that confer freezing, drought or other stress tolerances. In this fashion, artificial chromosomes can be used to discover regions of plant DNA that encode valuable traits.

The artificial chromosome can also be designed to allow the transfer and subsequent incorporation of these valuable traits now located on the artificial chromosome into the natural chromosomes of a plant species. In this fashion the artificial chromosomes can be used to transfer large regions of DNA encoding traits normally found in one plant species into another plant species. In this fashion, it is possible to derive a plant cell that no longer needs to carry an artificial chromosome to posses the new trait. Thus the artificial chromosome would serve as the transfer mechanism to permit the formation of plants with greater degree of genetic diversity.

An artificial chromosome can be designed in a variety of ways to accomplish the afore-mentioned purposes. An artificial chromosome can be modified to contain sequences that promote homologous recombination within plant cells, or be modified to contain a genetic system that functions as a site-specific recombination system. For example, the DNA sequence of Arabidopsis is now known. To construct an artificial chromosome capable of recombining with a specific region of Arabidopsis DNA, a sequence of Arabidopsis DNA, normally located near a chromosomal location encoding genes of potential interest can be introduced into an artificial chromosome by methods provided herein. It may be desireable to include a second region of DNA within the artificial chromosome that provides a second flanking sequence to the region encoding genes of potential interest, to promote a double recombination event which would ensure transfer of the entire chromosomal region encoding genes of potential interest to the artificial chromosome. The modified artificial chromosome, containing the DNA sequences capable of homologous recombination region can then be introduced into Arabidopsis cells and the homologous recombination event is selected.

It is convenient to include a marker gene to allow for the selection of a homologous recombination event. The marker gene is preferably inactive unless activated by an appropriate homologous recombination event. For example, U.S. Pat. No. 5,272,071, describes a method where an inactive plant gene is activated by a recombination event such that desired homologous recombination events can be easily scored. Similarly, U.S. Pat. No. 5,501,967 describes a method for the selection of homologous recombination events by activation of a silent selection gene first introduced into the plant DNA, the gene being activated by an appropriate homologous recombination event. Both of these methods can be applied to enable a selective process to be included in to select for recombination between an artificial chromosome and a plant chromosome. Once the homologous recombination event is detected, the artificial chromosome, once selected, is isolated and introduced into a recipient cell, for example, tobacco, corn, wheat or rice, and the expression of the newly introduced DNA sequences evaluated. Selection of recombinant events can take place in cell culture, or following seed formation and screening of seedling plants or seed itself.

Phenotypic changes in the recipient plant cells containing the artificial chromosome, or in regenerated plants containing the artificial chromosome, allows for the evaluation of the nature of the traits encoded by the genes of interest, for example, Arabidopsis DNA, under conditions naturally found in plant cells, including the naturally occurring arrangement of DNA sequences responsible for the developmental control of the traits in the normal chromosomal environment.

Traits such as durable fungal or bacterial disease resistance, new oil and carbohydrate compositions, valuable secondary metabolites such as phytosterols, flavonoids, efficient nitrogen fixation or mineral utilization, resistance to extremes of drought, heat or cold are all found within different populations of plant species and are often governed by multiple genes. The use of single gene transformation technologies does not permit the evaluation of the multiplicity of genes controlling many valuable traits. Thus, incorporation of these genes into artificial chromosomes allows the rapid evaluation of the utility of these genetic combinations in heterologous plant species.

The large scale order and structure of the artificial chromosome provides a number of unique advantages in screening for new utilities or new phenotypes within heterologous plant species. The size of new DNA that can be carried by an artificial chromosome can be millions of base pairs of DNA, representing potentially numerous genes that may have different or new utility in a heterologous plant cell. The artificial chromosome is a “natural” environment for gene expression, the problems of variable gene expression and silencing seen for genes transferred by random insertion into a genome should not be observed. Similarly, there is no need to engineer the genes for expression, and the genes inserted would not need to be recombinant genes. Thus, transferred genes are fully expected to be expressed in the typical temporal and spatial fashion as observed in the species from where the genes were initially isolated. A valuable feature for these utilities is the ability to isolate the artificial chromosomes and to further isolate, manipulate and introduce into other cells artificial chromosomes carrying unique genetic compositions.

Thus, the use of artificial chromosomes and homologous recombination in plant cells can be used to isolate and identify many valuable crop traits. In addition to the use of artificial chromosomes for the isolation and testing of large regions of naturally occurring DNA, methods for the use of artificial chromosomes and cloned DNA are also contemplated. Similar to that described above, artificial chromsomes can be used to carry large regions of cloned DNA, including that derived from other plant species.

The ability to incorporate DNA elements into artificial chromosomes as they are being formed allows for the development of artificial chromosomes specifically engineered as a platform for testing of new genetic combinations, or “genomic” discoveries for model species such as Arabidopsis. Specific “recombinase” systems can be used in plant cells to excise or re-arrange genes; these same systems can be used to derive new gene combinations contained on an artificial chromosome. In this regard, it is contemplated that the use of site specific recombination sequences can have considerable utility in developing artificial chromosomes containing DNA sequences recognized by recombinase enzymes and capable of accepting DNA sequences containing same. The use of site-specific recombination as a means to target an introduced DNA to a specific locus has been demonstrated in the art and such methods can be employed. The recombinase systems can also be used to transfer the cloned DNA regions contained within the artificial chromosome to the naturally occurring plant chromosomes.

Many site specific recombinases have been described in the literature (Kilby et al., Trends in Genetics, 9(12): 413-418, 1993). Among these are: an activity identified as R encoded by the pSR1 plasmid of Zygosaccharomyes rouxii, FLP encoded for the 2 um circular plasmid from Saccharomyces cerevisiae and Cre-lox from the phage P1.

The integration function of site specific recombinases is contemplated as a means to assist in the derivation of genetic combinations on artificial chromosomes. In order to accomplish this, it is contemplated that a first step of introducing site-specific recombinase sites into the genome of a plant cell in an essentially random manner is conducted, such that the plant cell has one or more site-specific recombinase recognition sequences on one or more of the plant chromosomes. An artificial chromosome is then introduced into the pant cell, the artificial chromosome engineered to contain a recombinase recognition site capable of being recognized by a site specific recombinase. Optionally a gene encoding a recombinase enzyme is also included, preferably under the control of an inducible promoter. Expression of the site specific recombinase enzyme in the plant cell, either by induction of a inducible recombinase gene, or transient expression of a recombinase sequence causes a site-specific recombination event to take place, leading to the insertion of a region of the plant chromosomal DNA containing the recombinase recognition site into the recombinase recognition site of the artificial chromosome, forming an artificial chromosome containing plant chromosomal DNA. The artificial chromosome can be isolated and introduced into a heterologous host, preferably a plant host, and expression of the newly introduced plant chromosomal DNA can be monitored and evaluated for desirable phenotypic changes. Accordingly, carrying out this recombination with a population of plant cells wherein the chromosomally located recombinase recognition site is randomly scattered throughout the chromosomes of the plant can lead to the formation of a population of artificial chromosomes, each with a different region of plant chromosomal DNA, each representing a new genetic combination.

This particular method involves the precise site-specific insertion of chromosomal DNA into the artificial chromosome. This precision has been demonstrated in the art. For example, Fukushige and Sauer (Proc. Natl. Acad. Sci. USA, 89:7905-7909, 1992) demonstrated that the Cre-lox homologous recombination system could be successfully employed to introduce DNA into a predefined locus in a chromosome of mammalian cells. In this demonstration a promoter-less antibiotic resistance gene modified to include a lox sequence at the 5′ end of the coding region was introduced into CHO cells. Cells were re-transformed by electroporation with a plasmid that contained a promoter with a lox sequence and a transiently expressed Cre recombinase gene. Under the conditions employed, the expression of the Cre enzyme catalyzed the homologous recombination between the lox site in the chromosomally located promoter-less antibiotic resistance gene and the lox site in the introduced promoter sequence leading to the formation of a functional antibiotic resistance gene. The authors demonstrated efficient and correct targeting of the introduced sequence, 54 of 56 lines analyzed corresponded to the predicted single copy insertion of the DNA due to Cre catalyzed site specific homologous recombination between the lox sequences.

The use of the same Cre-lox system has been demonstrated in plants (Dale and Ow, Gene 91:79-85, 1995) to specifically excise, delete or insert DNA. The precise event is controlled by the orientation of lox DNA sequences, in cis the lox sequences direct the Cre recombinase to either delete (lox sequences in direct orientation) or invert (lox sequences in inverted orientation) DNA flanked by the sequences, while in trans the lox sequences can direct a homologous recombination event resulting in the insertion of a recombinant DNA. Accordingly a lox sequence may be first added to a genome of a plant species capable of being transformed and regenerated to a whole plant to serve as a recombinase target DNA sequence for recombination with an artificial chromosome. The lox sequence may be optimally modified to further contain a selectable marker which is inactive but can be activated by insertion of the lox recombinase recognition sequence into the artificial chromosome.

A promoterless marker gene or selectable marker gene linked to the recombinase recognition sequence, which is first inserted into the chromosomes of a plant cell can be used to engineer a platform chromosome. A promoter is linked to a recombinase recognition site, in an orientation that allows the promoter to control the expression of the marker or selectable marker gene upon recombination within the artificial chromosome. Upon a site-specific recombination event between a recombinase recognition site in a plant chromosome and the recombinase recognition site within the the introduced artificial chromosome, a cell is derived with a recombined artificial chromosome, the artificial chromosome containing an active marker or selectable marker acitivity that permits the identification and or selection of the cell.

The artificial chromosomes can be transferred to other plant species and the functionality of the new combinations tested. The ability to conduct such an inter-chromosomal transfer of sequences has been demonstrated in the art. For example, the use of the Cre-lox recombinase system to cause a chromosome recombination event between two chromatids of different chromosomes has been shown

Any number of recombination systems may be employed (see, U.S. provisional application Serial No. filed the same day herewith under attorney docket no. 24601-P420). Such systems include, but are not limited to, bacterially derived systems such as the Int/att system of phage lambda and the Gin/gix system.

More than one recombination system may be employed, including, for example, one recombinase system for the introduction of DNA into an artificial chromosome, and a second recombinase system for the subsequent transfer of the newly introduced DNA contained within an artificial chromosome into the naturally occurring chromosome of a second plant species. The choice of the specific recombination system used will be dependent on the nature of the modification contemplated.

By having the ability to isolate an artificial chromosome and in particular artificial chromosomes containing plant chromosomal DNA introduced via site-specific recombination and re-introduce the chromosome into other cells, particularly plant cells, these new combinations can be evaluated in different crop species without the need to first isolate and modify the genes, or carry out multiple transformations or gene transfers to achieve the same combination isolation and testing combinations of the genes in plants. The use of a site specific recombinase and artificial chromosomes also allows the convenient recovery of the plant chromosomal region into other recombinant DNA vectors and systems for manipulation and study.

The artificial chromosomes can be engineered as platforms to accept large regions of cloned DNA, such as that contained in Bacterial Artificial Chromosomes (BACs) or Yeast Artificial Chromosomes (YACs). It is further contemplated, that as a result of the typical structure of amplification-based artificial chromosomes, such as, for example, SATACS (or ACes), containing tandemly repreated DNA blocks, that more than cloned DNA sequence can be introduced by recombination processes. In particular recombination within a predefined region of the tandemly repreated DNA within the artifical chromosome provides a mechanism to “stack” numerous regions of cloned DNA, including large regions of DNA contained within BACs or YACs clones. Thus, multiple combinations of genes can be introduced onto artificial chromosomes and these combinations tested for functionality. In particular, it is contemplated that multiple YACs or BACs can be stacked onto an artificial chromsomes, the BACs or YACs containing multiple genes of complex pathways or mutlipe genetic pathways. The BACs or YACs are typically selected based on genetic information available within the public domain, for example from the Arabidopsis Information Management System (http://aims.cps.msu.edu/aims/index.html) or the information related to the plant DNA sequences available from the Institute for Genomic Research (http://www.tigr.org) and other sites known to those skilled in the art. Alternatively, clones can be chosen at random and evaluated for functionality. It is contemplated that combinations providing a desired phenotype can be identified by isolation of the artificial chromosome containing the combination and analyzing the nature of the inserted cloned DNA.

In another embodiment of the methods provided herein for discovering genes associated with plant traits, the artificial chromosome used to transfer plant DNA to a host cell for evaluation therein will contain large regions of plant DNA, in particular plant euchromatin, as a result of the process by which the artificial chromosome is produced. In particular, the artificial chromosome may be an amplification-based artificial chromosome, including, but not limited to: (1) a minichromosome arising from breakage of a dicentric chromosome, (2) an artificial chromosome containing one or more regions of repeating nucleic acid units wherein the repeat region(s) contain substantially equivalent amounts of euchromatic and heterochromatic nucleic acid, (3) an artificial chromosome containing one or more regions of repeating nucleic acid units wherein the repeat region(s) is made up predominantly of euchromatic DNA or contains about 10%, 20%, 30%, 40%, 50%, 60%, 70%, 80%, 90% or greater than 90% euchromatic DNA, (4) an artificial chromosome containing one or more regions of repeating nucleic acid units wherein the artificial chromosome is made up of substantially equivalent amounts of heterochromatin and euchromatin, (5) an artificial chromosome that containing one or more regions of repeating nucleic acid units having common nucleic acid sequences that represent euchromatic and heterochromatic nucleic acid and (6) a sausage-like structure that contains a portion or all of a euchromatin-containing arm of a plant chromosome.

In these methods for discovering genes associated with plant traits, because the artificial chromosome used to transfer plant DNA to a host cell for evaluation therein is generated to already contain large amounts of plant DNA, in particular plant euchromatin, there is no need to introduce plant euchromatin into the artificial chromosomes, by homologous or site-specific recombination.

4. Use of Artificial Chromosomes for Preparation and Screening of Libraries

Since large fragments of DNA can be incorporated into artificial chromosomes (ACs), they are well-suited for use as cloning vehicles that can accommodate entire genomes in the preparation of genomic DNA libraries, which then can be readily screened for functionality as described above or for specific gene sequences for further modification and study. For example, it is possible to use artificial chromosomes to prepare artificial chromosome libraries containing plant genomic DNA library useful in the identification and isolation of functional DNA components such as genes, centromeric DNA and telomeric DNA from a variety of different species of plants.

The following examples are included for illustrative purposes only and are not intended to limit the scope of the invention.

### EXAMPLE 1

### Generation of Arabidopsis Protoplasts

Plant protoplasts are typically generated from plant cells following standard techniques (for example, Maheshwari et al., Crit. Rev. Plant Sci. 14:149-178, 1995; Ramulu et al., Methods in Molecular Biology 111 227-242, 1999). Typically plant protoplasts are prepared from fresh plant tissue, e.g., leaf, or can be prepared by converting cell suspension cultures to protoplasts by removal of the cell walls enzymatically. For production of Arabidopsis protoplasts, the methods of Karesh et al. (Plant Cell Reports 9:575-578, 1991) and Mathur et al. (Plant Cell Reports 14:21-226, 1995) were used to generate Arabidopsis suspension cultures by modifications thereof as described below. These cells were maintained in liquid culture and subcultured as required, usually between 7 and 10 days in culture.

Establishment of Suspension Cultures

Cell suspension cultures derived from root callus of Arabidopsis thaliana cv. Columbia, RLD and Landsburg I erecta' were used. Calli were induced from roots of 3 week-old seedlings on callus induction medium containing MS basic media (Murashige and Skoog (1962) Physiol. Plant 15:473-497) with 3% sucrose, 0.5 mg/l napthalene acetic acid (NAA), 0.05 mg/l Kinetin (Sigman Aldrich Canada). The cell suspension cultures were grown from the calli in liquid callus induction medium at 22° C. with shaking at 120 rpm. They were subcultured every 7 days.

Generation of Protoplasts

One gram of 4-5 day-old suspension culture was incubated in 6 ml enzyme solution containing 1% Cellulase ‘Onozuka’ R-10 and 0.25% Macerozyme R-10 in 35 g/l CaCl₂.2H₂O (Hartmann et al. (1998) Plant Mol. Biol. 36:741-754) and incubated at 22° C. in the dark with shaking at 70 rpm for 15 h. The protoplast mixture was poured through a 100 μm nylon mesh sieve and centrifuged at 250×g for 5 min. The protoplasts were washed with 35 g/l CaCl₂.2H₂O and resuspended in 10 ml floating medium containing B5 medium (Gamborg et al. (1968) Exp. Cell Res. 50:151-158) with 144 g/l sucrose and 1 mg/l 2,4-dichlorophenoxyacetic acid (2,4-D). The protoplasts were centrifuged at 80×g for 10 min, collected at the interface and used immediately for transfection.

### EXAMPLE 2

### Generation of Tobacco Mesophyll Protoplasts

Mesophyll protoplasts were generated from leaves of sterile plantlets of N. tabacum cv. Xanthi. The plantlets were grown aseptically on MSO medium (MS basal media, 3% sucrose, 0.05% morpholinoethanesulfonic acid (MES), 1.0 mg/l benzyl adenine (BA), 0.1 mg/l NAA and 0.8% agar, pH 5.8) at 22° C. under a 16/8 h photoperiod (see also Bilang et al. (1994) Plant Molecular Biology Manual A1:1-6). Fully expanded leaves (2×4 cm) were cut in half, the main vein removed and the upper epidermis scored with parallel cuts. Leaf pieces were immersed in 6 ml enzyme solution containing 1.2% Cellulase ‘Onozuka’ R-10 and 0.4% Macerozyme R-10 in K4 medium (Nagy and Maliga (1976) Z. Pflanzenpysiol. 78:453-455) and incubated at 22° C. for 15 h without shaking. The protoplasts were purified by pouring through a 100 μm nylon mesh sieve. Suspension of protoplasts was carefully overlayed with 1 ml W5 solution (Bilang et al. (1994) Plant Molecular Biology Manual A1:1-6) and centrifuged at 80×g for 10 min. Protoplasts were then resuspended in W5 solution at a density of 1×10⁶ protoplasts/ml and stored at 4° C. for 1 to 2 hours prior to treatment, for example, DNA uptake or chromosome transfer.

### EXAMPLE 3

### Production of Tobacco Protoplasts from Suspension Cultures

Tobacco BY-2 protoplasts are prepared from suspension cultures according to the method of Nagata et al. [(1981) Molecular and General Genetics, 184:161-165].

### EXAMPLE 4

### Generation of Brassica Hypoc tyl Protoplasts

Genotypes of Brassica napus, B. oleracea, B. juncea and B. carinata may be used to generate protoplasts. Seeds of Brassica napus were surface-sterilized (for 2 min with 70% ethanol, then for 20 min with 2.4% sodium hypochlorite containing one drop of Tween 20 per 100 ml). Seeds were rinsed thoroughly with sterile distilled water and grown aseptically on autoclaved germination medium (half-strength basal Murashige and Skoog's medium (MS), 1% sucrose, 0.8% agar, pH 5.8). Unless otherwise indicated, the protoplast generation procedures were performed aseptically and solutions and media were filter-sterilized. Alternatively, protoplasts can be generated and cultured successfully from different explants using various protocol modifications (for example, Kao et al. (1991) Plant Science 75:63-72; Kao et al. (1990) Plant Cell Rep. 9:311-315; Kao and Seguin-Swartz (1987) Plant Cell Tiss. Org. Cult. 10:79-90; Kao (1977) Mol. Gen. Genet. 150:225-230).

Generation of Hypocotyl Protoplasts

Hypocotyls were excised from 4 or 5 day-old seedlings grown aseptically in the dark with or without light exposure for a few hours prior to use. The explants were cut transversely into 2-5 mm pieces and incubated in enzyme solution (salts, vitamins and organic acids of Kao's medium (Kao (1977) Mol. Gen. Genet. 150:225-230), 0.4 g/l CaCl₂.2H₂O, 13% sucrose, 1% Cellulase ‘Onozuka R10’, 0.1% Pectolyase Y23, pH 5.6) in petri dishes, in darkness, without agitation for 14-18 hours, then with agitation on a rotary shaker (ca. 50 rpm) for 15-30 min.

The mixture was filtered through a 63 μm nylon screen into centrifuge tubes, and an equal volume of 17.5% sucrose was added to each tube. Following centrifugation (ca. 100×g, 8 min), the protoplast band that formed at the top of each tube was collected. Protoplasts were washed 3 times by resuspension in wash solution [solution W5 of Menczel and Wolfe (1984, Plant Cell Rep 3:196-198) at a reduced strength (0.8×)]followed by centrifugation at 100×g for 3-5 min and discarding the supernatant.

Protoplasts were cultured in Kao's medium containing the salts, vitamins and organic acids with 30 g/l sucrose, 68.4 g/l glucose, 0.5 mg/l NAA, 0.5 mg/l BA, 0.5 mg/l 2,4-D, pH 5.7, at a density of 1×10⁵ per ml and incubated at 25° C., 16 h photoperiod, in dim fluorescent light (25 μEm⁻² s⁻¹).

After 5-8 days in culture, 1-1.5 ml of feeder medium containing the above medium except with 55.8 g/l glucose instead of 68.4 g/l, were added to each dish, and the dishes were placed under brighter fluorescent light (50 μEm⁻² s⁻¹). At about 14 days, 1-2 ml of medium were removed from each dish, and 2-3 ml of feeder medium containing basal B5 medium (Gamborg et al. (1968) Exp. Cell Res. 50:151-158), 3% sucrose, 3.8% glucose, 0.5 mg/l BA, 0.5 mg/l NAA, and 0.5 mg/l 2,4-D, pH 5.7, were added. At about 21 days, if microcolonies have not yet formed, the cultures can be fed with the last feeder medium except with 2.2% glucose instead of 3.8%. Protoplast cultures can be washed when necessary by adding new feeder medium, gently swirling petri dishes, allowing cells to settle, removing most of the supernatant and adding fresh medium to the dishes.

At 3-5 weeks, microcolonies were embedded with medium containing a 1:1 mixture of the last feeder medium and proliferation medium which contains the components of the feeder medium with 0.9% glucose and 1.6% agarose to make a concentration of 0.8% in the final mixture. Cultures were incubated as described above in bright fluorescent light (80-100 μEm⁻² s⁻¹). After 10 days-2 weeks, green colonies were plated onto the regeneration medium.

### EXAMPLE 5

### Preparation of a Transformation Vector Useful for the Induction of Plant Artificial Chromosome Formation

Plant artificial chromosomes (PACs) can be generated by introducing nucleic acid, such as DNA, which can include an amplification-inducing DNA and/or a targeting DNA, for example rDNA or lambda DNA, into a plant cell, allowing the cell to grow, and then identifying from among the resulting cells those that include a chromosome with a structure that is distinct from that of any chromosome that existed in the cell prior to introduction of the nucleic acid. The structure of a PAC reflects amplification of chromosomal DNA, for example, segmented, repeat region-containing and heterochromatic structures. It is also possible to select cells that contain structures that are precursors to PACs, for example, chromosomes containing more than one centromere and/or fragments thereof, and culture and/or manipulate them to ultimately generate a PAC within the cell.

In the method of generating PACs, the nucleic acid can be introduced into a variety of plant cells. The nucleic acid can include targeting DNA and/or a plant expressable DNA encoding one or multiple selectable markers (e.g., DNA encoding bialophos (bar) resistance) or scorable markers (e.g., DNA encoding GFP). Examples of targeting DNA include, but are not limited to, N. tabacum rDNA intergenic spacer sequence (IGS) and Arabidopsis rDNA such as the 18S, 5.8S, 26S rDNA and/or the intergenic spacer sequence. The DNA can be introduced using a variety of methods, including, but not limited to Agrobacterium-mediated methods, PEG-mediated DNA uptake and electroporation using, for example, standard procedures according to Hartmann et al [(1998) Plant Molecular Biology 36:741]. The cell into which such DNA is introduced can be grown under selective conditions and can initially be grown under non-selective conditions and then transferred to selective media. The cells or protoplasts can be placed on plates containing a selection agent to grow, for example, individual calli. Resistant calli can be scored for scorable marker expression. Metaphase spreads of resistance cultures can be prepared, and the metaphase chromosomes examined by FISH analysis using specific probes in order to detect amplification of regions of the chromosomes. Cells that have artificial chromosomes with functioning centromeres or artificial chromosomal intermediate structures, including, but not limited to, dicentric chromosomes, formerly dicentric chromosomes, minichromosomes, heterochromatin structures (e.g. sausage chromosomes), and stable self-replicating artificial chromosomal intermediates as described herein, are identified and cultured. In particular, the cells containing self-replicating artificial chromosomes are identified.

The DNA introduced into a plant cell for the generation of PACs can be in any form, including in the form of a vector. An exemplary vector for use in methods of generating PACs can be prepared as follows.

For the production of artificial chromosomes, plant transformation vectors, as exemplified by pAgIIa and pAgIIb, containing a selectable marker, a targeting sequence, and a scorable marker were constructed using procedures well known in the art to combine the various fragments. The vectors can be prepared using vector pAg1 as a base vector and inserting the following DNA fragments into pAg1: DNA encoding β-glucoronidase under the control of the nopaline synthase (NOS) promoter fragment and flanked at the 3′ end by the NOS terminator fragment, a fragment of mouse satellite DNA and an N. tabacum rDNA intergenic spacer sequence (IGS). In constructing plant transformation vectors, vector pAg2 can also be used as the base vector.

1. Construction of pAG1

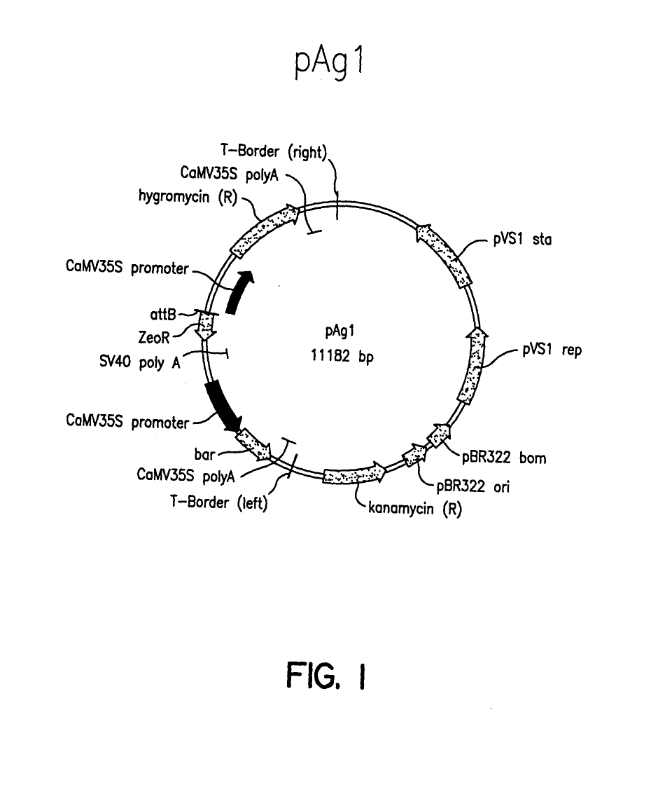

Vector pAg1 (SEQ. ID. NO: 1; see FIG. 1) is a derivative of the CAMBIA vector named pCambia 3300 (Center for the Application of Molecular Biology to International Agriculture, i.e., CAMBIA, Canberra, Australia; www.cambia.org), which is a modified version of vector pCambia 1300 to which has been added DNA from the bar gene confering resistance to phosphinothricin. The nucleotide sequence of pCambia 3300 is provided in SEQ. ID. NO: 2. pCambia 3300 also contains a lacZ alpha sequence containing a polylinker region.

pAg1 was constructed by inserting two new functional DNA fragments into the polylinker of pCambia 3300: one sequence containing an attB site and a promoterless zeomycin resistance-encoding DNA flanked at the 3′ end by a SV40 polyA signal sequence, and a second sequence containing DNA from the hygromycin resistance gene (hygromycin phosphotransferase) confering resistance to hygromycin for selection in plants. Although the zeomycin-SV40 polyA signal fusion is not expected to provide the basis for zeomycin selection in plant cells, it can be activated in mammalian cells by insertion of a functional promoter element into the attB site by site-specific recombination catalyzed by the Lambda att integrase. Thus, the inclusion of the attB-zeomycin sequences allows for evaluation of functionality of plant artificial chromosomes in mammalian cells by activation of the zeomycin resistance-encoding DNA, and provides an att site for further insertion of new DNA sequences into plant artificial chromosomes formed as a result of using pAg1 for plant transformation. The second functional DNA fragment allows for selection of plant cells with hygromycin. Thus, pAg1 contains DNA from the bar gene confering resistance to phosphinothricin, DNA from the hygromycin resistance gene, both resistance-encoding DNAs under the control of a separate cauliflower mosaic virus (CaMV) 35S promoter, and the attB-promoterless zeomycin resistance-encoding DNA.

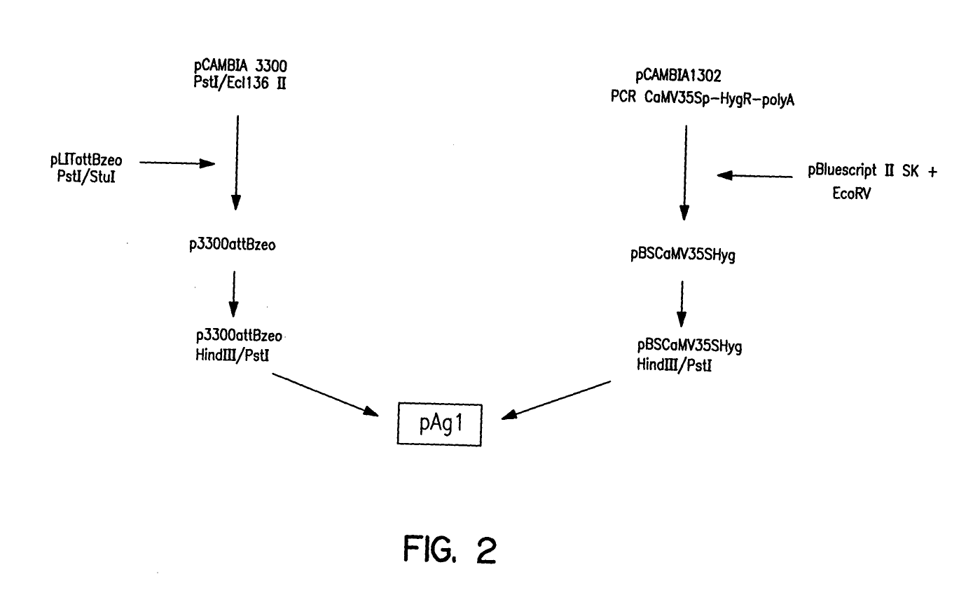

pAg1 is a binary vector containing Agrobacterium right and left T-DNA border sequences for use in Agrobacterium-mediated transformation of plant cells or protoplasts with the DNA located between the border sequences. pAg1 also contains the pBR322 Ori for replication in E coli. pAg1 was constructed by ligating HindIII/PstI-digested p3300attBZeo with HindIII/PstI-digested pBSCaMV35SHyg as follows (see FIG. 2).

a. Generation of p3300attBZeo

Plasmid pCambia 3300 was digested with PstI/Ecl136 II and ligated with PstI/StuI-digested pLITattBZeo (the nucleotide sequence of pLITattBZeo is provided in SEQ. ID. NO: 19 to generate p3300attBZeo which contains an attB site, a promoterless zeomycin resistance-encoding DNA flanked at the 3′ end by a SV40 polyA signal, and a reconstructed PstI site.

b. Generation of pBSCaMV35SHyg

A DNA fragment containing DNA encoding hygromycin phosphotransferase flanked by the CaMV 35S promoter and the CaMV 35S polyA signal sequence was obtained by PCR amplification of plasmid pCambia 1302 (GenBank Accession No. AF234298 and SEQ. ID. NO: 3). The primers used in the amplification reaction were as follows:

CaMV35SpolyA:

| | |
|---|---|
| 5′-CTGAATTAACGCCGAATTAATTCGGGGGATC | SEQ. ID. NO: 4 |
| TG-3′ | |

CaMV35Spr:

| | |
|---|---|
| 5′-CTAGAGCAGCTTGCCAACATGGTGGAGCA-3′ | SEQ. ID. NO: 5 |

The 2100-bp PCR fragment was ligated with EcoRV-digested pBluescript II SK+ (Stratagene, La Jolla, Calif., U.S.A.) to generate pBSCaMV35SHyg.

C. Generation of pAg1

To generate pAg1, pBSCaMV35SHyg was digested with HindIII/PstI and ligated with HindIII/PstI-digested p3300attBZeo. Thus, pAg1 contains the pCambia 3300 backbone with DNA conferring resistance to phophinothricin and hygromycin under the control of separate CaMV 35S promoters, an attB-promoterless zeomycin resistance-encoding DNA recombination cassette and unique sites for adding additional markers, e.g., DNA encoding GFP. The attB site facilitates the addition of new DNA sequences to plant or animal, e.g., mammalian, artificial chromosomes, including PACs formed as a result of using the pAg1 vector, or derivatives thereof, in the production of PACs. The attB site provides a convenient site for recombinase-mediated insertion of DNAs containing a homologous att site.

2. pAG2

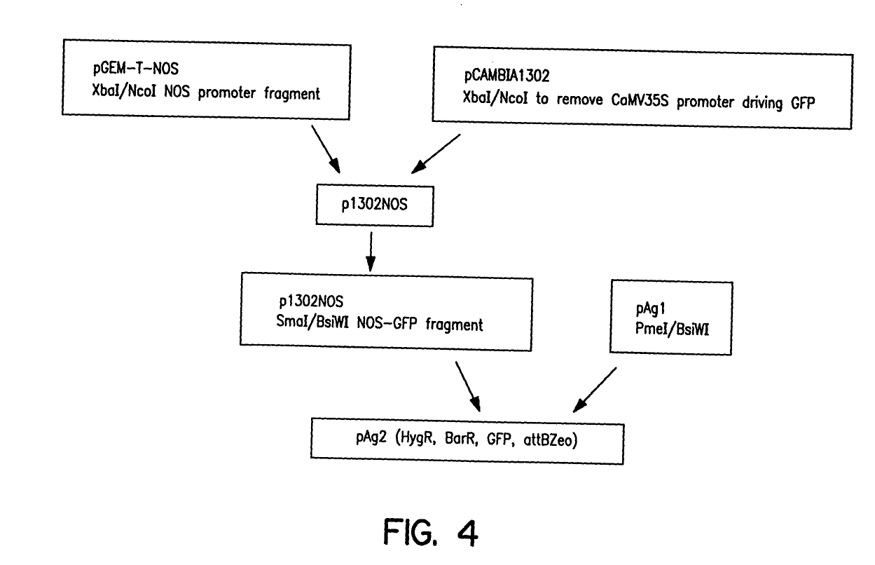

The vector pAg2 (SEQ. ID. NO: 6; see FIG. 3) is a derivative of vector pAg1 formed by adding DNA encoding a green fluorescent protein (GFP), under the control of a NOS promoter and flanked at the 3′ end by a NOS polyA signal, to pAg1. pAg2 was constructed as follows (see FIG. 4). A DNA fragment containing the NOS promoter was obtained by digestion of pGEM-T-NOS, or pGEMEasyNOS (SEQ. ID. NO: 7), containing the NOS promoter in the cloning vector pGEM-T-Easy (Promega Biotech, Madison, Wis., U.S.A.), with XbaI/NcoI and was ligated to an XbaI/NcoI fragment of pCambia 1302 containing DNA encoding GFP (without the CaMV 35S promoter) to generate p130 2NOS (SEQ. ID. NO: 8) containing GFP-encoding DNA in operable association with the NOS promoter. Plasmid p1302NOS was digested with SmaI/BsiWI to yield a fragment containing the NOS promoter and GFP-encoding DNA. The fragment was ligated with PmeI/BsiWI-digested pAg1 to generate pAg2. Thus, pAg2 contains DNA from the bar gene confering resistance to phosphinothricin, DNA conferring resistance to hygromycin, both resistance-encoding DNAs under the control of a cauliflower mosaic virus 35S promoter, DNA encoding kanamycin resistance, a GFP gene under the control of a NOS promoter and the attB-zeomycin resistance-encoding DNA. One of skill in the art will appreciate that other fragments can be used to generate the pAg1 and pAg2 derivatives and that other heterlogous DNA can be incorporated into pAg1 and pAg2 derivatives using methods well known in the art.

3. pAgIIa and pAgIIb Transformation Vectors

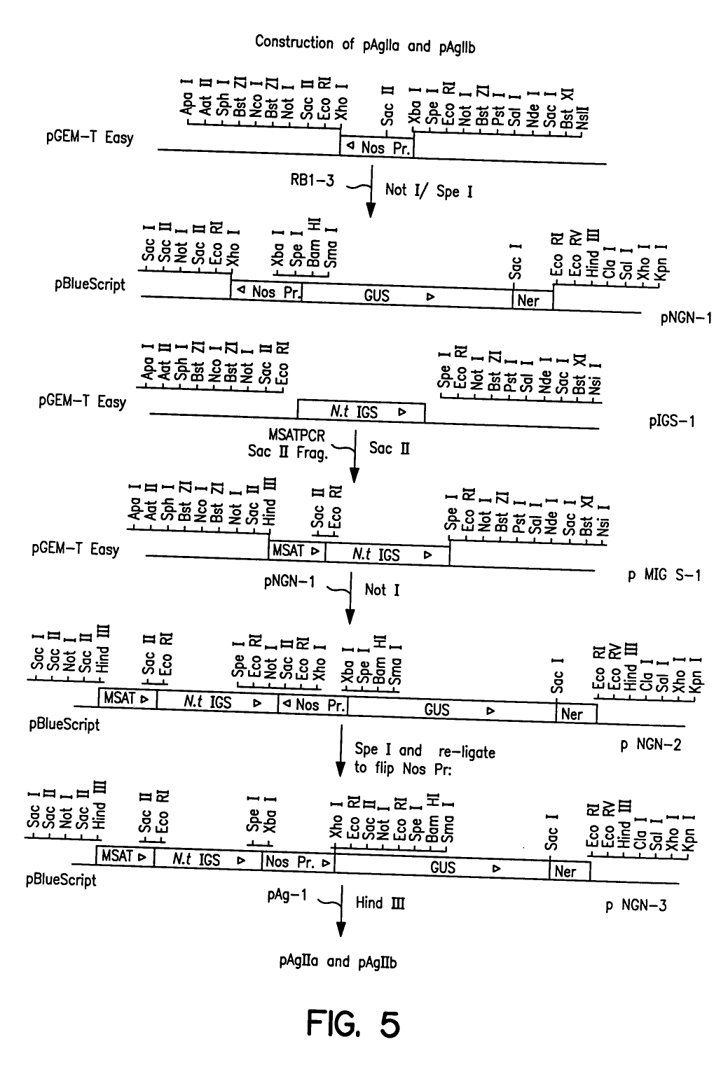

Vectors pAgIIa and pAgIIb were constructed by inserting the following DNA fragments into pAg1: DNA encoding β-glucoronidase, the nopaline synthase terminator fragment, the nopaline synthase (NOS) promoter fragment, a fragment of mouse satellite DNA and an N. tabacum rDNA intergenic spacer sequence (IGS). The construction of pAgIIa and pAgIIb was as follows (see FIG. 5).

An N. tabacum rDNA intergenic spacer (IGS) sequence (SEQ. ID. NO: 9); see also GenBank Accession No. YO8422; see also Borysyuk et al. (2000) Nature Biotechnology 18:1303-1306; Borysyuk et al. (1997) Plant Mol Biol.35:655-660; U.S. Pat. Nos. 6,100,092 and 6,355,860) was obtained by PCR amplification of tobacco genomic DNA. The IGS can be used as a targeting sequence by virtue of its homology to tobacco rDNA genes; the sequence is also an amplification promoter sequence in plants. This fragment was amplified using standard PCR conditions (e.g., as described by Promega Biotech, Madison, Wis., U.S.A.) from tobacco genomic DNA using the primers shown below:

| | |
|---|---|
| NTIGS-FI | (SEQ ID No. 10) |
| 5′-GTG CTA GCC AAT GTT TAA CAA GAT G- 3′ | |
| and | |
| NTIGS-RI | (SEQ ID No. 11) |
| 5′-ATG TCT TAA AAA AAA AAA CCC AAG TGA C- 3′ | |

Following amplification, the fragment was cloned into pGEM-T Easy to give pIGS-I.

A fragment of mouse satellite DNA (Msat1 fragment; GenBank Accession No. V00846; and SEQ ID No. 12) was amplified via PCR from pSAT-1 using the following primers:

| | |
|---|---|
| MSAT-F1 | |
| 5′-AAT ACC GCG GAA GCT TGA CCT GGA ATA TCG C -3′ | (SEQ ID No. 13) |
| and | |
| MSAT-Ri | |
| 5′-ATA ACC GCG GAG TCC TTC AGT GTG CA T- 3′ | (SEQ ID No. 14) |

This amplification added a SacII and a HindIII site at the 5′ end and a SacII site at the 3′ end of the PCR fragment. This fragment was then cloned into the SacII site in pIGS-1 to give pMIGS-1, providing a eukaryotic centromere-specific DNA and a convenient DNA sequence for detection via FISH.

A functional marker gene containing a NOS-promoter:GUS:NOS terminator fusion was then constructed containing the NOS promoter (GenBank Accession No. U09365; SEQ ID No. 15), E. coli β-glucuronidase coding sequence (from the GUS gene; GenBank Accession No. S69414; and SEQ ID No. 16), and the nopaline synthase terminator sequence (GenBank Accession No. U09365; SEQ ID No. 18). The NOS promoter in pGEM-T-NOS was added to a promoterless GUS gene in pBlueScript (Stratagene, La Jolla, Calif., U.S.A.) using NotI/SpeI to form pNGN-1, which has the NOS promoter in the opposite orientation relative to the GUS gene.

pMIGS-1 was digested with NotI/SpeI to yield a fragment containing the mouse major satellite DNA and the tobacco IGS which was then added to NotI-digested pNGN-1 to yield pNGN-2. The NOS promoter was then re-oriented to provide a functional GUS gene, yielding pNGN-3, by digestion and religation with SpeI. Plasmid pNGN-3 was then digested with HindIII, and the HindIII fragment containing the β-glucuronidase coding sequence and the rDNA intergenic spacer, along with the Msat sequence, was added to pAG-1 to form pAgIIa, using the unique HindIII site in pAg1 located near the right T-DNA border of pAg1, within the T-DNA region.

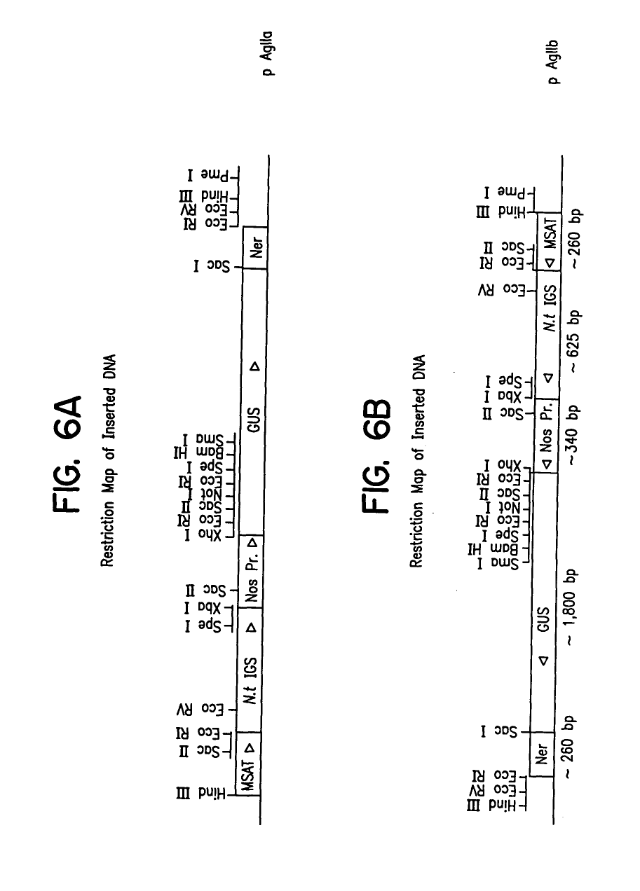

Another plasmid vector, referred to as pAgIIb, was also recovered, which contained the inserted HindIII fragment in the opposite orientation relative to that observed in pAgIIa. Thus, pAgIIa and pAgIIb differ only in the orientation of the HindIII fragment containing the mouse major satellite sequence, the GUS DNA sequence and the IGS sequence (see FIG. 6). The nucleotide sequence of pAgIIa is provided in SEQ. ID. NO: 21.

Vectors pAg1, pAg2, pAgIIa and pAgIIb, as well as similarly designed vectors containing a recombination site and a promoter (e.g., plant or animal promoter), and possibly other regulatory sequences, in operable association with DNA encoding a protein or other product for the expression in a host cell, such as a plant or animal cell, can be used in the transfer of any protein (or other product)-encoding nucleic acid of interest into a cell for expression thereof. For example, any protein (or other product)-encoding nucleic acid of interest (in operable association with transcriptional regulatory suitable for use in a particular host cell) can be inserted into any of the vectors pAg1, pAg2, pAgIIa and pAgIIb and thereby incorporated into a plant, animal or other artificial chromosome, particularly a platform artificial chromosome ACes, as desribed herein.

### EXAMPLE 6

### Agrobacterium-Mediated Transformation of Plant Cells

Plant cells were transformed via Agrobacterium-mediated transformation according to standard procedures (see, for example, Horsch et al. (1988) Plant Molecular Biology Manual, A5:1-9, Kluwer Academic Publisher, Dordrecht, Belgium). Briefly, Agrobacterium strain GV 3101/pMP90 (see Koncz and Schell (1986) Molecular and General Genetics 204:383-396) was transformed with pAgIIa and pAgIIb (see Example 5) by heat shock, and the plasmid integrity of pAgIIa and pAgIIb after transformation was verified by HindIII digest pattern. pAgIIa/pMP90 or pAgIIb/pMP90 were cultured in 5 ml AB minimum medium (Horsch et al. (1 988) Plant Molecular Biology Manual, A5:1-9, Kluwer Academic Publisher, Dordrecht, Belgium) containing 25 μg/ml kanamycin and 25 μg/ml gentamycin at 28° C. for two days.

Leaf disks of tobacco and Arabidopsis and root segments of Arabidopsis were prepared as follows: tobacco leaves from 3 to 4 week-old explants were cut into 1 cm in diameter, and Arabidopsis leaves were taken from 3 week-old seedlings and transversely cut in two halves. Roots of 3 week-old Arabidopsis were excised into segments of 1 cm in length. Co-cultivation was carried out by immersing leaf disks or root segments in bacterial culture for 2 minutes and then transferring the infected tissues to culture medium without antibiotics for 2 days at 22° C. for 16-hours/day under cool white fluorescent light. The leaf disks of tobacco and Arabidopsis were cultured on MS104 medium (MS, 3% sucrose, 0.05% MES, 1.0 mg/l BA, 0.1 mg/l NAA and 0.8% agar, pH 5.8) and root segments on callus-inducing medium, CIM 0.5/0.05 (B5, 2% glucose, 0.05% MES, 0.5 mg/l 2,4-D, 0.05 mg/l kinetin and 0.8% agar, pH 5.8).

The transformed leaf disks and root segments were then transferred to selection medium of MS104 or CIM 0.5/0.05, respectively, containing 20 mg/l hygromycin and 300 mg/l Timentin for the elimination of Agrobacterium. The selection medium was refreshed every two weeks and green shoots regenerated. Plants were analyzed for the expression of the DNA encoding GUS by standard histochemical and fluorescent assays and evidence of amplification of the inserted DNA by quantitative PCR. Numerous plants were obtained that expressed high levels of GUS, and multiple copies of the GUS gene were observed by Fluorescent In Situ Hybridization (FISH) and PCR analysis. Thus, amplification the chromosomal regions containing the inserted DNA was observed. One of skill in the art will appreciate that GUS expression, or the expression of any other gene, can be assessed using methods well known in the art.

### EXAMPL 7

### Transfection and Culture of Arabidopsis Protoplasts

E. coli strain Stb14 (Gibco Life Sciences) was transformed with pAgIIa, pAgIIb, and one of two targeting plasmids containing the rDNA repeat sequence from Arabidopsis (plasmid pJHD-14A or the 26S rDNA from Arabidopsis plasmid pJHD2-19A, as described by Doelling et al. [(1993) Proc. Natl. Acad. Sci. U.S.A. 90:7528-7532]) via electroporation according to standard procedures. A single colony was grown up in 250 ml LB medium containing 50 μg/ml kanamycin (for selection based on the kanamycin resistance-encoding DNA in pAgIIa and pAgIIb) or 50 μg/ml ampicillin (for selection based on the ampicillin resistance-encoding DNA in pJHD-14A & pJHD2-19A) and cultured at 30° C. with shaking at 225 rpm for 16 hours. The plasmids were isolated according to standard procedures well known in the art. The structural integrity of the plasmids was checked by restriction digestion pattern, and the plasmids were linearized with restriction enzymes. Plasmids were sterilized with chloroform and 70% ethanol before use for transfection.

Arabidopsis protoplasts were resuspended in the culture medium (see Example 1) at a density of 2×10⁶ protoplasts/ml. A 300 μl protoplast suspension was pipetted into a 15 ml tube, and 30 μl of plasmid (pAgIIa or pAgIIb) and targeting DNA (pJHD-14A or pJHD2-19A) was added containing 10 μg plasmid and 100 μg targeting sequence followed immediately by slowly adding 300 μl of 10% PEG. The targeting plasmids were included in the transfection procedure in order ensure that the amount of rDNA targeting DNA (i.e., tobacco rDNA from pAgIIa or b and Arabidopsis DNA from the targeting vectors) was sufficient to effect recombination of the introduced DNA at a homologous site in an Arabidopsis chromosome. DNA was typically used in a ratio of 10:1, targeting DNA (pJHD-14A or pJDH2-19A, or Lambda DNA) to plasmid DNA (pAgIIa or pAgIIb, or a selectable marker plasmid), or in a ratio of 5:1. Generally, the number of base pairs of targeting DNA to be sufficient for insertion into a plant chromosome is at least about 50 bp, or about 60 bp, or about 70 bp, or about 80 bp, or about 90 bp, or about 100 bp, or about 150 bp, or about 200 bp, or about 300 bp, or about 400 bp, or about 500 bp, or about 600 bp, or about 700 bp, or about 800 bp, or about 900 bp, or about 1 kb, or about 2 kb or about 3 kb, or about 4 kb, or about 5 kb, or about 6 kb, or about 7 kb, or about 8 kb, or about 9 kb, or about 10 kb or more. The amount and length of targeting DNA sufficient to effect introduction into a chromosome can be determined empirically and can vary for different plant species.

The mixture was shaken gently, and immediately 300 μl of 10% PEG solution was added slowly with gentle shaking. The protoplast mixture was incubated at 22° C. for 10-15 min with several cycles of gentle shaking. DNA uptake was quenched by the addition of 5 ml 72.4 g/l Ca(NO₃)₂. The protoplasts were then centrifuged at 80×g for 7 min and resuspended in culture medium. For selection, 10 to 40 mg/l hygromycin was added to protoplast cultures 14 days after transfection, and the culture medium was refreshed every 7 days. The protoplast cultures could also be selected after embedding in 0.6% agarose by transferring to a culture medium containing 20 mg/l hygromycin. The cultures were incubated for 14 days or longer at 22° C.

The Arabidopsis protoplasts were analyzed for the presence and expression of the DNA encoding GUS. Recovered microcalli strongly expressed GUS and were resistant to selective agents, indicating amplification of the inserted DNA. Alternatively, the transfection of Arabidopsis protoplasts can be conducted without using targeting DNA sequences since pAgIIa and pAgIIb include a region of rDNA (i.e. the tobacco rDNA IGS) that can act as a targeting sequence as long as a sufficient amount of pAgIIa/b plasmid is used in the transfection procedure.

### EXAMPLE 8

### Transfection and Culture of Tobacco Protoplasts

As described in Example 7, E. coli strain Stb14 was transformed with pAgIIa, pAgIIb, pJHD-14A (targeting DNA) and pJHD2-19A (targeting DNA) via electroporation, and plasmid DNA was recovered and linearized with restriction enzymes. Plasmids were sterilized with chloroform and 70% ethanol before use for transfection.

The tobacco protoplasts (see Examples 2 and 3) were resuspended in the culture medium (see Example 2) at a density of 2×10⁶ protoplasts/ml. A 300 μl protoplast suspension was pipetted into a 15 ml tube, and 30 μl of plasmid and targeting DNA was added as described in Example 7. The mixture was shaken gently, and immediately 300 μl of 10% PEG solution was added slowly with gentle shaking. The tobacco protoplast mixture was incubated at 22° C. for 10-15 min with several cycles of gentle shaking. DNA uptake was quenched by the addition of 5 ml 72.4 g/L Ca(NO₃)₂. The protoplasts were then centrifuged at 80×g for 7 min and resuspended in culture medium.

The recovery of viable tobacco protoplasts following DNA uptake ranged from 65-75% following treatment. Typically greater than 35% of the protoplasts initiated cell division within 7 days of treatment. Protoplast cells were analyzed for gene expression (in this case for the expression of the reporter DNA GUS, but alternatively, the expression of other genes can be monitored). Between 4% and 6% of the recovered cells exhibited GUS expression.

The protoplasts were subject to selection procedures to recover transformed cells. For selection of tobacco cells, 10 to 40 mg/l hygromycin was added to protoplast cultures 10-14 days after transfection, and the culture medium was refreshed every 7 days. Leaf disc selection was performed in the presence of 40 mg/l hygromycin. Transformed microcalli were recovered and analyzed for the expression of the GUS reporter gene. GUS positive calli were isolated and subjected to FISH analysis (see Example 13). Plant cells that exhibited amplification of the inserted DNA were identified.

### EXAMPLE 9

### Transfection and Cultur of Brassica Protoplasts

Brassica protoplasts (see Example 4), following the final washing step after filtering through a 63 μm nylon screen and centrifugation, are collected and used for DNA transfection as described in Example 8. Brassica protoplast cultures following DNA uptake or transformation by Agrobacterium can be selected with either hygromycin or glufosinate ammonium in liquid culture or in embedded semi-solid cultures. The effective concentration of hygromycin is 10 to 40 mg/l for 2 to 4 weeks or continuously, whereas that for glufosinate ammonium is 2 to 60 mg/l for 5 days to 2 weeks. Selection can impede growth, and additional transfers to similar media may be required.

### EXAMPLE 10

### Plant Regeneration from Brassica Protoplasts

Colonies of Brassica protoplasts (1 mm or larger in diameter) are plated onto regeneration medium (basal Murashige and Skoog's medium, 1% sucrose, 2 mg/l BA, 0.01 mg/l NAA, 0.8% agarose, pH 5.6). Cultures are incubated under the conditions described in Example 4. Cultures are transferred onto fresh regeneration medium every 2 weeks. Regenerated shoots are transferred onto autoclaved rooting medium (basal Murashige and Skoog's medium, 1% sucrose, 0.1 mg/l NAA, 0.8% agar, pH 5.8) and incubated under dim fluorescent light (25 μEm⁻² S⁻¹). Plantlets are potted in a soil-less mix (for example, Terra-lite Redi-Earth, W.R. Grace & Co., Canada Ltd., Ajax, Ontario) containing fertilizer (Nutricote 1414-14 type 100, Plant Products Co. Ltd, Brampton, Ontario) and grown in a growth room (20° C./15° C., 16 h photoperiod, 100-140 μEm⁻² S⁻¹) with fluorescent and incandescent light at soil level. Plantlets are covered with transparent plastic cups for one week to allow for acclimatization.

### EXAMPLE 11

### Isolation of Nuclei from Protoplasts

To facilitate analysis, plant cells can be subjected to nuclei isolation, and the isolated nuclei can be analyzed by FISH or PCR. To isolate the nuclei, protoplast calli were reprotoplasted according to the procedure of Mathur et al. with modifications (see Mathur et al. Plant Cell Report (1995) 14: 221-226). The protoplast calli were digested with 1.2% Cellulase ‘Onozuka’ R-10 and 0.4% w/v Macerozyme R-10 in nuclei isolation buffer (10 mM MES-pH 5.5, 0.2M sucrose, 2.5 mM EDTA, 2.5 mM DTT, 0.1 mM spermine, 10 mM NaCl, 10 mM KCl and 0.15% Triton X-100) for 3 hours. After centrifugation at 80×g for 10 minutes, the pellets of protoplasts were resuspended in hypertonic buffer of 1 2.5% W5 solution (Hinnisdaels et al. (1994) Plant Molecular Biology Manual G2:1-13, Kluwer Academic Publisher, Belgium) for 10 minutes. To promote disruption of protoplasts, the protoplast suspension was forced through a syringe needle four times. The disrupted protoplasts were filtered through 5 μm meshes to remove debris and centrifuged at 200×g for 10 min. By repeated washing of the pellet in a nuclei isolation buffer containing phenylmethylsulfonylfluoride (PMSF) and centrifugation at 200×g for 10 minutes, nuclei were collected as a white pellet freed from cytoplasm contamination and cellular debris. Samples were fixed in 3:1 methanol:glacial acetic acid and were analyzed by FISH.

### EXAMPLE 12

### Mitotic Arrest of Plant Cells for Detection of Amplification and Artificial Chromosome Formation

In general, plant cells or protoplasts are typically cultured for two or more generations prior to mitotic arrest. Typically, 5 μg/ml colchicine is added to the cultures for 12 hours to accumulate mitotic plant cells. The mitotic cells are harvested by gentle centrifugation. Alternatively, plant cells (grown on plastic or in suspension) can be arrested in different stages of the cell cycle with chemical agents other than colchicine, such as, but not limited to, hydroxyurea, vinblastine, colcemid or aphidicolin or through the deprivation of nutrients, hormones, or growth factors. Chemical agents that arrest the cells in stages other than mitosis, such as, but not limited to, hydroxyurea and aphidicolin, are used to synchronize the cycles of all cells in the population and are then removed from the cell medium to allow the cells to proceed, more or less simultaneously, to mitosis at which time they can be harvested to disperse the chromosomes.

### EXAMPLE 13

### Detection of Amplification and Artificial Chromosome Formation by Fluorescence in Situ Hybridization (FISH)

A variety of plant cells can analyzed by fluorescence in situ hybridization (FISH) methods (Fransz et al. (1996) Plant J. 9:421-430; Fransz et al. (1998) Plant J. 13:867-876; Wilkes et al. (1995) Chromosome Research 3:466-472; Busch et al. (1994) Chromosome Research 2:15-20; Nkongolo (1993) Genome 36:701-705; Leitch et al. (1994) Methods in Molecular Biology 28:177-185; Murata et aL. (1997) Plant J. 12:31-37) to identify amplification events and artificial chromosome formation.

FISH is used to detect specific DNA sequences on chromosomes, in particular to detect regions of plant chromosomes that have undergone amplification as a result of the introduction of heterologous DNA as described herein, or to detect artificial chromosome formation in plant cells. FISH chromosome spreads of Arabidopsis and tobacco plant cells into which heterologous DNA has been introduced are generated using colchicine or similar cell cycle arresting agents and various DNA probes (e.g. rDNA probe, Lambda DNA probe, selectable marker probe). The cells are analyzed for the presence of amplified regions of chromosomes, in particular amplification of the rDNA regions, and those cells exhibiting amplification are further cultured and analyzed for the formation of artificial chromosomes.

The chromosomes of plant cells subjected to introduction of heterologous DNA and growth to generate artificial chromosomes can also be analyzed by scanning electron microscopy. Preparation of mitotic chromosomes for scanning electron microscopy can be performed using methods known in the art (see, e.g., Sumner (1991) Chromosome 100:410-418). The chromosomes can be observed, for example, with a Hitachi S-800 field emission scanning electron microscope operated with an accelerating voltage of 25 kV.

### EXAMPLE 14

### Detection of Amplification and Artificial Chromosome Formation by Idu Labeling of Chromosomes

The structure of the chromosomes in plant cells can be analyzed by labeling the chromosomes with iododeoxyuridine (IdU), or other nucleotide analog, and using an IdU-specific antibody to visualize the chromosome structure. Plant cell cultures selected following introduction of heterologous DNA are labeled with IdU following standard protocols (Fujishige and Taniguchi (1998) Chromosome Research 6:611-619; Yanpaisan et al. (1998) Biotechnology and Bioengineering, 58:515-528; Trick and Bates (1996) Plant Cell Reports, 15:986-990; Binarova et al. (1993) Theoretical and Applied Genetics, 87:9-16; Wang et al. (1991) Journal of Plant Physiology, 138:200-203). Plant cells in culture, typically suspension culture, are used. A series of sub-cultures are initiated, and IdU labeling is performed as described above. Cells are allowed to incorporate IdU for up to a week, depending on the doubling time of the culture. Labeled chromosomes can be detected in plant cells (Fujishige and Taniguchi (1998) Chromosome Research 6:611-619; Binarova et al. (1993) Theoretical and Applied Genetics 87:9-16) and in mammalian cells (Gratzner and Leif (1981) Cytometry 1:385-393) using procedures well known in the art. IdU-labeled chromosomes are detected by immunocytochemical techniques. An anti-IdU fluorescein isothiocyanate (FITC)-conjugated B44 clone antibody (Becton Dickinson) is used to bind the IdU-DNA adduct in the DNA and is detected by fluorescence microscopy (490 nm excitation, 519 nm emission). Analysis of labeled chromosomes reveals the presence of amplified DNA regions and the formation of artificial chromosomes.

### EXAMPLE 15

### Isolation of Metaphase Chromosomes from Protoplasts

Artificial chromosomes, once detected in plant cells, may be isolated for transfer to other organisms and in particular other plant species. Several procedures may be used to isolate metaphase chromosomes from mitotic-arrested plant cells, including, but not limited to, a polyamine-based buffer system (Cram et al. (1990) Methods in Cell Biology 33:377-3821), a modified hexylene glycol buffer system (Hadlaczky et al. (1982) Chromosoma 86:643-65), a magnesium sulfate buffer system (Van den Engh et al. (1988) Cytometry 9:266-270 and Van den Engh et al. (1984) Cytometry 5:108), an acetic acid fixation buffer system (Stoehr et al. (1982)

Histochemistry 74:57-61), and a technique utilizing hypotonic KCl and propidium iodide (Cram et al. (1994) XVII meeting of the International Society for Analytical Cytology, October 16-21, Tutorial IV Chromosome Analysis and Sorting with Commerical Flow Cytometers; Cram et al. (1990) Methods in Cell Biology 33:376; de Jong et al. (1999) Cytometry 35:129-133).

In an exemplary procedure, a hexylene glycol buffer is used to isolate plant chromosomes from mitotic-arrested plant cells that have been converted to protoplasts (Hadlaczky et al. (1982) Chromosoma 86:643-659). Chromosomes are isolated from about 10⁶ mitotic cells re-suspended in a glycine-hexylene glycol buffer (100 mM glycine, 1% hexylene glycol, pH 8.4-8.6, adjusted with a solution of saturated Ca(OH)₂) supplemented with 0.1% Triton X-100 (GHT buffer). The cells are incubated for 10 minutes at 37° C., and the chromosomes are purified by differential centrifugation to pellet the nuclei (200×g for 20 min) and sucrose gradient centrifugation (5-30% sucrose, 5600×g for 60 min, 0-4° C.). To avoid proteolytic degradation of chromosomal proteins, 1 mM PMSF (phenylmethylsulfonylfluoride) is used in the presence of 1% isopropyl alcohol. The proteins can be extracted from the isolated chromosomes using dextran sulfate-heparin (DSH) extraction, and the chromosomes can be visualized via electron microscopy using techniques known in the art (Hadlaczky et al. (1982) Chromosoma (Berl.) 86:643-659; Hadlaczky et al. (1981) Chromosoma (Berl.) 81:537-555). Additionally, modifications of these procedures, including, but not limited to, modification of the buffer composition (Carrano et al. (1979) Proc. Natl. Acad. Sci. U.S.A. 76:1382-1384) and variation of the centrifugation time or speed, to accommodate different plant species can be implemented by any skilled artisan.

### EXAMPLE 16

### Transfer of Artificial Chromosomes into Plant Cells: Transfer of Mammalian Artificial Chromosomes into a Dicot Plant: Arabidopsis

One method of delivery of mammalian artificial chromosomes (MACs) into plant cells is the formation of microcells containing murine MACs and the CaPO₄-mediated uptake or the PEG-mediated fusion of these microcells with plant protoplasts. In this example, microcells and plant protoplasts, such as but not limited to tobacco and Arabidopsis protoplasts, were mixed (in a series of 25:1, 10:1, 5:1, or 2:1 microcells:protoplasts ratio) and fusion was observed. Protocols for the formation of microcells are known in the art and are described, for example, in U.S. Pat. Nos. 5,240,840, 4,806,476 and 5,298,429 and in Fournier Proc. Natl. Acad. Sci. U.S.A. (1981) 78:6349-6353 and Lambert et al. Proc. Natl. Acad. Sci. U.S.A. (1991) 88: 5907-5912. The murine microcells can be labeled with Idu or the IVIACs stained with a specific dye such as, but not limited to, e.g., propidium iodide or DAPI, prior to fusion with plant protoplasts including, but not limited to, Arabidopsis and tobacco protoplasts, to facilitate detection of the presence of IVIACs in the protoplasts.

In this example, MACs were introduced into Arabidopsis cells using microcell-PEG mediated fusion. Microcells were formed from murine cells containing an artificial chromosome (see U.S. Pat. No. 6,077,697) and were fused with freshly prepared Arabidopsis protoplasts in a ratio of 10:1, microcells to protoplasts. Fusion occurred in the presence of 25% PEG 6000, 204 mM CaCl₂, pH 6.9 within the first 5 minutes of mixing. Typically less than about one minute of mixing is required to observe fusion between microcells and protoplasts. Fused cells were washed with 240 mM CaCl₂, then floated on top of a solution of 204 mM sucrose in B5 salts. Cells were then transferred to cell suspension culture media (MS, 87 mM sucrose, 2.7 μM napthalene acetic acid, 0.23 μM kinetin, pH 5.8). Empirical observations can be used to determine the optimal concentration and composition of PEG and the concentration of calcium that provides the highest degree of fusion with the least toxicity.

Fused protoplasts were allowed to grow for one or more generations. The presence of a mouse chromosomal sequence, including MACs, was demonstrated by southern hybridization with MAC probes, by FISH analysis and by PCR analysis using, for example, satellite sequences known to exist on the MAC chromosome. Thus, the mouse sequences were detected in the Arabidopsis protoplasts.

To further demonstrate the transfer of mouse chromosomal sequence to Arabidopsis protoplasts, Arabidopsis plant cell nuclei were isolated according to Example 11 and were subjected to FISH analysis according to Example 13, using the mouse major satellite DNA (SEQ ID No. 12). A portion of the nuclei contained a significant signal using the mouse major satellite DNA, indicating successful transfer of at least a mouse chromosome and/or MAC to the Arabidopsis nuclei.

Similarly, PACs may be introduced into Arabidopsis protoplasts using PEG- and/or calcium-mediated fusion procedures. Generation of microprotoplasts and protoplasts can be conducted as described, for example, in Example 1. Microprotoplasts formed from plant cells containing a plant artificial chromosome are fused with freshly prepared Arabidopsis protoplasts, for example, in a ratio of 10:1, microprotoplasts to protoplasts. Protoplasts from other plants, including but not limited to, tobacco, wheat, maize and rice, can also be used as the recipient of MACs and/or PACs. Fused protoplasts are recovered and allowed to grow for one or more generations. The presence of the transferred PACs can be analyzed using methods such as, for example, those described herein (including Southern hybridization with PAC probes, FISH analysis and PCR analysis using DNA sequences specific to the PAC).

### EXAMPLE 17

### Transfer of Artificial Chromosomes into Plant Cells: Transfer of Mammalian Artificial Chromosomes into a Second Dicot Plant: Tobacco

MACs were introduced into tobacco cells using microcell-PEG-mediated fusion using the same microcells, MAC, and protocol as described in Example 16. Microcells were formed from murine cells containing an artificial chromosome and were fused with freshly prepared tobacco BY-2 protoplasts in a ratio of 10:1, microcells to protoplasts. Fusion occurred in the presence of 20% PEG 4000 and 100-200 mM calcium chloride. Empirical observations are used to determine the optimal concentration and composition of PEG and the concentration of calcium that provides the highest degree of fusion with the least toxicity.

DAPI staining of the microcells (e.g. by preincubation of the microcells with DAPI by adding DAPI to the microcells to a final concentration of 1 μg/ml) allowed visualization of the fusion and transfer of the chromosomes to the tobacco protoplasts. Fused protoplasts were recovered and allowed to grow for one or more generations. The fused protoplasts can be analyzed for the presence of a MAC in a number of ways, including those described herein. Fused tobacco cell nuclei were isolated from tobacco protoplasts that had been fused with microcells according to Example 11 and were subjected to FISH analysis according to Example 13, using the mouse major satellite DNA (SEQ ID No. 12). Numerous nuclei were found to have incorporated a mouse chromosome.

### EXAMPLE 18

### Transfer of isolated Artificial Chromosomes by Lipid-Mediated Transfer into a Monocot Plant: Rice

Isolated murine artificial chromosomes (MACs) prepared by sorting through a FACS apparatus (de Jong et al. Cytometry (1999) 35:129-133) were transferred into rice plant protoplasts by cationic lipid-mediated transfection of the purified MAC. Purified MACs (see Example 15 and U.S. Pat. No. 6,077,697) were mixed with LipofectAMINE 2000 (Gibco, Md., USA) as follows. Typically, 15 μl of LipofectAMINE 2000 were added to 1×10⁶ artificial chromosomes in liquid buffer, the solution allowed to complex for up to three hours, and then the solution was added to freshly prepared 1×10⁵ rice protoplasts prepared using standard protoplast methods well known in the art. The uptake of the lipid-complexed artificial chromosome was monitored by adding to the mixture of protoplasts and purified artificial chromosomes a fluorescent dye that stains DNA. Microscopic examination of the protoplast/artificial chromosome mixture over the next several hours allowed the visualization of the artificial chromosome being transported across the protoplast cellular membrane and the presence of the readily identifiable MAC in the cytoplasm of the rice plant cell.

The same procedure as described in this Example for cationic lipid-mediated transfer of an isolated MAC into rice protoplasts can be used to transfer isolated MACs, as well as PACs, into rice and other plant protoplasts, including but not limited to, tobacco, wheat, maize and Arabidopsis. Fused protoplasts are recovered and allowed to grow for one or more generations. The presence of the transferred MACs and PACs can be analyzed using methods such as, for example, those described herein (including, but not limited to, Southern hybridization with PAC probes, FISH analysis and PCR analysis using DNA sequences specific to the PAC).

### EXAMPLE 19

### Delivery of Plant Regulatory and Coding Sequences via a Promoterless attBZeo Marker Gene in pAg2 onto a MAC Platform

As described in Examples 6-15, the plasmid pAg2, comprising plant regulatory and selectable marker genes (SEQ ID NO: 6; prepared as set forth in Example 5) can be used for the production of a MAC containing said plant expressible genes. In this example, pAg2, by virtue of the attBZeo DNA sequences contained on the plasmid, is used for the loading of plant regulatory and selectable marker genes onto MACs in mammalian cells using the attB sequences to recombine with attP sequences present on a platform MAC. In this example, platform MACs are produced with attP sequences and the plasmid pAg2 is then loaded onto the platform MAC. New MACs so produced are useful for introduction into plan cells by virtue of the plant expressible markers contained therein.

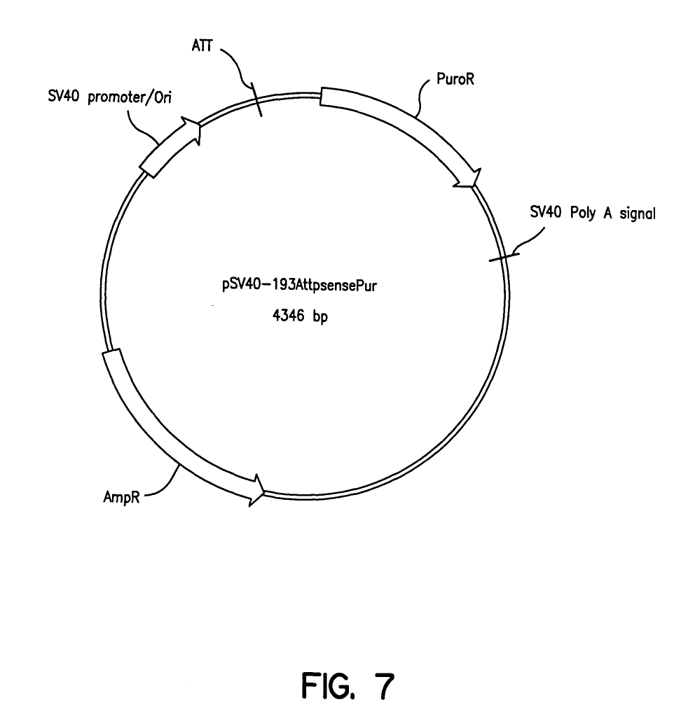

A. Construction of Platform MAC Containing pSV40attPsensePUR (FIG. 7; SEQ ID NO: 26).

An example of a selectable marker system for the creation of a MAC-based platform into which the plasmid pAg2 can target plant regulatory and coding sequences is shown in FIG. 7. This system includes a vector containing the SV40 early promoter immediately followed by (1) a 282 base pair (bp) sequence containing the bacteriophage lambda attP site and (2) the puromycin resistance marker. Initially a PvuII/StuI fragment containing the SV40 early promoter from plasmid pPUR (Clontech Laboratories, Inc., Palo Alto, Calif.; SEQ ID No. 22) was subcloned into the EcoRI/CRI site of pNEB193 (a PUC19 derivative obtained from New England Biolabs, Beverly, Mass.; SEQ ID No. 23) generating the plasmid pSV40193.

The attP site was PCR amplified from lambda genome (GenBank Accession # NC 001416) using the following primers:

| | | |
|---|---|---|
| attPUP: | CCTTGCGCTAATGCTCTGTTACAGG | SEQ ID No. 24 |
| attPDWN: | CAGAGGCAGGGAGTGGGACAAAATTG | SEQ ID No. 25 |

After amplification and purification of the resulting fragment, the attP site was cloned into the SmaI site of pSV40193 and the orientation of the attP site was determined by DNA sequence analysis (plasmid pSV40193attP). The gene encoding puromycin resistance (Puro) was isolated by digesting the plasmid pPUR (Clontech Laboratories, Inc. Palo Alto, Calif.) with AgeI/BamHI followed by filling in the overhangs with Klenow and subsequently cloned into the AscI site downstream of the attP site of pSV40193attP generating the plasmid pSV40193attPsensePUR (FIG. 7; SEQ ID NO:26)).

The plasmid pSV40193attPsensePUR was digested with ScaI and co-transfected with the plasmid pFK161 into mouse LMtk-cells and platform artificial chromosomes were identified and isolated as described herein. Briefly, Puromycin resistant colonies were isolated and subsequently tested for artificial chromosome formation via fluorescent in situ hybridization (FISH) (using mouse major and minor DNA repeat sequences, the puromycin gene and telomeres sequences as probes), and their fluorescent activating cell sorted (FACS). From this sort, a subclone was isolated containing an artificial chromosome, designated B19-38. FISH analysis of the B19-38 subclone demonstrated the presence of telomeres and mouse minor on the MAC. DOT PCR has been done revealing the absence of uncharacterized euchromatic regions on the MAC. The process for generating this exemplary MAC platform containing multiple site-specific recombination sites is summarized in FIG. 5. This MAC chromosome may subsequently be engineered to contain target gene expression nucleic acids using the lambda integrase mediated site-specific recombination system as described below.

B. Construction of Targeting Vector.

The construction of the targeting vector pAg2 is set forth in Example 5 herein.

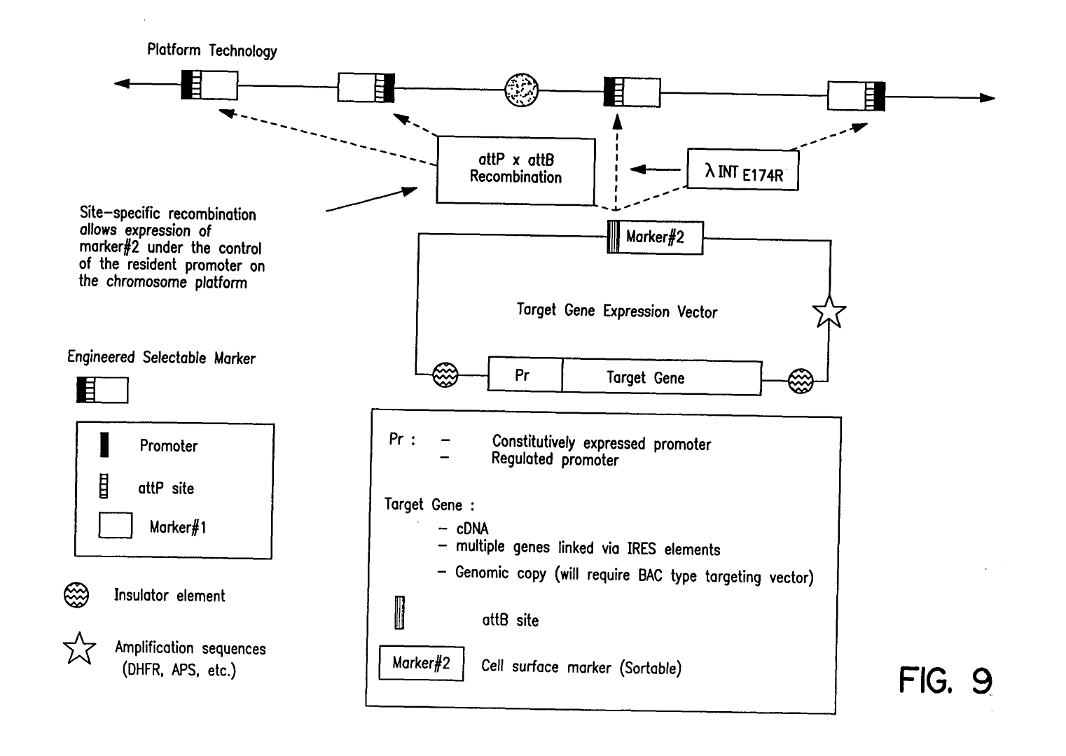

C. Transfection of Promotorless Marker and Selection With Drug (See FIG. 9).

The mouse LMtk-cell line containing the MAC B19-38 (constructed as set forth above and also referred to as a 2^{nd} generation platform ACE), is plated onto four 10 cm dishes at approximately 5 million cells per dish. The cells are incubated overnight in DMEM with 10% fetal calf serum at 37° C. and 5% CO₂. The following day the cells are transfected with 5 μg of the vector pAg2 (prepared as described in Example 5 above) and 5 μg of pCXLamIntR (encoding a lambda integrase having an E to R amino acid substitution at position 174), for a total of 10 μg per 10 cm dish. Lipofectamine Plus reagent is used to transfect the cells according to the manufacturers protocol. Two days post-transfection zeocin is added to the medium at 500 ug/ml. The cells are maintained in selective medium until colonies are formed. The colonies are then ring-cloned and genomic DNA is analyzed.

D. Analysis Of Clon s (PCR, SEQUENCING).

Genomic DNA (including MACs) is isolated from each of the candidate clones with the Wizard kit (Promega) and following the manufacturers protocol. The following primer set is used to analyze the genomic DNA isolated from the zeocin resistant clones: 5PacSV40—CTGTTAATTAACTGTGGAATGTGTG TCAGTTAGGGTG (SEQ ID NO: 28); Antisense Zeo—TGAACAGGGTCACGTCGTCC (SEQ ID NO: 29). PCR amplification using the above primers and genomic DNA, which included MACs, from the candidate clones results in a PCR product indicating the correct sequence for the desired site-specific integration event.

The MACs containing the pAg2 vector are identified and used for transfer into plant (such as described in Examples 16 and 17) or animal cells for the expression of the desired coding sequences contained therein. The MACs containing pAg2 carry two plan selectable markers (hygromycin resistance, resistance to phosphinothricin) and a visual selectable marker (green fluorescent protein).

### EXAMPLE 20

Construction of Plant-Derived Shuttle Artificial Chromosome.

In another embodiment, the plant artificial chromosomes provided herein are useful as selectable shuttle vectors that are able to move one or more desired genes back and forth between plant and mammalian cells. In this particular embodiment, the plant artificial chromosome is bi-functional in that proper integration of donor nucleic acid can be selected for in both plant and mammalian cells.

For example, a plant artificial chromosome is prepared as described in Examples 6-15 above using ing the plasmid pAg2 (Example 5; SEQ ID NO: 6) that has been modified to include the SV40attPsensePur coding region from the plasmid pSV40193attPsensePur (described above in Example 19.A.). Thus, the resulting plant-derived shuttle artificial chromosome contains DNA from the bar gene confering resistance to phosphinothricin in plant cells, DNA from the hygromycin resistance gene conferring resistance to hygromycin in plant cells, both resistance-encoding DNAs under the control of a separate cauliflower mosaic virus (CaMV) 35S promoter, the attB-promoterless zeomycin resistance-encoding DNA, and DNA conferring resistance to puromycin under the control of a mammalian SV40 promoter. Accordingly, the presence of the shuttle PAC in either a plant or mammalian cell can be selected for by treatment with, for example, either hygromycin (plant) or puromycin (mammalian).

Because the resulting plant-derived shuttle artificial chromosome contains at least one SV40attP site therein similar to the platform MAC prepared in Example 19.A. above, a donor vector containing an attB-selectable marker sequence, such as a plasmid comprising an attBzeo (e.g. pAg2) can be used to selectively introduce desired heterologous nucleic acids from any species (such as plants, animals, insects and the like) into the shuttle artificial chromosome that is present in a mammalian cell.

Likewise, a plant promoter region, such as CaMV35S, can be used to replace the SV40 promoter in the SV40attPPur region of the modified pAg2 plasmid described above. In this embodiment, because the resulting plant-derived shuttle artificial chromosome contains at least one CaMV35SattP site therein analogous to the platform MAC prepared in Example 19.A. above, a donor vector containing an attB-selectable marker sequence, such as a plasmid having attBkanamycin, or other plant selectable or scorable marker can be used to selectively introduce desired heterologous nucleic acids from any species (such as plants, animals, insects and the like) into the shuttle artificial chromosome that is present in a plant cell.

Since modifications will be apparent to those of skill in this art, it is intended that this invention be limited by only the scope of the appended claims.

1. A method for producing an artificial chromosome, comprising:
introducing nucleic acid into a cell comprising one or more plant chromosomes; and
selecting a cell comprising an artificial chromosome that comprises one or more repeat regions wherein:
one or more nucleic acid units is (are) repeated in a repeat region;
repeats of a nucleic acid unit have common nucleic acid sequences; and
the repeat region(s) contain substantially equivalent amounts of euchromatic and heterochromatic nucleic acid.

2. The method of claim 1, wherein the artificial chromosome is predominantly made up of one or more repeat regions.

3. The method of claim 1, wherein the nucleic acid introduced into the cell comprises a nucleic acid sequence that facilitates amplification of a region of a plant chromosome or targets the nucleic acid to an amplifiable region of a plant chromosome.

4. The method of claim 1, wherein the nucleic acid introduced into the cell comprises one or more nucleic acids selected from the group consisting of rDNA, lambda phage DNA and satellite DNA.

5. The method of claim 4, wherein the nucleic acid comprises plant rDNA.

6. The method of claim 5, wherein the rDNA is from a plant selected from the group consisting of Arabidopsis, Nicotiana, Solanum, Lycopersicon, Daucus, Hordeum, Zea mays, Brassica, Triticum and Oryza.

7. The method of claim 4, wherein the nucleic acid comprises animal rDNA.

8. The method of claim 7, wherein the rDNA is mammalian rDNA.

9. The method of claim 4, wherein the nucleic acid comprises rDNA comprising sequence of an intergenic spacer region.

10. The method of claim 9, wherein the intergenic spacer region is from DNA from a plant selected from the group consisting of Arabidopsis, Solanum, Lycopersicon, Hordeum, Zea, Oryza, rye, wheat, radish and mung bean.

11. The method of claim 1, wherein the nucleic acid introduced into the cell comprises a nucleic acid sequence that facilitates identification of cells containing the nucleic acid.

12. The method of claim 11, wherein the nucleic acid sequence encodes a fluorescent protein.

13. The method of claim 12, wherein the protein is a green fluorescent protein.

14. The method of claim 1, wherein the step of selecting a cell comprising an artificial chromosome comprises sorting of cells into which nucleic acid was introduced.

15. The method of claim 1, wherein the step of selecting a cell comprising an artificial chromosome comprises fluorescent in situ hybridization (FISH) analysis of cells into which nucleic acid was introduced.

16. The method of claim 1, wherein the one or more plant chromosomes contained in the cell is (are) selected from the group consisting of Arabidopsis, tobacco and Helianthus chromosomes.

17. The method of claim 16, wherein the cell is a plant protoplast.

18. The method of claim 1, wherein the nucleic acid introduced into the cell comprises nucleic acid encoding a selectable marker.

19. The method of claim 18, wherein the selectable marker confers resistance to phosphinothricin, ammonium glufosinate, glyphosate, kanamycin, hygromycin, dihydrofolate or sulfonylurea.

20. An isolated plant artificial chromosome comprising one or more repeat regions, wherein:
one or more nucleic acid units is (are) repeated in a repeat region;
repeats of a nucleic acid unit have common nucleic acid sequences; and
the repeat region(s) contain substantially equivalent amounts of euchromatic and heterochromatic nucleic acid.

21. The plant artificial chromosome of claim 20, wherein the artificial chromosome is predominantly made up of one or more repeat regions.

22. A plant cell comprising an artificial chromosome, wherein the artificial chromosome is produced by the method of claim 1.

23. A method of producing a transgenic plant, comprising introducing the artificial chromosome of claim 20 into a plant cell.

24. The method of claim 23, wherein the artificial chromosome comprises heterologous nucleic acid encoding a gene product.

25. The method of claim 24, wherein the heterologous nucleic acid encodes a product selected from the group consisting of enzymes, antisense RNA, tRNA, rDNA, structural proteins, marker proteins, ligands, receptors, ribozymes, therapeutic proteins and biopharmaceutical proteins.

26. The method of claim 24, wherein the heterologous nucleic acid encodes a product selected from the group consisting of vaccines, blood factors, antigens, hormones, cytokines, growth factors and antibodies.

27. The method of claim 24, wherein the heterologous nucleic acid encodes a product that provides for resistance to diseases, insects, herbicides or stress in the plant.

28. The method of claim 24, wherein the heterologous nucleic acid encodes a product that provides for an agronomically important trait in the plant.

29. The method of claim 24, wherein the heterologous nucleic acid encodes a product that alters the nutrient utilization and/or improves the nutrient quality of the plant.

30. The method of claim 24, wherein the heterologous nucleic acid is contained within a bacterial artificial chromosome (BAC) or a yeast artificial chromosome (YAC).

31. A method of identifying plant genes encoding particular traits, comprising:
generating an artificial chromosome comprising euchromatic DNA from a first species of plant;
introducing the artificial chromosome into a plant cell of a second species of plant; and
detecting phenotypic changes in the plant cell comprising the artificial chromosome and/or a plant generated from the plant cell comprising the artificial chromosome.

32. The method of claim 31, wherein the artificial chromosome is a plant artificial chromosome or a mammalian artificial chromosome.

33. The method of claim 31, wherein the artificial chromosome is produced by a method comprising:
introducing nucleic acid into a cell comprising one or more plant chromosomes; and
selecting a plant cell comprising an artificial chromosome that comprises one or more repeat regions, wherein:
repeats of a nucleic acid unit have common nucleic acid sequences; and
the repeat region(s) contain substantially equivalent amounts of euchromatic and heterochromatic nucleic acid.

34. The method of claim 31, wherein the artificial chromosome is produced by a method comprising:
introducing nucleic acid into a plant cell; and
selecting a plant cell comprising a SATAC.

35. The method of claim 31, wherein the artificial chromosome is a minichromosome produced by a method comprising:
introducing nucleic acid into a plant cell; and
selecting a cell comprising a minichromosome comprising a neo-centromere and euchromatin.

36. The method of claim 33, wherein the nucleic acid introduced into the plant cell comprises DNA encoding a selectable marker.

37. The method of claim 36, wherein the selectable marker confers resistance to phosphinothricin, ammonium glufosinate, glyphosate, kanamycin, hygromycin, dihydrofolate or sulfonylurea.

38. The method of claim 31, wherein the artificial chromosome comprising euchromatic DNA from a first plant species is produced by a method comprising:
introducing into a plant cell of a first plant species an artificial chromosome capable of undergoing homologous recombination with the DNA of the first plant species;
selecting for a recombination event between the artificial chromosome and the DNA of the first plant species; and
selecting an artificial chromosome comprising euchromatic DNA from the first plant species.

39. The method of claim 31, wherein the artificial chromosome comprising euchromatic DNA from a first plant species is produced by a method comprising:
introducing into a plant cell of a first species an artificial chromosome capable of undergoing site-specific recombination with the DNA of the first plant species;
selecting for a site-specific recombination event between the artificial chromosome and the DNA of the first plant species, and
selecting an artificial chromosome comprising euchromatic DNA from the first plant species.

40. The method of claim 39, wherein the DNA of the plant cell of a first species is modified to comprise a site-specific recombination sequence.

41. The method of claim 39, wherein the artificial chromosome comprises a site-specific recombination sequence.

42. The method of claim 39, wherein the DNA of the plant cell of a first species is modified to comprise a site-specific recombination sequence and the artificial chromosome comprises a site-specific recombination sequence.

43. The method of claim 39, wherein the DNA of the plant cell of a first species is modified to comprise a site-specific recombination sequence and the artificial chromosome comprises a site-specific recombination sequence that is complementary to the site-specific recombination sequence of the plant cell of a first plant species.

44. The method of claim 39, wherein the site-specific recombination is catalyzed by a recombinase enzyme.

45. A method for producing an acrocentric plant chromosome, comprising:
introducing a first nucleic acid comprising a site-specific recombination site into a first chromosome of a plant cell;
introducing a second nucleic acid comprising a site-specific recombination site into a second chromosome of the plant cell;
introducing a recombinase activity into the plant cell, wherein the activity catalyzes recombination between the first and second chromosomes and whereby an acrocentric plant chromosome is produced.

46. The method of claim 45, wherein the first nucleic acid is introduced into the pericentric heterochromatin of the first chromosome.

47. The method of claim 45, wherein the second nucleic acid is introduced into the distal end of the arm of the second chromosome.

48. The method of claim 45, wherein the first nucleic acid is introduced into the pericentric heterochromatin of the first chromosome and the second nucleic acid is introduced into the distal end of the arm of the second chromosome.

49. A method for producing an acrocentric plant chromosome, comprising:
introducing a first nucleic acid comprising a site-specific recombination site into the pericentric heterochromatin of a chromosome in a plant cell;
introducing a second nucleic acid comprising a site-specific recombination site into the distal end of the chromosome, wherein the first and second recombination sites are located on the same arm of the chromosome;
introducing a recombinase activity into the cell, wherein the activity catalyzes recombination between the first and second recombination sites in the chromosome and whereby an acrocentric plant chromosome is produced.

50. A method for producing an acrocentric plant chromosome, comprising:
introducing nucleic acid comprising a recombination site adjacent to nucleic acid encoding a selectable marker into a first plant cell;
generating a first transgenic plant from the first plant cell;
introducing nucleic acid comprising a promoter functional in a plant cell, a recombination site and a recombinase coding region in operative linkage into a second plant cell;
generating a second transgenic plant from the second plant cell;
crossing the first and second plants;
obtaining plants resistant to an agent that selects for cells containing the nucleic acid encoding the selectable marker; and
selecting a resistant plant that contains cells comprising an acrocentric plant chromosome.

51. The method of claim 45, wherein the DNA of the short arm of the acrocentric chromosome contains less than 5% euchromatic DNA.

52. The method of claim 45, wherein the DNA of the short arm of the acrocentric chromosome contains less than 1 % euchromatic DNA.

53. The method of claim 45, wherein the short arm of the acrocentric chromosome does not contain euchromatic DNA.

54. The method of claim 45, wherein the nucleic acid introduced into a chromosome comprises nucleic acid encoding a selectable marker.

55. An acrocentric plant artificial chromosome, wherein the short arm of the acrocentric chromosome does not contain euchromatic DNA.

56. A method of producing a plant artificial chromosome, comprising:
introducing nucleic acid into a plant acrocentric chromosome in a cell, wherein the short arm of the acrocentric chromosome does not contain euchromatic DNA;
culturing the cell through at least one cell division; and
selecting a cell comprising an artificial chromosome that is predominantly heterochromatic.

57. The method of claim 56, wherein the acrocentric chromosome is produced by a method, comprising:
introducing a first nucleic acid comprising a site-specific recombination site into a first chromosome of a plant cell;
introducing a second nucleic acid comprising a site-specific recombination site into a second chromosome of the plant cell;
introducing a recombinase activity into the plant cell, wherein the activity catalyzes recombination between the first and second chromosomes and whereby an acrocentric plant chromosome is produced.

58. A method for producing an artificial chromosome, comprising:
introducing nucleic acid into a plant cell; and
selecting a plant cell comprising an artificial chromosome that comprises one or more repeat regions wherein:
one or more nucleic acid units is (are) repeated in a repeat region;
repeats of a nucleic acid unit have common nucleic acid sequences; and
the common nucleic acid sequences comprise sequences that represent euchromatic and heterochromatic nucleic acid.

59. The method of claim 4, wherein the nucleic acid comprises plant rDNA from a dicot plant species.

60. The method of claim 4, wherein the nucleic acid comprises plant rDNA from a monocot plant species.

61. The method of claim 9, wherein the intergenic spacer region is from DNA from a Nicotiana plant.

62. The method of claim 9, wherein the rDNA is plant rDNA.

63. The method of claim 62, wherein the plant is a dicot plant species.

64. The method of claim 62, wherein the plant is a monocot plant species.

65. The method of claim 1, wherein the cell is a dicot plant cell.

66. The method of claim 1, wherein the cell is a monocot plant cell.

67. An isolated plant artificial chromosome comprising one or more repeat regions, wherein:
one or more nucleic acid units is (are) repeated in a repeat region;
repeats of a nucleic acid unit have common nucleic acid sequences; and
the common nucleic acid sequences comprise sequences that represent euchromatic and heterochromatic nucleic acid.

68. The method of claim 31, wherein the artificial chromosome is produced by a method comprising:
introducing nucleic acid into a plant cell; and
selecting a plant cell comprising an artificial chromosome that comprises one or more repeat regions, wherein:
repeats of a nucleic acid unit have common nucleic acid sequences; and
the common nucleic acid sequences comprise sequences that represent euchromatic and heterochromatic nucleic acid.

69. The method of claim 44, wherein the recombinase is selected from the group consisting of a bacteriophage P1 Cre recombinase, a yeast R recombinase and a yeast FLP recombinase.

70. The method of claim 50, further comprising selecting first and second transgenic plants wherein:
one of the plants comprises a chromosome comprising a recombination site located on a short arm of the chromosome in a region adjacent to the pericentric heterochromatin; and
the other plant comprises a chromosome comprising a recombination site located in rDNA of the chromosome.

71. The method of claim 70, wherein the recombination sites on the two chromosomes are in the same orientation.

72. A method for producing an acrocentric plant chromosome, comprising:
introducing nucleic acid comprising two site-specific recombination sites into a cell comprising one or more plant chromosomes;
introducing a recombinase activity into the cell, wherein the activity catalyzes recombination between the two recombination sites, whereby a plant acrocentric chromosome is produced.

73. The method of claim 72, wherein the two site-specific recombination sites are contained on separate nucleic acid fragments.

74. The method of claim 73, wherein the separate nucleic acid fragments are introduced into the cell simultaneously or sequentially.

75. The method of claim 56, wherein the artificial chromosome is predominantly heterochromatic.

76. A method of producing a plant artificial chromosome, comprising:
introducing nucleic acid into a plant chromosome in a cell, wherein the chromosome contains adjacent regions of rDNA and heterochromatic DNA;
culturing the cell through at least one cell division; and
selecting a cell comprising an artificial chromosome.

77. The method of claim 76, wherein the artificial chromosome is predominantly heterochromatic.

78. The method of claim 76, wherein the plant chromosome into which the nucleic acid is introduced is an acrocentric chromosome.

79. The method of claim 78, wherein the short arm of the chromosome contains adjacent regions of rDNA and heterochromatic DNA.

80. The method of claim 76, wherein the heterochromatic DNA is pericentric heterochromatin.

81. A vector, comprising:
nucleic acid encoding a selectable marker that is not operably associated with any promoter, wherein the selectable marker permits growth of animal cells in the presence of an agent normally toxic to the animal cells; and wherein the agent is not toxic to plant cells;
a recognition site for recombination; and
a sequence of nucleotides that facilitates amplification of a region of a plant chromosome or targets the vector to an amplifiable region of a plant chromosome.

82. The vector of claim 81, wherein the amplifiable region comprises heterochromatic nucleic acid.

83. The vector of claim 81, wherein the amplifiable region comprises rDNA.

84. The vector of claim 81, wherein the sequence of nucleotides that facilitates amplification of a region of a plant chromosome or targets the vector to an amplifiable region of a plant chromosome comprises a sufficient portion of an intergenic spacer region of rDNA to facilitate amplification or effect the targeting.

85. The vector of claim 84, wherein the sufficient portion contains at least 14, 20, 30, 50, 100, 150, 300 or 500 contiguous nucleotides from an intergenic spacer region.

86. The vector of claim 81, wherein the selectable marker encodes a product that confers resistance to zeomycin.

87. The vector of claim 81, further comprising DNA encoding β-glucuronidase.

88. The vector of claim 81, wherein the recognition site comprises at att site.

89. The vector claim 81, that is pAglla or pAgllb.

90. A vector, comprising:
nucleic acid encoding a selectable marker that is not operably associated with any promoter, wherein the selectable marker permits growth of animal cells in the presence of an agent normally toxic to the animal cells; and wherein the agent is not toxic to plant cells;
a recognition site for recombination; and
nucleic acid encoding a protein operably linked to a plant promoter.

91. The vector of claim 90, wherein the recognition site comprises an att site.

92. The vector of claim 90, further comprising a sequence of nucleotides that facilitates amplification of a region of a plant chromosome or targets the vector to an amplifiable region of a plant chromosome.

93. The vector of claim 90, wherein the promoter is nopaline synthase (NOS) or CaMV35S.

94. The vector of claim 93 that is pAg1 or pAg 2.

95. The vector of claim 92, wherein the amplifiable region comprises heterochromatic nucleic acid.

96. The vector of claim 92, wherein the amplifiable region comprises rDNA.

97. The vector of claim 92, wherein the sequence of nucleotides that facilitates amplification of a region of a plant chromosome or targets the vector to an amplifiable region of a plant chromosome comprises a sufficient portion of an intergenic spacer region of rDNA to effect the amplification or the targeting.

98. The vector of claim 90, wherein the protein is a selectable marker that permits growth of plant cells in the presence of an agent normally toxic to the plant cells.

99. The vector of claim 98, wherein the selectable marker confers resistance to hygromycin or to phosphothricin.

100. The vector of claim 90, wherein the protein is a fluorescent protein.

101. The vector of claim 100, wherein the fluorescent protein is selected from the group consisting of green, blue and red fluorescent proteins.

102. A vector, comprising:
nucleic acid encoding a selectable marker that is not operably associated with any promoter, wherein the selectable marker permits growth of plant cells in the presence of an agent normally toxic to the plant cells; and wherein the agent is not toxic to animal cells;
a recognition site for recombination; and
nucleic acid encoding a protein operably linked to a plant promoter.

103. A vector, comprising:
a recognition site for recombination; and
a sequence of nucleotides that facilitates amplification of a region of a plant chromosome or targets the vector to an amplifiable region of a plant chromosome, wherein the plant is selected from the group consisting of Arabidopsis, Nicotiana, Solanum, Lycopersicon, Daucus, Hordeum, Zea mays, Brassica, Triticum, Helianthus, Glycine, soybean, Gossypium, cotton, Helianthus, sunflower and Oryza.

104. The vector of claim 103, wherein the recognition site comprises an att site.

105. A cell, comprising a vector of claim 81.

106. The cell of claim 105 that is a plant cell.

107. A method, comprising:
introducing a vector of claim 90 into a cell, wherein: the cell comprises an animal platform ACes that contains a recognition site that recombines with the recognition site in the vector in the presence of the recombinase, thereby incorporating the selectable marker that is not operably associated with any promoter and the nucleic acid encoding a protein operably linked to a plant promoter into the platform ACes to produce a resulting platform ACes.

108. The method of claim 107, wherein the recombination sites are att sites.

109. The method of claim 107, wherein the animal is a mammal.

110. The method of claim 107, wherein the platform ACes comprises a promoter that upon recombination is operably linked to the selectable marker that in the vector is not operably associated with a promoter.

111. The method of claim 107, further comprising, transferring the resulting platform ACes into a plant cell to produce a plant cell that comprises the platform Aces.

112. The method of claim 111, wherein the resulting platform ACes is isolated prior to transfer.

113. The method of claim 111, wherein the isolated ACes is introduced into a plant cell by a method selected from the group consisting of protoplast transfection, lipid-mediated delivery, liposomes, electroporation, sonoporation, microinjection, particle bombardment, silicon carbide whisker-mediated transformation, polyethylene glycol (PEG)-mediated DNA uptake, lipofection and lipid-mediated carrier systems.

114. The method of claim 111, wherein the resulting platform ACes is transferred by fusion of the cells.

115. The method of claim 111, wherein the cells are plant protoplasts.

116. The method of claim 107, wherein the cell is an animal cell.

117. The method of claim 116, wherein the animal cell is a mammalian cell.

118. The method of claim 111, further comprising culturing the plant cell that comprises the platform Aces under conditions whereby the protein encoded by the nucleic acid that is operably linked to a plant promoter is expressed.

119. A method, comprising:
introducing a vector of claim 81 into a plant cell;
culturing the plant cells; and
selecting a plant cell comprising an artificial chromosome that comprises one or more repeat regions.

120. The method of claim 119, wherein sufficient portion of the vector integrates into a chromosome in the plant cell to result in amplification of chromosomal DNA.

121. The method of claim 119, wherein:
one or more nucleic acid units is (are) repeated in a repeat region;
repeats of a nucleic acid unit have common nucleic acid sequences; and
the repeat region(s) contain substantially equivalent amounts of euchromatic and heterochromatic nucleic acid.

122. The method of claim 119, further comprising isolating the artificial chromosome.

123. A method, comprising:
introducing a vector into a cell, wherein:
i) the vector comprises:
a) nucleic acid encoding a selectable marker that is not operably associated with any promoter, wherein the selectable marker permits growth of animal cells in the presence of an agent normally toxic to the animal cells; and wherein the agent is not toxic to plant cells;
b) a recognition site for recombination; and
c) nucleic acid encoding a protein operably linked to an animal promoter;
ii) the cell comprises:
a platform plant artificial chromosome (PAC) that comprises a recombination site and an animal promoter that upon recombination is operably linked to the selectable marker that, in the vector, is not operably associated with a promoter;
iii) introduction is effected under conditions whereby the vector recombines with the PAC to produce a plant platform PAC that contains the selectable marker operably linked to the promoter; and
culturing the resulting cell under conditions, whereby the protein encoded by nucleic acid operably linked to an animal promoter is expressed.

124. The method of claim 119, wherein the artificial chromosome is an ACes.

125. The method of claim 123, wherein the plant platform PAC is an ACes.

126. The method of claim 1, wherein the nucleic acid introduced into the cell comprises nucleic acid encoding a selectable marker.

127. The vector of claim 81, further comprising one or more selectable markers that when expressed in the plant cell permit the selection of the cell.

128. A plant transformation vector, comprising:
a recognition site for recombination;
a sequence of nucleotides that facilitates amplification of a region of a plant chromosome or targets the vector to an amplifiable region of a plant chromosome; and
one or more selectable markers that when expressed in a plant cell permit the selection of the cell; wherein
the plant transformation vector is for Agrobacterium-mediated transformation of plants.

129. A method of producing a plant artificial chromosome, comprising:
introducing the vector of claim 81 into a cell comprising one or more plant chromosomes; and
selecting a cell comprising an artificial chromosome that comprises one or more repeat regions; wherein
one or more nucleic acid units is (are) repeated in a repeat region;
repeats of a nucleic acid unit have common nucleic acid sequences; and
the common nucleic acid sequences comprise sequences that represent euchromatic and heterochromatic nucleic acid.

130. A method of producing a plant artificial chromosome, comprising:
introducing the vector of claim 81 into a cell comprising one or more plant chromosomes; and
selecting a cell comprising an artificial chromosome that comprises one or more repeat regions; wherein
one or more nucleic acid units is (are) repeated in a repeat region;
repeats of a nucleic acid unit have common nucleic acid sequences; and
the repeat region(s) contain substantially equivalent amounts of euchromatic and heterochromatic nucleic acid.

131. The method of claim 123, wherein the cell into which the vector is introduced is an animal cell.

132. The method of claim 131, wherein the cell is a mammalian cell.

133. A plant cell comprising an artificial chromosome, wherein the artificial chromosome is produced by the method of claim 2.

134. A cell, comprising a vector of claim 90.

135. A cell, comprising a vector of claim 102.

136. A cell, comprising a vector of claim 103.

137. The cell of claim 134, that comprises at least one plant chromosome.

138. The cell of claim 135, that comprises at least one plant chromosome.

139. The cell of claim 136, that comprises at least one plant chromosome.


SEQUENCE LISTING (51 sequences)

SEQ ID NO: 1 - DNA, 11182 bp, Artificial Sequence
  pAg1 plasmid
catgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcgggggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagtcaaatctcggtgacgggcaggaccggacggggcggtaccggcaggctgaagtccagctgccagaaacccacgtcatgccagttcccgtgcttgaagccggccgcccgcagcatgccgcggggggcatatccgagcgcctcgtgcatgcgcacgctcgggtcgttgggcagcccgatgacagcgaccacgctcttgaagccctgtgcctccagggacttcagcaggtgggtgtagagcgtggagcccagtcccgtccgctggtggcggggggagacgtacacggtcgactcggccgtccagtcgtaggcgttgcgtgccttccaggggcccgcgtaggcgatgccggcgacctcgccgtccacctcggcgacgagccagggatagcgctcccgcagacggacgaggtcgtccgtccactcctgcggttcctgcggctcggtacggaagttgaccgtgcttgtctcgatgtagtggttgacgatggtgcagaccgccggcatgtccgcctcggtggcacggcggatgtcggccgggcgtcgttctgggctcatggtagactcgagagagatagatttgtagagagagactggtgatttcagcgtgtcctctccaaatgaaatgaacttccttatatagaggaaggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttggcaagctgctctagccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcgagccttgactagagggtcgacggtatacagacatgataagatacattgatgagtttggacaaaccacaactagaatgcagtgaaaaaaatgctttatttgtgaaatttgtgatgctattgctttatttgtaaccattataagctgcaataaacaagttggggtgggcgaagaactccagcatgagatccccgcgctggaggatcatccagccggcgtcccggaaaacgattccgaagcccaacctttcatagaaggcggcggtggaatcgaaatctcgtagcacgtgtcagtcctgctcctcggccacgaagtgcacgcagttgccggccgggtcgcgcagggcgaactcccgcccccacggctgctcgccgatctcggtcatggccggcccggaggcgtcccggaagttcgtggacacgacctccgaccactcggcgtacagctcgtccaggccgcgcacccacacccaggccagggtgttgtccggcaccacctggtcctggaccgcgctgatgaacagggtcacgtcgtcccggaccacaccggcgaagtcgtcctccacgaagtcccgggagaacccgagccggtcggtccagaactcgaccgctccggcgacgtcgcgcgcggtgagcaccggaacggcactggtcaacttggccatggatccagatttcgctcaagttagtataaaaaagcaggcttcaatcctgcaggaattcgatcgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgataacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggacacgctgaaatcaccagtctctctctacaaatctatctctctcgagctttcgcagatccgggggggcaatgagatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggagtttagcgagagcctgacctattgcatctcccgccgtgcacagggtgtcacgttgcaagacctgcctgaaaccgaactgcccgctgttctacaaccggtcgcggaggctatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccacgactccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaagaaatagagtagatgccgaccggatctgtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgttaattcagatcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtg

SEQ ID NO: 2 - DNA, 8428 bp, Artificial Sequence
  pCambia3300 plasmid
catgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcgggggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagtcaaatctcggtgacgggcaggaccggacggggcggtaccggcaggctgaagtccagctgccagaaacccacgtcatgccagttcccgtgcttgaagccggccgcccgcagcatgccgcggggggcatatccgagcgcctcgtgcatgcgcacgctcgggtcgttgggcagcccgatgacagcgaccacgctcttgaagccctgtgcctccagggacttcagcaggtgggtgtagagcgtggagcccagtcccgtccgctggtggcggggggagacgtacacggtcgactcggccgtccagtcgtaggcgttgcgtgccttccaggggcccgcgtaggcgatgccggcgacctcgccgtccacctcggcgacgagccagggatagcgctcccgcagacggacgaggtcgtccgtccactcctgcggttcctgcggctcggtacggaagttgaccgtgcttgtctcgatgtagtggttgacgatggtgcagaccgccggcatgtccgcctcggtggcacggcggatgtcggccgggcgtcgttctgggctcatggtagactcgagagagatagatttgtagagagagactggtgatttcagcgtgtcctctccaaatgaaatgaacttccttatatagaggaaggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttggcaagctgctctagccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcgagctcggtacccggggatcctctagagtcgacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtg

SEQ ID NO: 3 - DNA, 10549 bp, Artificial Sequence
  pCambia1302 plasmid
  ref <308>: DATABASE ACCESSION NUMBER: Genbank #AF234298
  ref <309>: DATABASE ENTRY DATE: 2000-04-24
catggtagatctgactagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccgtggccaacacttgtcactactttctcttatggtgttcaatgcttttcaagatacccagatcatatgaagcggcacgacttcttcaagagcgccatgcctgagggatacgtgcaggagaggaccatcttcttcaaggacgacgggaactacaagacacgtgctgaagtcaagtttgagggagacaccctcgtcaacaggatcgagcttaagggaatcgatttcaaggaggacggaaacatcctcggccacaagttggaatacaactacaactcccacaacgtatacatcatggccgacaagcaaaagaacggcatcaaagccaacttcaagacccgccacaacatcgaagacggcggcgtgcaactcgctgatcattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaagctagccaccaccaccaccaccacgtgtgaattggtgaccagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcgggggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagcttgtcgatcgacagatccggtcggcatctactctatttctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagtcgtggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatagcctccgcgaccggttgtagaacagcgggcagttcggtttcaggcaggtcttgcaacgtgacaccctgtgcacggcgggagatgcaataggtcaggctctcgctaaactccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatatctcattgccccccgggatctgcgaaagctcgagagagatagatttgtagagagagactggtgatttcagcgtgtcctctccaaatgaaatgaacttccttatatagaggaaggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttggcaagctgctctagccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcgagctcggtacccggggatcctctagagtcgacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttagcttcatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactcttgac

SEQ ID NO: 4 - DNA, 33 bp, Artificial Sequence
  CaMV35SpolyA Primer
ctgaattaacgccgaattaattcgggggatctg

SEQ ID NO: 5 - DNA, 29 bp, Artificial Sequence
  CaMV35Spr Primer
ctagagcagcttgccaacatggtggagca

SEQ ID NO: 6 - DNA, 12592 bp, Artificial Sequence
  pAg2 Plasmid
gtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcgggggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagtcaaatctcggtgacgggcaggaccggacggggcggtaccggcaggctgaagtccagctgccagaaacccacgtcatgccagttcccgtgcttgaagccggccgcccgcagcatgccgcggggggcatatccgagcgcctcgtgcatgcgcacgctcgggtcgttgggcagcccgatgacagcgaccacgctcttgaagccctgtgcctccagggacttcagcaggtgggtgtagagcgtggagcccagtcccgtccgctggtggcggggggagacgtacacggtcgactcggccgtccagtcgtaggcgttgcgtgccttccaggggcccgcgtaggcgatgccggcgacctcgccgtccacctcggcgacgagccagggatagcgctcccgcagacggacgaggtcgtccgtccactcctgcggttcctgcggctcggtacggaagttgaccgtgcttgtctcgatgtagtggttgacgatggtgcagaccgccggcatgtccgcctcggtggcacggcggatgtcggccgggcgtcgttctgggctcatggtagactcgagagagatagatttgtagagagagactggtgatttcagcgtgtcctctccaaatgaaatgaacttccttatatagaggaaggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttggcaagctgctctagccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcgagccttgactagagggtcgacggtatacagacatgataagatacattgatgagtttggacaaaccacaactagaatgcagtgaaaaaaatgctttatttgtgaaatttgtgatgctattgctttatttgtaaccattataagctgcaataaacaagttggggtgggcgaagaactccagcatgagatccccgcgctggaggatcatccagccggcgtcccggaaaacgattccgaagcccaacctttcatagaaggcggcggtggaatcgaaatctcgtagcacgtgtcagtcctgctcctcggccacgaagtgcacgcagttgccggccgggtcgcgcagggcgaactcccgcccccacggctgctcgccgatctcggtcatggccggcccggaggcgtcccggaagttcgtggacacgacctccgaccactcggcgtacagctcgtccaggccgcgcacccacacccaggccagggtgttgtccggcaccacctggtcctggaccgcgctgatgaacagggtcacgtcgtcccggaccacaccggcgaagtcgtcctccacgaagtcccgggagaacccgagccggtcggtccagaactcgaccgctccggcgacgtcgcgcgcggtgagcaccggaacggcactggtcaacttggccatggatccagatttcgctcaagttagtataaaaaagcaggcttcaatcctgcaggaattcgatcgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgataacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggacacgctgaaatcaccagtctctctctacaaatctatctctctcgagctttcgcagatccgggggggcaatgagatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggagtttagcgagagcctgacctattgcatctcccgccgtgcacagggtgtcacgttgcaagacctgcctgaaaccgaactgcccgctgttctacaaccggtcgcggaggctatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccacgactccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaagaaatagagtagatgccgaccggatctgtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgttaattcagatcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttggggatcctctagactgaaggcgggaaacgacaatctgatcatgagcggagaattaagggagtcacgttatgacccccgccgatgacgcgggacaagccgttttacgtttggaactgacagaaccgcaacgttgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaatccaaataatctgcaccggatctcgagaatcgaattcccgcggccgccatggtagatctgactagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccgtggccaacacttgtcactactttctcttatggtgttcaatgcttttcaagatacccagatcatatgaagcggcacgacttcttcaagagcgccatgcctgagggatacgtgcaggagaggaccatcttcttcaaggacgacgggaactacaagacacgtgctgaagtcaagtttgagggagacaccctcgtcaacaggatcgagcttaagggaatcgatttcaaggaggacggaaacatcctcggccacaagttggaatacaactacaactcccacaacgtatacatcatggccgacaagcaaaagaacggcatcaaagccaacttcaagacccgccacaacatcgaagacggcggcgtgcaactcgctgatcattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaagctagccaccaccaccaccaccacgtgtgaattggtgaccagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagc

SEQ ID NO: 7 - DNA, 3357 bp, Artificial Sequence
  pGEMEasyNOS Plasmid
tatcactagtgaattcgcggccgcctgcaggtcgaccatatgggagagctcccaacgcgttggatgcatagcttgagtattctatagtgtcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgatgcggtgtgaaataccgcacagatgcgtaaggagaaaataccgcatcaggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggcccgacgtcgcatgctcccggccgccatggcggccgcgggaattcgattctcgagatccggtgcagattatttggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctccgctcatgatcagattgtcgtttcccgccttcagtctaga

SEQ ID NO: 8 - DNA, 10122 bp, Artificial Sequence
  p1302NOS Plasmid
catggtagatctgactagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccgtggccaacacttgtcactactttctcttatggtgttcaatgcttttcaagatacccagatcatatgaagcggcacgacttcttcaagagcgccatgcctgagggatacgtgcaggagaggaccatcttcttcaaggacgacgggaactacaagacacgtgctgaagtcaagtttgagggagacaccctcgtcaacaggatcgagcttaagggaatcgatttcaaggaggacggaaacatcctcggccacaagttggaatacaactacaactcccacaacgtatacatcatggccgacaagcaaaagaacggcatcaaagccaacttcaagacccgccacaacatcgaagacggcggcgtgcaactcgctgatcattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaagctagccaccaccaccaccaccacgtgtgaattggtgaccagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcgggggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagcttgtcgatcgacagatccggtcggcatctactctatttctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagtcgtggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatagcctccgcgaccggttgtagaacagcgggcagttcggtttcaggcaggtcttgcaacgtgacaccctgtgcacggcgggagatgcaataggtcaggctctcgctaaactccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatatctcattgcccccccggatctgcgaaagctcgagagagatagatttgtagagagagactggtgatttcagcgtgtcctctccaaatgaaatgaacttccttatatagaggaaggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttggcaagctgctctagccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcgagctcggtacccggggatcctctagactgaaggcgggaaacgacaatctgatcatgagcggagaattaagggagtcacgttatgacccccgccgatgacgcgggacaagccgttttacgtttggaactgacagaaccgcaacgttgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaatccaaataatctgcaccggatctcgagaatcgaattcccgcggccgc

SEQ ID NO: 9 - DNA, 621 bp, Artificial Sequence
  N. tabacum rDNA intergnic spacer (IGS) sequence
  ref <308>: DATABASE ACCESSION NUMBER: Genbank #Y08422
  ref <309>: DATABASE ENTRY DATE: 1997-10-31
gtgctagccaatgtttaacaagatgtcaagcacaatgaatgttggtggttggtggtcgtggctggcggtggtggaaaattgcggtggttcgagcggtagtgatcggcgatggttggtgtttgcagcggtgtttgatatcggaatcacttatggtggttgtcacaatggaggtgcgtcatggttattggtggttggtcatctatatatttttataataatattaagtattttacctattttttacatattttttattaaatttatgcattgtttgtatttttaaatagtttttatcgtacttgttttataaaatattttattattttatgtgttatattattacttgatgtattggaaattttctccattgttttttctatatttataataattttcttatttttttttgttttattatgtattttttcgttttataataaatatttattaaaaaaaatattatttttgtaaaatatatcatttacaatgtttaaaagtcatttgtgaatatattagctaagttgtacttctttttgtgcatttggtgttgtacatgtctattatgattctctggccaaaacatgtctactcctgtcacttgggtttttttttttaagacat

SEQ ID NO: 10 - DNA, 25 bp, Artificial Sequence
  PCR Primer NTIGS-F1
gtgctagccaatgtttaacaagatg

SEQ ID NO: 11 - DNA, 28 bp, Artificial Sequence
  PCR Primer NTIGS-RI
atgtcttaaaaaaaaaaacccaagtgac

SEQ ID NO: 12 - DNA, 233 bp, Mus musculus
  ref <308>: DATABASE ACCESSION NUMBER: Genbank #V00846
  ref <309>: DATABASE ENTRY DATE: 1989-07-06
gacctggaatatggcgagaaaactgaaaatcacggaaaatgagaaatacacactttaggacgtgaaatatggcgaggaaaactgaaaaaggtggaaaatttagaaatgtccactgtaggacgtggaatatggcaagaaaactgaaaatcatggaaaatgagaaacatccacttgacgacttgaaaaatgacgaaatcactaaaaaacgtgaaaaatgagaaatgcacactgaa

SEQ ID NO: 13 - DNA, 31 bp, Artificial Sequence
  Primer MSAT-F1
aataccgcggaagcttgacctggaatatcgc

SEQ ID NO: 14 - DNA, 27 bp, Artificial Sequence
  Primer MSAT-RI
ataaccgcggagtccttcagtgtgcat

SEQ ID NO: 15 - DNA, 277 bp, Artificial Sequence
  Nopaline Synthase Promoter Fragment
  ref <308>: DATABASE ACCESSION NUMBER: Genebank #U09365
  ref <309>: DATABASE ENTRY DATE: 1997-10-17
gagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattc

SEQ ID NO: 16 - DNA, 1812 bp, Escherichia coli
  CDS (1)...(1812): Beta-glucuronidase
  ref <308>: DATABASE ACCESSION NUMBER: Genbank #S69414
  ref <309>: DATABASE ENTRY DATE: 1994-09-23
atgttacgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatga

SEQ ID NO: 17 - protein, 603 aa, Escherichia coli
  ref <308>: DATABASE ACCESSION NUMBER: Genbank #S69414
  ref <309>: DATABASE ENTRY DATE: 1994-09-23
MLRPVETPTREIKKLDGLWAFSLDRENCGIDQRWWESALQESRAIAVPGSFNDQFADADIRNYAGNVWYQREVFIPKGWAGQRIVLRFDAVTHYGKVWVNNQEVMEHQGGYTPFEADVTPYVIAGKSVRITVCVNNELNWQTIPPGMVITDENGKKKQSYFHDFFNYAGIHRSVMLYTTPNTWVDDITVVTHVAQDCNHASVDWQVVANGDVSVELRDADQQVVATGQGTSGTLQVVNPHLWQPGEGYLYELCVTAKSQTECDIYPLRVGIRSVAVKGEQFLINHKPFYFTGFGRHEDADLRGKGFDNVLMVHDHALMDWIGANSYRTSHYPYAEEMLDWADEHGIVVIDETAAVGFNLSLGIGFEAGNKPKELYSEEAVNGETQQAHLQAIKELIARDKNHPSVVMWSIANEPDTRPQGAREYFAPLAEATRKLDPTRPITCVNVMFCDAHTDTISDLFDVLCLNRYYGWYVQSGDLETAEKVLEKELLAWQEKLHQPIIITEYGVDTLAGLHSMYTDMWSEEYQCAWLDMYHRVFDRVSAVVGEQVWNFADFATSQGILRVGGNKKGIFTRDRKPKSAAFLLQKRWTGMNFGEKPQQGGKQ

SEQ ID NO: 18 - DNA, 277 bp, Artificial Sequence; identical to SEQ ID NO: 15
  Nopaline Synthase Terminator Sequence
  ref <308>: DATABASE ACCESSION NUMBER: Genbank #U09365
  ref <309>: DATABASE ENTRY DATE: 1995-10-17

SEQ ID NO: 19 - DNA, 3438 bp, Artificial Sequence
  pLIT38attBZeo Plasmid
tcgaccctctagtcaaggccttaagtgagtcgtattacggactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcttcgcttggtaataaagcccgcttcggcgggcttttttttgttaactacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttctccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaccccggttgataatcagaaaagccccaaaaacaggaagattgtataagcaaatatttaaattgtaaacgttaatattttgttaaaattcgcgttaaatttttgttaaatcagctcattttttaaccaataggccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtaatgtgagttagctcactcattaggcaccccaggctttacactttatgcttccggctcgtatgttgtgtggaattgtgagcggataacaatttcacacaggaaacagctatgaccatgattacgccaagctacgtaatacgactcactagtggggcccgtgcaattgaagccggctggcgccaagcttctctgcaggattgaagcctgcttttttatactaacttgagcgaaatctggatccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccg

SEQ ID NO: 20 - DNA, 3451 bp, Artificial Sequence
  HindIII Fragment containing the beta- lucuronidase coding sequence, the rDNA intergenic spacer, and the Mast1 sequence
aagcttgacctggaatatcgcgagtaaactgaaaatcacggaaaatgagaaatacacactttaggacgtgaaatatggcgaggaaaactgaaaaaggtggaaaatttagaaatgtccactgtaggacgtggaatatggcaagaaaactgaaaatcatggaaaatgagaaacatccacttgacgacttgaaaaatgacgaaatcactaaaaaacgtgaaaaatgagaaatgcacactgaaggactccgcgggaattcgattgtgctagccaatgtttaacaagatgtcaagcacaatgaatgttggtggttggtggtcgtggctggcggtggtggaaaattgcggtggttcgagcggtagtgatcggcgatggttggtgtttgcagcggtgtttgatatcggaatcacttatggtggttgtcacaatggaggtgcgtcatggttattggtggttggtcatctatatatttttataataatattaagtattttacctattttttacatattttttattaaatttatgcattgtttgtatttttaaatagtttttatcgtacttgttttataaaatattttattattttatgtgttatattattacttgatgtattggaaattttctccattgttttttctatatttataataattttcttatttttttttgttttattatgtattttttcgttttataataaatatttattaaaaaaaatattatttttgtaaaatatatcatttacaatgtttaaaagtcatttgtgaatatattagctaagttgtacttctttttgtgcatttggtgttgtacatgtctattatgattctctggccaaaacatgtctactcctgtcacttgggtttttttttttaagacataatcactagtgattatatctagactgaaggcgggaaacgacaatctgatcatgagcggagaattaagggagtcacgttatgacccccgccgatgacgcgggacaagccgttttacgtttggaactgacagaaccgcaacgttgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaatccaaataatctgcaccggatctcgagatcgaattcccgcggccgcgaattcactagtggatccccgggtacggtcagtcccttatgttacgtcctgtagaaaccccaacccgtgaaatcaaaaaactcgacggcctgtgggcattcagtctggatcgcgaaaactgtggaattgagcagcgttggtgggaaagcgcgttacaagaaagccgggcaattgctgtgccaggcagttttaacgatcagttcgccgatgcagatattcgtaattatgtgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtatcacagtttgtgtgaacaacgaactgaactggcagactatcccgccgggaatggtgattaccgacgaaaacggcaagaaaaagcagtcttacttccatgatttctttaactacgccgggatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcaccagcgggactttgcaagtggtgaatccgcacctctggcaaccgggtgaaggttatctctatgaactgtacgtcacagccaaaagccagacagagtgtgatatctacccgctgcgcgtcggcatccggtcagtggcagtgaagggcgaacagttcctgatcaaccacaaaccgttctactttactggctttggccgtcatgaagatgcggatttgcgcggcaaaggattcgataacgtgctgatggtgcacgatcacgcattaatggactggattggggccaactcctaccgtacctcgcattacccttacgctgaagagatgctcgactgggcagatgaacatggcatcgtggtgattgatgaaactgcagctgtcggctttaacctctctttaggcattggtttcgaagcgggcaacaagccgaaagaactgtacagcgaagaggcagtcaacggggaaactcagcaggcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgatccgacgcgtccgatcacctgcgtcaatgtaatgttctgcgacgctcacaccgataccatcagcgatctctttgatgtgctgtgcctgaaccgttattacggttggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaggggatcttcacccgcgaccgcaaaccgaagtcggcggcttttctgctgcaaaaacgctggactggcatgaacttcggtgaaaaaccgcagcagggaggcaaacaatgaatcaacaactctcctggcgcaccatcgtcggctacagcctcgggaattgcgtaccgagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattcgatatcaagctt

SEQ ID NO: 21 - DNA, 14627 bp, Artificial Sequence
  pAg11a Plasmid
catgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcgggggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagtcaaatctcggtgacgggcaggaccggacggggcggtaccggcaggctgaagtccagctgccagaaacccacgtcatgccagttcccgtgcttgaagccggccgcccgcagcatgccgcggggggcatatccgagcgcctcgtgcatgcgcacgctcgggtcgttgggcagcccgatgacagcgaccacgctcttgaagccctgtgcctccagggacttcagcaggtgggtgtagagcgtggagcccagtcccgtccgctggtggcggggggagacgtacacggtcgactcggccgtccagtcgtaggcgttgcgtgccttccaggggcccgcgtaggcgatgccggcgacctcgccgtccacctcggcgacgagccagggatagcgctcccgcagacggacgaggtcgtccgtccactcctgcggttcctgcggctcggtacggaagttgaccgtgcttgtctcgatgtagtggttgacgatggtgcagaccgccggcatgtccgcctcggtggcacggcggatgtcggccgggcgtcgttctgggctcatggtagactcgagagagatagatttgtagagagagactggtgatttcagcgtgtcctctccaaatgaaatgaacttccttatatagaggaaggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttggcaagctgctctagccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcgagccttgactagagggtcgacggtatacagacatgataagatacattgatgagtttggacaaaccacaactagaatgcagtgaaaaaaatgctttatttgtgaaatttgtgatgctattgctttatttgtaaccattataagctgcaataaacaagttggggtgggcgaagaactccagcatgagatccccgcgctggaggatcatccagccggcgtcccggaaaacgattccgaagcccaacctttcatagaaggcggcggtggaatcgaaatctcgtagcacgtgtcagtcctgctcctcggccacgaagtgcacgcagttgccggccgggtcgcgcagggcgaactcccgcccccacggctgctcgccgatctcggtcatggccggcccggaggcgtcccggaagttcgtggacacgacctccgaccactcggcgtacagctcgtccaggccgcgcacccacacccaggccagggtgttgtccggcaccacctggtcctggaccgcgctgatgaacagggtcacgtcgtcccggaccacaccggcgaagtcgtcctccacgaagtcccgggagaacccgagccggtcggtccagaactcgaccgctccggcgacgtcgcgcgcggtgagcaccggaacggcactggtcaacttggccatggatccagatttcgctcaagttagtataaaaaagcaggcttcaatcctgcaggaattcgatcgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgataacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggacacgctgaaatcaccagtctctctctacaaatctatctctctcgagctttcgcagatccgggggggcaatgagatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggagtttagcgagagcctgacctattgcatctcccgccgtgcacagggtgtcacgttgcaagacctgcctgaaaccgaactgcccgctgttctacaaccggtcgcggaggctatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccacgactccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaagaaatagagtagatgccgaccggatctgtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgttaattcagatcaagcttgacctggaatatcgcgagtaaactgaaaatcacggaaaatgagaaatacacactttaggacgtgaaatatggcgaggaaaactgaaaaaggtggaaaatttagaaatgtccactgtaggacgtggaatatggcaagaaaactgaaaatcatggaaaatgagaaacatccacttgacgacttgaaaaatgacgaaatcactaaaaaacgtgaaaaatgagaaatgcacactgaaggactccgcgggaattcgattgtgctagccaatgtttaacaagatgtcaagcacaatgaatgttggtggttggtggtcgtggctggcggtggtggaaaattgcggtggttcgagcggtagtgatcggcgatggttggtgtttgcagcggtgtttgatatcggaatcacttatggtggttgtcacaatggaggtgcgtcatggttattggtggttggtcatctatatatttttataataatattaagtattttacctattttttacatattttttattaaatttatgcattgtttgtatttttaaatagtttttatcgtacttgttttataaaatattttattattttatgtgttatattattacttgatgtattggaaattttctccattgttttttctatatttataataattttcttatttttttttgttttattatgtattttttcgttttataataaatatttattaaaaaaaatattatttttgtaaaatatatcatttacaatgtttaaaagtcatttgtgaatatattagctaagttgtacttctttttgtgcatttggtgttgtacatgtctattatgattctctggccaaaacatgtctactcctgtcacttgggtttttttttttaagacataatcactagtgattatatctagactgaaggcgggaaacgacaatctgatcatgagcggagaattaagggagtcacgttatgacccccgccgatgacgcgggacaagccgttttacgtttggaactgacagaaccgcaacgttgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaatccaaataatctgcaccggatctcgagatcgaattcccgcggccgcgaattcactagtggatccccgggtacggtcagtcccttatgttacgtcctgtagaaaccccaacccgtgaaatcaaaaaactcgacggcctgtgggcattcagtctggatcgcgaaaactgtggaattgagcagcgttggtgggaaagcgcgttacaagaaagccgggcaattgctgtgccaggcagttttaacgatcagttcgccgatgcagatattcgtaattatgtgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtatcacagtttgtgtgaacaacgaactgaactggcagactatcccgccgggaatggtgattaccgacgaaaacggcaagaaaaagcagtcttacttccatgatttctttaactacgccgggatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcaccagcgggactttgcaagtggtgaatccgcacctctggcaaccgggtgaaggttatctctatgaactgtacgtcacagccaaaagccagacagagtgtgatatctacccgctgcgcgtcggcatccggtcagtggcagtgaagggcgaacagttcctgatcaaccacaaaccgttctactttactggctttggccgtcatgaagatgcggatttgcgcggcaaaggattcgataacgtgctgatggtgcacgatcacgcattaatggactggattggggccaactcctaccgtacctcgcattacccttacgctgaagagatgctcgactgggcagatgaacatggcatcgtggtgattgatgaaactgcagctgtcggctttaacctctctttaggcattggtttcgaagcgggcaacaagccgaaagaactgtacagcgaagaggcagtcaacggggaaactcagcaggcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgatccgacgcgtccgatcacctgcgtcaatgtaatgttctgcgacgctcacaccgataccatcagcgatctctttgatgtgctgtgcctgaaccgttattacggttggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaggggatcttcacccgcgaccgcaaaccgaagtcggcggcttttctgctgcaaaaacgctggactggcatgaacttcggtgaaaaaccgcagcagggaggcaaacaatgaatcaacaactctcctggcgcaccatcgtcggctacagcctcgggaattgcgtaccgagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattcgatatcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtg

SEQ ID NO: 22 - DNA, 4257 bp, Artificial Sequence
  pPUR Plasmid
ctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttgcatgcctgcaggtcggccgccacgaccggtgccgccaccatcccctgacccacgcccctgacccctcacaaggagacgaccttccatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgacgcccgccccacgacccgcagcgcccgaccgaaaggagcgcacgaccccatggctccgaccgaagccgacccgggcggccccgccgaccccgcacccgcccccgaggcccaccgactctagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatccccaggaagctcctctgtgtcctcataaaccctaacctcctctacttgagaggacattccaatcataggctgcccatccaccctctgtgtcctcctgttaattaggtcacttaacaaaaaggaaattgggtaggggtttttcacagaccgctttctaagggtaattttaaaatatctgggaagtcccttccactgctgtgttccagaagtgttggtaaacagcccacaaatgtcaacagcagaaacatacaagctgtcagctttgcacaagggcccaacaccctgctcatcaagaagcactgtggttgctgtgttagtaatgtgcaaaacaggaggcacattttccccacctgtgtaggttccaaaatatctagtgttttcatttttacttggatcaggaacccagcactccactggataagcattatccttatccaaaacagccttgtggtcagtgttcatctgctgactgtcaactgtagcattttttggggttacagtttgagcaggatatttggtcctgtagtttgctaacacaccctgcagctccaaaggttccccaccaacagcaaaaaaatgaaaatttgacccttgaatgggttttccagcaccattttcatgagttttttgtgtccctgaatgcaagtttaacatagcagttaccccaataacctcagttttaacagtaacagcttcccacatcaaaatatttccacaggttaagtcctcatttaaattaggcaaaggaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccag

SEQ ID NO: 23 - DNA, 2713 bp, Artificial Sequence
  pNEB193 Plasmid
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccgggggcgcgccggatccttaattaagtctagagtcgactgtttaaacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 24 - DNA, 25 bp, Artificial Sequence
  attPUP Primer
ccttgcgctaatgctctgttacagg

SEQ ID NO: 25 - DNA, 26 bp, Artificial Sequence
  attPDWN Primer
cagaggcagggagtgggacaaaattg

SEQ ID NO: 26 - DNA, 4346 bp, Artificial Sequence
  pSV40193attPsensePUR Plasmid
ccggtgccgccaccatcccctgacccacgcccctgacccctcacaaggagacgaccttccatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgacgcccgccccacgacccgcagcgcccgaccgaaaggagcgcacgaccccatggctccgaccgaagccgacccgggcggccccgccgaccccgcacccgcccccgaggcccaccgactctagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatccgcgccggatccttaattaagtctagagtcgactgtttaaacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggctcggtacccccttgcgctaatgctctgttacaggtcactaataccatctaagtagttgattcatagtgactgcatatgttgtgttttacagtattatgtagtctgttttttatgcaaaatctaatttaatatattgatatttatatcattttacgtttctcgttcagcttttttatactaagttggcattataaaaaagcattgcttatcaatttgttgcaacgaacaggtcactatcagtcaaaataaaatcattatttgatttcaattttgtcccactccctgcctctggggggcgcg

SEQ ID NO: 27 - DNA, 5855 bp, Artificial Sequence
  pCXLamIntR Plasmid
gtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacagctcctgggcaacgtgctggttgttgtgctgtctcatcattttggcaaagaattcatgggaagaaggcgaagtcatgagcgccgggatttaccccctaacctttatataagaaacaatggatattactgctacagggacccaaggacgggtaaagagtttggattaggcagagacaggcgaatcgcaatcactgaagctatacaggccaacattgagttattttcaggacacaaacacaagcctctgacagcgagaatcaacagtgataattccgttacgttacattcatggcttgatcgctacgaaaaaatcctggccagcagaggaatcaagcagaagacactcataaattacatgagcaaaattaaagcaataaggaggggtctgcctgatgctccacttgaagacatcaccacaaaagaaattgcggcaatgctcaatggatacatagacgagggcaaggcggcgtcagccaagttaatcagatcaacactgagcgatgcattccgagaggcaatagctgaaggccatataacaacaaaccatgtcgctgccactcgcgcagcaaaatctagagtaaggagatcaagacttacggctgacgaatacctgaaaatttatcaagcagcagaatcatcaccatgttggctcagacttgcaatggaactggctgttgttaccgggcaacgagttggtgatttatgcgaaatgaagtggtctgatatcgtagatggatatctttatgtcgagcaaagcaaaacaggcgtaaaaattgccatcccaacagcattgcatattgatgctctcggaatatcaatgaaggaaacacttgataaatgcaaagagattcttggcggagaaaccataattgcatctactcgtcgcgaaccgctttcatccggcacagtatcaaggtattttatgcgcgcacgaaaagcatcaggtctttccttcgaaggggatccgcctacctttcacgagttgcgcagtttgtctgcaagactctatgagaagcagataagcgataagtttgctcaacatcttctcgggcataagtcggacaccatggcatcacagtatcgtgatgacagaggcagggagtgggacaaaattgaaatcaaataagaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgccatatgctggctgccatgaacaaaggtggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatgaagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctggatccgctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctg

SEQ ID NO: 28 - DNA, 37 bp, Artificial Sequence
  5PacSV40 Primer
ctgttaattaactgtggaatgtgtgtcagttagggtg

SEQ ID NO: 29 - DNA, 20 bp, Artificial Sequence
  Antisense Zeo Primer
tgaacagggtcacgtcgtcc

SEQ ID NO: 30 - DNA, 1032 bp, Escherichia Coli
  CDS (1)...(1032): nucleotide sequence encoding Cre recombinase
atgtccaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattag

SEQ ID NO: 31 - protein, 343 aa, Escherichia Coli
MSNLLTVHQNLPALPVDATSDEVRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVMRRIRKENVDAGERAKQALAFERTDFDQVRSLMENSDRCQDIRNLAFLGIAYNTLLRIAEIARIRVKDISRTDGGRMLIHIGRTKTLVSTAGVEKALSLGVTKLVERWISVSGVADDPNNYLFCRVRKNGVAAPSATSQLSTRALEGIFEATHRLIYGAKDDSGQRYLAWSGHSARVGAARDMARAGVSIPEIMQAGGWTNVNIVMNYIRNLDSETGAMVRLLEDGD

SEQ ID NO: 32 - DNA, 33 bp, Artificial Sequence
  attB1 recognition sequence
tgaagcctgcttttttatactaacttgagcgaa

SEQ ID NO: 33 - DNA, 25 bp, Artificial Sequence
  m-att recognition sequence
  misc_difference 18: n is a or g or c or t/u
rkycwgctttyktrtacnaastsgb

SEQ ID NO: 34 - DNA, 25 bp, Artificial Sequence
  m-attB recognition sequence
  misc_difference 18: n is a or c or g or t/u
agccwgctttyktrtacnaactsgb

SEQ ID NO: 35 - DNA, 25 bp, Artificial Sequence
  m-attR recognition sequence
  misc_difference 18: n is a or g or c or t/u
gttcagctttcktrtacnaactsgb

SEQ ID NO: 36 - DNA, 25 bp, Artificial Sequence
  m-attL recognition sequence
  misc_difference 18: n is a or g or c or t/u
agccwgctttcktrtacnaagtsgb

SEQ ID NO: 37 - DNA, 25 bp, Artificial Sequence
  m-attP1 recognition sequence
  misc_difference 18: n is a or g or c or t/u
gttcagctttyktrtacnaagtsgb

SEQ ID NO: 38 - DNA, 25 bp, Artificial Sequence
  attB2 recognition sequence
agcctgctttcttgtacaaacttgt

SEQ ID NO: 39 - DNA, 25 bp, Artificial Sequence
  attB3 recognition sequence
acccagctttcttgtacaaacttgt

SEQ ID NO: 40 - DNA, 25 bp, Artificial Sequence
  attR1 recognition sequence
gttcagcttttttgtacaaacttgt

SEQ ID NO: 41 - DNA, 25 bp, Artificial Sequence
  attR2 recognition sequence
gttcagctttcttgtacaaacttgt

SEQ ID NO: 42 - DNA, 25 bp, Artificial Sequence
  attR3 recognition sequence
gttcagctttcttgtacaaagttgg

SEQ ID NO: 43 - DNA, 25 bp, Artificial Sequence
  attL1 recognition sequence
agcctgcttttttgtacaaagttgg

SEQ ID NO: 44 - DNA, 25 bp, Artificial Sequence
  attL2 recognition sequence
agcctgctttcttgtacaaagttgg

SEQ ID NO: 45 - DNA, 25 bp, Artificial Sequence
  attL3 recognition sequence
acccagctttcttgtacaaagttgg

SEQ ID NO: 46 - DNA, 25 bp, Artificial Sequence
  attP1 recognition sequence
gttcagcttttttgtacaaagttgg

SEQ ID NO: 47 - DNA, 25 bp, Artificial Sequence
  attP2,P3 recognition sequence
gttcagctttcttgtacaaagttgg

SEQ ID NO: 48 - DNA, 282 bp, Artificial Sequence
  attP recognition sequence
ccttgcgctaatgctctgttacaggtcactaataccatctaagtagttgattcatagtgactgcatatgttgtgttttacagtattatgtagtctgttttttatgcaaaatctaatttaatatattgatatttatatcattttacgtttctcgttcagcttttttatactaagttggcattataaaaaagcattgcttatcaatttgttgcaacgaacaggtcactatcagtcaaaataaaatcattatttgatttcaattttgtcccactccctgcctctg

SEQ ID NO: 49 - DNA, 1071 bp, Artificial Sequence
  nucleotide sequence encoding Integrase E174R
  CDS (1)...(1071): Integrase E174R
atgggaagaaggcgaagtcatgagcgccgggatttaccccctaacctttatataagaaacaatggatattactgctacagggacccaaggacgggtaaagagtttggattaggcagagacaggcgaatcgcaatcactgaagctatacaggccaacattgagttattttcaggacacaaacacaagcctctgacagcgagaatcaacagtgataattccgttacgttacattcatggcttgatcgctacgaaaaaatcctggccagcagaggaatcaagcagaagacactcataaattacatgagcaaaattaaagcaataaggaggggtctgcctgatgctccacttgaagacatcaccacaaaagaaattgcggcaatgctcaatggatacatagacgagggcaaggcggcgtcagccaagttaatcagatcaacactgagcgatgcattccgagaggcaatagctgaaggccatataacaacaaaccatgtcgctgccactcgcgcagcaaaatctagagtaaggagatcaagacttacggctgacgaatacctgaaaatttatcaagcagcagaatcatcaccatgttggctcagacttgcaatggaactggctgttgttaccgggcaacgagttggtgatttatgcgaaatgaagtggtctgatatcgtagatggatatctttatgtcgagcaaagcaaaacaggcgtaaaaattgccatcccaacagcattgcatattgatgctctcggaatatcaatgaaggaaacacttgataaatgcaaagagattcttggcggagaaaccataattgcatctactcgtcgcgaaccgctttcatccggcacagtatcaaggtattttatgcgcgcacgaaaagcatcaggtctttccttcgaaggggatccgcctacctttcacgagttgcgcagtttgtctgcaagactctatgagaagcagataagcgataagtttgctcaacatcttctcgggcataagtcggacaccatggcatcacagtatcgtgatgacagaggcagggagtgggacaaaattgaaatcaaataa

SEQ ID NO: 50 - protein, 356 aa, Artificial Sequence
  Integrase E174R
MGRRRSHERRDLPPNLYIRNNGYYCYRDPRTGKEFGLGRDRRIAITEAIQANIELFSGHKHKPLTARINSDNSVTLHSWLDRYEKILASRGIKQKTLINYMSKIKAIRRGLPDAPLEDITTKEIAAMLNGYIDEGKAASAKLIRSTLSDAFREAIAEGHITTNHVAATRAAKSRVRRSRLTADEYLKIYQAAESSPCWLRLAMELAVVTGQRVGDLCEMKWSDIVDGYLYVEQSKTGVKIAIPTALHIDALGISMKETLDKCKEILGGETIIASTRREPLSSGTVSRYFMRARKASGLSFEGDPPTFHELRSLSARLYEKQISDKFAQHLLGHKSDTMASQYRDDRGREWDKIEIK

SEQ ID NO: 51 - DNA, 34 bp, Artificial Sequence
  Lox P Site
ataacttcgtataatgtatgctatacgaagttat